(19) United States
(12) Patent Application Publication
(10) Pub. No.: US 2025/0066353 A1
(43) Pub. Date: Feb. 27, 2025

(54) VANIN-1 INHIBITORS

(71) Applicant: ATHOS THERAPEUTICS, INC., Torrance, CA (US)

(72) Inventors: Dimitrios ILIOPOULOS, Los Angeles, CA (US); David G. HO, Monterey Park, CA (US); Sharif ASAD, Los Angeles, CA (US); Iordanis KARAGIANNIDIS, Los Angeles, CA (US)

(21) Appl. No.: 18/892,257

(22) Filed: Sep. 20, 2024

Related U.S. Application Data
(63) Continuation-in-part of application No. PCT/US2024/020364, filed on Mar. 18, 2024.
(60) Provisional application No. 63/491,415, filed on Mar. 21, 2023.

(51) Int. Cl. C07D 471/04; A61K 9/00; A61K 9/02; A61K 9/20; A61K 9/48; A61K 31/437; A61K 31/444; A61K 31/497; A61K 31/506; A61K 47/02; A61K 47/12; A61K 47/14; A61K 47/26

(52) U.S. Cl. CPC C07D 471/04; A61K 9/0019; A61K 9/02; A61K 9/2013; A61K 9/2018; A61K 9/2054; A61K 9/2059; A61K 9/4858; A61K 9/4891; A61K 31/437; A61K 31/444; A61K 31/497; A61K 31/506; A61K 47/02; A61K 47/12; A61K 47/14; A61K 47/26

(57) ABSTRACT

Disclosed herein are bicyclic compounds, including pharmaceutical compositions that include one or more of such compounds. Also disclosed are methods of making functionalized bicyclic compounds. Also disclosed herein are methods of treating diseases and/or conditions (e.g., inflammation and/or cancer) with the bicyclic compounds disclosed herein.

### FIELD

Disclosed herein are bicyclic compounds, including pharmaceutical compositions that include one or more of such compounds. Also disclosed are methods of making functionalized bicyclic compounds.

### BACKGROUND

The enzyme vascular non-inflammatory molecule-1 (vanin-1) is prominent in many organs, such as the liver, intestine, and kidney. Its major function is the metabolization of pantetheine into cysteamine and pantothenic acid. Vanin-1 plays a complex role in disease, having a protective effect or acting a sensitizer, depending on with which organ it is associated.

### SUMMARY

Several embodiments disclosed herein pertain to bicyclic compounds, their use as inhibitors of the enzymatic activity of vanin-1. Some embodiments pertain to methods of manufacturing bicyclic compounds, and methods of using bicyclic compounds as therapeutics for treating inflammatory disease states. In several embodiments, the bicyclic compound comprises at least one aromatic ring substituted with at least one amine group. Several embodiments comprise, consist of, or consist essentially of a bicyclic compound of Formula (I) (or any other structure disclosed herein), their pharmaceutically acceptable salts, enantiomers, methods of manufacture, and/or their methods of use in treating disease states. In several embodiments, by using one or more compounds of Formula (I) (or any other structure disclosed herein) to inhibit the activity of vanin-1 in a subject, a disease state can be treated. In several embodiments, the disease state is associated with inflammation. In several embodiments, the disease state is associated with an autoimmune disorder. In several embodiments, the disease state is a cancer mediated at least in part through vanin-1.

Several embodiments pertain to vanin-1 inhibitors. In several embodiments, a vanin-1 inhibitor comprises a compound, or a pharmaceutically acceptable salt thereof, having a structure represented by Formula (I):

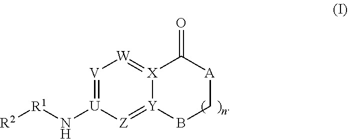

In several embodiments, A, B, U, V, W, X, Y, and Z are each independently selected from the group consisting of C, C(R⁵-R³), N, N(R⁵-R⁴), S, and S(R⁵-R⁴) and at least one instance of A, B, U, V, W, X, Y, and Z is N, N(R⁵-R⁴), S, or S(R⁵-R⁴) with the remaining variables being C or C(R⁵-R³); each instance of R¹ and R² is independently selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 5-10 membered heteroaryl, optionally substituted 3-10 membered heteroarylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent; each instance of R³ and R⁴, where present, is independently selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 5-10 membered heteroaryl, optionally substituted 3-10 membered arylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent; each instance of R⁵, where present, is independently selected from the group consisting of —(CR^{x}₂)ₘ and optionally substituted C₁₋₂₀ alkylene or is absent; each R^{x} is independently —H, -D, or an optionally substituted C₁₋₆ alkyl; m is an integer in the range of 0 to 20; and n is 0, 1, or 2.

Some embodiments pertain to compounds that contain one or more deuterium atom(s).

Some embodiments pertain to a compound of Formula (I) or a pharmaceutically acceptable salt thereof:

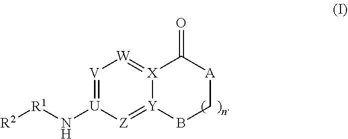

In several embodiments, A, B, U, V, W, X, Y, and Z are each independently selected from the group consisting of C, C(R⁵-R³), N, N(R⁵-R⁴), S, and S(R⁵-R⁴) and at least one instance of A, B, U, V, W, X, Y, and Z is N, N(R⁵-R⁴), S, or S(R⁵-R⁴) with the remaining variables being C or C(R⁵-R³); each instance of R¹ and R² is independently selected from the group consisting of optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 5-10 membered heteroaryl, optionally substituted 3-10 membered heteroarylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent; each instance of R³ and R⁴, where present, is independently selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 5-10 membered heteroaryl, optionally substituted 3-10 membered arylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent; each instance of R⁵, where present, is independently selected from the group consisting of —(CR^{x}₂)ₘ— and optionally substituted C₁₋₂₀ alkylene or is absent; each R^{x} is independently —H, -D, or an optionally substituted C₁₋₆ alkyl; m is an integer in the range of 0 to 20; and n is 0, 1, or 2.

In some embodiments, Z is N. In some embodiments, V is N. In some embodiments, n is 0. In some embodiments, n is 1. In some embodiments, A is N(R⁵-R⁴). In some embodiments, R⁵ is absent. In some embodiments, R⁵ is an optionally substituted C₁₋₂₀ alkylene.

Several embodiments pertain to a compound of Formula (I), or a pharmaceutically acceptable salt thereof, further represented by one or more of Formulae (Ia) or (Ib):

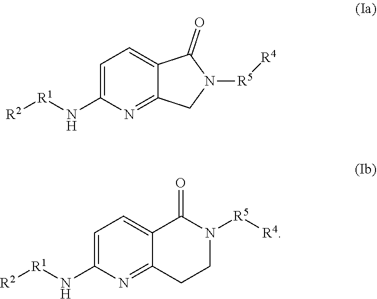

In some embodiments, R⁵ is —(CR^{x}₂)ₘ. In some embodiments, R⁵ is —(CH₂)ₘ or —(CD₂)ₘ-. In some embodiments, m is 0, 1, 2, 3, 4, 5, or 6. In some embodiments, m is 0, 1, 2, or 3. In some embodiments, m is 2. In some embodiments, m is 1.

In some embodiments, the structure of Formula (I) is further represented by Formula (Ic):

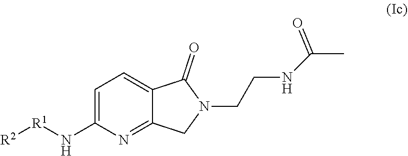

or a pharmaceutically acceptable salt thereof. In some embodiments, the structure of Formula (I) is further represented by Formula (Id):

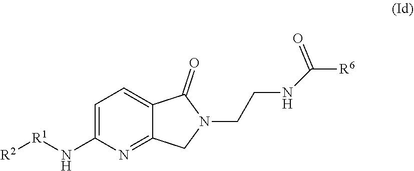

or a pharmaceutically acceptable salt thereof. In some embodiments, R⁶ is selected from the group consisting of —CD₃, —CD₂CD₃,

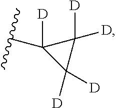

methyl, —N(CH₃)₂, —NHCH₃, and cyclopropyl. In some embodiments, R¹ is —CH₂—, —CHD-, or —CD₂-. In some embodiments, R¹ is absent. In some embodiments, R² is an optionally substituted phenyl or an optionally substituted 6-membered heterocyclyl with 1 to 3 heteroatoms selected from N, O, and S. In some embodiments, R² is pyridinyl, pyrimidinyl, or pyrazinyl. In some embodiments, the optionally substituted group is substituted with between one and five groups selected from: —F, —C₁, -D, methyl, methoxy, oxo, —CN, and —CF₃.

In some embodiments, R⁴ is an optionally substituted C₁₋₆ alkyl or an optionally substituted C₁₋₆ heteroalkyl.

In some embodiments, R⁴ is selected from the group consisting of —H,

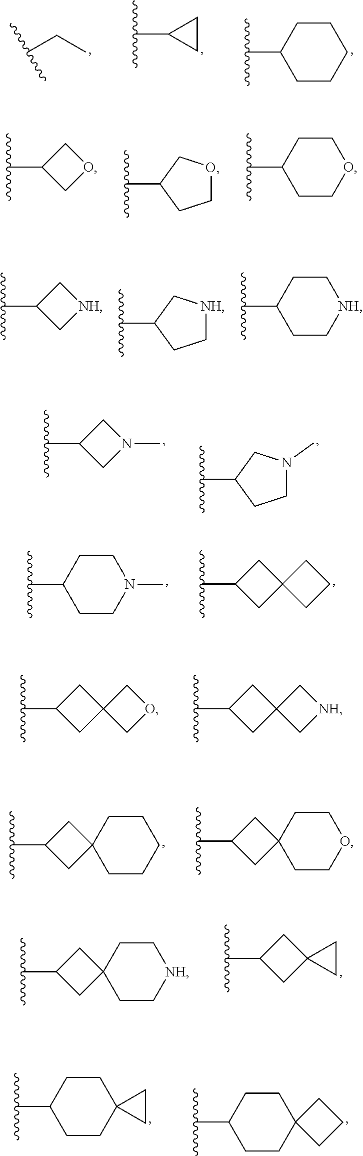

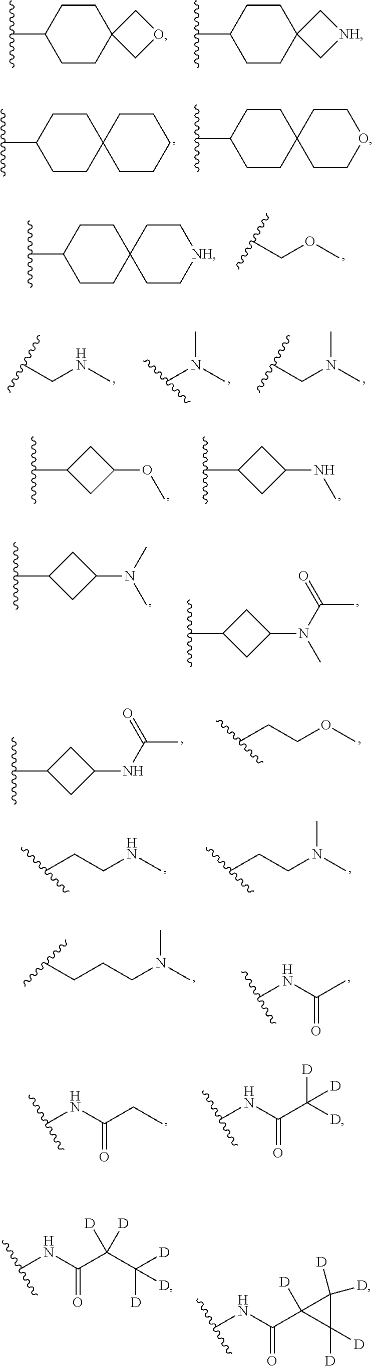

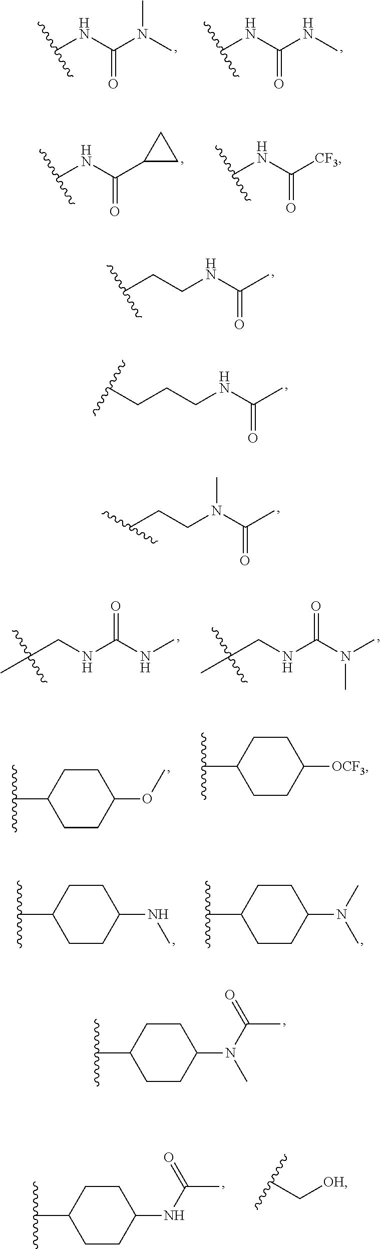

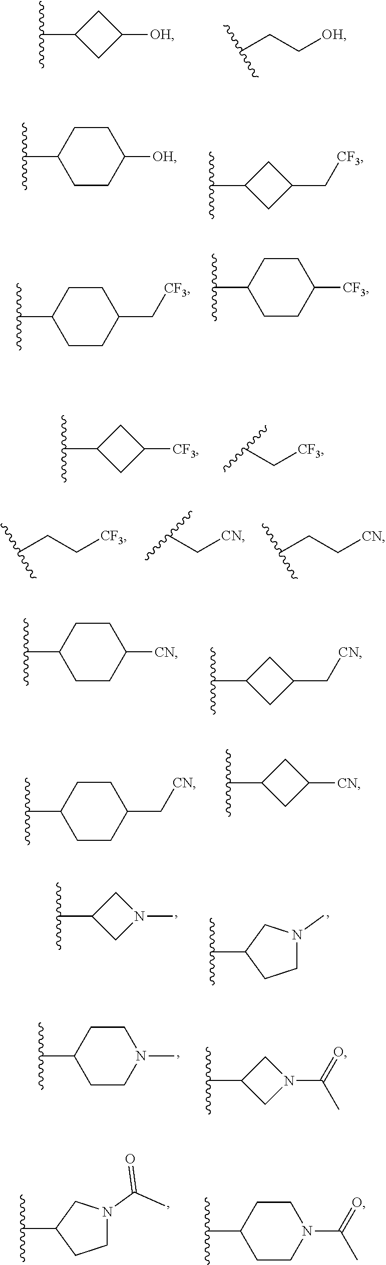

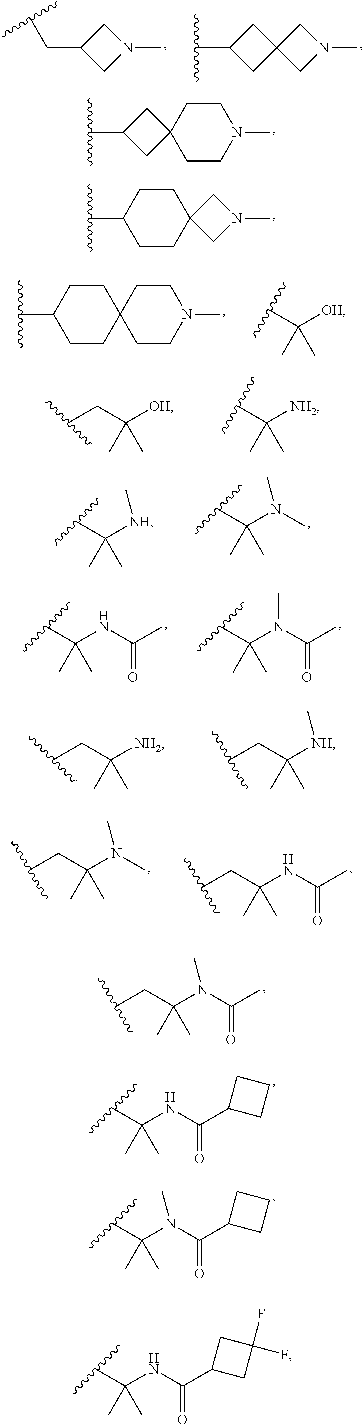

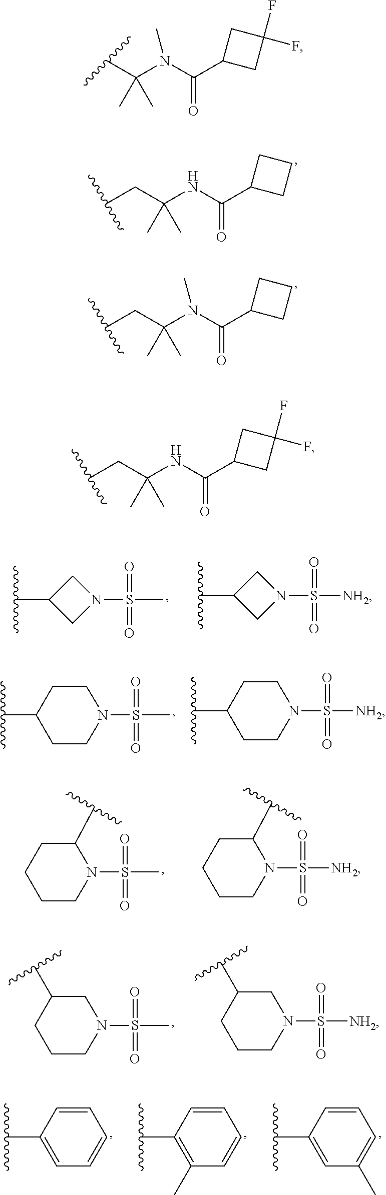

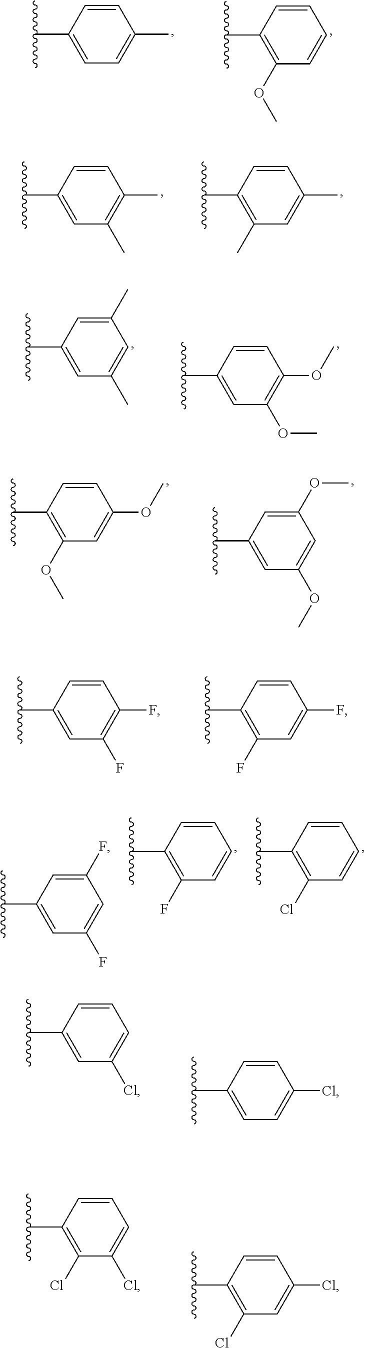

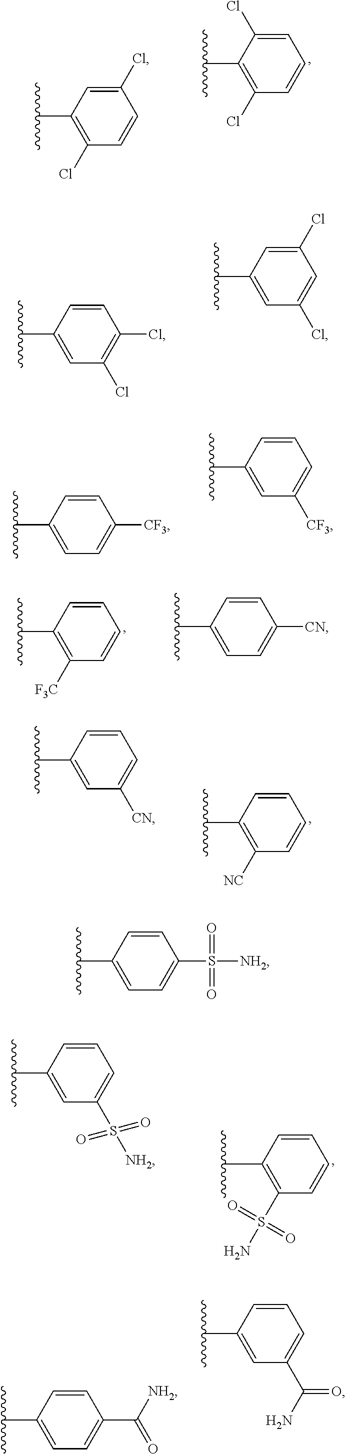

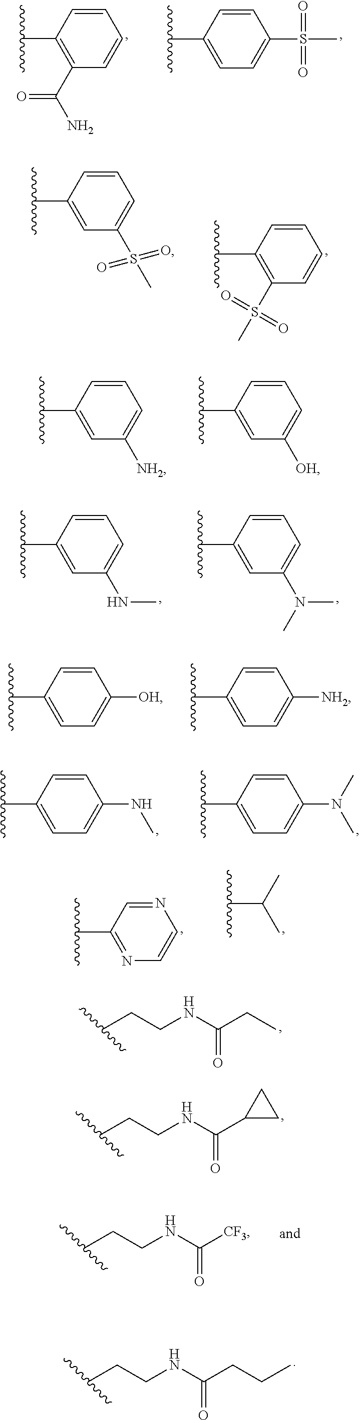

In some embodiments, R⁴ is selected from the group consisting of —H,

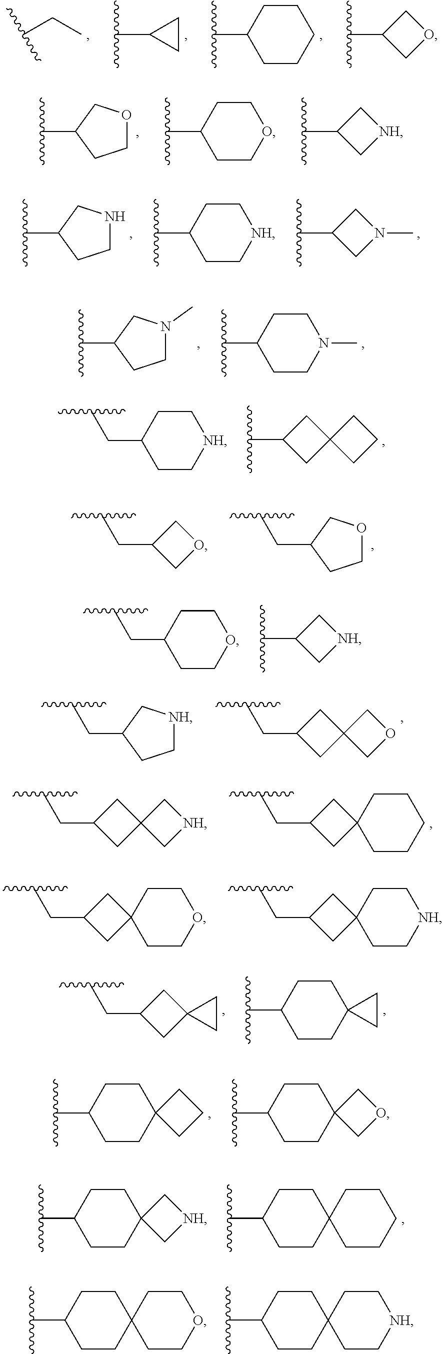

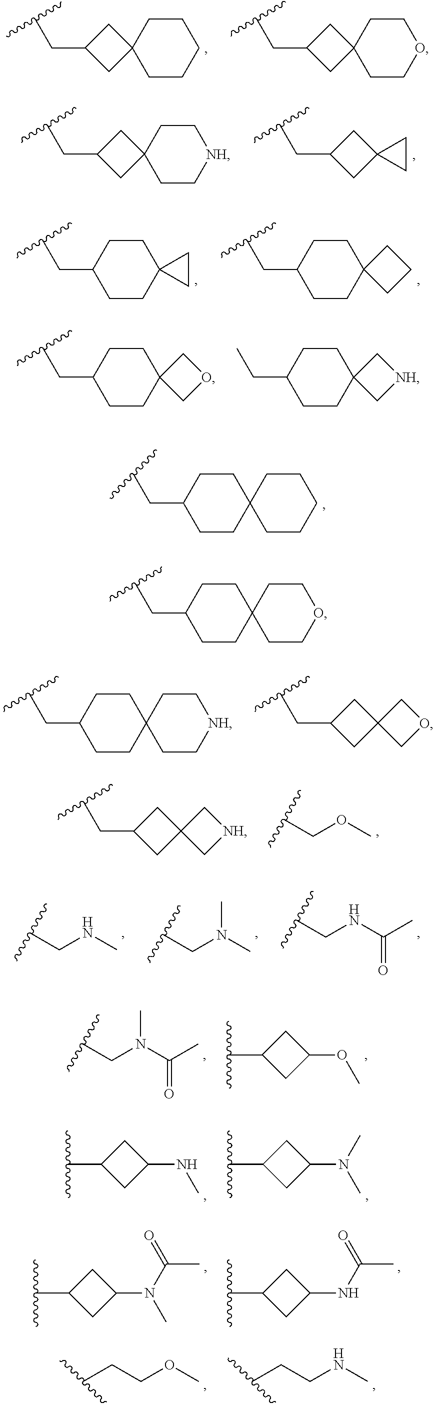

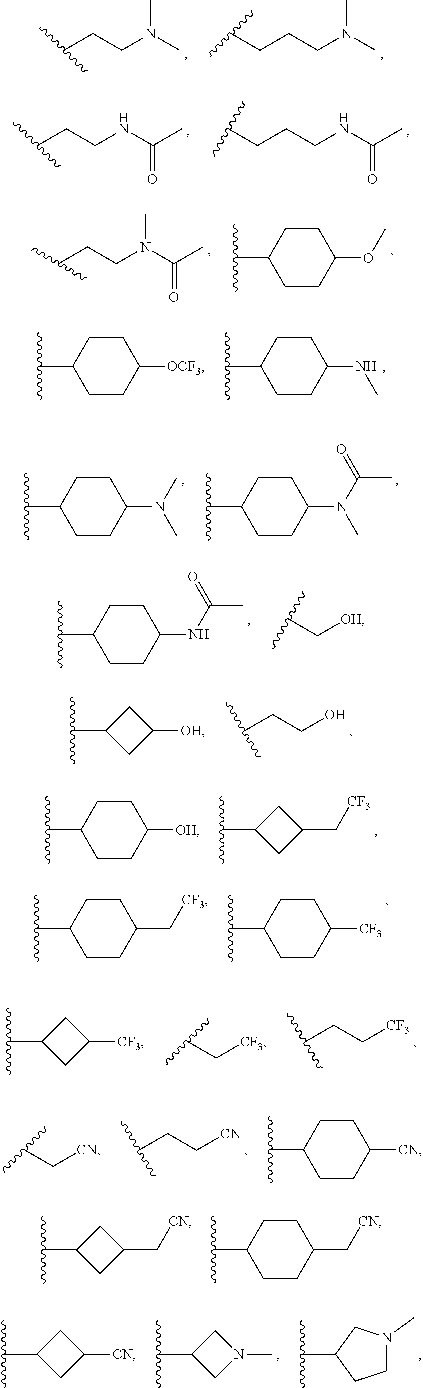

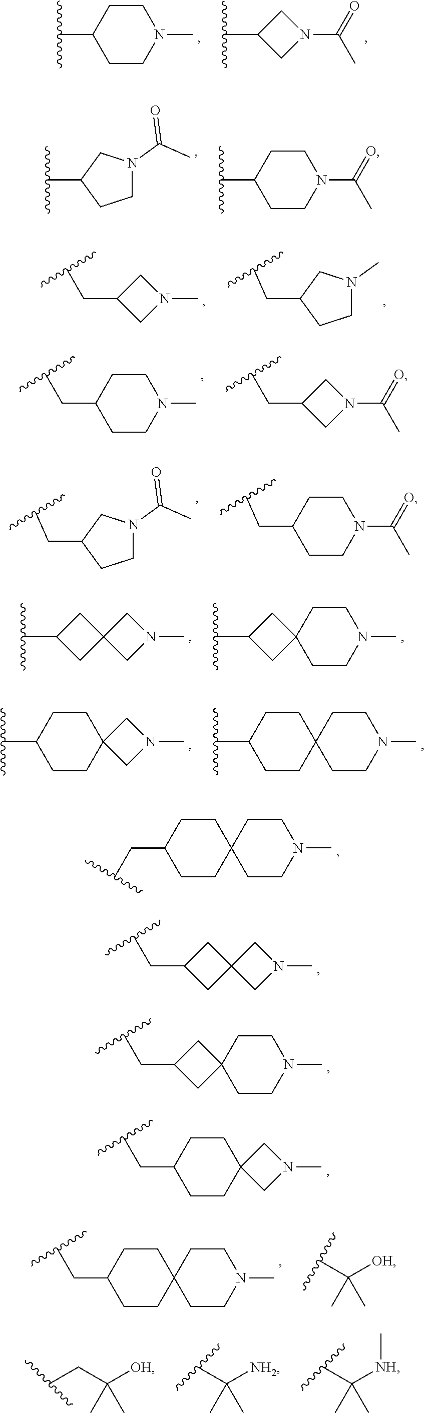

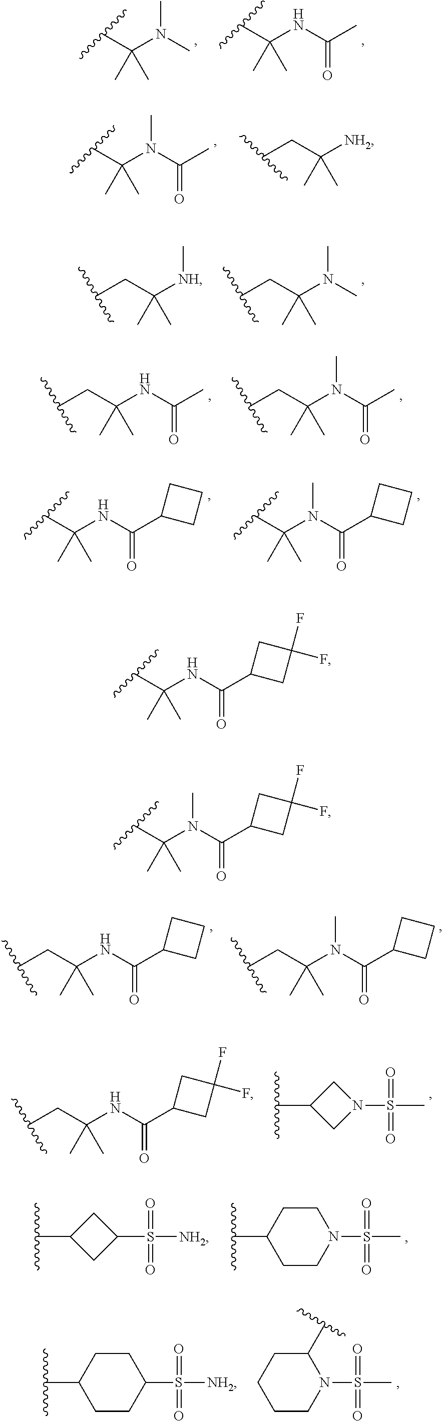

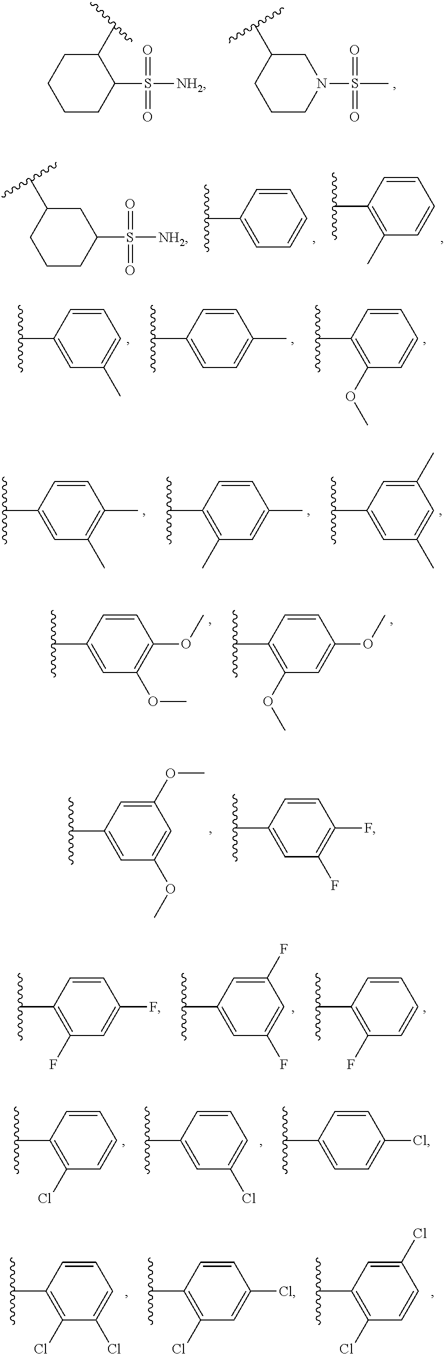

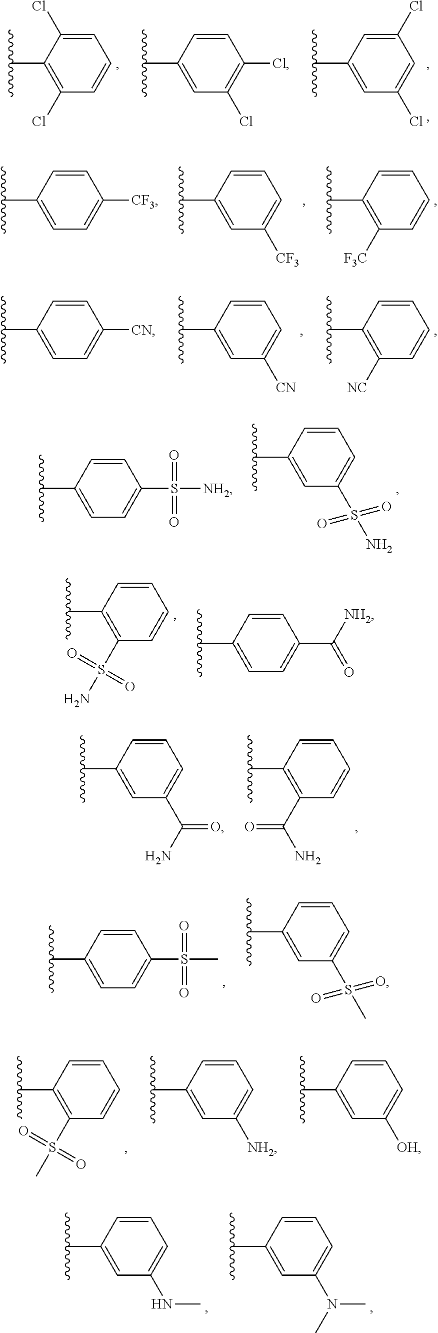

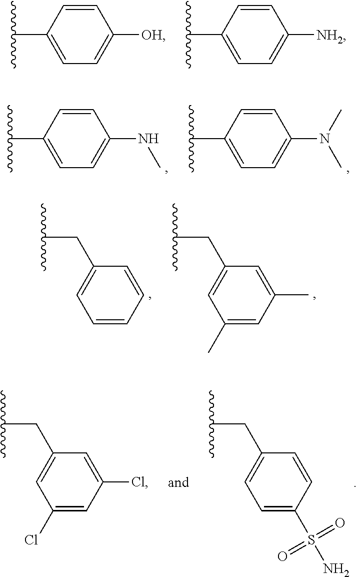

In some embodiments, R⁴ is selected from the group consisting of

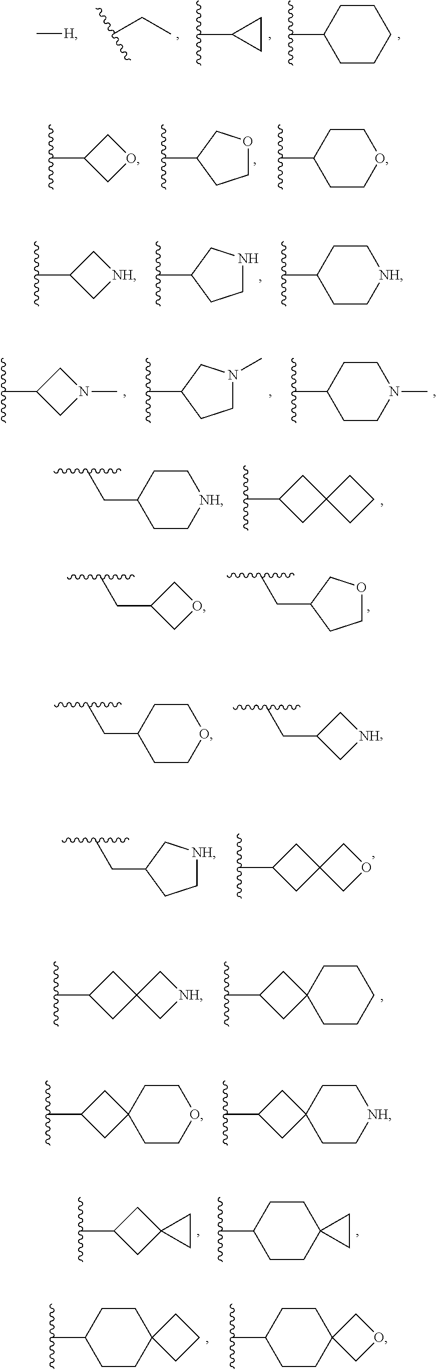

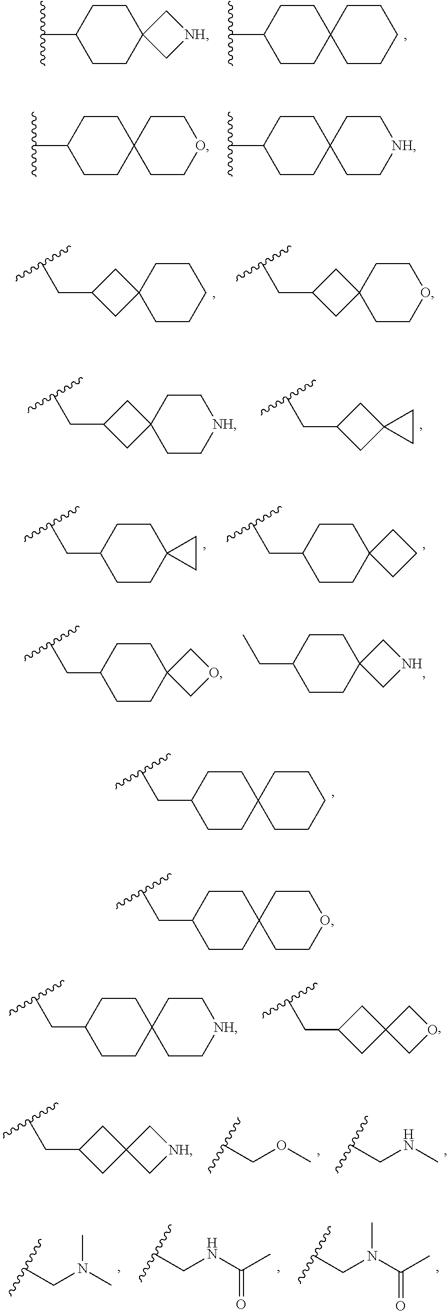

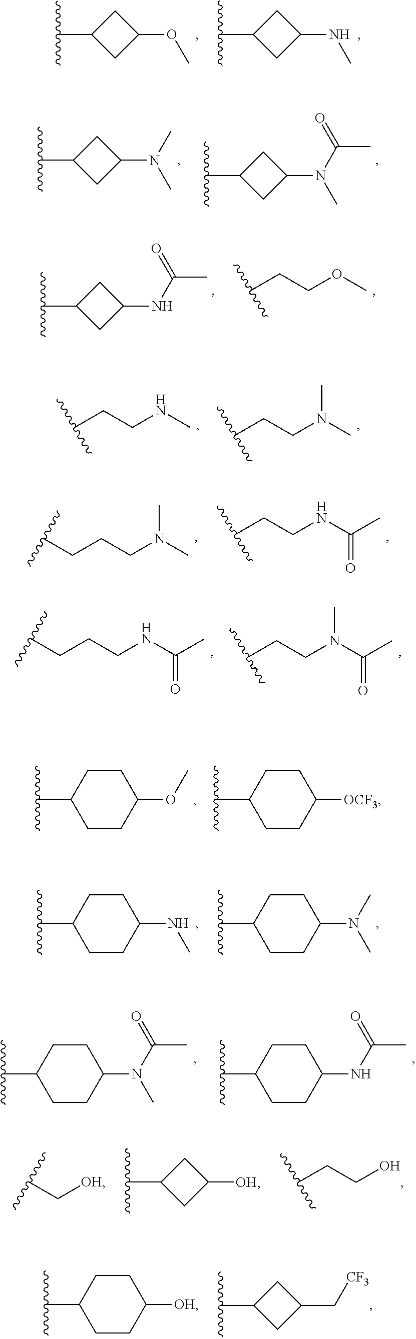

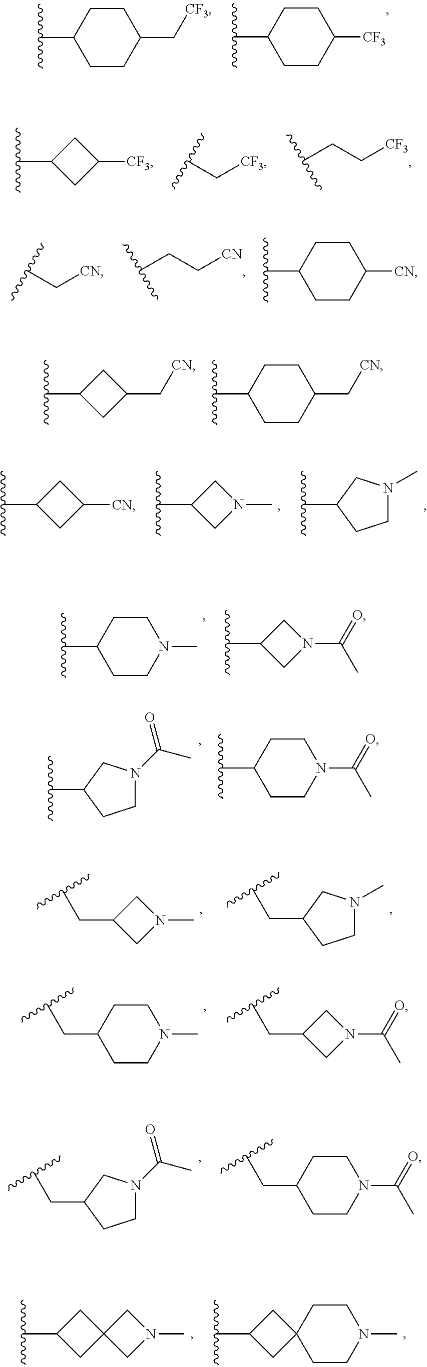

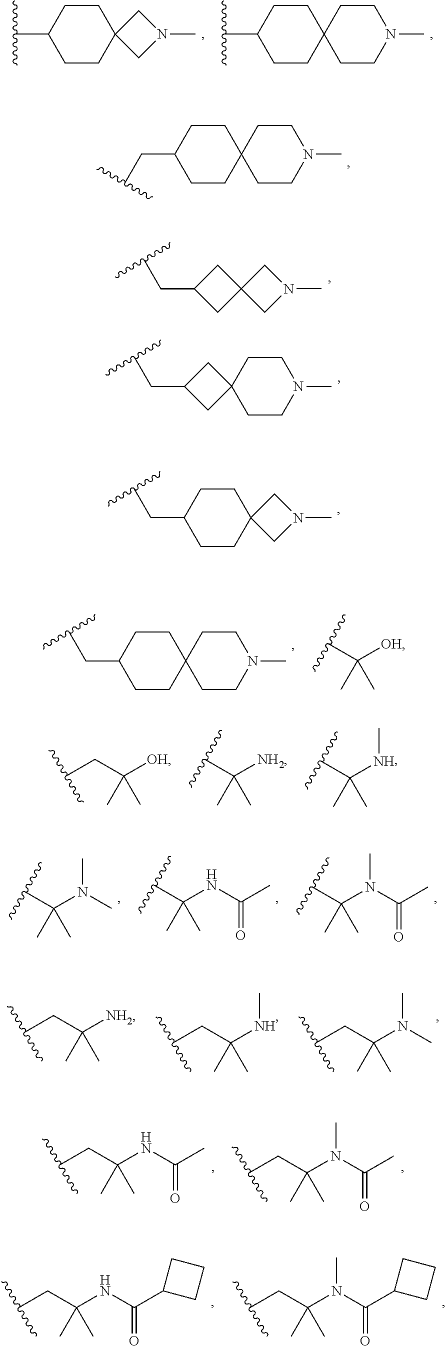

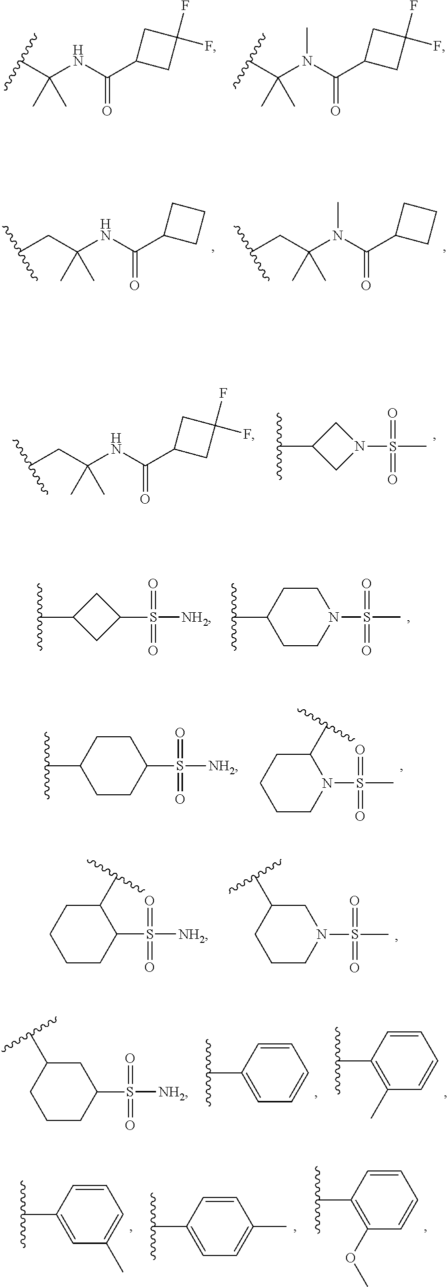

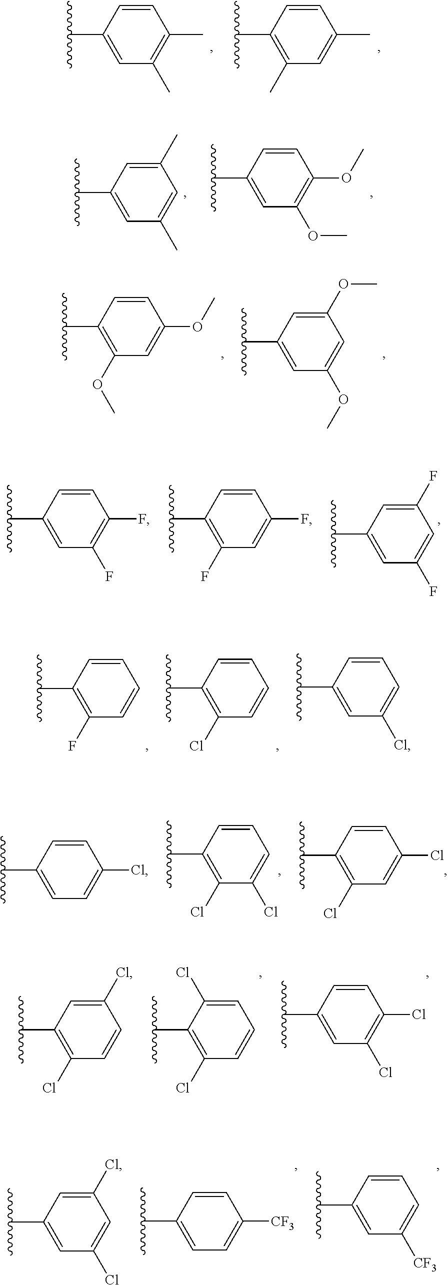

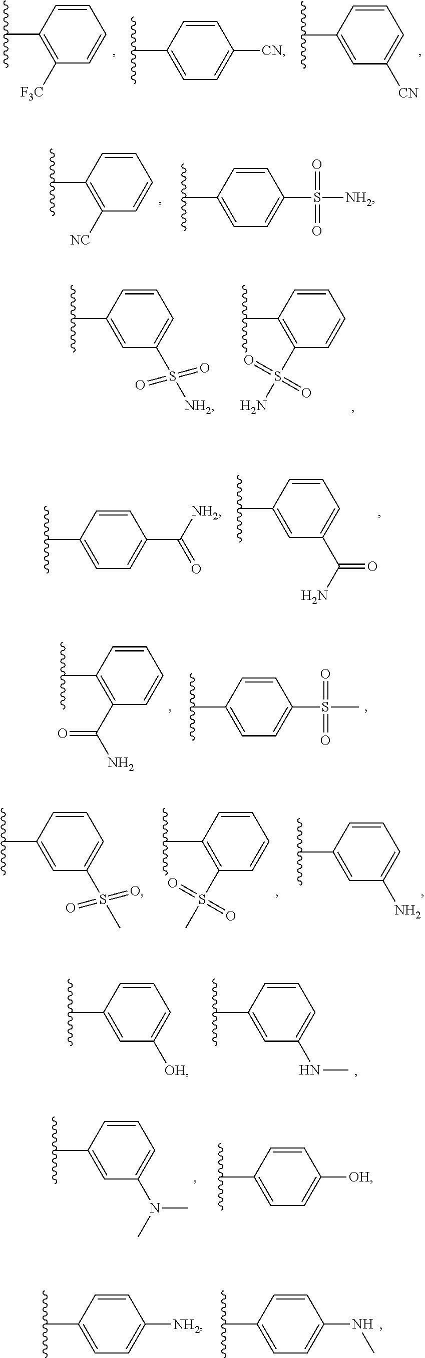

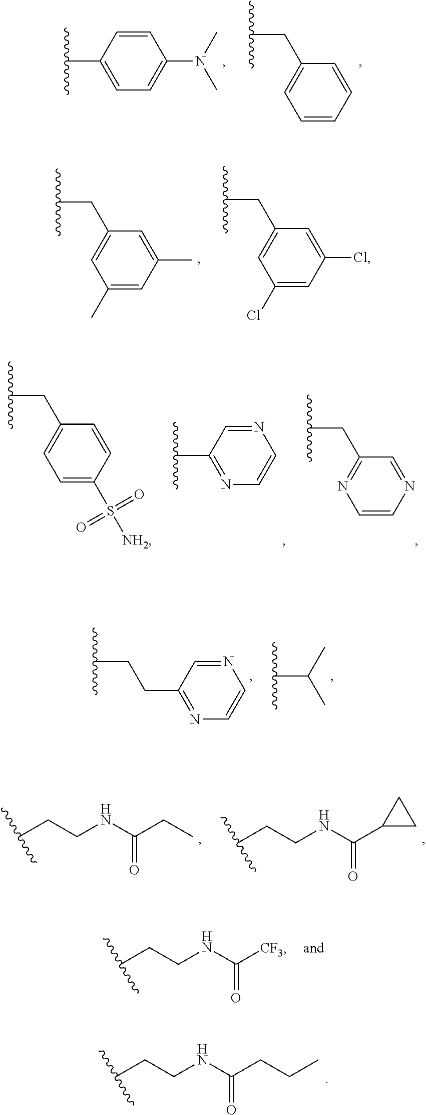

In certain embodiments, R⁴ is selected from the group consisting of —H,

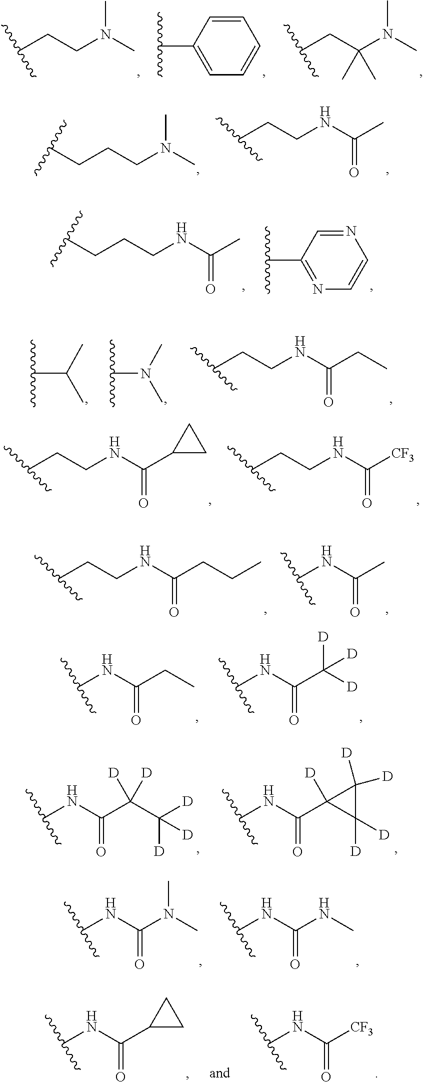

In some embodiments, R⁴ is selected from the group consisting of —H,

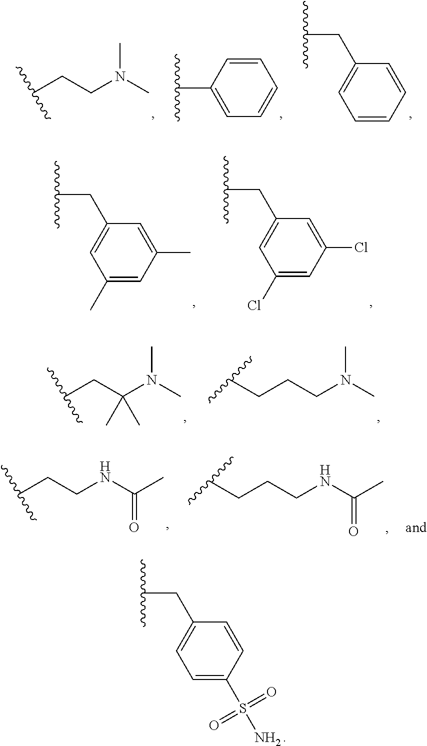

In some embodiments, R⁴ is selected from the group consisting of

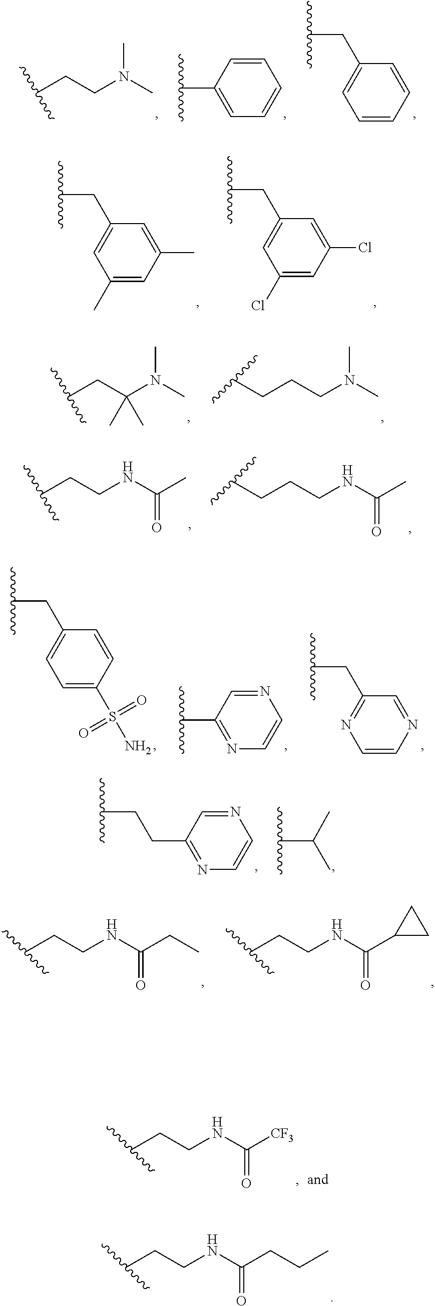

In certain embodiments, R⁴ is —H.

In certain embodiments, R⁴ is selected from the group consisting of

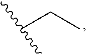 [SMILES: *CC]

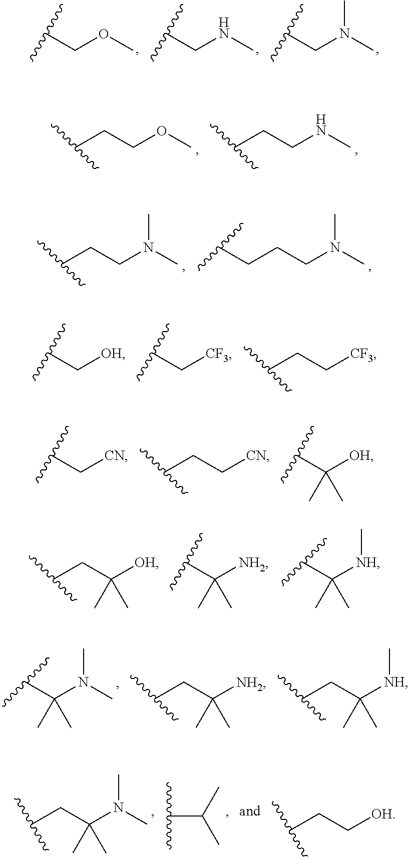

In certain embodiments, R⁴ is selected from the group consisting of

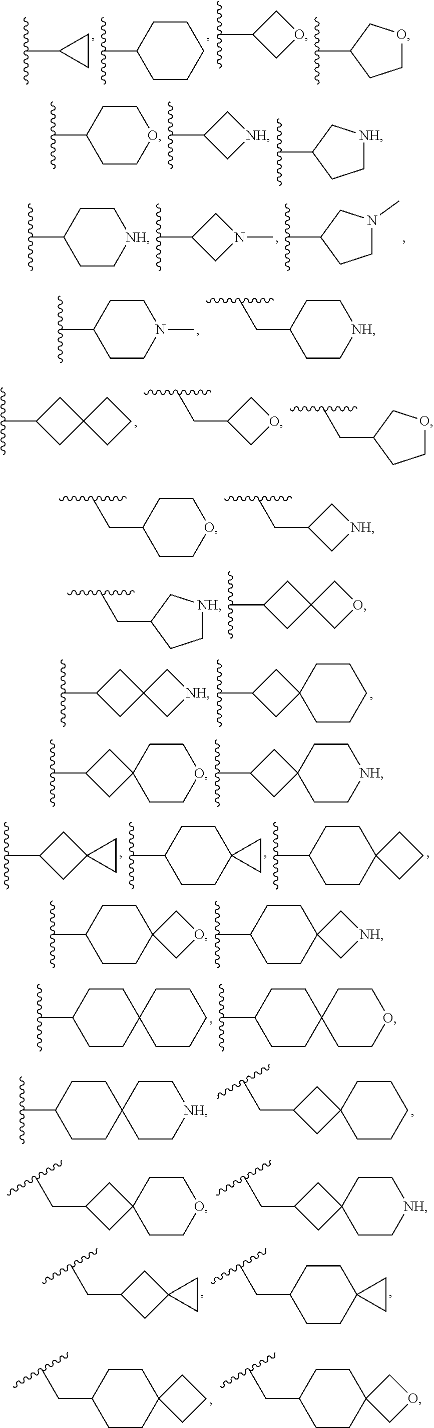

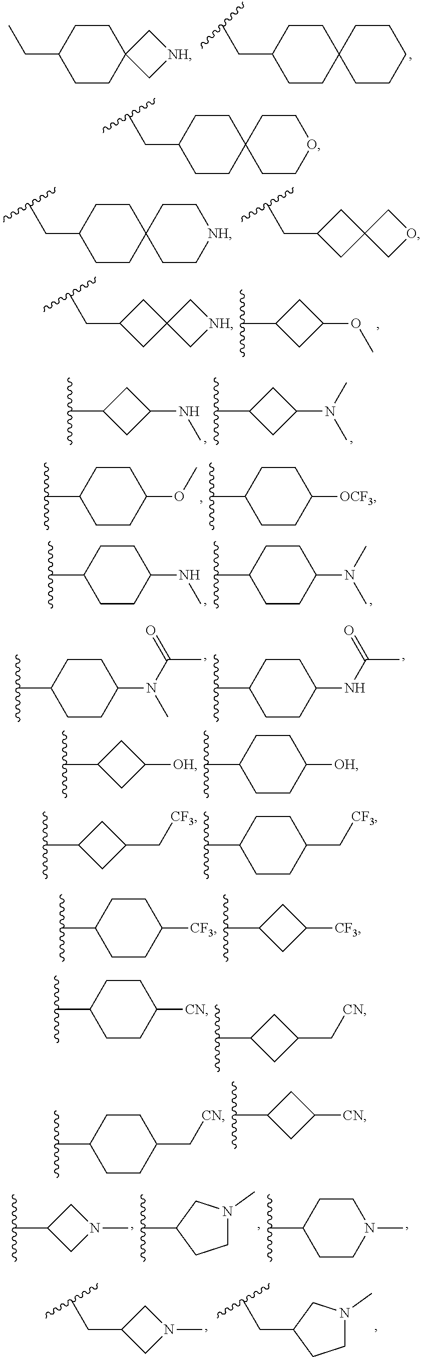

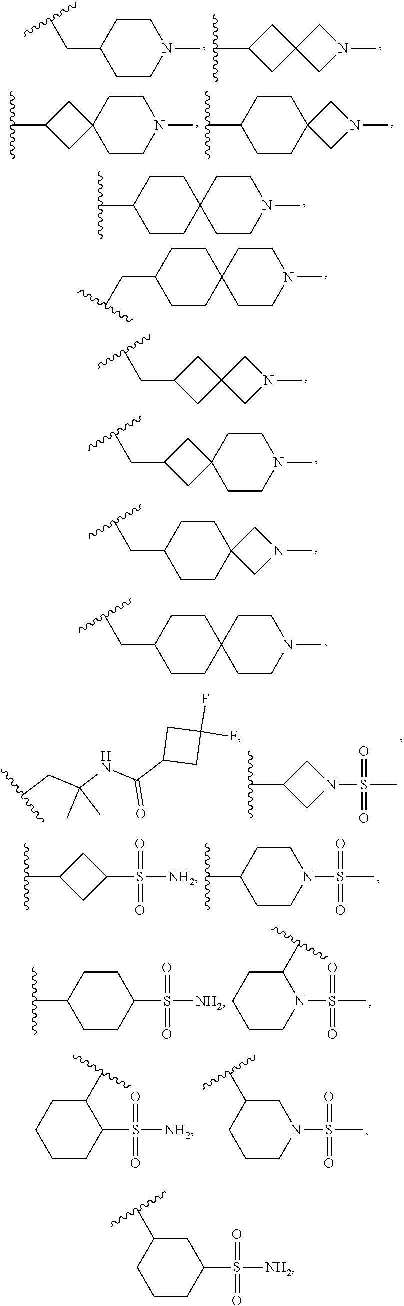

In certain embodiments, R⁴ is selected from the group consisting of

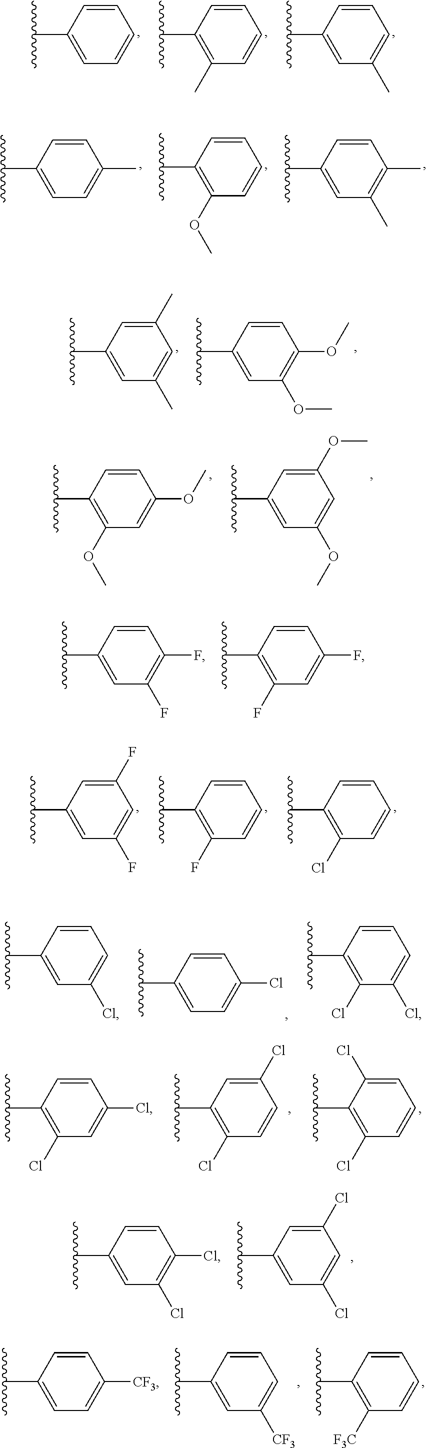

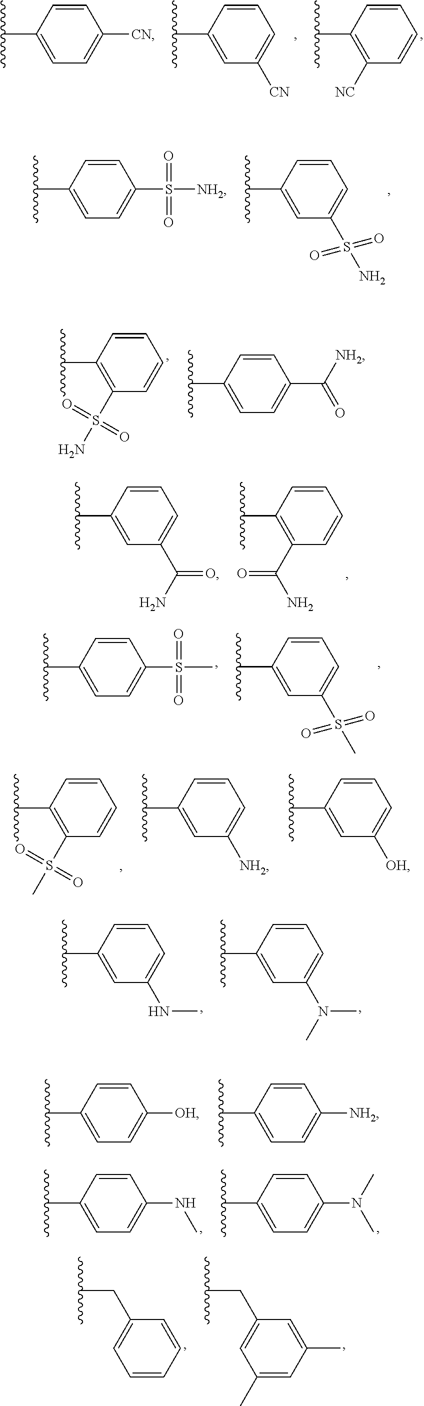

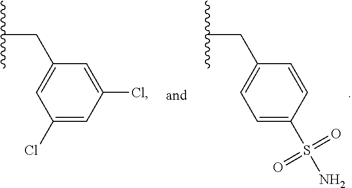 [SMILES: *Cc1cc(Cl)cc(Cl)c1 ; *Cc1ccc(S(N)(=O)=O)cc1]

In certain embodiments, R⁴ is selected from the group consisting of

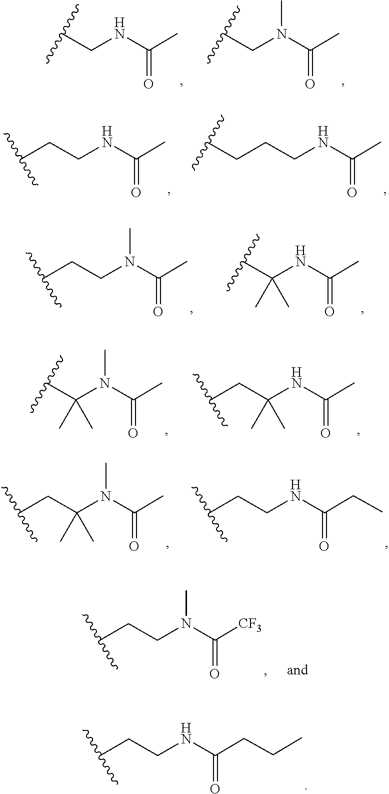

In certain embodiments, R⁴ is selected from the group consisting of

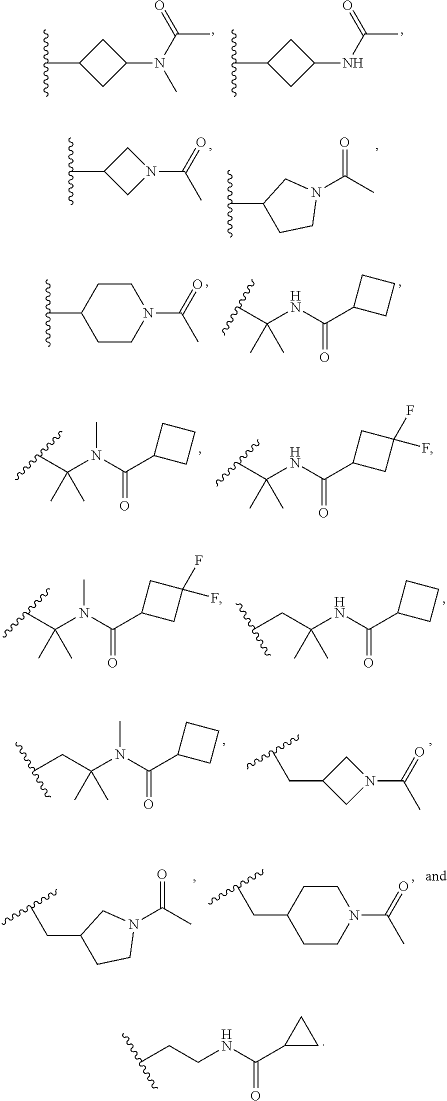

In certain embodiments, R⁴ is selected from the group consisting of

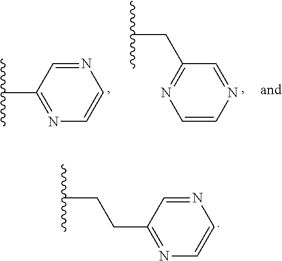 [SMILES: *c1cnccn1 ; *Cc1cnccn1 ; *CCc1cnccn1]

In some embodiments, R¹ is an optionally substituted C₁₋₆ alkyl or optionally substituted C₁₋₂₀ alkylene. In some embodiments, R¹ is an optionally substituted C₁₋₂₀ alkylene. In further embodiments, R¹ is selected from the group consisting of —CHD-, CD₂-, —CH₂—, —CH₂CH₂—, —CH(CH₃)—, and —CHCH₂(OH)—. In other embodiments, R¹ is —(CH₂)ₚ— and p is an integer in the range of 0 to 20. In some embodiments, p is 0, 1, 2, 3, 4, 5, or 6. In some embodiments, R¹ is —(CR^{y}₂)ₚ—, wherein each R^{y} is independently —H or -D.

In some embodiments, R² is selected from the group consisting of R² is selected from the group consisting of:

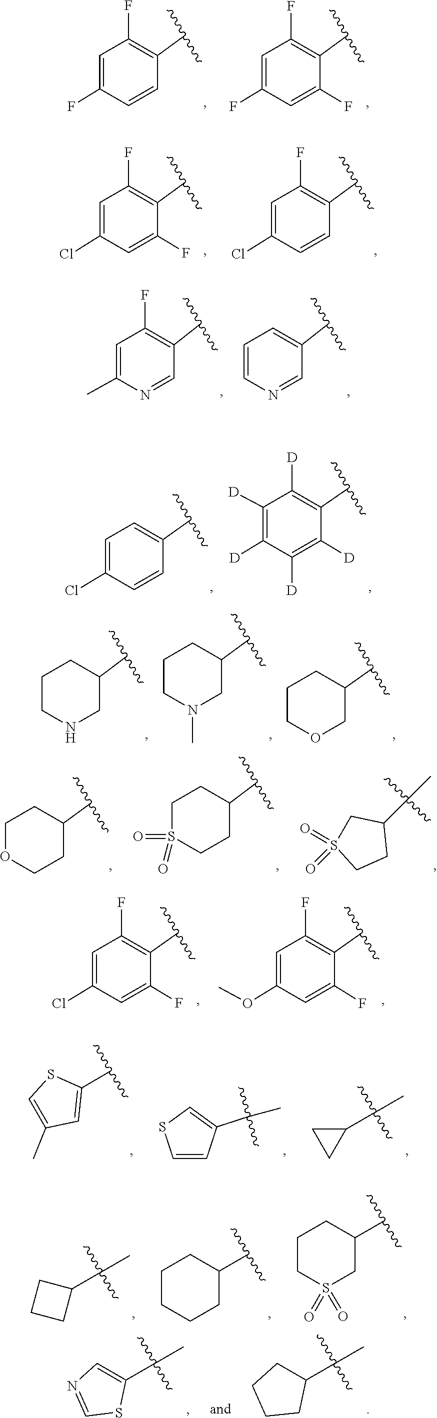

In some embodiments, R² is selected from the group consisting of:

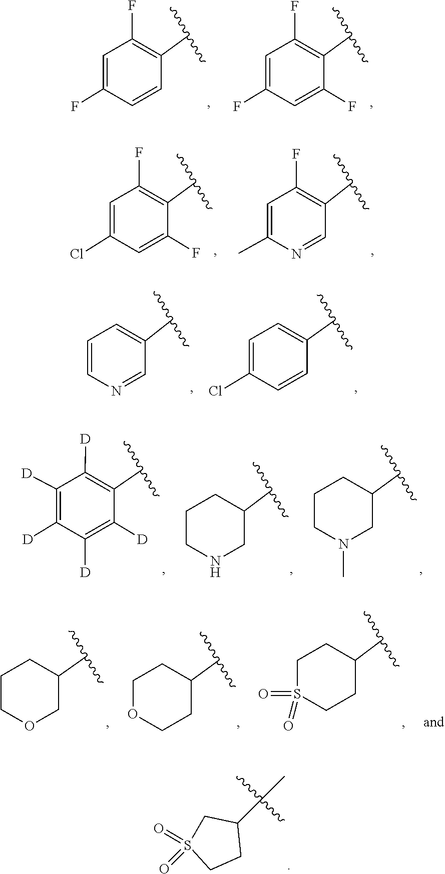

In some embodiments, R² is selected from the group consisting of

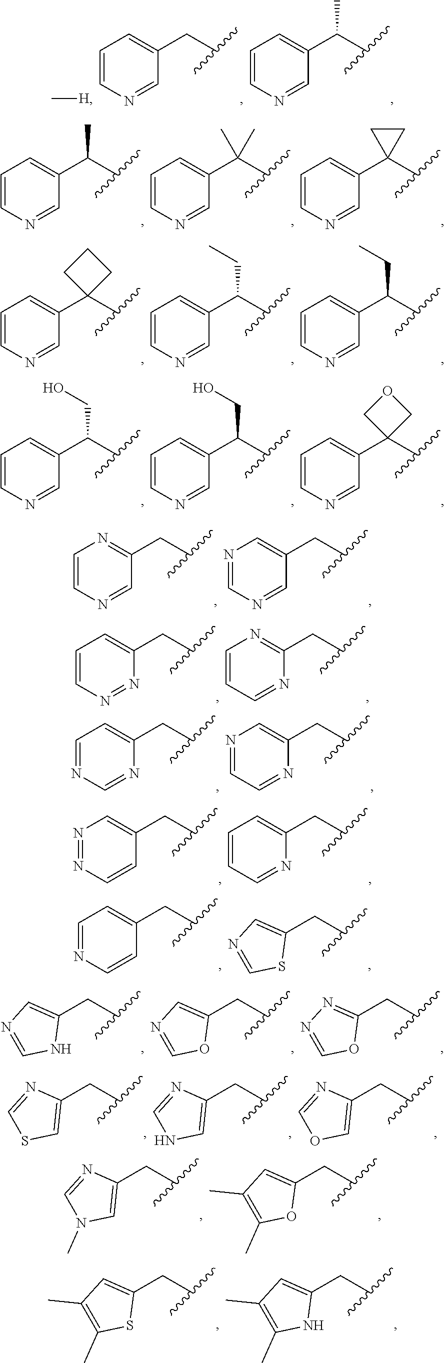

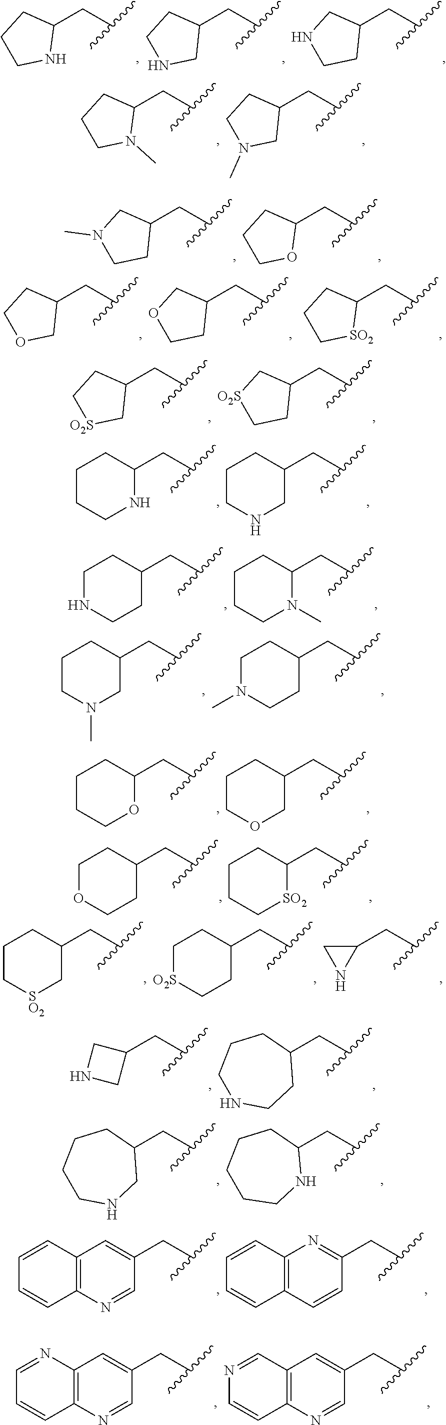

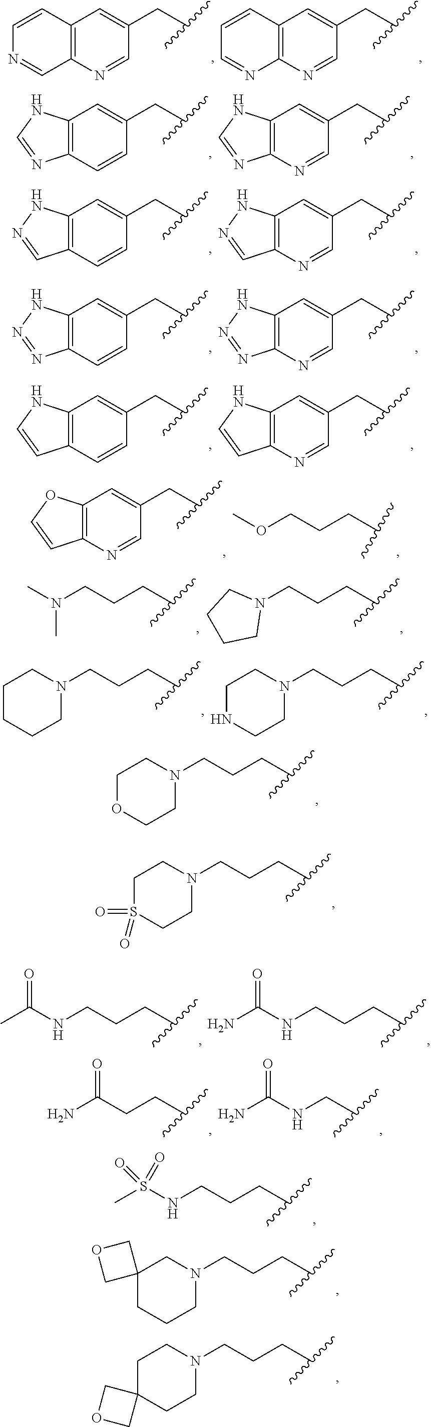

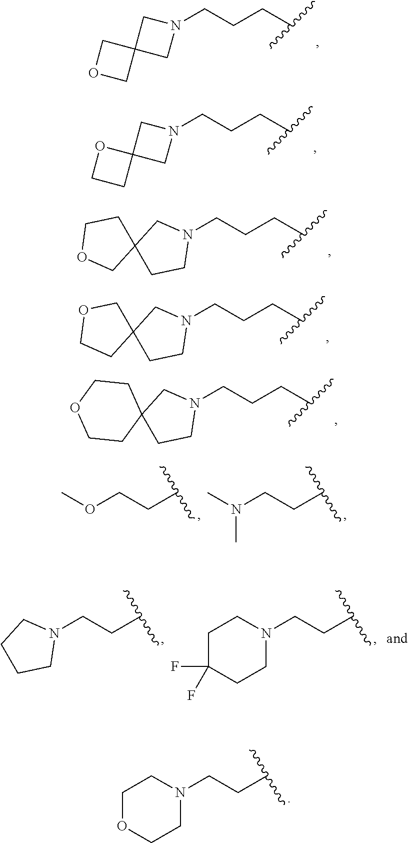

In some embodiments, R is selected from the group consisting of —H,

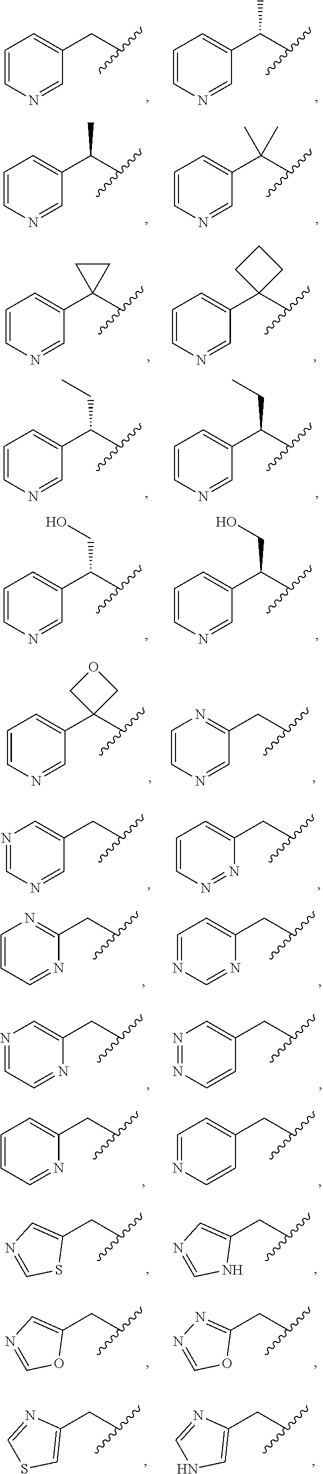

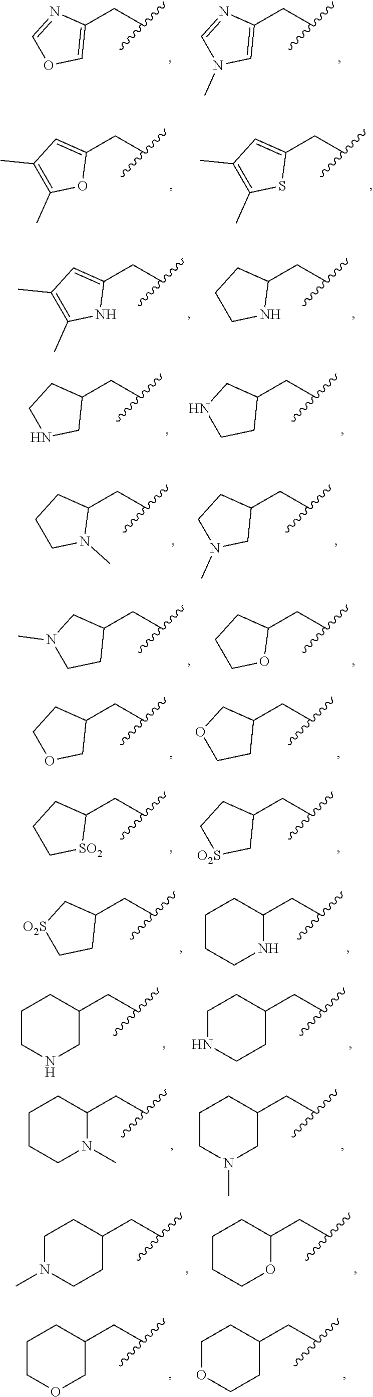

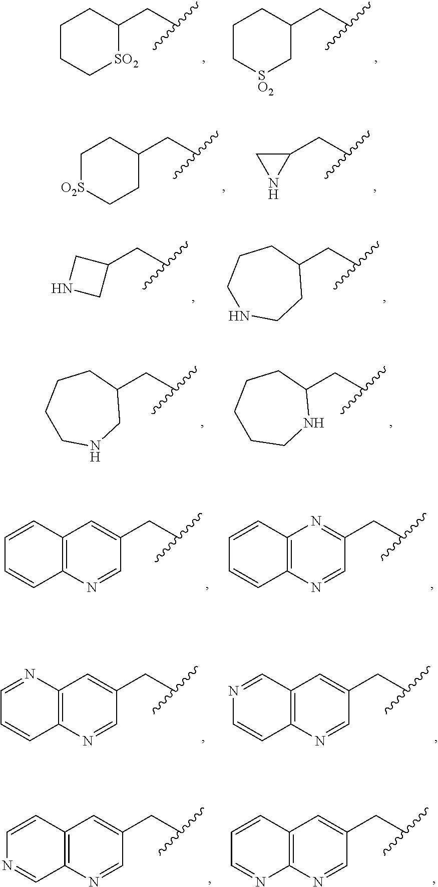

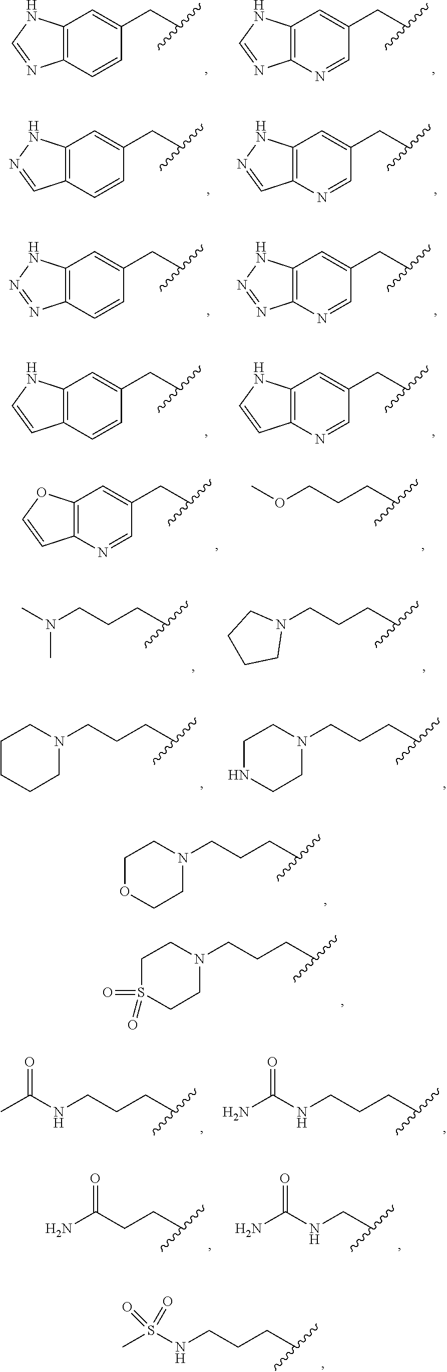

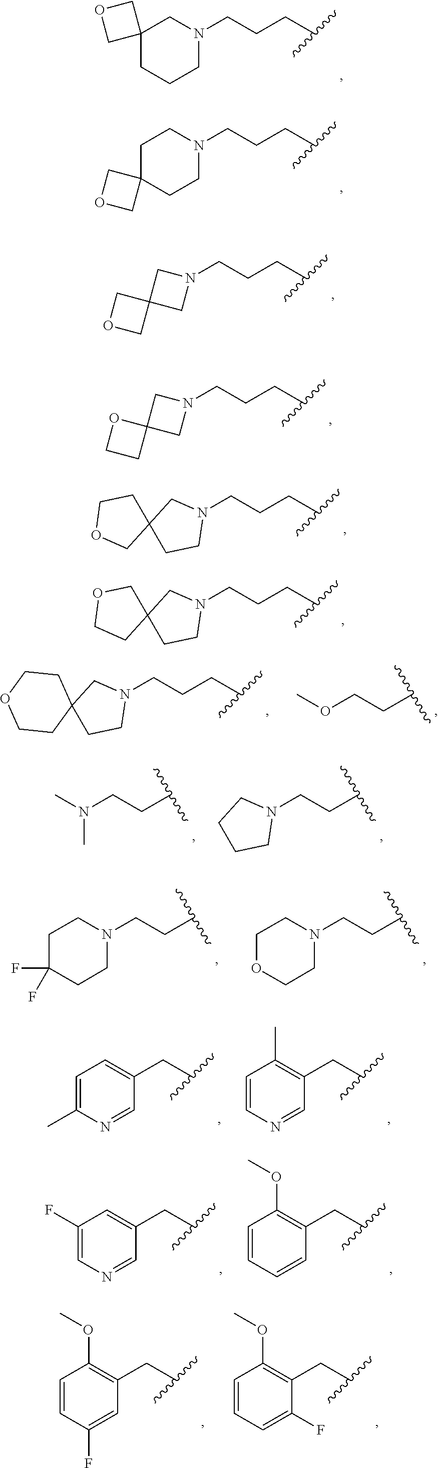

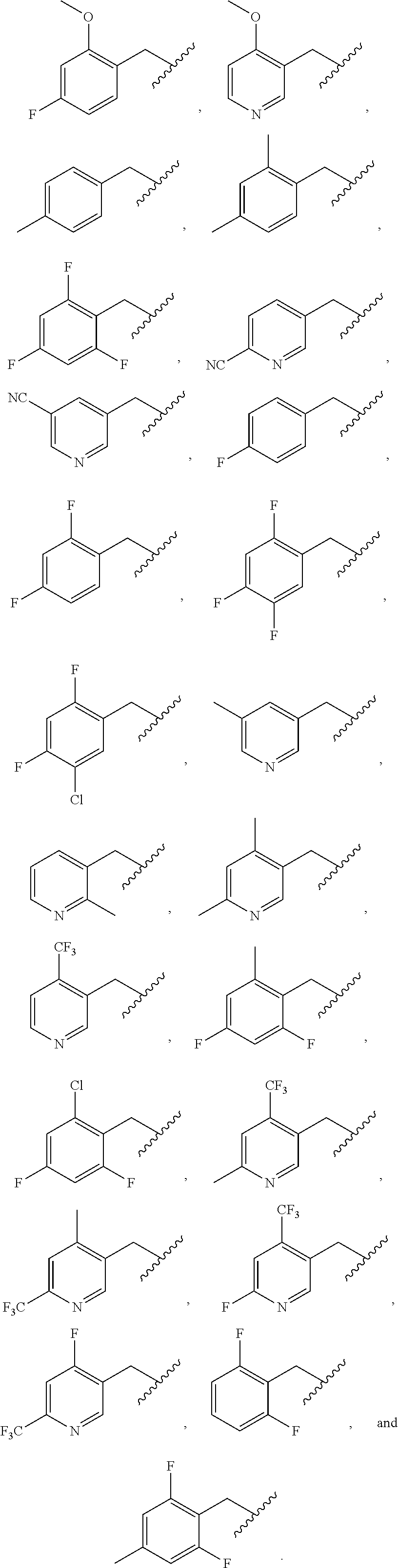

In certain embodiments, R² is selected from the group consisting of —H,

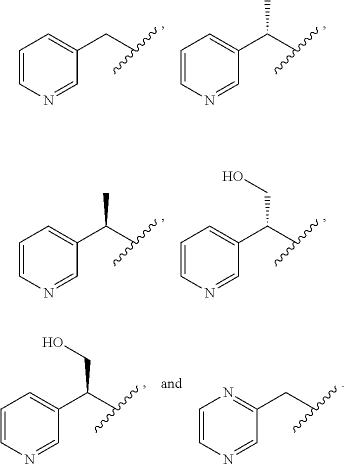

In certain embodiments, R² is selected from the group consisting of —H,

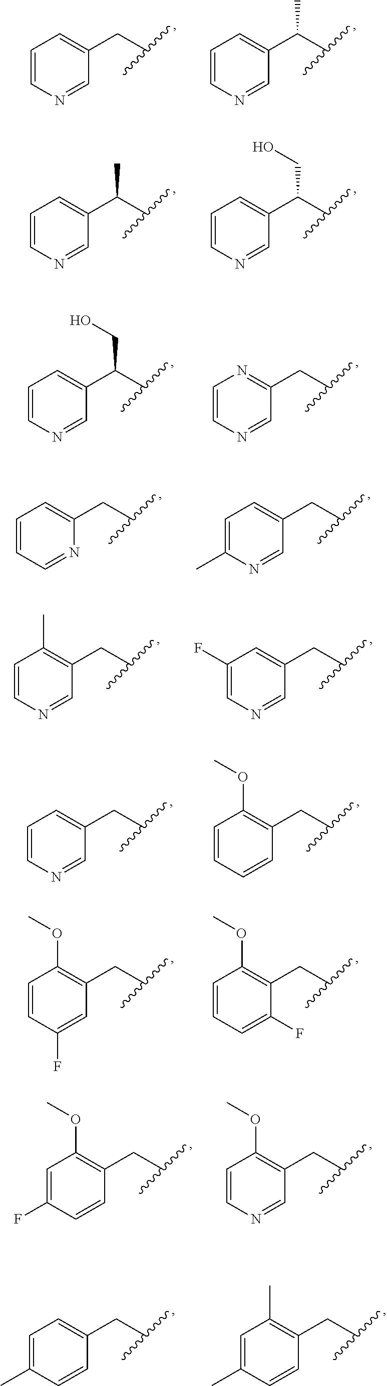

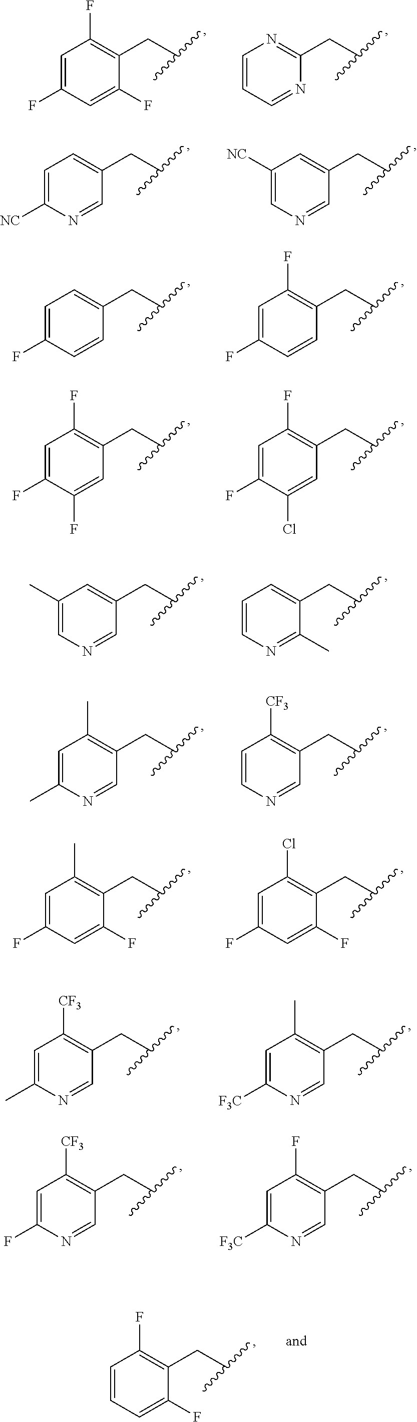

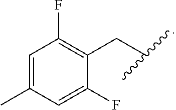 [SMILES: *Cc1c(F)cc(C)cc1F]

In some embodiments, R² is —H.

In some embodiments, R² is selected from the group consisting of

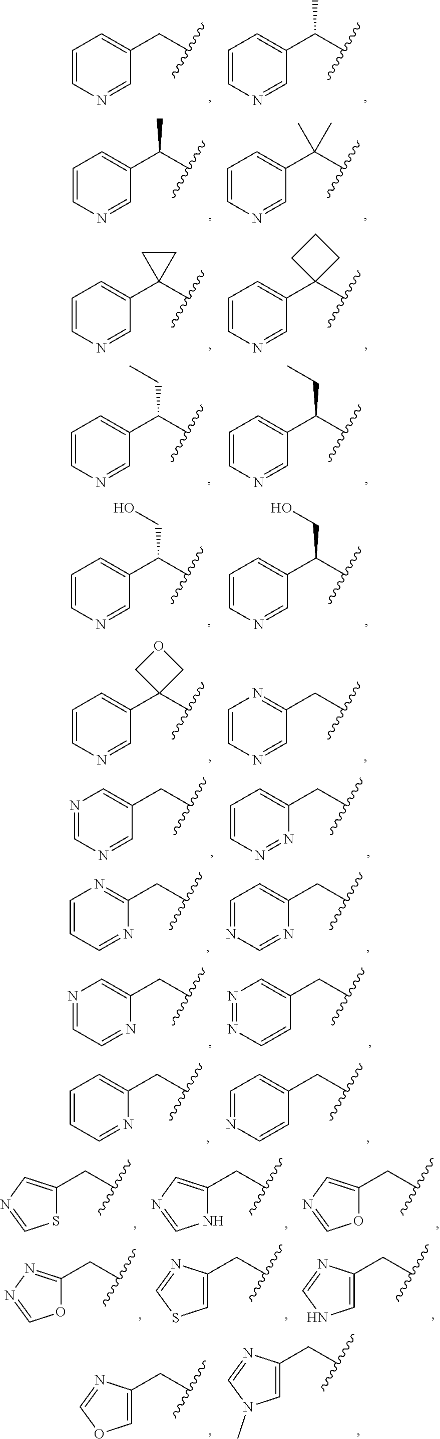

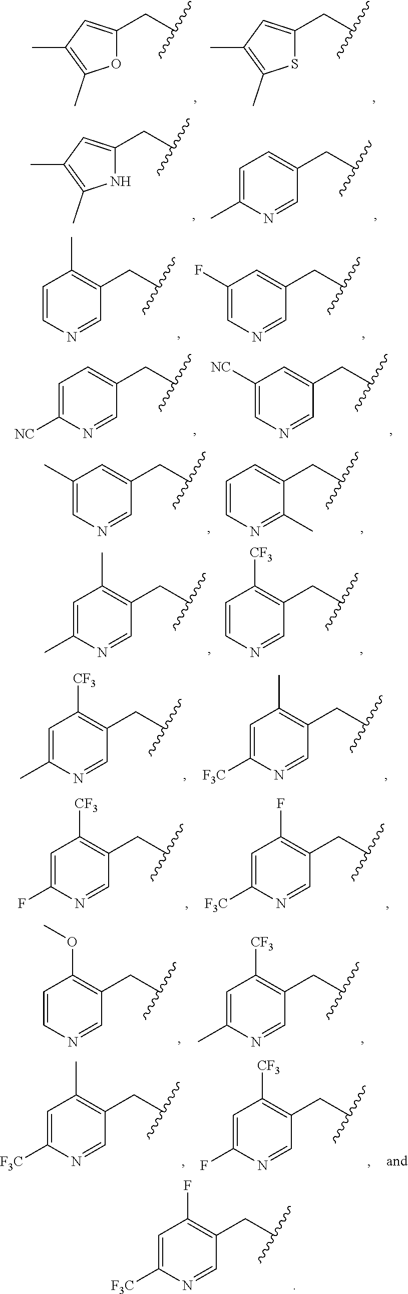

In some embodiments, R² is selected from the group consisting of

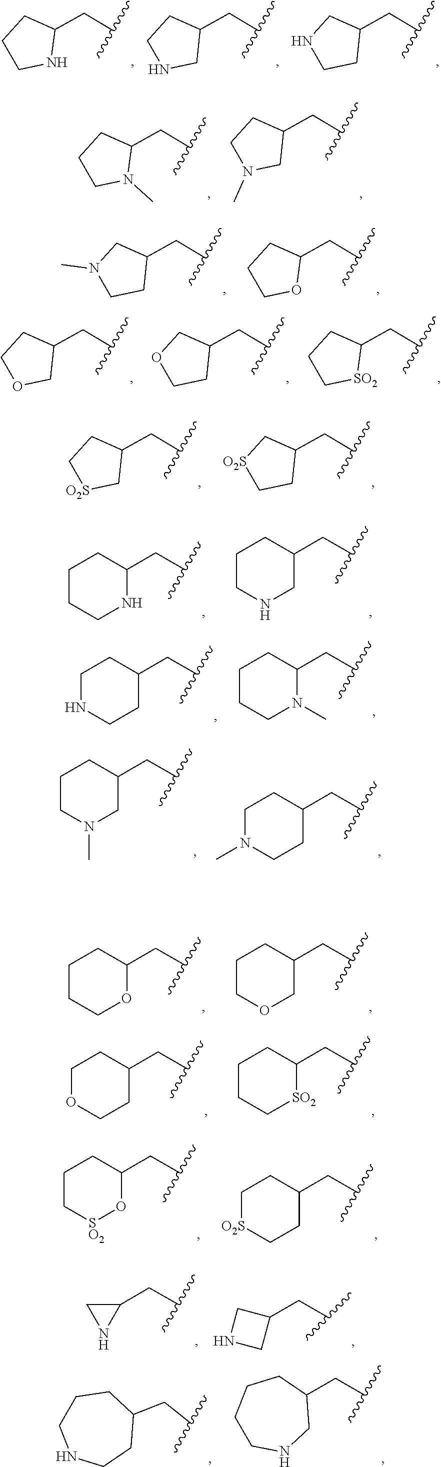

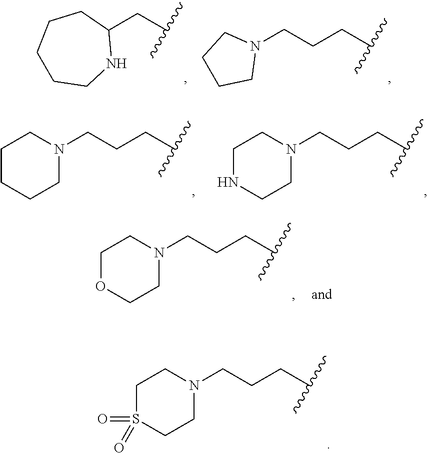

In some embodiments, R² is selected from the group consisting of

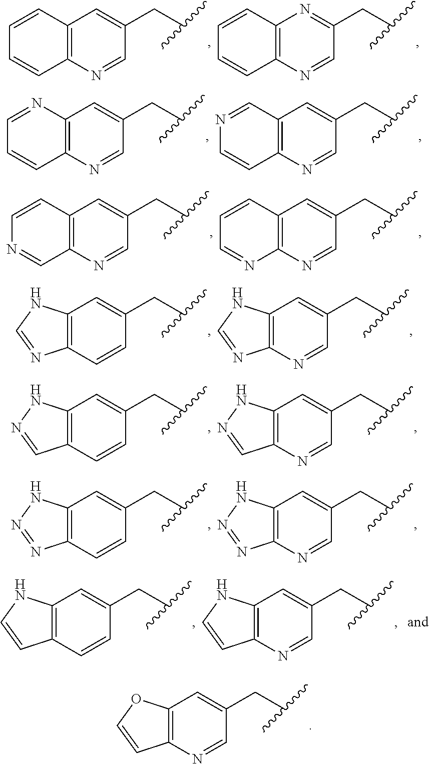

In some embodiments R² is selected from the group consisting of

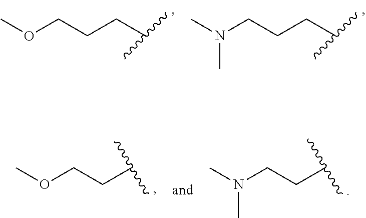

In some embodiments, R² is selected from the group consisting of

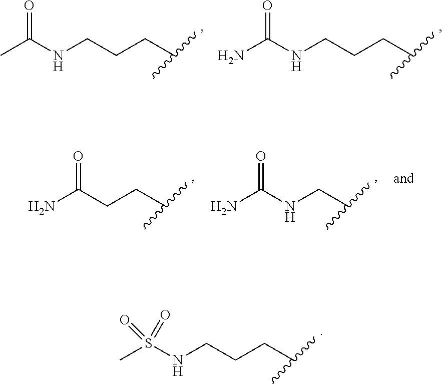

In some embodiments, R² is selected from the group consisting of

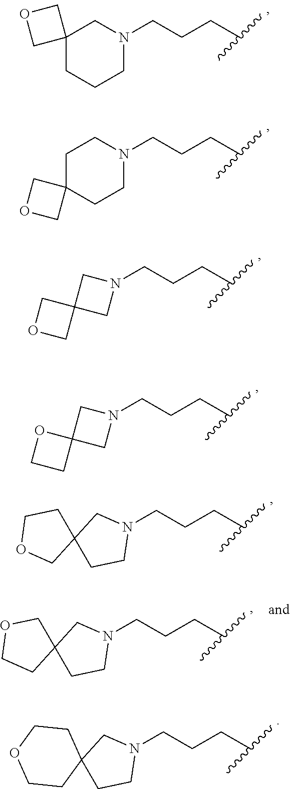

In some embodiments, R² is selected from the group consisting of

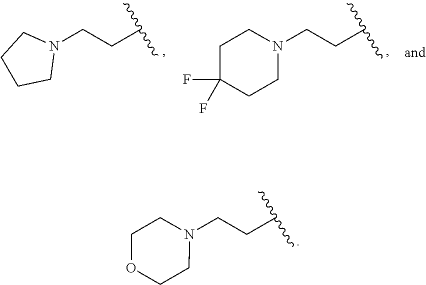 [SMILES: *CCN1CCCC1 ; *CCN1CCC(F)(F)CC1 ; *CCN1CCOCC1]

In some embodiments, R² is selected from the group consisting of

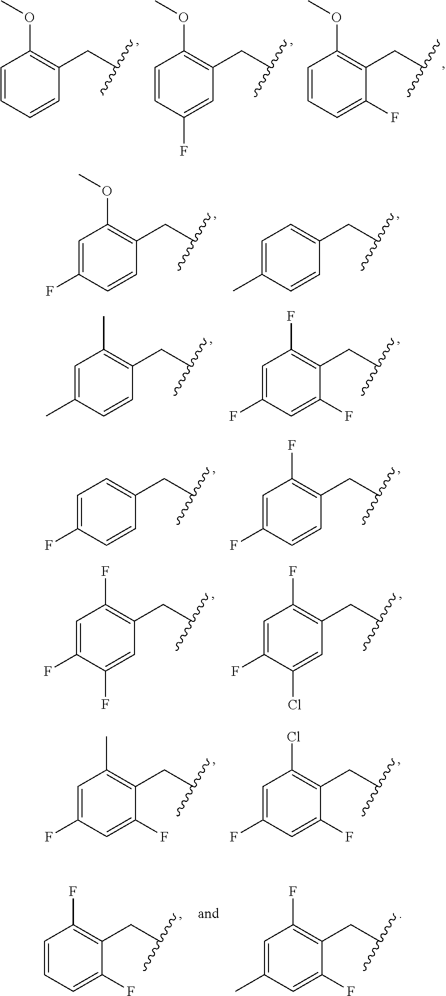

In some embodiments, the structure of Formula (I) is further represented by a formula selected from any one of the following compounds:

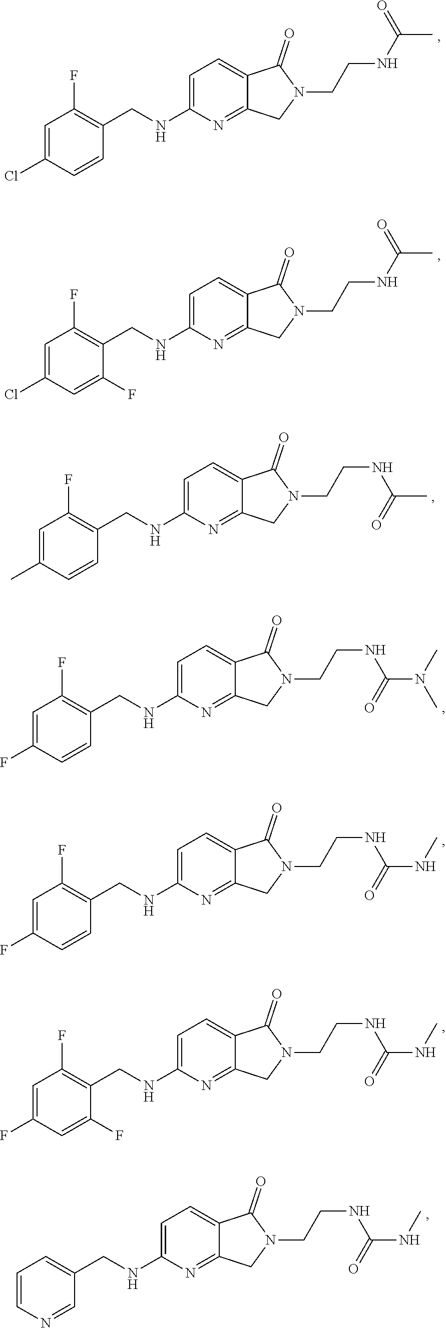

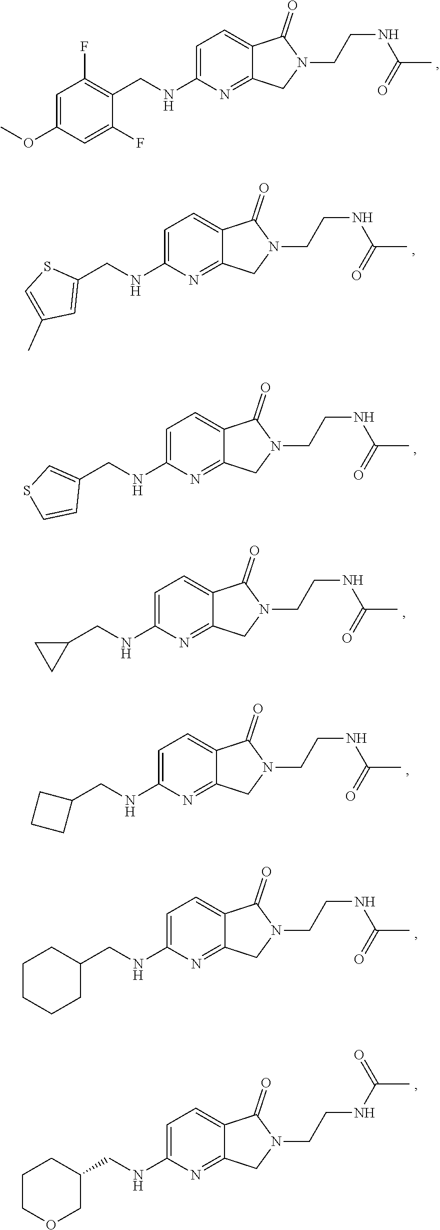

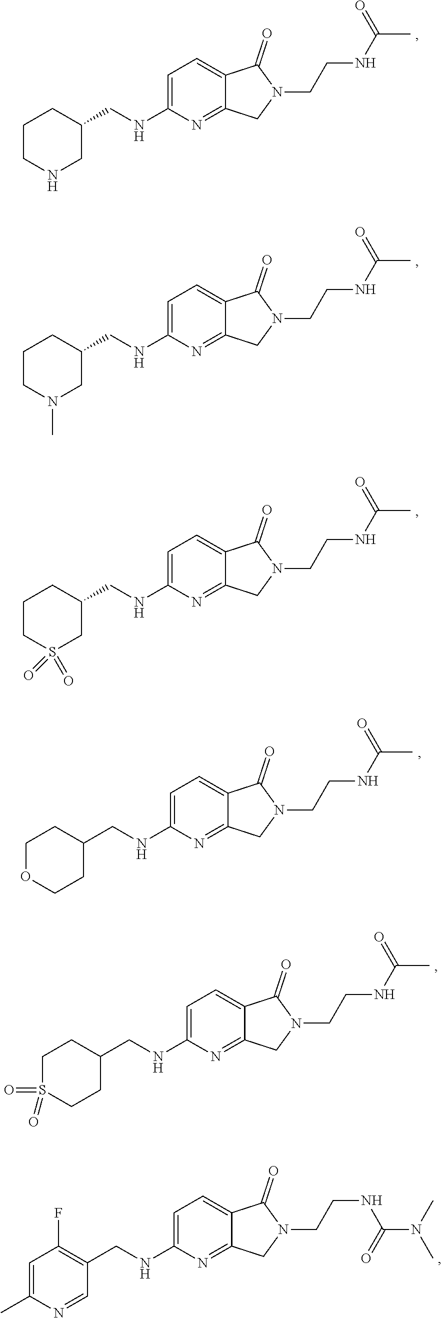

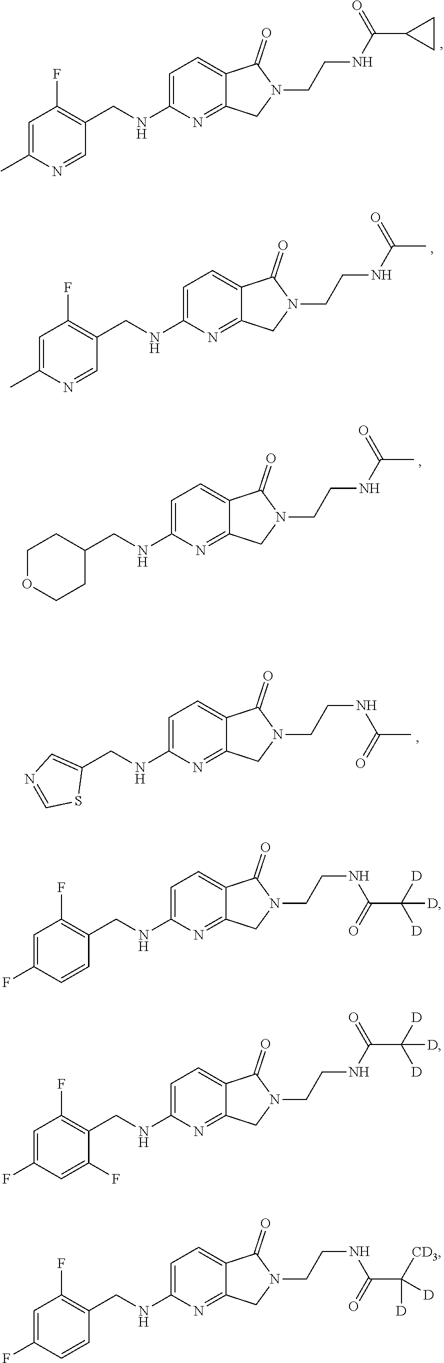

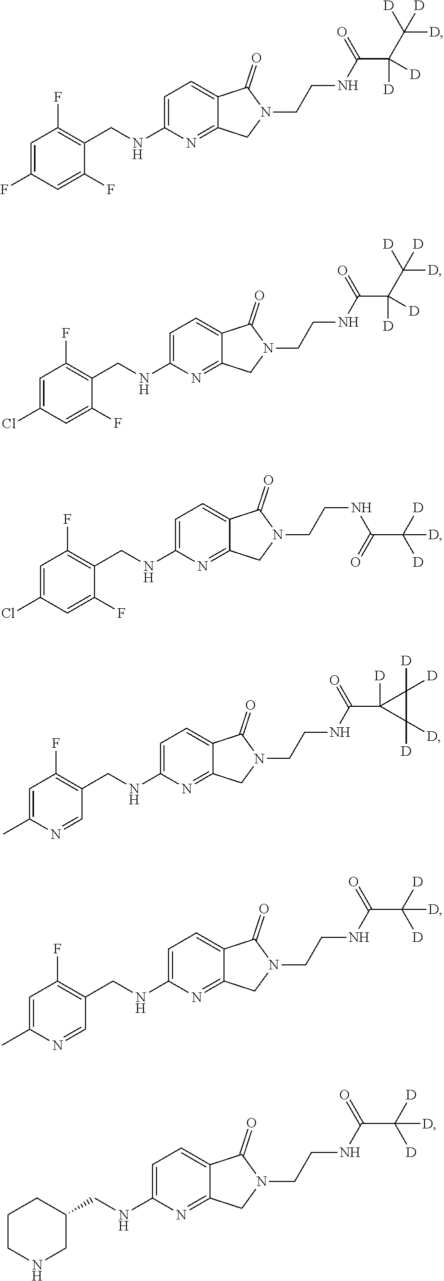

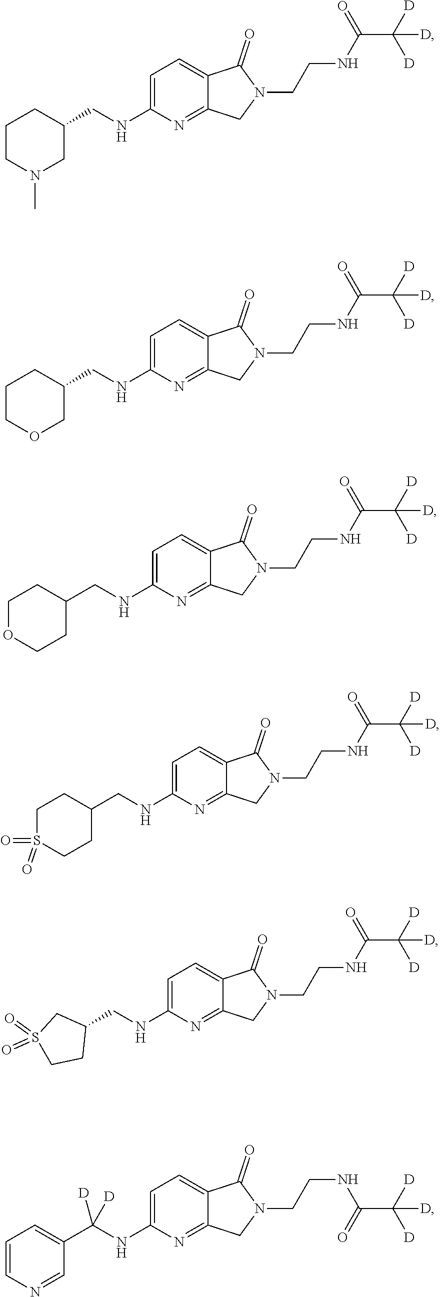

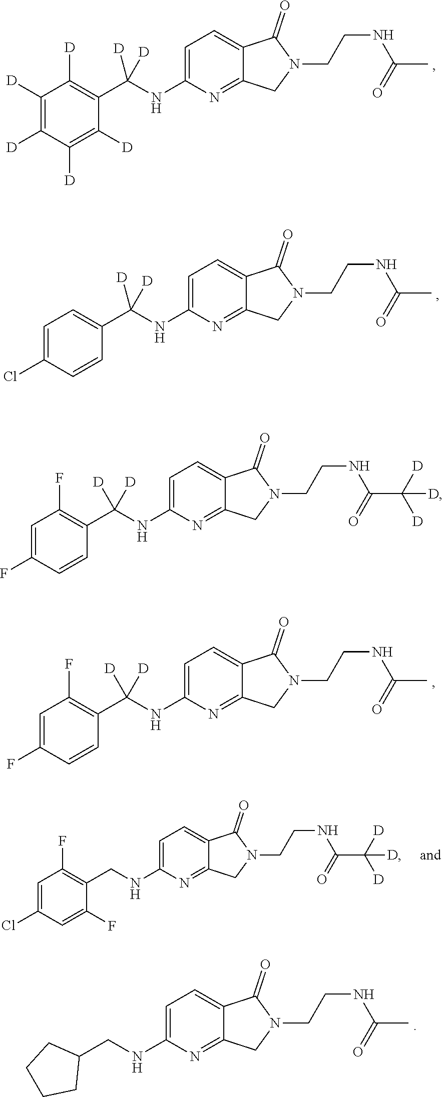

In some embodiments, the structure of Formula (I) is further represented by a formula selected from any one of the following compounds:

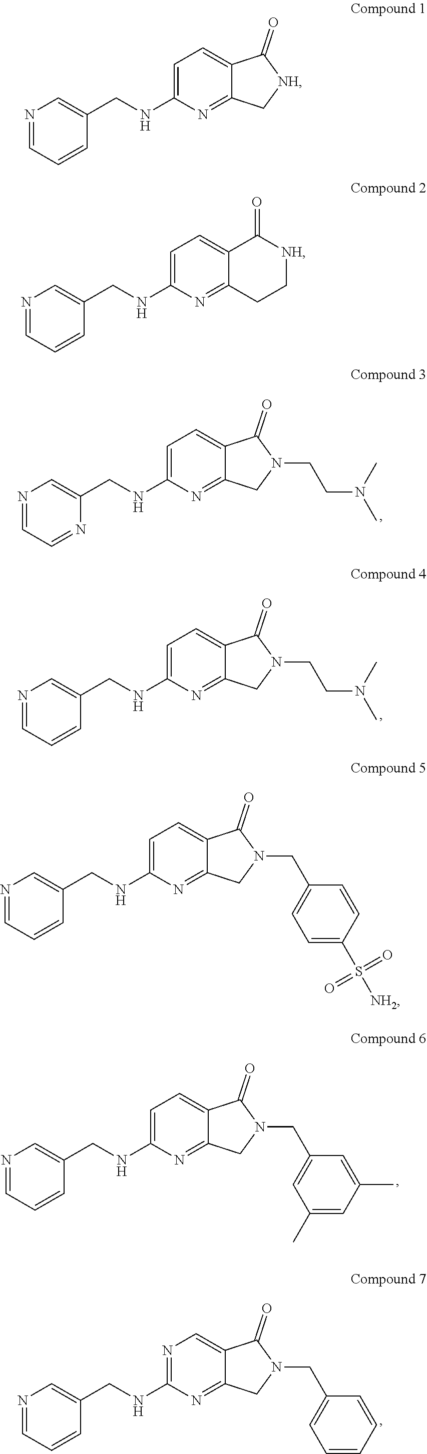

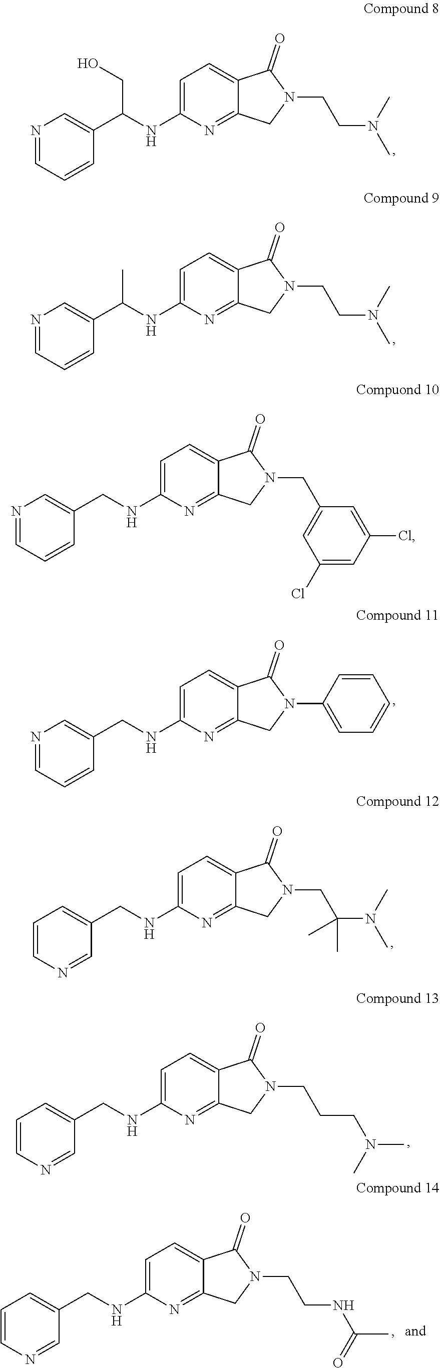

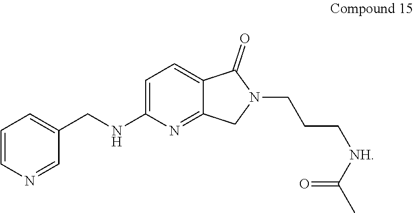 [SMILES: CC(=O)NCCCN1Cc2nc(NCc3cccnc3)ccc2C1=O]

Several embodiments pertain to a compound of Formula (I), or a pharmaceutically acceptable salt thereof, wherein the structure of Formula (I) is further represented by a structure selected from:

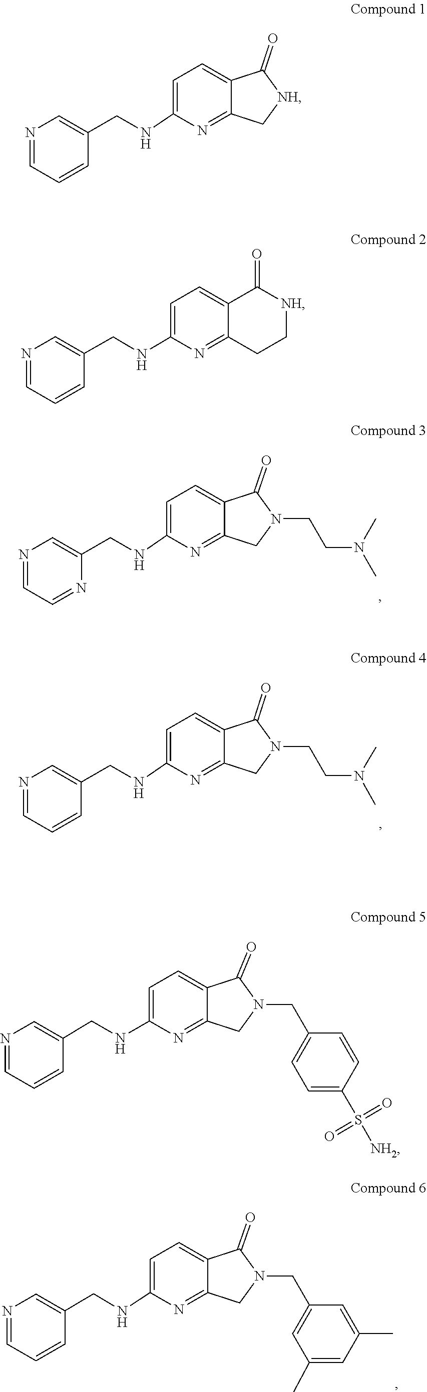

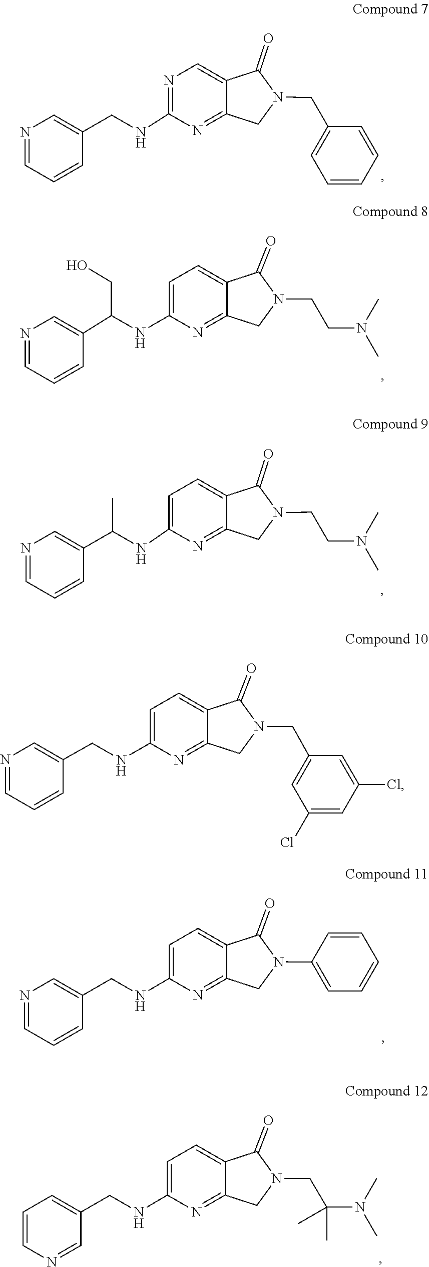 [SMILES: O=C1c2cnc(NCc3cccnc3)nc2CN1Cc1ccccc1 ; CN(C)CCN1Cc2nc(NC(CO)c3cccnc3)ccc2C1=O ; CC(Nc1ccc2c(n1)CN(CCN(C)C)C2=O)c1cccnc1 ; O=C1c2ccc(NCc3cccnc3)nc2CN1Cc1cc(Cl)cc(Cl)c1 ; O=C1c2ccc(NCc3cccnc3)nc2CN1c1ccccc1 ; CN(C)C(C)(C)CN1Cc2nc(NCc3cccnc3)ccc2C1=O]

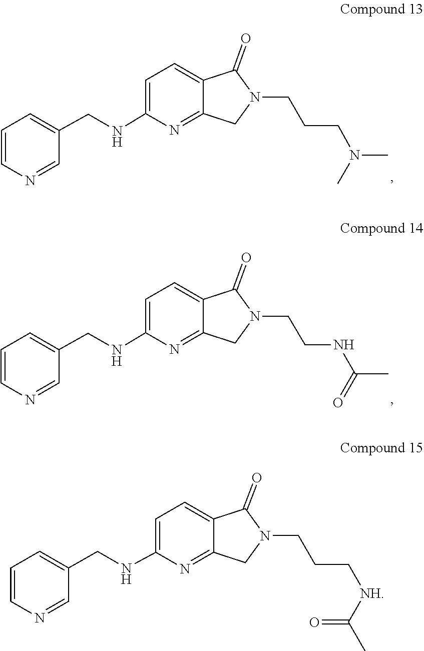 [SMILES: CN(C)CCCN1Cc2nc(NCc3cccnc3)ccc2C1=O ; CC(=O)NCCN1Cc2nc(NCc3cccnc3)ccc2C1=O ; CC(=O)NCCCN1Cc2nc(NCc3cccnc3)ccc2C1=O]

Some embodiments related to a compound of Formula (I), or a pharmaceutically acceptable salt thereof, wherein the compound is selected from:

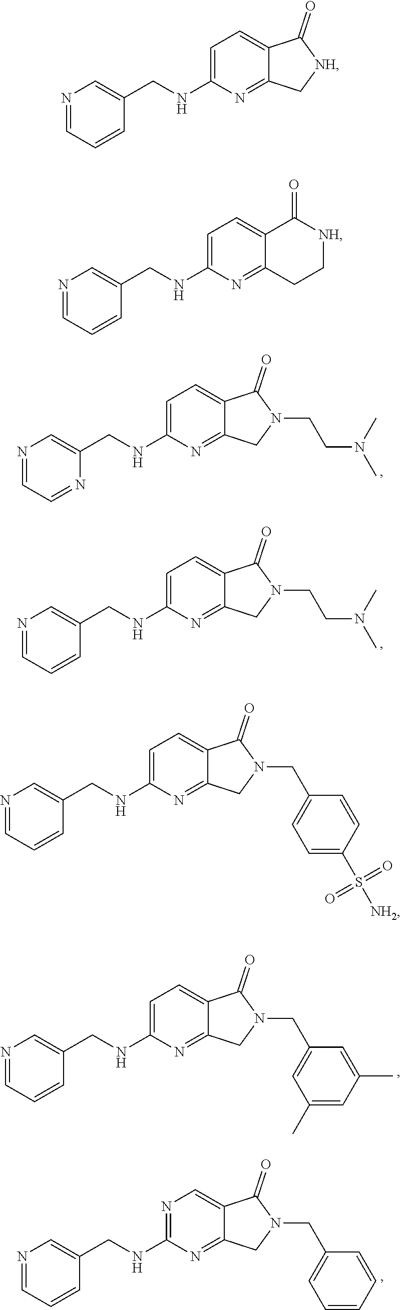

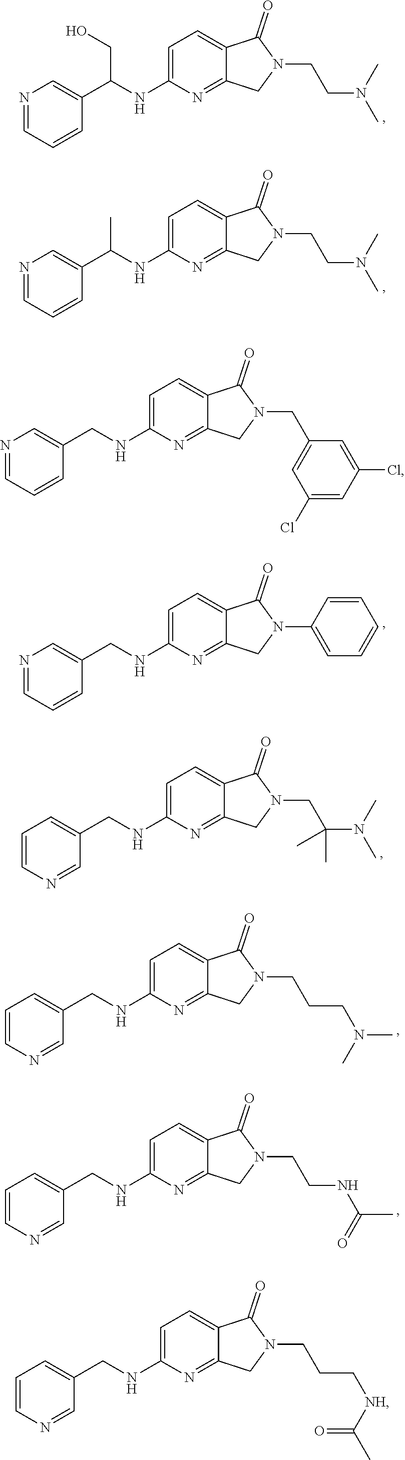

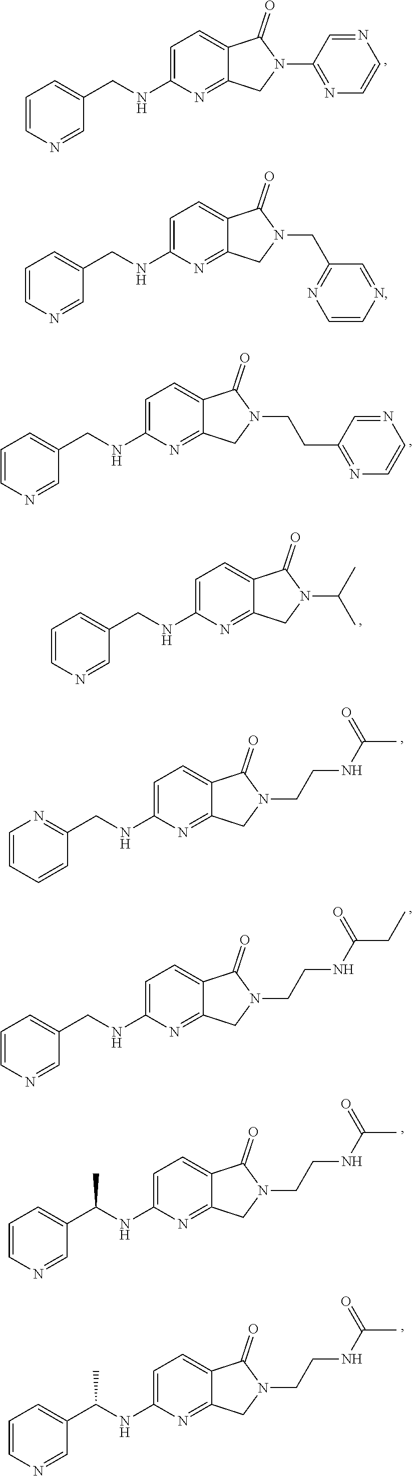

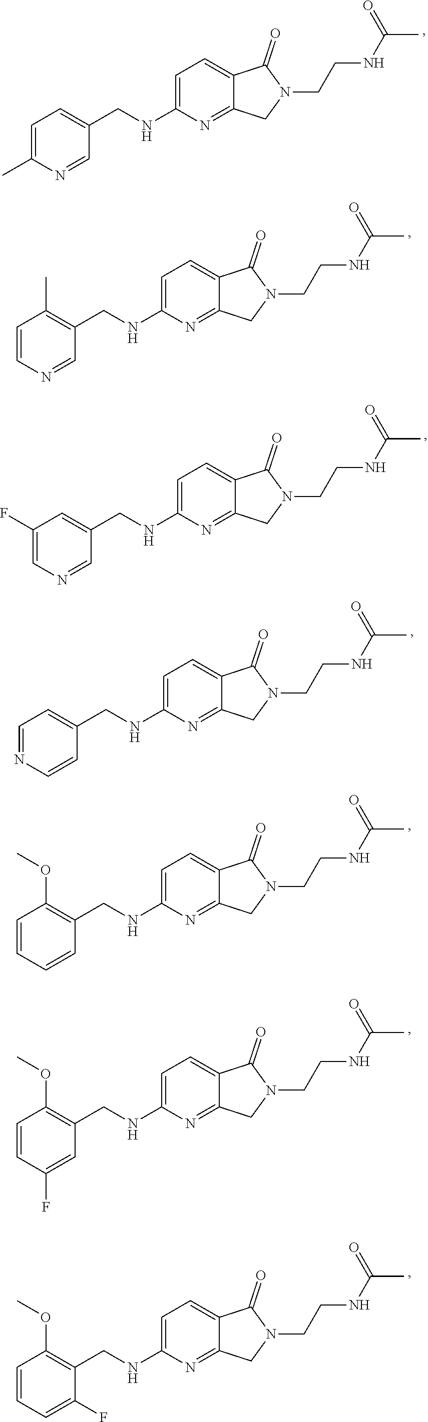

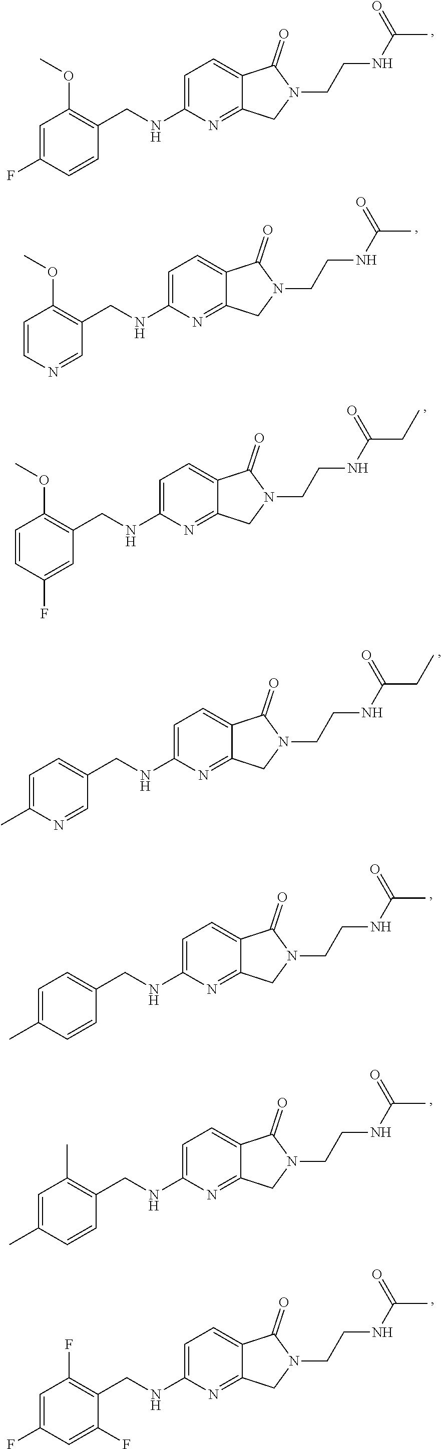

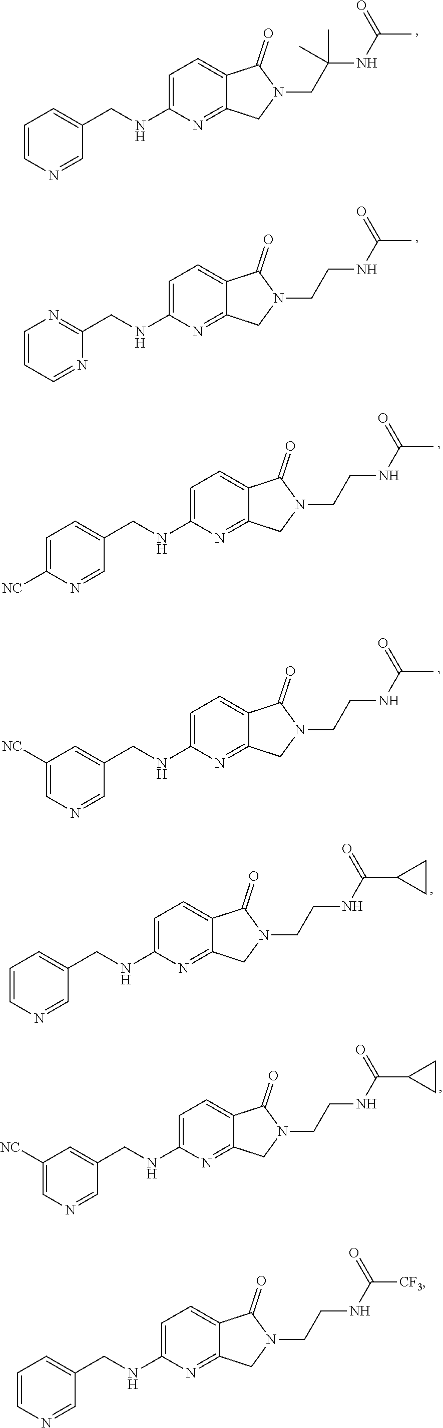

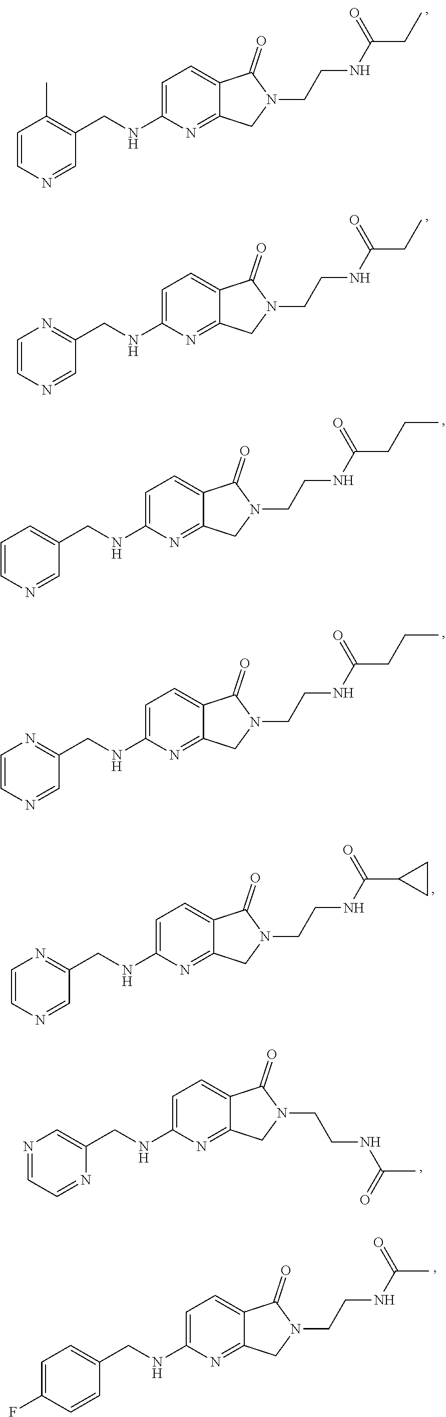

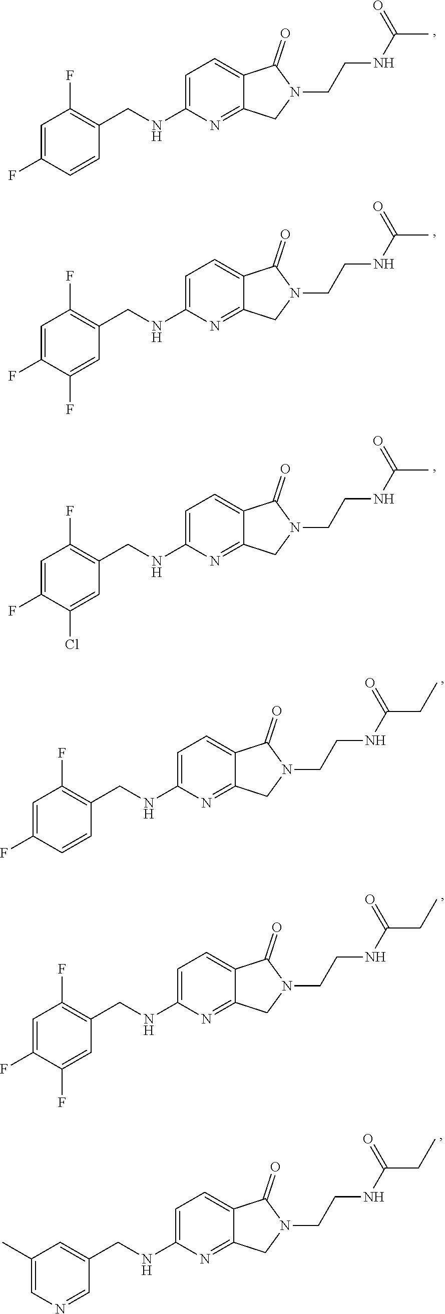

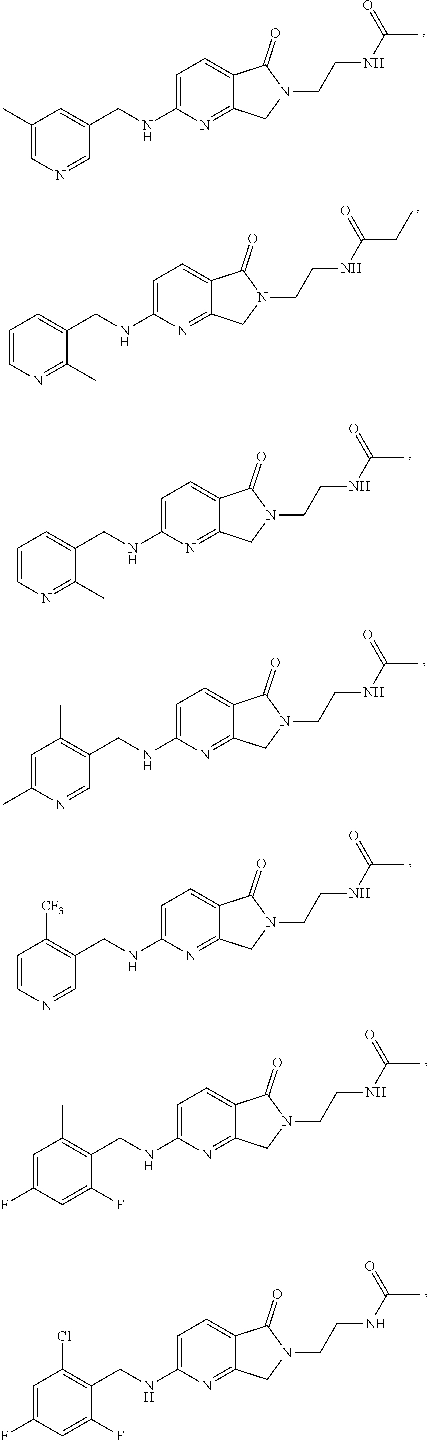

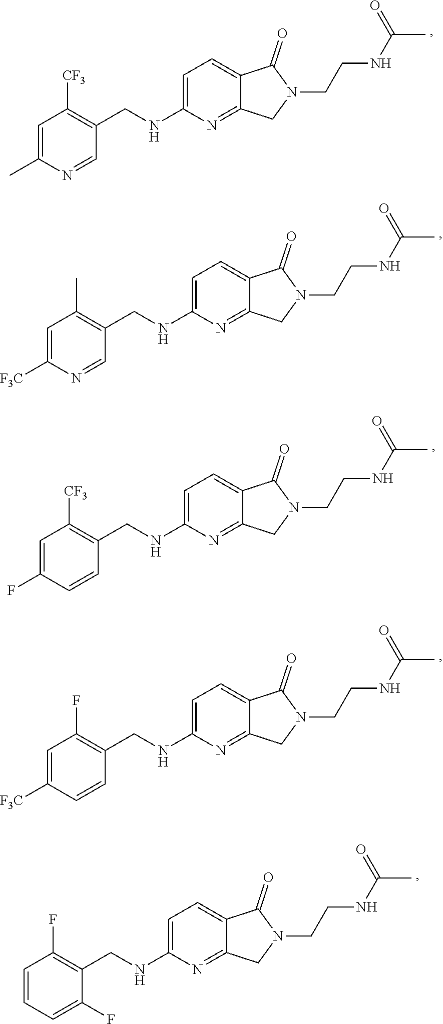 [SMILES: CC(=O)NCCN1Cc2nc(NCc3cnc(C)cc3C(F)(F)F)ccc2C1=O ; CC(=O)NCCN1Cc2nc(NCc3cnc(C(F)(F)F)cc3C)ccc2C1=O ; CC(=O)NCCN1Cc2nc(NCc3ccc(F)cc3C(F)(F)F)ccc2C1=O ; CC(=O)NCCN1Cc2nc(NCc3ccc(C(F)(F)F)cc3F)ccc2C1=O ; CC(=O)NCCN1Cc2nc(NCc3c(F)cccc3F)ccc2C1=O]

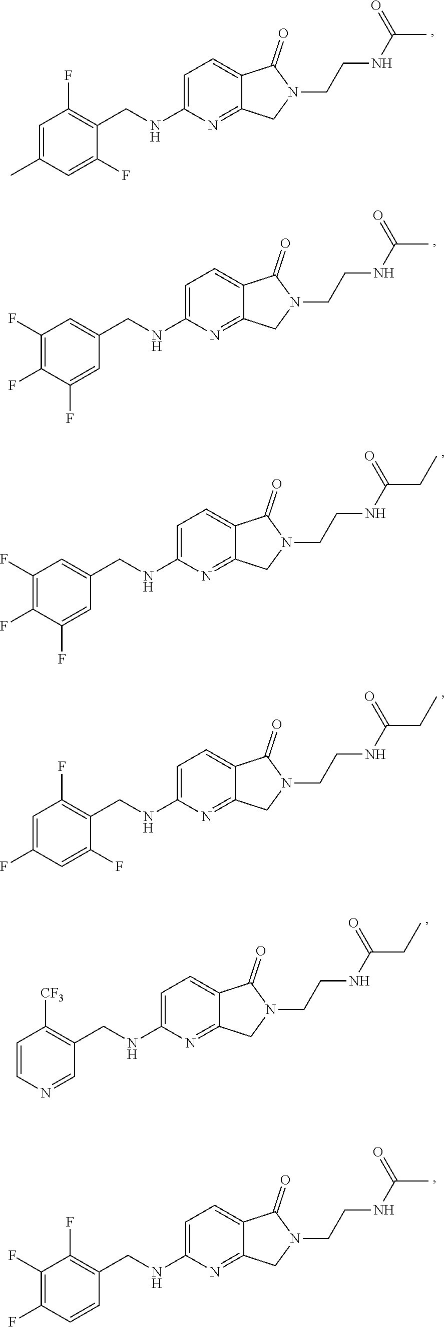 [SMILES: CC(=O)NCCN1Cc2nc(NCc3c(F)cc(C)cc3F)ccc2C1=O ; CC(=O)NCCN1Cc2nc(NCc3cc(F)c(F)c(F)c3)ccc2C1=O ; CCC(=O)NCCN1Cc2nc(NCc3cc(F)c(F)c(F)c3)ccc2C1=O ; CCC(=O)NCCN1Cc2nc(NCc3c(F)cc(F)cc3F)ccc2C1=O ; CCC(=O)NCCN1Cc2nc(NCc3cnccc3C(F)(F)F)ccc2C1=O ; CC(=O)NCCN1Cc2nc(NCc3ccc(F)c(F)c3F)ccc2C1=O]

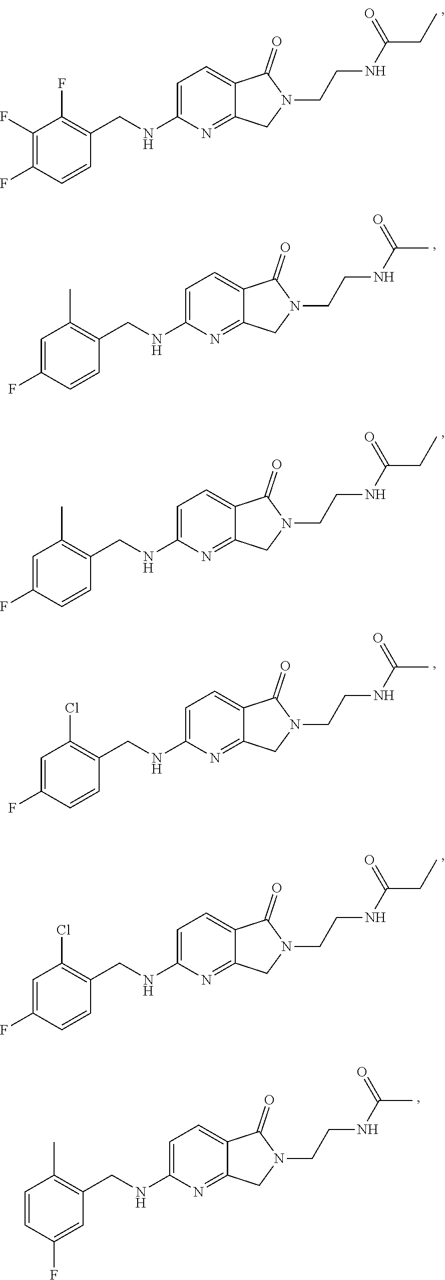 [SMILES: CCC(=O)NCCN1Cc2nc(NCc3ccc(F)c(F)c3F)ccc2C1=O ; CC(=O)NCCN1Cc2nc(NCc3ccc(F)cc3C)ccc2C1=O ; CCC(=O)NCCN1Cc2nc(NCc3ccc(F)cc3C)ccc2C1=O ; CC(=O)NCCN1Cc2nc(NCc3ccc(F)cc3Cl)ccc2C1=O ; CCC(=O)NCCN1Cc2nc(NCc3ccc(F)cc3Cl)ccc2C1=O ; CC(=O)NCCN1Cc2nc(NCc3cc(F)ccc3C)ccc2C1=O]

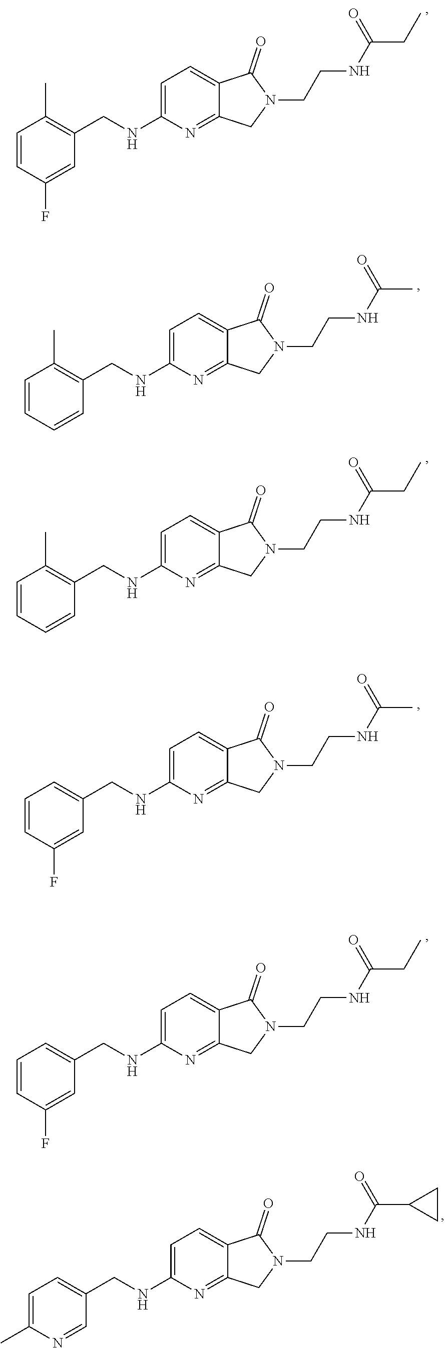

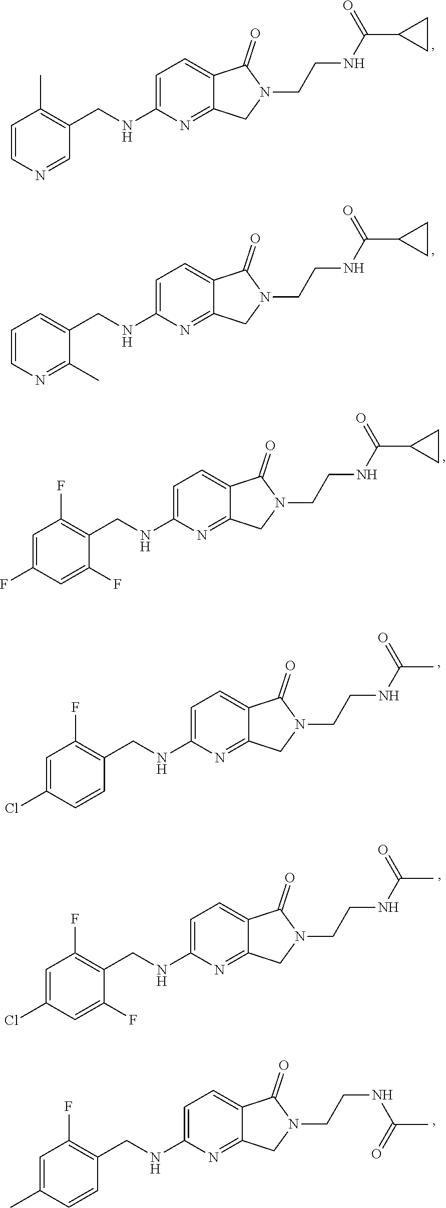

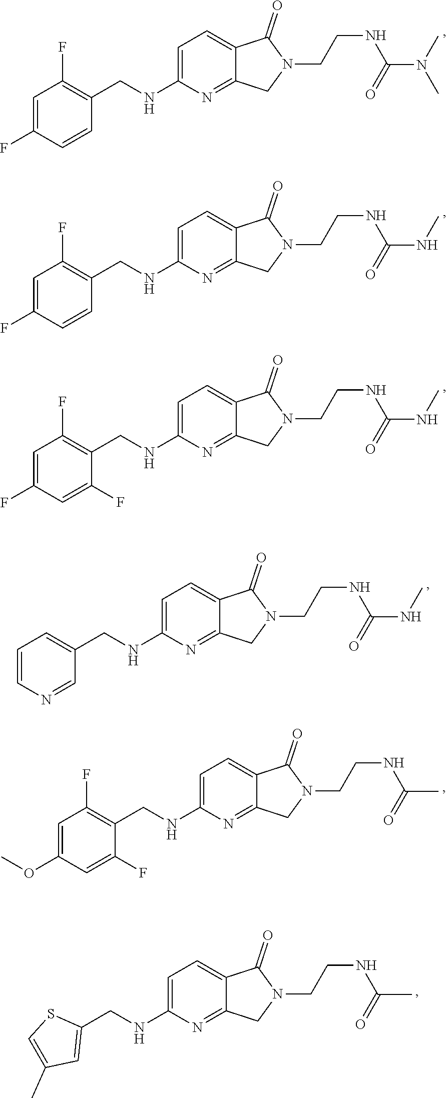

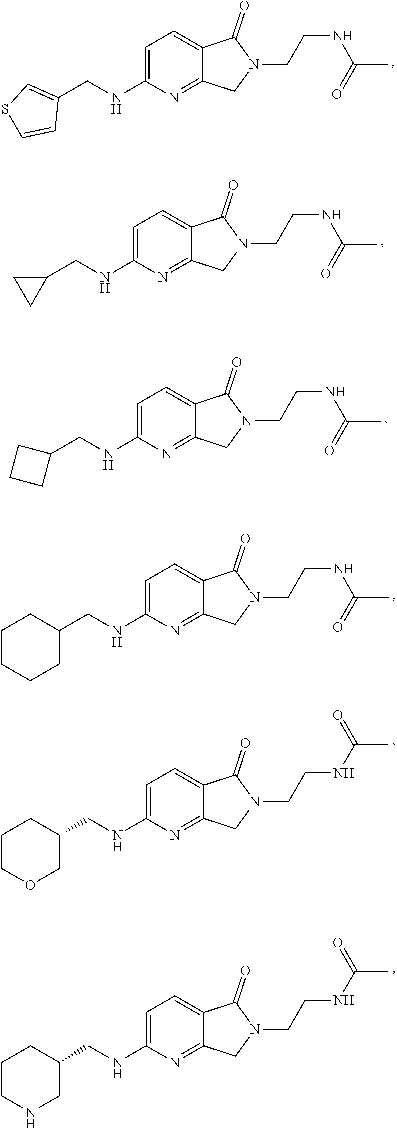 [SMILES: CC(=O)NCCN1Cc2nc(NCc3ccsc3)ccc2C1=O ; CC(=O)NCCN1Cc2nc(NCC3CC3)ccc2C1=O ; CC(=O)NCCN1Cc2nc(NCC3CCC3)ccc2C1=O ; CC(=O)NCCN1Cc2nc(NCC3CCCCC3)ccc2C1=O ; CC(=O)NCCN1Cc2nc(NC[C@H]3CCCOC3)ccc2C1=O ; CC(=O)NCCN1Cc2nc(NC[C@H]3CCCNC3)ccc2C1=O]

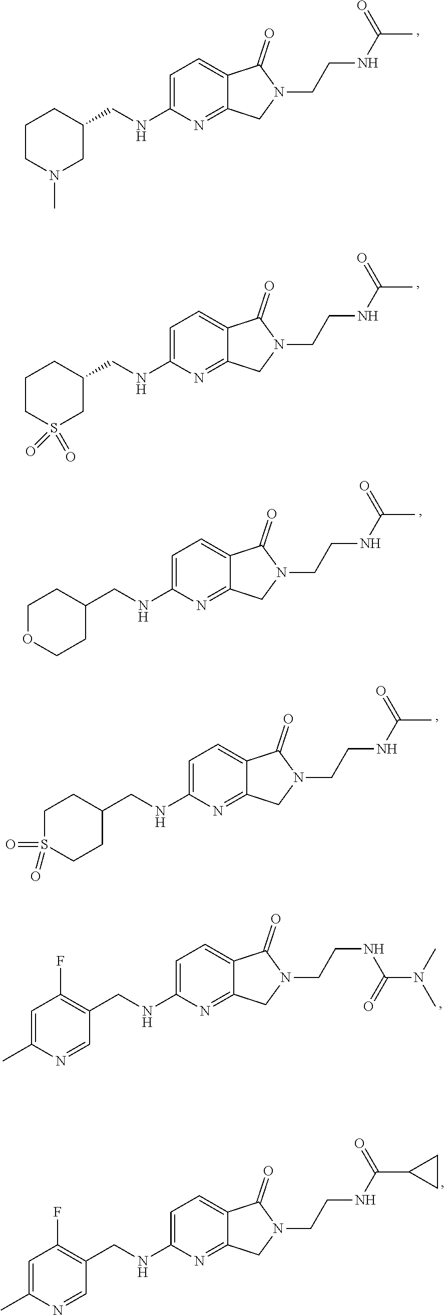

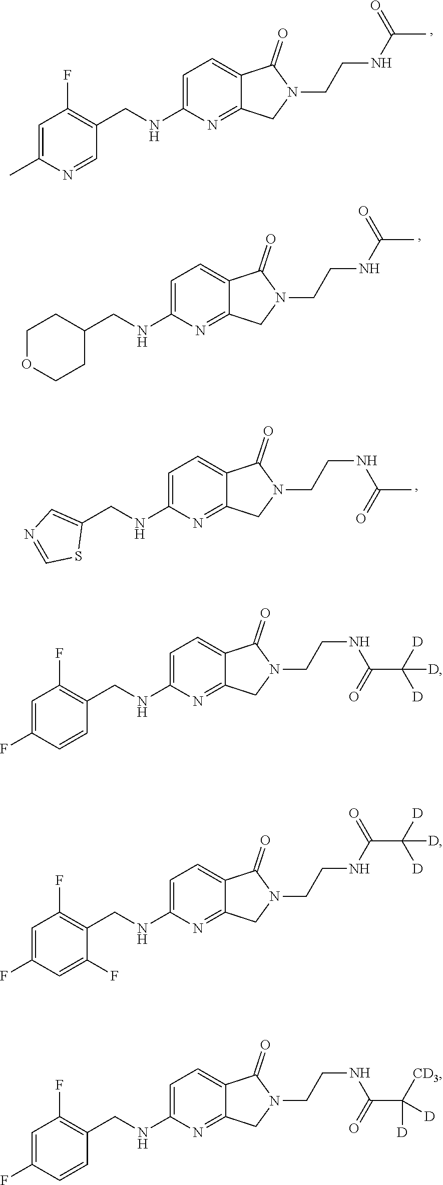

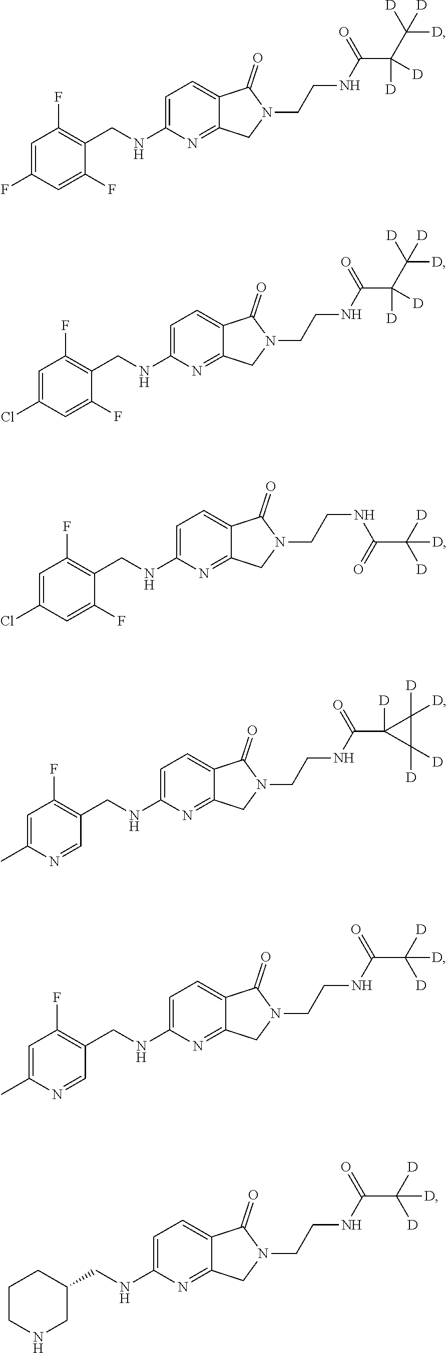

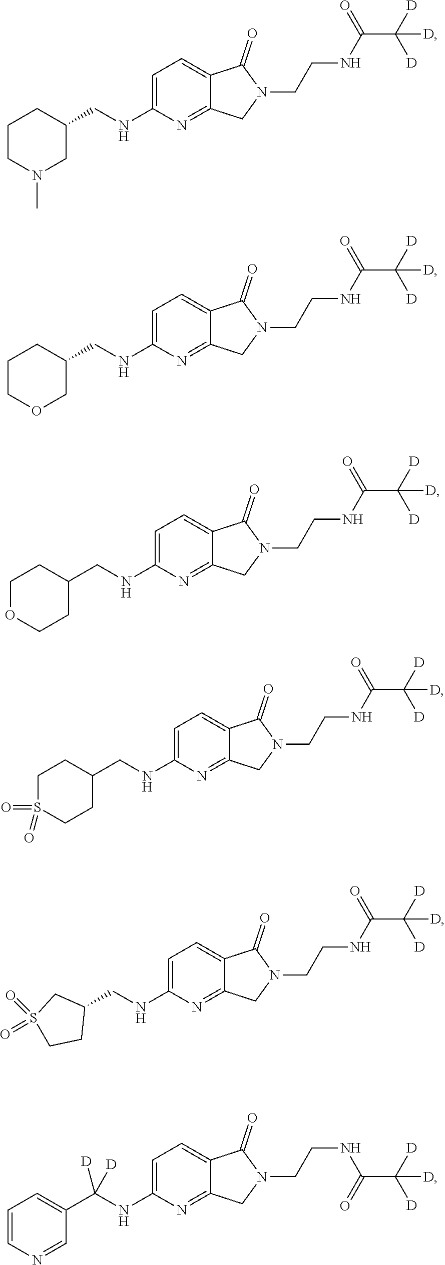

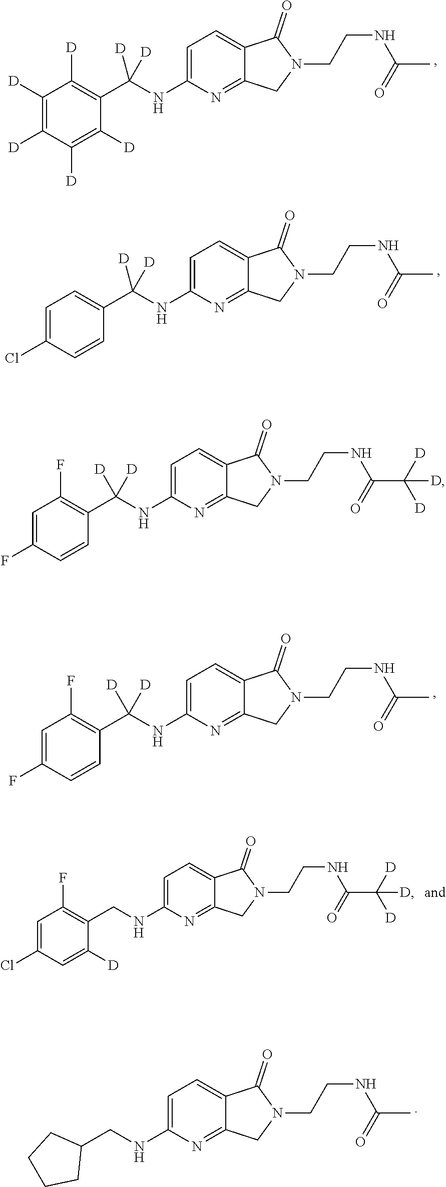

Some embodiments pertain to a method of treating a VNN1 mediated condition comprising administering to a subject requiring treatment a compound as disclosed herein. Several embodiments pertain to a method of inhibiting the enzymatic activity of the enzyme vascular non-inflammatory molecule. In several embodiments, the method pertains to the inhibition of the enzymatic activity of the enzyme vascular non-inflammatory molecule-1 (vanin-1). In several embodiments, the method comprises administering a compound as disclosed herein to a patient in need of treatment thereby treating the patient. In several embodiments, the method comprises administering an effective amount of a compound as disclosed herein to a patient in need of treatment. In several embodiments, in response to a determination of the presence of inflammatory and autoimmune disease in a sample from a subject, the subject is administered an effective amount the compound, thereby treating the inflammatory and autoimmune disease in the subject. In several embodiments, the subject is suffering from an autoimmune or inflammatory disorder. In several embodiments, the subject is suffering from a form of cancer. In some embodiments, the compound as disclosed herein has a half-maximal inhibitory concentration (IC₅₀) of less than about 5 μM. In some embodiments, the compound as disclosed herein has a half-maximal inhibitory concentration (IC₅₀) of less than about 1 μM.

Some embodiments pertain to a method of manufacturing a compound as disclosed herein, the method comprising functionalizing a bicyclic compound starting material with one or more substituents. Some embodiments pertain to the use of a compound as disclosed herein, for the preparation of a medicament to treat an autoimmune disorder, inflammatory disorder, or a form of cancer. Some embodiments pertain to the use of a compound as disclosed herein, for the treatment of an autoimmune disorder, inflammatory disorder, or a form of cancer.

### DETAILED DESCRIPTION

Several embodiments disclosed herein provide compounds useful in inhibiting the activity of vanin-1 in a subject. Several embodiments also provide methods of treating diseases utilizing these compounds or pharmaceutical compositions comprising these compounds. In several embodiments, the compounds are bicyclic compounds. In some embodiments, the bicyclic compound comprises at least one aromatic ring. In several embodiments, multiple functionalities are bound the at least one aromatic ring. In several embodiments, the bicyclic compound comprises at least one aromatic ring substituted with at least one amine group. Several embodiments comprise, consist of, or consist essentially of a bicyclic compound of Formula (I) (or any other structure disclosed herein), their pharmaceutically acceptable salts, enantiomers, methods of manufacture, and/or their methods of use in treating a VNN1 mediated condition.

The following description provides context and examples, but should not be interpreted to limit the scope of the inventions covered by the claims that follow in this specification or in any other application that claims priority to this specification. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

When referring to numerical values, the terms “or ranges including and/or spanning the aforementioned values” (and variations thereof) is meant to include any range that includes or spans the aforementioned values. For example, when the temperature of a reaction is expressed as “20° C., 30° C., 40° C., 50° C., or ranges including and/or spanning the aforementioned values,” this includes the particular temperature provided or temperature ranges spanning from 20° C. to 50° C., 20° C. to 40° C., 20° C. to 30° C., 30° C. to 50° C., 30° C. to 40° C., or 40° C. or 50° C.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, or heteroaryl group. That is, the alkyl, alkenyl, alkynyl, ring of the cycloalkyl, ring of the cycloalkenyl, ring of the cycloalkynyl, ring of the aryl, or the ring of the heteroaryl can contain from “a” to “b”, inclusive, carbon atoms. Thus, for example, a “C₁ to C₄ alkyl” group refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁ to C₆ alkyl” group refers to all alkyl groups having from 1 to 6 carbons (e.g., 1, 2, 3, 4, 5, or 6). If no “a” and “b” are designated with regard to an alkyl, alkenyl, alkynyl, cycloalkyl cycloalkenyl, cycloalkynyl, aryl, or heteroaryl group, the ranges described in these definitions are included (including the broadest ranges).

As used herein, “alkyl” refers to a straight or branched hydrocarbon chain that is fully saturated (i.e., contains no double or triple bonds). The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 20 carbon atoms (as disclosed elsewhere herein, whenever it appears herein, a numerical range such as “1 to 20” refers to each integer in the given range; for example, “1 to 20 carbon atoms” means that the alkyl group may consist of 1, 2, 3, 4, 5, 6, 7, 8, 9, 10, 11, 12, 13, 14, 15, 16, 17, 18, 19, or 20 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. The alkyl group of the compounds may be designated as “C₁₋₆ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl (“Me” or —CH₃), ethyl, propyl, isopropyl, butyl, isobutyl, tertiary butyl, pentyl, hexyl, and the like. An alkyl group may be unsubstituted or substituted.

As used herein, “alkenyl” refers to an alkyl group that contains in the straight or branched hydrocarbon chain one or more double bonds. As noted in the definition of “alkyl”, an alkenyl group may be unsubstituted or substituted.

As used herein, “alkynyl” refers to an alkyl group that contains in the straight or branched hydrocarbon chain one or more triple bonds. As noted in the definition of “alkyl”, an alkynyl group may be unsubstituted or substituted.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be re resented by [symbol] , followed by the number of carbon atoms, followed by a “*”. For example,

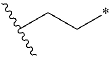 [SMILES: *CC*]

to represent ethylene. The alkylene group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. For example, a lower alkylene group can be substituted by replacing one or more hydrogen of the lower alkylene group and/or by substituting both hydrogens on the same carbon with a C₃₋₆ monocyclic cycloalkyl group (e.g., C).

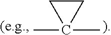

It also is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.” An alkylene group may be substituted or unsubstituted.

The term “halogen” or “halo,” as used herein, means any one of the radio-stable atoms of column 7 of the Periodic Table of the Elements, e.g., fluorine (—F), chlorine (—Cl), bromine (—Br), or iodine (—I).

As used herein, “haloalkyl” refers to a straight- or branched-chain alkyl group, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples. The haloalkyl may be a medium sized or lower haloalkyl. An haloalkyl group may be substituted or unsubstituted.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like. An alkoxy group may be substituted or unsubstituted.

As used herein, “polyethylene glycol” refers to the formula

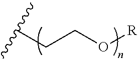

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain (e.g., alkyl) containing one or more heteroatoms. A heteroatom is given its plain and ordinary meaning in organic chemistry, which includes an element other than carbon, including but not limited to, nitrogen (e.g., amino, etc.), oxygen (e.g., alkoxy, ether, hydroxyl, etc.), sulfur, and halogens. The heteroalkyl group may have 1 to 20 carbon atoms although the present definition also covers the occurrence of the term “heteroalkyl” where no numerical range is designated. The heteroalkyl group may also be a medium size heteroalkyl having 1 to 12 carbon atoms. The heteroalkyl group could also be a lower heteroalkyl having 1 to 6 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain. A heteroalkyl group may be substituted or unsubstituted.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. For example, the aryl group can be a C₆-C₁₄ aryl group, a C₆-C₁₀ aryl group, or a C₆ aryl group. Examples of aryl groups include, but are not limited to, phenyl, naphthyl, azulenyl, and anthracenyl. An aryl group may be substituted or unsubstituted.

As used herein, “aryloxy” and “arylthio” refers to RO- and RS-, in which R is an aryl as is defined above, such as “C₆₋₁₀ aryloxy” or “C₆₋₁₀ arylthio” and the like, including but not limited to phenyloxy. An aryloxy or arylthio group may be substituted or unsubstituted.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₆ alkylene group). An aralkyl or arylalkyl group may be substituted or unsubstituted.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system can be aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. For example, the heteroaryl group can contain 4 to 14 ring members (atoms in the ring(s)), 5 to 10 ring members (atoms in the ring(s)), 5 to 7 ring members (atoms in the ring(s)), 5 to 6 ring members (atoms in the ring(s)). The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furan (e.g., furyl), furazan (e.g., furazanyl), thiophene (e.g., thienyl), benzothiophene (e.g., benzothienyl), phthalazine (e.g., phthalazinyl), pyrrole (e.g., pyrrolyl), oxazole (e.g., oxazolyl), benzoxazole (e.g., benzoxazolyl), 1,2,3-oxadiazole, 1,2,4-oxadiazole, thiazole (e.g., thiazolyl), 1,2,3-thiadiazole, 1,2,4-thiadiazole, benzothiazole (e.g., benzothiazolyl), imidazole (e.g., imidazolyl), benzimidazole (e.g., benzimidazolyl), indole (e.g., indolyl), isoindole (e.g., isoindolyl), indazole, pyrazole (e.g., pyrazolyl), benzopyrazole, isoxazole (e.g., isoxazolyl), benzoisoxazole, isothiazole (e.g., isothiazolyl), triazole (e.g., triazolyl), benzotriazole, thiadiazole (e.g., thiadiazolyl), tetrazole, pyridine (e.g., pyridinyl), pyridazine (e.g., pyridazinyl), pyrimidine (e.g., pyrimidinyl), pyrazine (e.g., pyrazinyl), purine, pteridine, quinoline (e.g., quinolinyl), isoquinoline (e.g., isoquinlinyl), quinazoline, quinoxaline, cinnoline, and triazine (e.g., triazinyl). Heteroaryl rings may also include bridge head nitrogen atoms. For example but not limited to: pyrazolo[1,5-a]pyridine, imidazo[1,2-a]pyridine, and pyrazolo[1,5-a]pyrimidine. A heteroaryl group may be substituted or unsubstituted.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. A heteroaralkyl group may be substituted or unsubstituted.

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl. A carbocyclyl group may be substituted or unsubstituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s), or as otherwise noted herein. A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkenyl” means a carbocyclyl ring or ring system having at least one double bond, wherein no ring in the ring system is aromatic. An example is cyclohexenyl. cycloalkenyl groups can contain 4 to 10 atoms in the ring(s). A cycloalkenyl group may be substituted or unsubstituted.

As used herein, “heterocyclyl” or “heteroalicyclyl” refers to three-, four-, five-, six-, seven-, eight-, nine-, ten-, eleven-, twelve-, thirteen-, up to 20-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) may be present in either a non-aromatic or aromatic ring in the ring system. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl). The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. A heterocyclyl group may be substituted or unsubstituted.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline. A sulfur of the heterocyclyl ring may be provided as a dioxide (e.g., —S(O)₂—).

A “(heterocyclyl)alkyl” is a heterocyclyl group connected, as a substituent, via an alkylene group. Examples include, but are not limited to, imidazolinylmethyl and indolinylethyl.

A “(heterocyclyl)alkynyl” is a heterocyclyl group connected, as a substituent, via an alkynylene group.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl. An acyl group may be substituted or unsubstituted.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. An O-carboxy can be substituted or unsubstituted.

A “C-carboxy” group (or “ester”) refers to a “—C(═O)OR” group in which R is selected from hydrogen, —NH₂, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH). A C-carboxy can be substituted or unsubstituted.

As used herein, the term “hydroxy” refers to a —OH group.

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A sulfinyl can be substituted or unsubstituted.

A “sulfonyl” group refers to an “—SO₂R” or “—S02-” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A sulfonyl can be provided in a heterocyclyl ring. A sulfonyl can be substituted or unsubstituted.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. A S-sulfonamido can be substituted or unsubstituted.

An “N-sulfonamido” group refers to a “—N(R_{A})SO₂R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An N-sulfonamido can be substituted or unsubstituted.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. An O-carbamyl can be substituted or unsubstituted.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An N-carbamyl can be substituted or unsubstituted.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. An O-thiocarbamyl can be substituted or unsubstituted.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂-6 alkynyl, C₃₋₇ carbocyclyl, C₆₋₁₀ aryl, 5-10 membered heteroaryl, and 5-10 membered heterocyclyl, as defined herein. An N-thiocarbamyl can be substituted or unsubstituted.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. A C-amido can be substituted or unsubstituted.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. An N-amido can be substituted or unsubstituted.

A “carbamido” or “carbamide” group refers to a “(RARB)NC(═O)N(RC)” group in which RA, RB, and RC can be independently hydrogen, alkyl, alkenyl, alkynyl, carbocyclyl, aryl, heteroaryl, heterocyclyl, aralkyl, or heterocyclyl(alkyl), as defined herein. A carbamido may be substituted or unsubstituted.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. R_{A} and R_{B} may be taken together to provide a heteroaryl or heterocycle. An amino can be substituted or unsubstituted.

An “alkamino” group refers to a “—NR_{A}R_{B}” group in which R_{A} is alkyl and R_{B} is independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An alkamino can be substituted or unsubstituted.

An “aminoalkyl” group refers to an amino group connected via an alkylene group. An aminoalkyl can be substituted or unsubstituted.

An “alkoxyalkyl” group refers to an alkoxy group connected via an alkylene group, such as a “C₂₋₈ alkoxyalkyl” and the like. An alkoxyalkyl can be substituted or unsubstituted.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl (optionally substituted with —OH or C-carboxy), C₁-C₆ alkenyl, C₁-C₆ alkynyl, C₁-C₆ heteroalkyl, C₃-C₇ carbocyclyl (optionally substituted with halo, deuterium, —OH, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with N-amido, —OH, halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), haloalkoxy, cycloalkenyl, halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, a mono-substituted amine group, a di-substituted amine group, a mono-substituted amine(alkyl), a di-substituted amine(alkyl), nitro, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfenyl, sulfinyl, sulfonyl, —O—NH₂, oxo (═O), deuterium, a diamino-group, a polyamino, a diether-group, and a polyether (e.g., diethylene glycol, triethylene glycol, oligoethylene glycol, polyethylene glycol, etc.). Wherever a group is described as “optionally substituted” (or other similar language) or as comprising one or more “optional substitutions,” that group can be substituted with the above substituents or can be unsubstituted.

In some embodiments, substituted group(s) is (are) substituted with one or more substituent(s) individually and independently selected from C₁-C₄ alkyl, amino, hydroxy, and halogen.

Two substituents may come together with the atom or atoms to which they are attached to form a ring that is spiro or fused with the rest of the compound.

As used herein, any “R” group(s) such as, without limitation, R¹, R², R³, etc., represent substituents that can be attached to the indicated atom. An R group may be substituted or unsubstituted. If two “R” groups are described as being “taken together” (or similar language), the R groups and the atoms they are attached to can form a cycloalkyl, aryl, heteroaryl or heterocycle. When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

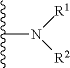

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

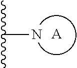

where ring A is a heterocyclyl ring containing the depicted nitrogen. As further illustration, without limitation, if R^{1a} and R^{1b} of an NR^{1a}R^{1b} group are indicated to be “taken together,” it means that they are covalently bonded to one another to form a ring:

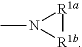

A cyclic structure may be shown using provided using the following structure (or a similar structure with a different ring size, heteroatoms, etc.):

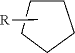

When a cyclic structure is illustrated using this type of structure, what is meant is that the R group may be attached to any position of the ring by replacing an —H of the ring with —R. For example, for the following ring:

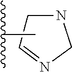

includes any of the following ring structures:

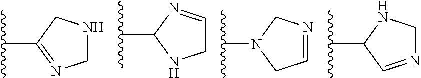

where “ [symbol] ” indicates a bond to a remaining portion of the structure. Likewise, the following structure:

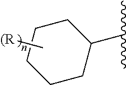

where “ [symbol] ” indicates a bond to a remaining portion of the structure and n is 1 to 5, any of the following structures are envisioned or other variations (as would be readily appreciated by the one of skill in the art):

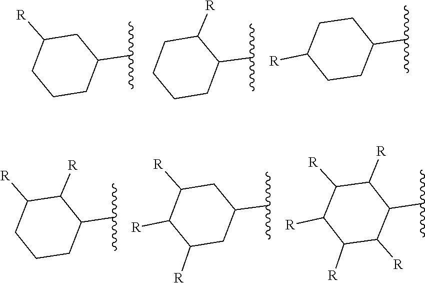

When two “adjacent” R groups are said to form a ring “together with the atoms to which they are attached,” it is meant that the collective unit of the atoms, intervening bonds, and the two R groups are the recited ring. For example, when the following substructure is present:

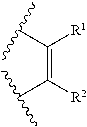

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

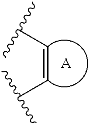

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

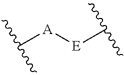

includes the substituent being oriented such that the “A” is attached at the leftmost attachment point of the molecule as well as the case in which “A” is attached at the rightmost attachment point of the molecule.

As noted in the definition for alkylene, it also is to be understood that certain radical naming conventions can include either a mono-radical or a di-radical, depending on the context. For example, where a substituent (e.g., in a genus structure) requires two points of attachment to the rest of the molecule, it is understood that the substituent is a di-radical. For example, a substituent identified as aminoalkyl that requires two points of attachment includes di-radicals such as —NHCH₂—, —NHCH₂CH₂—, —NHCH₂CH(CH₃)CH₂—, and the like. Other examples a substituent may require two points of attachment include alkoxy, aryl, heteroaryl, carbocyclyl, heterocyclyl, etc.

As used herein, a radical indicates species with a single, unpaired electron such that the species containing the radical can be covalently bonded to another species. Hence, in this context, a radical is not necessarily a free radical. Rather, a radical indicates a specific portion of a larger molecule.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof. It is understood that, in any compound described herein having one or more chiral centers, all possible diastereomers are also envisioned. It is understood that, in any compound described herein all tautomers are envisioned. It is also understood that, in any compound described herein, all isotopes of the included atoms are envisioned. For example, any instance of hydrogen, may include hydrogen-1 (protium), hydrogen-2 (deuterium), hydrogen-3 (tritium) or other isotopes; any instance of carbon may include carbon-12, carbon-13, carbon-14, or other isotopes; any instance of oxygen may include oxygen-16, oxygen-17, oxygen-18, or other isotopes; any instance of fluorine may include one or more of fluorine-18, fluorine-19, or other isotopes; any instance of sulfur may include one or more of sulfur-32, sulfur-34, sulfur-35, sulfur-36, or other isotopes.

As used herein, the term “inhibitor” means any compound, molecule or composition that inhibits or reduces the activity of a target biomolecule. The inhibition can be achieved by, for example, blocking phosphorylation of the target (e.g., competing with adenosine triphosphate (ATP), a phosphorylating entity), by binding to a site outside the active site, affecting its activity by a conformational change, or by depriving kinases of access to the molecular chaperoning systems on which they depend for their cellular stability, leading to their ubiquitylation and degradation.

As used herein, the term “vanin-1” shall be given its ordinary meaning in the art and shall refer to an enzyme with pantetheinase activity, which catalyzes the hydrolysis of pantetheine into pantothenic acid and cysteamine. Vanin-1 is member of a larger vanin family, consisting of three human (VNN1, VNN2 and VNN3) and two mouse (Vnn1 and Vnn3) orthologous genes. Vanins play a role in inflammation, oxidative stress and cell migration processes that are mediated via vanin-dependent cysteamine production.

As used herein, “subject,” “host,” “patient,” and “individual” are used interchangeably and shall be given its ordinary meaning and shall also refer to an organism that has VNN1 proteins. This includes mammals, e.g., a human, a non-human primate, ungulates, canines, felines, equines, mice, rats, and the like. The term “mammal” includes both human and non-human mammals.

“Diagnosis” as used herein shall be given its ordinary meaning and shall also include determination of a subject's susceptibility to a disease or disorder, determination as to whether a subject is presently affected by a disease or disorder, prognosis of a subject affected by a disease or disorder (e.g., identification of cancer or cancerous states, stages of cancer, or responsiveness of cancer to therapy), and use of therametrics (e.g., monitoring a subject's condition to provide information as to the effect or efficacy of therapy).

The term “sample” or “biological sample” shall be given its ordinary meaning and also encompasses a variety of sample types obtained from an organism and can be used in an imaging, a diagnostic, a prognostic, or a monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Non-limiting examples of non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject.

As used herein, the term “weight percent,” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

The term “control” refers shall be given its ordinary meaning and shall also include a sample or standard used for comparison with a sample which is being examined, processed, characterized, analyzed, etc. In several embodiments, the control is a sample obtained from a healthy patient or a non-tumor tissue sample obtained from a patient diagnosed with a tumor. In several embodiments, the control is a historical control or standard reference value or range of values. In several embodiments, the control is a comparison to a wild-type VNN1 arrangement or scenario.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide non-limiting instances of the item in discussion, not an exhaustive or limiting list thereof, and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

The section headings used herein are for organizational purposes only and are not to be construed as limiting the subject matter described. Features disclosed under one heading (such as a composition) can be used in combination with features disclosed under a different heading (a method of treating).

### INTRODUCTION

Vanin-1's enzymatic activity is associated with several inflammatory diseases, including ulcerative colitis, Crohn's disease, lupus, atherosclerosis, Type 1 diabetes, atopic dermatitis and psoriasis. Various small molecules inhibitors have been developed in the past to regulate and/or block the activity of vanin-1. Small molecule inhibitors of the enzyme vascular non-inflammatory molecule-1 (vanin-1), have been used to treat inflammatory and autoimmune disease. However, despite the fact that various inhibitors of vanin-1 are known, there remains a need for selective inhibitors to be used for the treatment of diseases which offer one or more advantages over current compounds. Those advantages include: improved activity and/or efficacy; beneficial target selectivity profile according to the respective therapeutic need; improved side effect profile, such as fewer undesired side effects, lower intensity of side effects; improved targeting of mutant receptors in diseased cells; improved physicochemical properties, such as solubility/stability in water, body fluids, and/or pharmaceutical formulations; improved pharmacokinetic properties, allowing e.g. for dose reduction or an easier dosing scheme; easier drug substance manufacturing e.g. by shorter synthetic routes or easier purification. Several embodiments disclosed herein pertain to compounds that achieve one or more of these advantages (or others). Several embodiments disclosed herein pertain to compounds that address one or more deficiencies of known drug substances.

### Compounds of Formula (I)

Several embodiments pertain to bicyclic compounds. In several embodiments, the carboxy pyrrole is a compound having the structure of Formula (I) (or a pharmaceutically acceptable salt thereof):

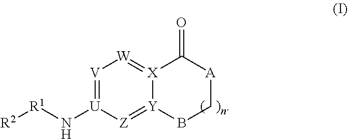

In some embodiments, A, B, U, V, W, X, Y, and Z are each independently selected from the group consisting of C, C(R⁵-R³), N, N(R⁵-R⁴), S, and S(R⁵-R⁴) and at least one instance of A, B, U, V, W, X, Y, and Z is N, N(R⁵-R⁴), S, or S(R⁵-R⁴) with the remaining variables being C or C(R⁵-R³); each instance of R¹ and R² is independently selected from the group consisting of optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 5-10 membered heteroaryl, optionally substituted 3-10 membered heteroarylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent; each instance of R³ and R⁴, where present, is independently selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent; each instance of R⁵, where present, is independently selected from the group consisting of —(CR^{x}₂)ₘ and optionally substituted C₁₋₂₀ alkylene or is absent; each R^{x} is independently —H, -D, or an optionally substituted C₁₋₆ alkyl; m is an integer in the range of 0 to 20; and n is 0, 1, or 2.

In some embodiments, A, B, U, V, W, X, Y, and Z are each independently selected from the group consisting of C, C(R⁵-R³), N, N(R⁵-R⁴), S, and S(R⁵-R⁴). In some embodiments, A is N. In some embodiments, B is N. In some embodiments, U is N. In some embodiments, V is N. In some embodiments, W is N. In some embodiments, X is N. In some embodiments, Y is N. In some embodiments, Z is N. In some embodiments, A is N(R⁵-R⁴). In some embodiments, B is N(R⁵-R⁴). In some embodiments, V is N(R⁵-R⁴). In some embodiments, W is N(R⁵-R⁴). In some embodiments, Z is N(R⁵-R⁴). In some embodiments, A is S. In some embodiments, B is S. In some embodiments, U is S. In some embodiments, V is S. In some embodiments, W is S. In some embodiments, X is S. In some embodiments, Y is S. In some embodiments, Z is S. In some embodiments, A is S(R⁵-R⁴). In some embodiments, B is S(R⁵-R⁴). In some embodiments, V is S(R⁵-R⁴). In some embodiments, W is S(R⁵-R⁴). In some embodiments, Z is S(R⁵-R⁴).

In some embodiments, R¹ is selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 5-10 membered heteroaryl, optionally substituted 3-10 membered heteroarylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent. In some embodiments, R¹ is —H. In some embodiments, R¹ is an optionally substituted C₁₋₆ alkyl. In some embodiments, R¹ is an optionally substituted C₁₋₂₀ alkylene. In some embodiments, R¹ is an optionally substituted C₁₋₆ heteroalkyl. In some embodiments, R¹ is an optionally substituted C₆₋₁₀ aryl. In some embodiments, R¹ is an optionally substituted 3-10 membered arylalkyl. In some embodiments, R¹ is an optionally substituted 5-10 membered heteroaryl. In some embodiments, R¹ is an optionally substituted 3-10 membered heteroarylalkyl. In some embodiments, R¹ is an optionally substituted 3-20 membered carbocyclyl. In some embodiments, R¹ is an optionally substituted 3-20 membered (carbocyclyl)alkyl. In some embodiments, R¹ is an optionally substituted 3-20 membered heterocyclyl. In some embodiments, R¹ is an optionally substituted 3-20 membered (heterocyclyl)alkyl. In some embodiments, R¹ is absent. In some embodiments, R¹ is —CH₂—, —CHD-, or —CD₂-. In further embodiments, R¹ is selected from the group consisting of —CHD-, CD₂-, —CH₂—, —CH₂CH₂—, —CH(CH₃)—, and —CHCH₂(OH)—. In some embodiments, R¹ is —(CR^{y}₂)ₚ—, wherein each R^{y} is independently —H or -D.

In some embodiments, R² is selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 5-10 membered heteroaryl, optionally substituted 3-10 membered heteroarylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent. In some embodiments, R² is —H. In some embodiments, R² is an optionally substituted C₁₋₆ alkyl. In some embodiments, R² is an optionally substituted C₁₋₂₀ alkylene. In some embodiments, R² is an optionally substituted C₁₋₆ heteroalkyl. In some embodiments, R² is an optionally substituted C₆₋₁₀ aryl. In some embodiments, R² is an optionally substituted 3-10 membered arylalkyl. In some embodiments, R² is an optionally substituted 5-10 membered heteroaryl. In some embodiments, R² is an optionally substituted 3-10 membered heteroarylalkyl. In some embodiments, R² is an optionally substituted 3-20 membered carbocyclyl. In some embodiments, R² is an optionally substituted 3-20 membered (carbocyclyl)alkyl. In some embodiments, R² is an optionally substituted 3-20 membered heterocyclyl. In some embodiments, R² is an optionally substituted 3-20 membered (heterocyclyl)alkyl. In some embodiments, R² is absent. In some embodiments, the optionally substituted group is substituted with between one and five groups selected from: —F, —Cl, -D, methyl, methoxy, oxo, —CN, and —CF₃.

In some embodiments, R³, where present, is independently selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent. In some embodiments, R³ is —H. In some embodiments, R³ is an optionally substituted C₁₋₆ alkyl. In some embodiments, R³ is an optionally substituted C₁₋₂₀ alkylene. In some embodiments, R³ is an optionally substituted C₁₋₆ heteroalkyl. In some embodiments, R³ is an optionally substituted C₆₋₁₀ aryl. In some embodiments, R³ is an optionally substituted 3-10 membered arylalkyl. In some embodiments, R³ is an optionally substituted 3-20 membered carbocyclyl. In some embodiments, R³ is an optionally substituted 3-20 membered (carbocyclyl)alkyl. In some embodiments, R³ is an optionally substituted 3-20 membered heterocyclyl. In some embodiments, R³ is an optionally substituted 3-20 membered (heterocyclyl)alkyl. In some embodiments, R³ is absent.

In some embodiments, R⁴, where present, is independently selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent. In some embodiments, R⁴ is —H. In some embodiments, R⁴ is an optionally substituted C₁₋₆ alkyl. In some embodiments, R⁴ is an optionally substituted C₁₋₂₀ alkylene. In some embodiments, R⁴ is an optionally substituted C₁₋₆ heteroalkyl. In some embodiments, R⁴ is an optionally substituted C₆₋₁₀ aryl. In some embodiments, R⁴ is an optionally substituted 3-10 membered arylalkyl. In some embodiments, R⁴ is an optionally substituted 3-20 membered carbocyclyl. In some embodiments, R⁴ is an optionally substituted 3-20 membered (carbocyclyl)alkyl. In some embodiments, R⁴ is an optionally substituted 3-20 membered heterocyclyl. In some embodiments, R⁴ is an optionally substituted 3-20 membered (heterocyclyl)alkyl. In some embodiments, R⁴ is absent. In some embodiments, R⁴ is an optionally substituted C₁₋₆ alkyl or an optionally substituted C₁₋₆ heteroalkyl.

In some embodiments, R⁵, where present, is independently selected from the group consisting of optionally substituted C₁₋₂₀ alkylene and absent. In some embodiments, R⁵ is an optionally substituted C₁₋₂₀ alkylene. In some embodiments, R⁵ is —(CH₂)ₘ—, —(CD₂)ₘ-, or —(CR^{x}₂)ₘ. In some embodiments, R⁵ is absent. In some embodiments, R⁵ is —(CH₂)ₘ— and m is an integer in the range of 0 to 20. In some embodiments, m is 0, 1, 2, 3, 4, 5, 6, 7, 8, 9, 10, 11, 12, 13, 14, 15, 16, 17, 18, 19, or 20. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, m is 17. In some embodiments, m is 18. In some embodiments, m is 19. In some embodiments, m is 20.

In some embodiments, n is 0, 1, or 2. In some embodiments, n is 0. In some embodiments, n is 1. In some embodiments, n is 2.

In several embodiments, the structure of Formula (I) is further represented by a formula selected from any one of Formulae (Ia) or (Ib):

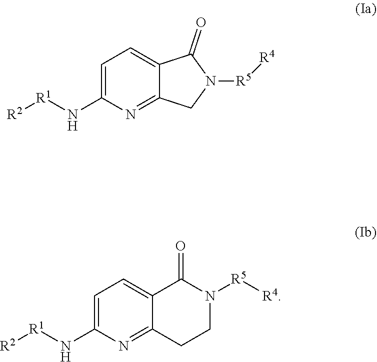

In some embodiments, R is an optionally substituted C₁₋₆ alkyl or optionally substituted C₁₋₂₀ alkylene. In further embodiments, R¹ is selected from the group consisting of —CH₂—, —CH₂CH₂—, —CH(CH₃)—, and —CHCH₂(OH)—. In some embodiments, R¹ is —CH₂—. In some embodiments, R¹ is —CH₂CH₂—. In some embodiments, R¹ is —CH(CH₃)—. In some embodiments, R¹ is —CHCH₂(OH)—. In some embodiments, R¹ is —(CH₂)ₚ— and p is an integer in the range of 0 to 20. In some embodiments, p is 0, 1, 2, 3, 4, 5, 6, 7, 8, 9, 10, 11, 12, 13, 14, 15, 16, 17, 18, 19, or 20. In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12. In some embodiments, p is 13. In some embodiments, p is 14. In some embodiments, p is 15. In some embodiments, p is 16. In some embodiments, p is 17. In some embodiments, p is 18. In some embodiments, p is 19. In some embodiments, p is 20.

In certain embodiments, each instance of R¹ and R² is independently selected from the group consisting of —H,

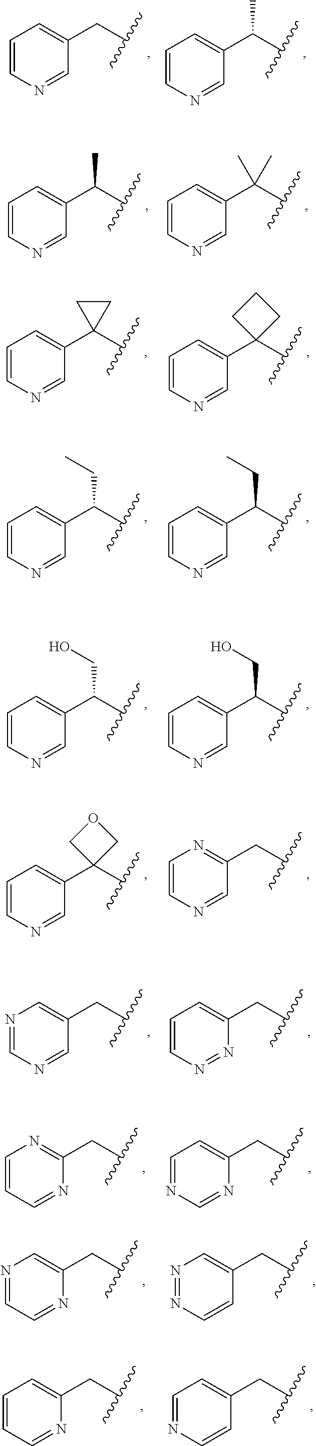

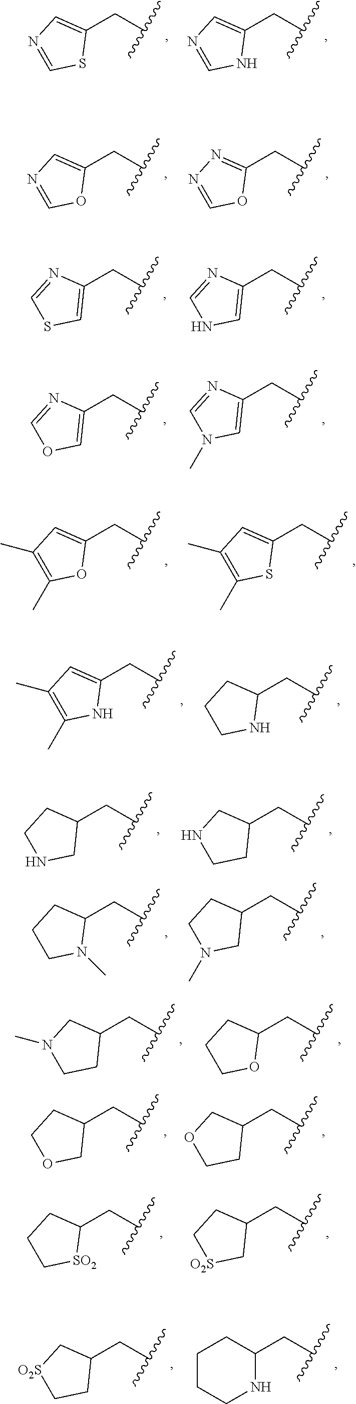

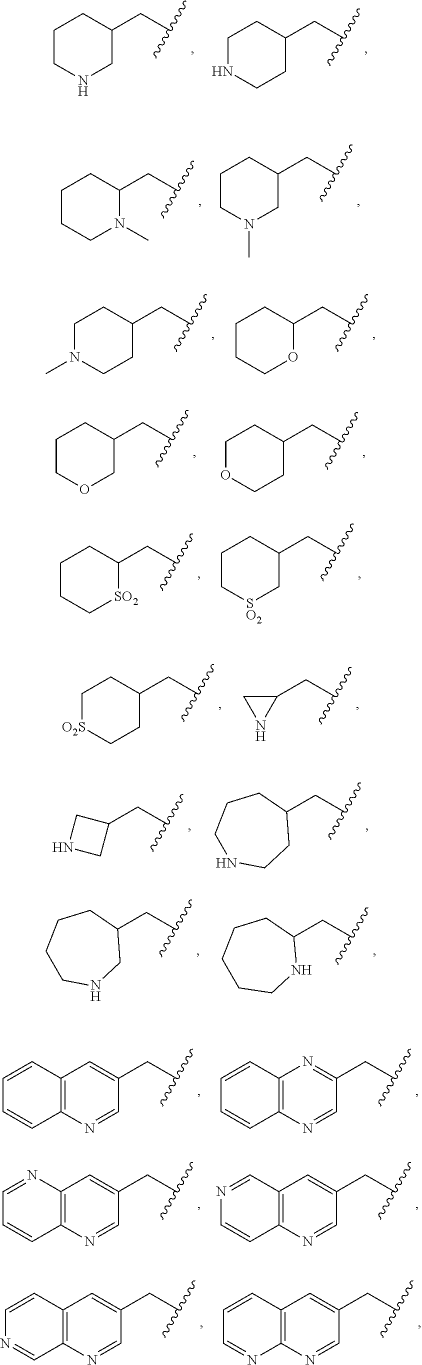

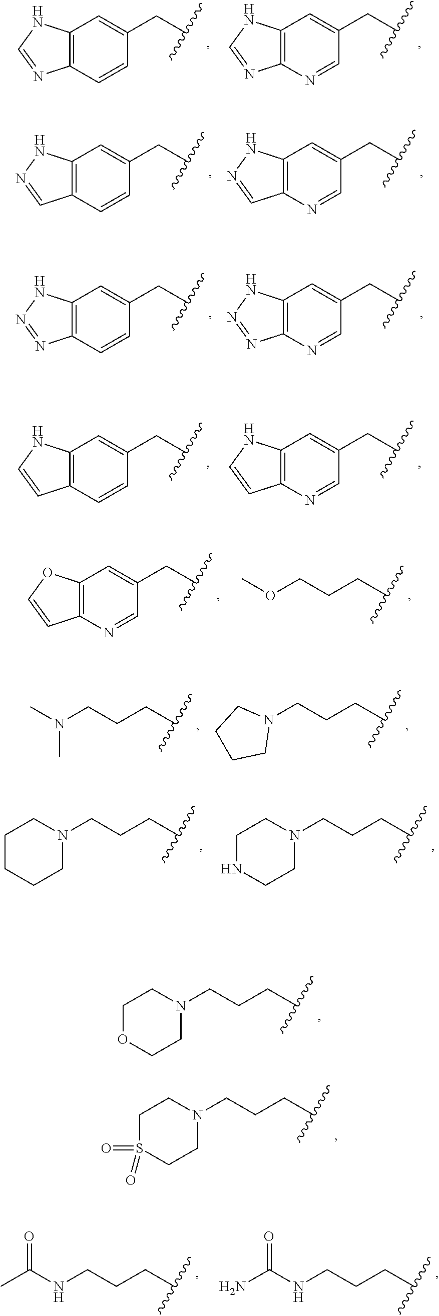

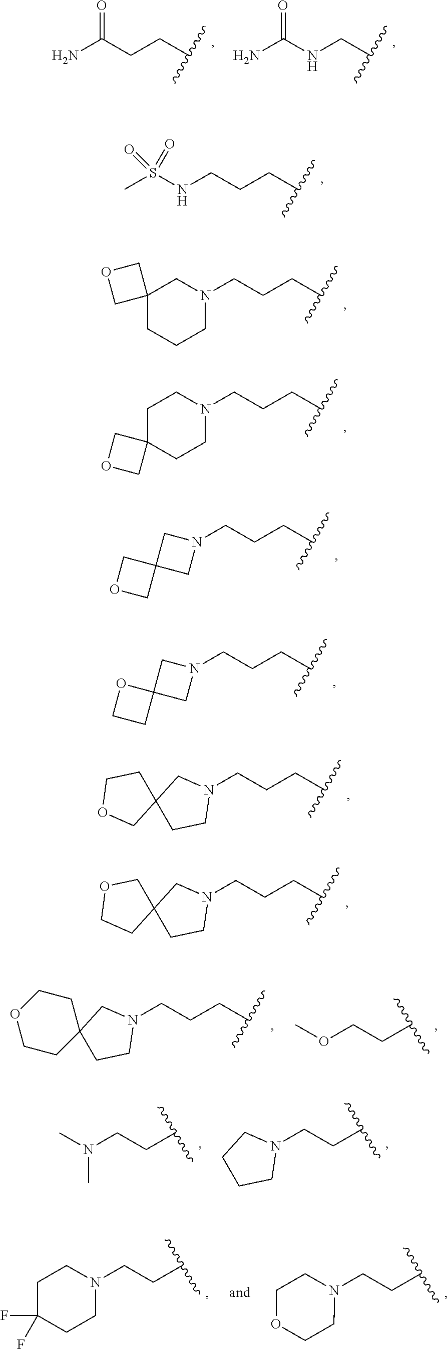

In some embodiments, R² is selected from the group consisting of —H,

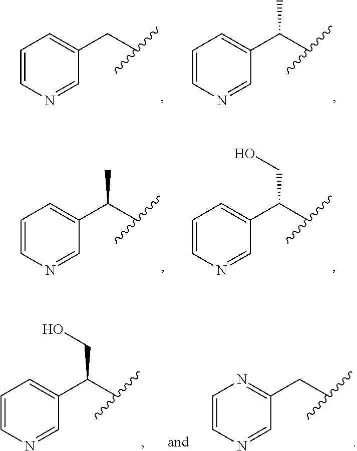

In some embodiments, R² is —H. In some embodiments, R² is

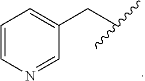 [SMILES: *Cc1cccnc1]

In some embodiments, R² is

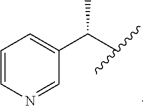 [SMILES: *[C@@H](C)c1cccnc1]

In some embodiments, R² is

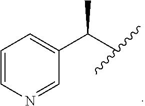 [SMILES: *[C@H](C)c1cccnc1]

In some embodiments, R² is

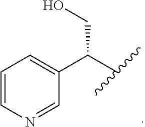 [SMILES: *[C@@H](CO)c1cccnc1]

In some embodiments, R² is

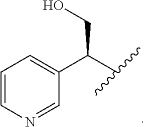 [SMILES: *[C@H](CO)c1cccnc1]

In some embodiments, R² is

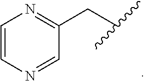 [SMILES: *Cc1cnccn1]

In certain embodiments, each instance of R³ and R⁴, where present, is independently selected from the group consisting of —H,

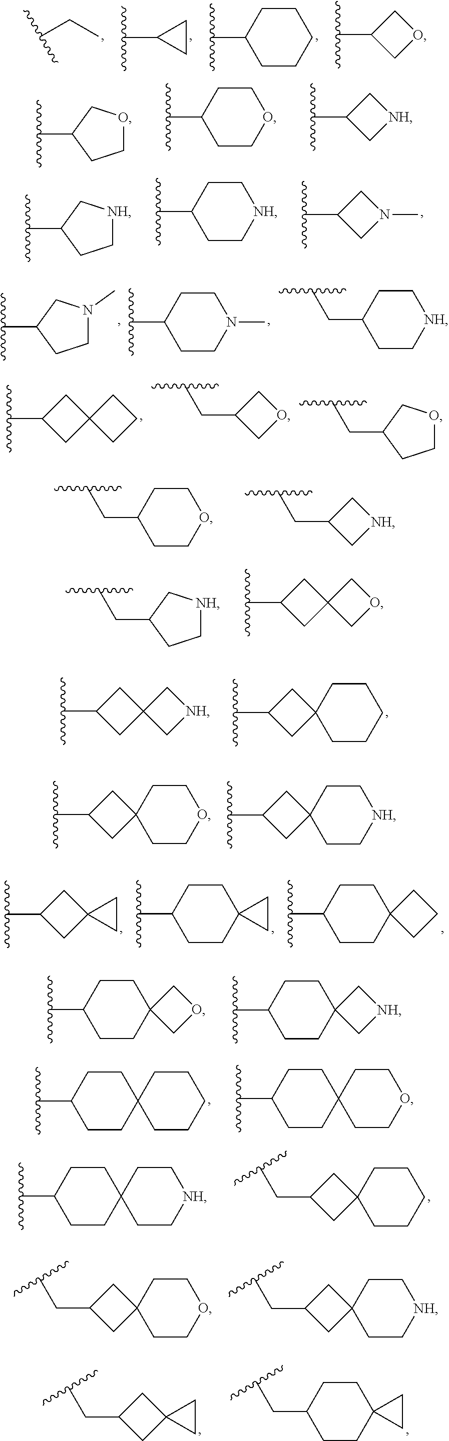

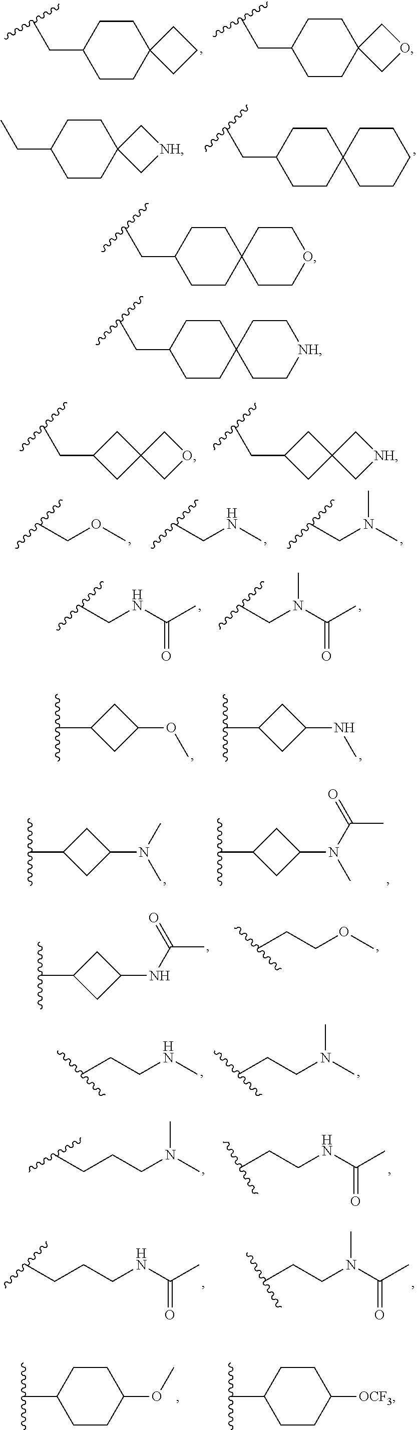

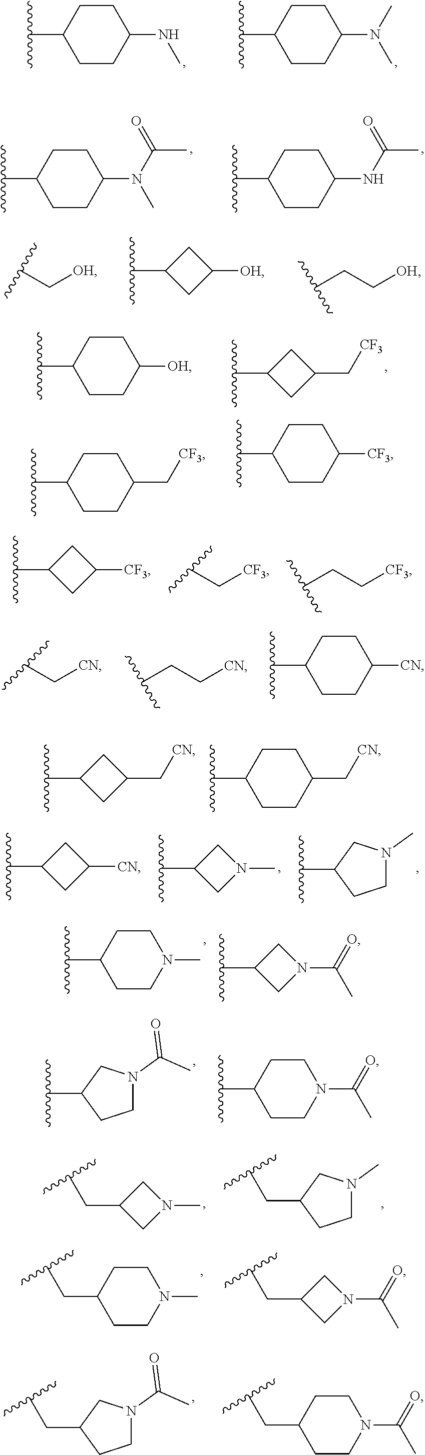

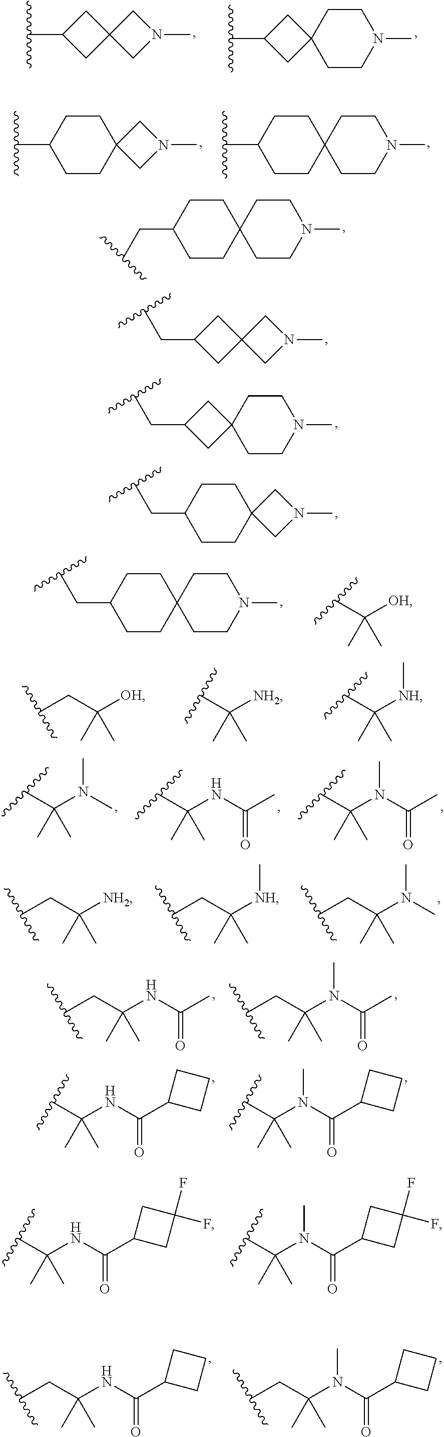

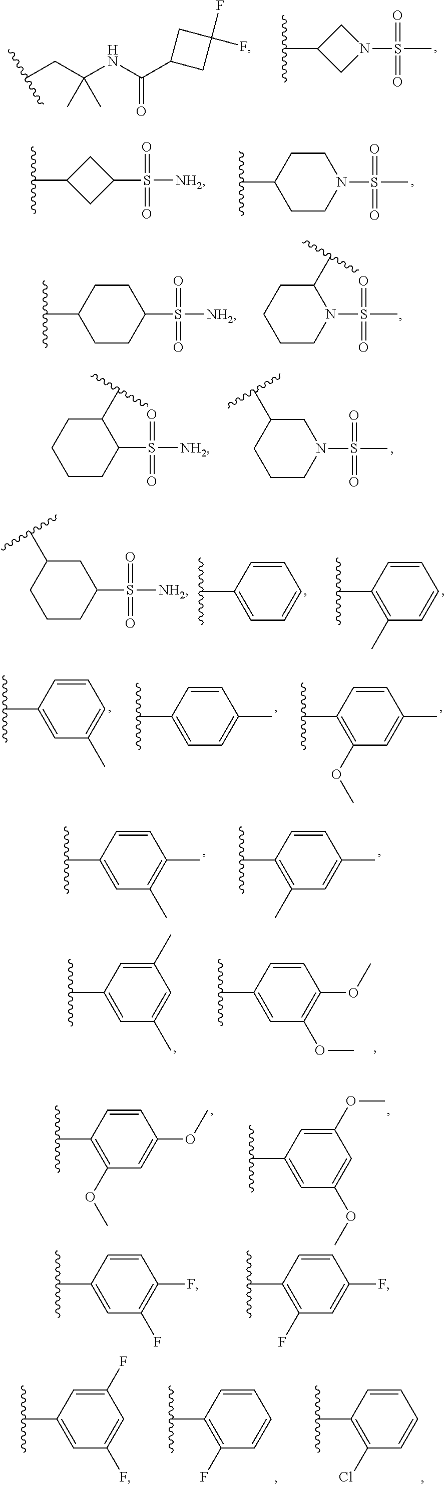

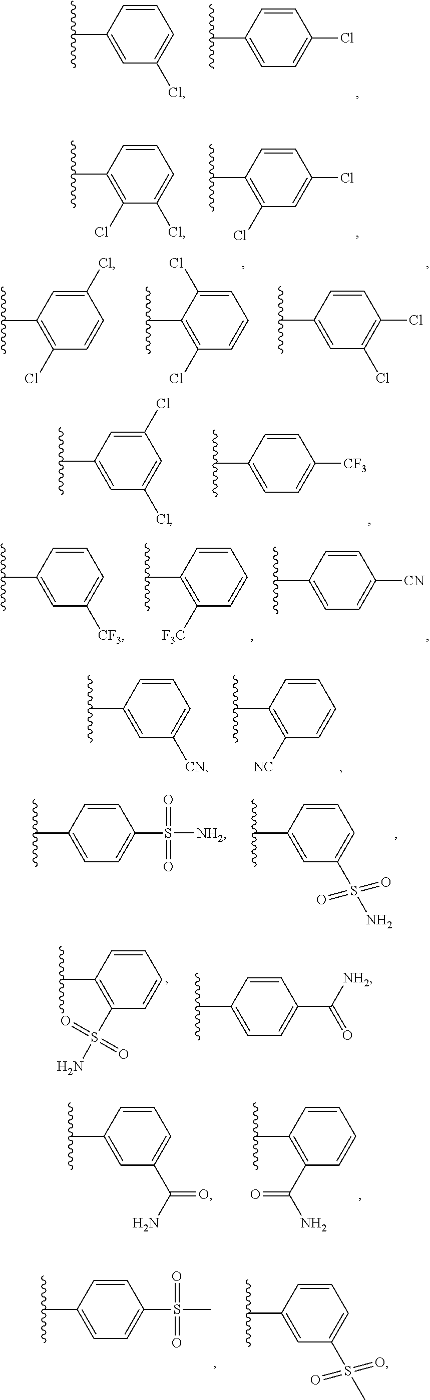

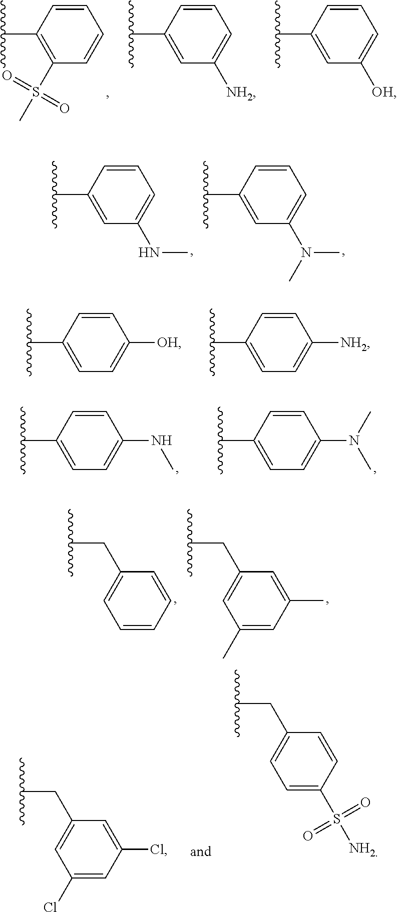

In some embodiments, for example, A is N(R⁵R⁴). In further embodiments, R⁴ is selected from the group consisting of —H,

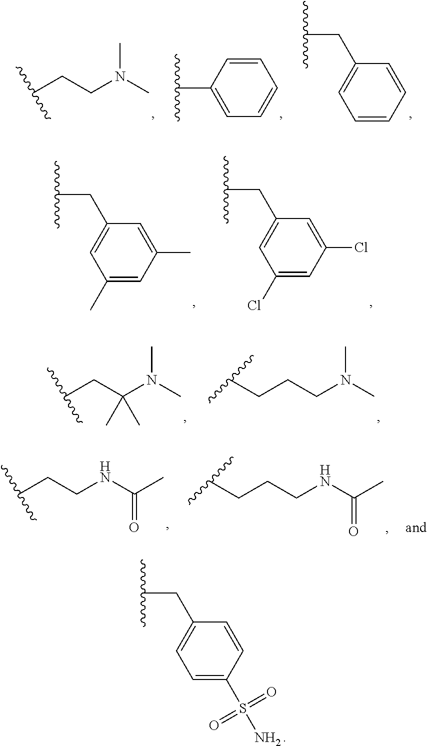

In some embodiments, R⁴ is —H. In some embodiments, R⁴ is

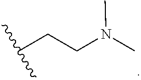 [SMILES: *CCN(C)C]

In some embodiments, R⁴ is

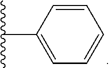 [SMILES: *c1ccccc1]

In some embodiments, R⁴ is

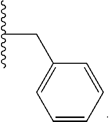 [SMILES: *Cc1ccccc1]

In some embodiments, R⁴ is

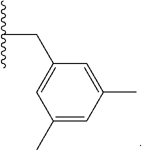 [SMILES: *Cc1cc(C)cc(C)c1]

In some embodiments, R⁴ is

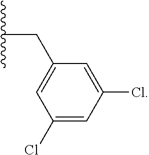 [SMILES: *Cc1cc(Cl)cc(Cl)c1]

In some embodiments, R⁴ is

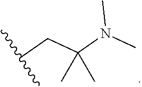 [SMILES: *CC(C)(C)N(C)C]

In some embodiments, R⁴ is

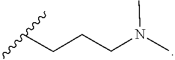 [SMILES: *CCCN(C)C]

In some embodiments, R⁴ is

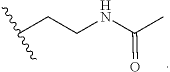 [SMILES: *CCNC(C)=O]

In some embodiments, R⁴ is

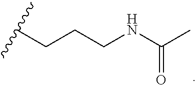 [SMILES: *CCCNC(C)=O]

In some embodiments, R⁴ is

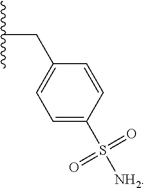 [SMILES: *Cc1ccc(S(N)(=O)=O)cc1]

In some embodiments, the bicyclic compound comprises at least one aromatic ring. In some embodiments, the at least one aromatic ring is aryl. In some embodiments, the at least one aromatic ring is heteroaryl. In some embodiments, the at least one aromatic ring is optionally substituted. In further embodiments, the at least one aromatic ring is substituted with at least one amine group.

In some embodiments, for example, the at least one aromatic ring comprises a pyridine ring. In some embodiments, the at least one aromatic ring is a pyridine ring. In some embodiments, the pyridine ring is substituted with at least one amine group. Without being bound by a particular theory, the at least one amine group attached to the at least one aromatic ring provides superior docking of the compound of Formula (I) disclosed herein, and/or pharmaceutically acceptable salts thereof, to effectively act as inhibitors of the enzymatic activity of vanin enzymes (e.g., vanin-1).

In several embodiments, Formula (I) may be represented by one or more of the following compounds (or others):

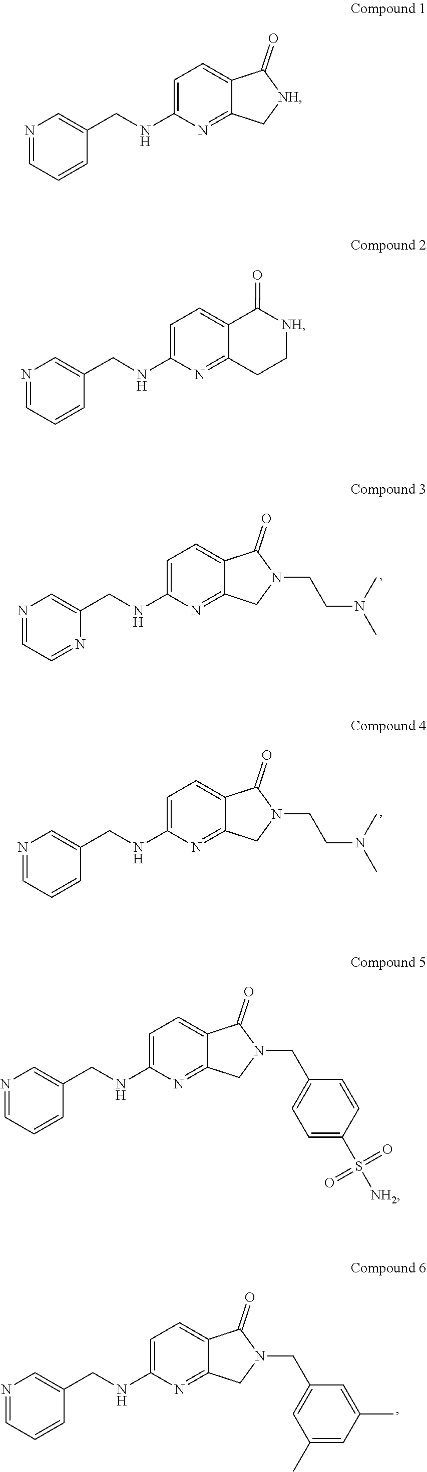 [SMILES: O=C1NCc2nc(NCc3cccnc3)ccc21 ; O=C1NCCc2nc(NCc3cccnc3)ccc21 ; CN(C)CCN1Cc2nc(NCc3cnccn3)ccc2C1=O ; CN(C)CCN1Cc2nc(NCc3cccnc3)ccc2C1=O ; NS(=O)(=O)c1ccc(CN2Cc3nc(NCc4cccnc4)ccc3C2=O)cc1 ; Cc1cc(C)cc(CN2Cc3nc(NCc4cccnc4)ccc3C2=O)c1]

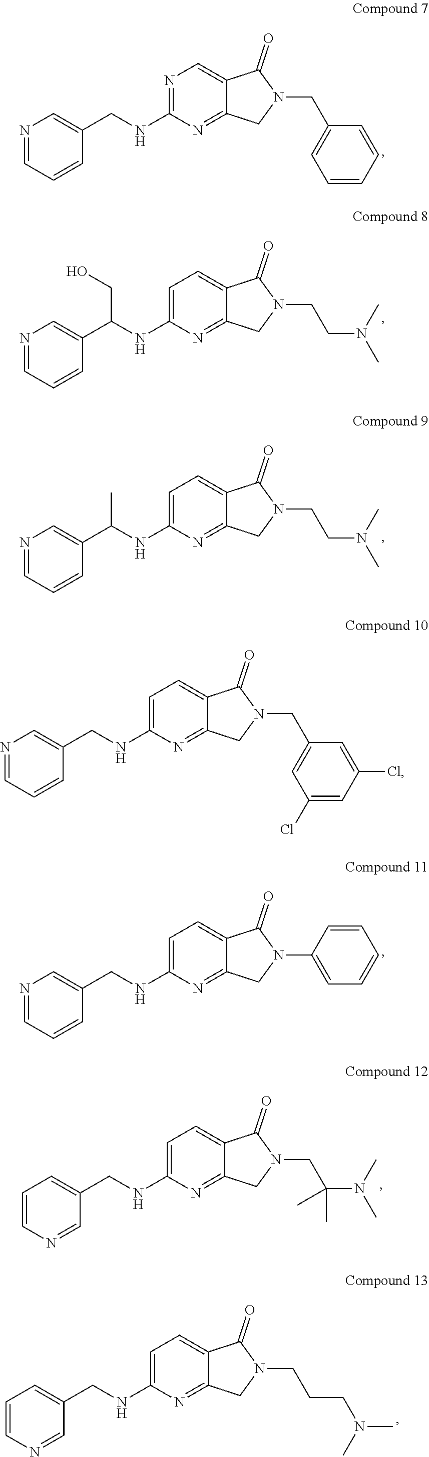

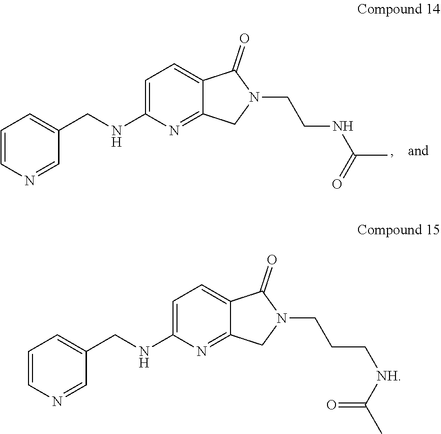 [SMILES: CC(=O)NCCN1Cc2nc(NCc3cccnc3)ccc2C1=O ; CC(=O)NCCCN1Cc2nc(NCc3cccnc3)ccc2C1=O]

In some embodiments, the compound having the structure of Formula (I) is selected from one or more of the following:
2-((pyridin-3-ylmethyl)amino)-6,7-dihydro-5H-pyrrolo[3,4-b]pyridin-5-one;
2-((pyridin-3-ylmethyl)amino)-7,8-dihydro-1,6-naphthyridin-5(6H)-one;
6-(2-(dimethylamino)ethyl)-2-((pyrazin-2-ylmethyl)amino)-6,7-dihydro-5H-pyrrolo[3,4-b]pyridin-5-one;
6-(2-(dimethylamino)ethyl)-2-((pyridin-3-ylmethyl)amino)-6,7-dihydro-5H-pyrrolo[3,4-b]pyridin-5-one;
4-((5-oxo-2-((pyridin-3-ylmethyl)amino)-5,7-dihydro-6H-pyrrolo[3,4-b]pyridin-6-yl)methyl)benzenesulfonamide;
6-(3,5-dimethylbenzyl)-2-((pyridin-3-ylmethyl)amino)-6,7-dihydro-5H-pyrrolo[3,4-b]pyridin-5-one;
6-benzyl-2-((pyridin-3-ylmethyl)amino)-6,7-dihydro-5H-pyrrolo[3,4-d]pyrimidin-5-one;
6-(2-(dimethylamino)ethyl)-2-((2-hydroxy-1-(pyridin-3-yl)ethyl)amino)-6,7-dihydro-5H-pyrrolo[3,4-b]pyridin-5-one;
6-(2-(dimethylamino)ethyl)-2-((1-(pyridin-3-yl)ethyl)amino)-6,7-dihydro-5H-pyrrolo[3,4-b]pyridin-5-one;
6-(3,5-dichlorobenzyl)-2-((pyridin-3-ylmethyl)amino)-6,7-dihydro-5H-pyrrolo[3,4-b]pyridin-5-one;
6-phenyl-2-((pyridin-3-ylmethyl)amino)-6,7-dihydro-5H-pyrrolo[3,4-b]pyridin-5-one;
6-(2-(dimethylamino)-2-methylpropyl)-2-((pyridine-3-ylmethyl)amino)-6,7-dihydro-5H-pyrrolo[3,4-b]pyridine-5-one;
6-(3-(dimethylamino)propyl)-2-((pyridin-3-ylmethyl)amino)-6,7-dihydro-5H-pyrrolo[3,4-b]pyridin-5-one;
N-(2-(5-oxo-2-((pyridin-3-ylmethyl)amino)-5,7-dihydro-6H-pyrrolo[3,4-b]pyridin-6-yl)ethyl)acetamide; and
N-(3-(5-oxo-2-((pyridin-3-ylmethyl)amino)-5,7-dihydro-6H-pyrrolo[3,4-b]pyridin-6-yl)propyl)acetamide.

### Methods of Making Compounds of Formula (I)

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(1) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

Several non-limiting embodiments pertain to methods of synthesizing compounds of Formula (I) (e.g., Compound 1, Compound 2, etc.) and intermediates of compounds of Formula (I).

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

In some embodiments, the compound of Formula (I), or a pharmaceutically acceptable salt thereof, has a half-maximal inhibitory concentration (IC₅₀) activity of, of about, less than, or less than about 20 μM, 15 μM, 10 μM, 5 μM, 4 μM, 3 μM, 2 μM, 1.9 μM, 1.8 μM, 1.7 μM, 1.6 μM, 1.5 μM, 1.4 μM, 1.3 μM, 1.2 μM, 1.1 μM, 1 μM, 0.9 μM, 0.8 μM, 0.7 μM, 0.6 μM, 0.5 μM, 0.4 μM, 0.3 μM, 0.2 μM, 0.1 μM, 0.09 μM, 0.08 μM, 0.07 μM, 0.06 μM, 0.05 μM, 0.04 μM, 0.03 μM, 0.02 μM, or 0.01 μM, or any range of values therebetween. For example, in some embodiments, the compound of Formula (I), or a pharmaceutically acceptable salt thereof, has a half-maximal inhibitory concentration (IC₅₀) activity of or of about in any one of the following ranges: about 1 μM to about 10 μM, about 5 μM to about 15 μM, about 5 μM to about 10 μM, about 10 μM to about 20 μM, about 0.5 μM to about 1 μM, about 1 μM to about 5 μM, about 0.1 μM to about 1 μM, about 0.01 μM to about 0.9 μM, about 1 μM to about 2 μM, or about 0.8 μM to about 0.9 μM.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof, and (b) a pharmaceutically acceptable carrier, diluent, excipient or combination thereof.

In addition to the selected compound useful as described above, some embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, subcutaneous, or other parental routes of administration. In some embodiments, the compositions may be in a form suitable for subcutaneous administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one or more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 to about 99.99 wt % of a compound of the present technology based on the total formulation, with the balance being one or more suitable pharmaceutical excipients. Preferably, the compound is present at a level of about 1-80 wt %. Representative pharmaceutical formulations are described below.

### FORMULATION EXAMPLES

The following are representative pharmaceutical formulations containing a compound of Formula (I).

### Formulation Example 1—Tablet Formulation

The following ingredients are mixed intimately and pressed into single scored tablets.

TABLE 1
Ingredients to be included in a tablet formulation.

| Ingredient | Quantity per tablet, mg |
|---|---|
| Compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

TABLE 2
Ingredients to be included in a capsule formulation.

| Ingredient | Quantity per capsule, mg |
|---|---|
| Compounds disclosed herein | 200 |
| lactose, spray-dried | 148 |
| magnesium stearate | 2 |

### Formulation Example 3—Suspension Formulation

The following ingredients are mixed to form a suspension for oral administration.

TABLE 3
Ingredients to be included in a suspension formulation.

| Ingredient | Amount | |
|---|---|---|
| Compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4—Injectable Formulation

The following ingredients are mixed to form an injectable formulation.

TABLE 4
Ingredients to be included in an injectable formulation.

| Ingredient | Amount |
|---|---|
| Compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

TABLE 5
Ingredients to be included in a suppository formulation.

| Ingredient | Amount |
|---|---|
| Compounds disclosed herein | 500 mg |
| Witepsol ® H-15 | balance |

### Methods of Treatment

The compounds of Formula (I) disclosed herein or their tautomers and/or pharmaceutically acceptable salts thereof can effectively act as inhibitors of the enzymatic activity of vanin enzymes (e.g., vanin-1). Some embodiments provide pharmaceutical compositions comprising one or more compounds disclosed herein and a pharmaceutically acceptable excipient.

Some embodiments provide a method of preventing, treating, or ameliorating one or more inflammatory or autoimmune diseases in a subject. In some embodiments, the method includes administering one or more of the compounds disclosed herein to a subject in need thereof. In some embodiments, the method includes administering a pharmaceutically acceptable salt thereof of one or more of the compounds disclosed herein to a subject in need thereof.

In several embodiments, the disclosed compound is used to prevent, treat, or ameliorate ulcerative colitis, Crohn's disease, rheumatoid arthritis, atopic dermatitis, psoriasis, lupus (e.g., systemic erythematosus lupus), atherosclerosis, and Type 1 diabetes. Some embodiments provide a method preventing, treating, or ameliorating Lupus, rheumatoid arthritis, atopic dermatitis, and psoriasis. In some embodiments, the method includes administering one or more of the compounds disclosed herein to a subject in need thereof. In some embodiments, the method includes administering a pharmaceutically acceptable salt thereof of one or more of the compounds disclosed herein to a subject in need thereof.

In several embodiments, the disclosed compound is used to treat a cancer. In some embodiments, the cancer is selected from the group consisting of colon cancer, liver cancer, pancreatic cancer, gastric cancer, esophageal cancer, prostate cancer, breast cancer, cholangiocarcinomas, sarcomas, or acute myeloid leukemia. In some embodiments, the method includes administering one or more of the compounds disclosed herein to a subject in need thereof. In some embodiments, the method includes administering a pharmaceutically acceptable salt thereof of one or more of the compounds disclosed herein to a subject in need thereof.

In some embodiments, the method of administering one or more of the compounds disclosed herein results in the prevention, treatment, or amelioration, of an inflammatory or autoimmune disease. In some embodiments, the method of administering one or more of the compounds disclosed herein results in the prevention, treatment, or amelioration, of ulcerative colitis, Crohn's disease, atopic dermatitis, psoriasis, systemic erythematosus lupus, atherosclerosis, and Type 1 diabetes. In some embodiments, the method includes administering a pharmaceutically acceptable salt thereof of one or more of the compounds disclosed herein.

In some embodiments, the method of administering one or more of the compounds disclosed herein results in the inhibition of the activity of vanin-1 in one or more organs of said subject. In several embodiments, inhibiting the activity of vanin-1 suppresses the expression and/or activity of pro-inflammatory signals. In several embodiments, the pro-inflammatory signals may one or more cytokines. In several embodiments, the cytokine includes TNF-alpha (TNFA or TNFα), interleukin 6 (IL6), interleukin lbeta (IL1B), MCP1 and interleukin 8 (IL8). In some embodiments, the method includes administering a pharmaceutically acceptable salt thereof of one or more of the compounds disclosed herein.

Some embodiments include co-administering a compound, composition, and/or pharmaceutical composition described herein, with an additional medicament. By “co-administration,”it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered subcutaneously, another being administered orally and another being administered i.v.

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### EXAMPLES

### Example 1: Synthesis of Bicyclic Compounds

All reactions were carried out under an atmosphere of argon. Reagents and solvents were used from commercial sources without additional purification. Hydrogenation reactions were run under a balloon. Microwave reactions were performed using a CEM Discover SP microwave synthesizer. Sample purification was conducted on a Buchi Pureflash with ELSD purification system using pre-packed commercially available silica gel columns. Thin layer chromatography (TLC) was performed on aluminum plates using Merck Kieselgel 60 F254 (230-400 mesh) fluorescent treated silica which were visualized under ultraviolet light (254 nm), or by staining with potassium permanganate or ninhydrin solution as appropriate. All Nuclear Magnetic Resonance (NMR) spectra were acquired on a Bruker Avance III HD 400 MHz NMR spectrometer; chemical shifts are reported in ppm (δ). HPLC/MS was performed on a Shimadzu-2020 single quad mass spectrometry coupled with Shimadzu LC40B XR UHPLC using Shimadzu Nexcol C18 column (50×2.1 mm, 1.8 μm particle size) via following method: The gradient mobile phase A contains 0.1% formic acid in water and mobile phase B contains 0.1% formic acid in acetonitrile; A/B (95:5) from 0 to 0.9 minutes; to A/B (5:95) from 0.9 to 2.2 minutes; A/B (5:95) from 2.2 to 4.14 minutes; to A/B (95:5) from 4.14 to 4.20 minutes; A/B (95:5) from 4.2 to 6 minutes. The flow rate was 0.4 mL/min and the column temperature maintained at 35° C. and autosampler temperature at 4° C. Ion spray voltage, drying gas temperature, ion source gas 1, and ion source gas 2 settings were 4500V, 500° C., 35V, and 45V with ESI set in positive mode using full scan. All compounds purity was analyzed on Agilent 1260 Infinity II Lab LC Series HPLC (1260 Quat pum, 1260 vial autosampler, ICC column oven, 1260 DAD WR detector). Samples were injected into Phenomenex Synergi Polar RP column (150×4.6 mm, 4 μm, 80 Å). The gradient mobile phase (A: water with 0.1% trifluoroacetic acid, B: acetonitrile with 0.1% trifluoroacetic acid; A/B (99:1) from 0 minute; to A/B (1:99) from 0 to 15 minutes; A/B (1:99) from 15 to 18 minutes; A/B (99:1) from 18 to 18.1 minutes; A/B (99:1) from 18.1 to 20 minutes) pumped at a flow rate of 1 mL/min. UV detector was set to 254 nm with column oven at 35° C. Injection volume was 10 μL, unless otherwise specified. All compounds that were evaluated in biological assay had >90% and animal study had >95% purity.

### Example 2: Synthesis of 2-((Pyridin-3-ylmethyl)amino)-6,7-dihydro-5H-pyrrolo[3,4-b]pyridin-5-one (Compound 1)

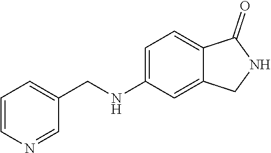 [SMILES: O=C1NCc2cc(NCc3cccnc3)ccc21]

Pyridin-3-ylmethanamine (128 mg, 1.18 mmol) was added to a solution of 2-chloro-6,7-dihydro-5H-pyrrolo[3,4-b]pyridin-5-one (100 mg, 593.19 μmol) and DIPEA (0.52 mL, 2.99 mmol) in 1-BuOH (2 mL) at room temperature. The mixture was then heated to 140° C. overnight and cooled to room temperature. The reaction mixture was partitioned between water and Ethyl acetate. The layers were separated, and the aq. layer was further extracted with EA (2×25 mL). The combined organic layers were washed with brine, dried over anhydrous Na₂SO₄, filtered and concentrated. The residue was purified by Buchi Pureflash chromatography (silica, 40 g) using 0-5% MeOH in DCM as eluent to obtain desired product 2-((pyridin-3-ylmethyl)amino)-6,7-dihydro-5H-pyrrolo[3,4-b]pyridin-5-one (Compound 1) (103 mg, 72% yield) as white solid. ¹H NMR (400 MHz, DMSO) δ 8.57 (dd, J=2.3, 0.9 Hz, 1H), 8.45 (dd, J=4.8, 1.7 Hz, 1H), 8.03 (s, 1H), 7.84 (t, J=5.9 Hz, 1H), 7.73 (ddd, J=7.8, 2.3, 1.7 Hz, 1H), 7.60 (d, J=8.6 Hz, 1H), 7.35 (ddd, J=7.8, 4.8, 0.9 Hz, 1H), 6.56 (d, J=8.6 Hz, 1H), 4.58 (d, J=5.9 Hz, 2H), 4.15 (d, J=1.1 Hz, 2H). MS (ESI): Calcd. For C₁₃H₁₂N₄O: 240. found 241 (M+H)+.

### Example 3: Synthesis of 2-((Pyridin-3-ylmethyl)amino)-7,8-dihydro-1,6-naphthyridin-5(6H)-one (Compound 2)

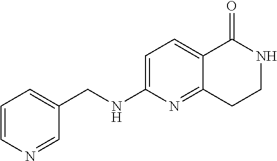 [SMILES: O=C1NCCc2nc(NCc3cccnc3)ccc21]

Pyridin-3-ylmethanamine (267 mg, 2.47 mmol) was added to a solution of 2-chloro-7,8-dihydro-1,6-naphthyridin-5(6H)-one (300 mg, 1.64 mmol) and DIPEA (0.58 mL, 3.33 mmol) in 1-BuOH (2 mL) at room temperature. The mixture was then heated to 140° C. overnight and cooled to room temperature. The reaction mixture was partitioned between water and Ethyl acetate. The layers were separated, and the aq. layer was further extracted with EA (2×25 mL). The combined organic layers were washed with brine, dried over anhydrous Na₂SO₄, filtered and concentrated. The residue was purified by Buchi Pureflash chromatography (silica, 40 g) using 0-5% MeOH in DCM as eluent to obtain desired product 2-((pyridin-3-ylmethyl)amino)-7,8-dihydro-1,6-naphthyridin-5(6H)-one (Compound 2) (150 mg, 36% yield) as beige solid. ¹H NMR (400 MHz, DMSO) δ 8.57 (dd, J=2.3, 0.8 Hz, 1H), 8.44 (dd, J=4.8, 1.7 Hz, 1H), 7.72 (dd, J=8.3, 6.2 Hz, 3H), 7.50 (s, 1H), 7.34 (ddd, J=7.8, 4.8, 0.9 Hz, 1H), 6.44 (d, J=8.6 Hz, 1H), 4.54 (d, J=6.0 Hz, 2H), 3.35 (d, J=2.7 Hz, 1H), 3.32 (d, J=2.7 Hz, 1H), 2.78 (t, J=6.7 Hz, 2H). MS (ESI): Calcd. For C₁₄H₁₄N₄O: 254. found 255 (M+H)+.

### Example 4: Synthesis of 2-Chloro-6-(2-(dimethylamino)ethyl)-6,7-dihydro-5H-pyrrolo[3,4-b]pyridin-5-one (Compound 3A)

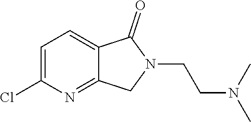 [SMILES: CN(C)CCN1Cc2nc(Cl)ccc2C1=O]

Methyl 6-chloro-2-(chloromethyl)nicotinate (50 mg, 227.22 μmol) and N1,N1-dimethylethane-1,2-diamine (100 mg, 1.13 mmol) was stirred in THE (3 mL) at room temperature overnight. Water was added and the mixture extracted with EtOAc (2×10 mL). The combined organic extracts were washed with brine, dried over MgSO₄, filtered, and concentrated in vacuo. The residue was purified by Buchi Pureflash chromatography (silica, 24 g) using 0-60% EtOAc in Hexane to obtain the desired product 2-chloro-6-(2-(dimethylamino)ethyl)-6,7-dihydro-5H-pyrrolo[3,4-b]pyridin-5-one (Compound 3A) (50 mg, 93% yield) as white solid. ¹H NMR (400 MHz, DMSO) δ 8.11 (d, J=8.0 Hz, 1H), 7.63 (d, J=8.0 Hz, 1H), 4.58 (s, 2H), 3.63 (t, J=6.3 Hz, 2H), 2.48 (d, J=6.6 Hz, 2H), 2.17 (s, 6H). MS (ESI): Calcd. For C₁₁H₁₄ClN₃₀: 239. found 240 (M+H)⁺.

### Example 5: Synthesis of 6-(2-(Dimethylamino)ethyl)-2-((pyridin-3-ylmethyl)amino)-6,7-dihydro-5H-pyrrolo[3,4-b]pyridin-5-one (Compound 4)

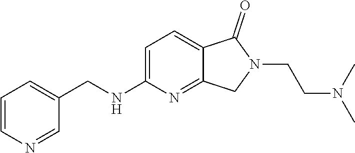 [SMILES: CN(C)CCN1Cc2nc(NCc3cccnc3)ccc2C1=O]

Pyridin-3-ylmethanamine (315 mg, 2.91 mmol) was added to a solution of 2-chloro-6-(2-(dimethylamino)ethyl)-6,7-dihydro-5H-pyrrolo[3,4-b]pyridin-5-one (70 mg, 292.03 μmol) and DIPEA (0.25 mL, 1.44 mmol) in 1-BuOH (2 mL) at room temperature. The mixture was then heated to 140° C. overnight and cooled to room temperature. The reaction mixture was concentrated under vacuo and the residue was purified by Buchi Pureflash chromatography (silica, 24 g) using 0-10% 0.7N NH₃-MeOH in DCM as eluent to obtain desired product 6-(2-(dimethylamino)ethyl)-2-((pyridin-3-ylmethyl)amino)-6,7-dihydro-5H-pyrrolo[3,4-b]pyridin-5-one (Compound 4) (33 mg, 47% yield) as white solid. ¹H NMR (400 MHz, DMSO) δ 8.56 (dd, J=2.2, 0.9 Hz, 1H), 8.45 (dd, J=4.8, 1.7 Hz, 1H), 7.86 (t, J=5.9 Hz, 1H), 7.73 (dt, J=7.8, 2.0 Hz, 1H), 7.59 (d, J=8.5 Hz, 1H), 7.35 (ddd, J=7.8, 4.8, 0.9 Hz, 1H), 6.56 (d, J=8.6 Hz, 1H), 4.57 (d, J=5.9 Hz, 2H), 4.27 (s, 2H), 3.51 (t, J=6.4 Hz, 2H), 2.42 (t, J=6.4 Hz, 2H), 2.15 (s, 6H). MS (ESI): Calcd. For C₁₇H₂₁N₅O: 311. found 312 (M+H)⁺.

### Example 6: Synthesis of 2-Chloro-6-(3,5-dimethylbenzyl)-6,7-dihydro-5H-pyrrolo[3,4-b]pyridin-5-one (Compound 6A)

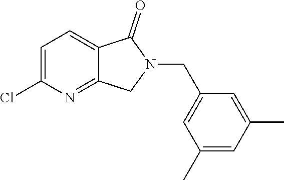 [SMILES: Cc1cc(C)cc(CN2Cc3nc(Cl)ccc3C2=O)c1]

Lithium bis(trimethylsilyl) amide (0.60 mL, 1 M in THF, 600 μmol) was added dropwise at 0° C. to a solution of 2-chloro-6,7-dihydro-5H-pyrrolo[3,4-b]pyridin-5-one (100 mg, 593.19 μmol) in THF (8 mL). After stirring for 10 min, 1-(bromomethyl)-3,5-dimethylbenzene (142 mg, 713.24 μmol) was added dropwise. The resulting reaction mixture was allowed to stir at room temperature for 48 hours before quenching with saturated aqueous ammonium chloride solution. After extraction with EtOAc, the organic layer was washed with brine, dried over anhydrous Na₂SO₄, filtered and concentrated in vacuo to give the crude product. It was further purified by Buchi Pureflash chromatography (silica, 24 g) using 0-60% EtOAc in hexane to obtain the desired product 2-chloro-6-(3,5-dimethylbenzyl)-6,7-dihydro-5H-pyrrolo[3,4-b]pyridin-5-one (Compound 6A) (70 mg, 41% yield) as greenish solid. MS (ESI): Calcd. For C₁₆H₁₅ClN₂O: 286. found 287 (M+H)⁺.

### Example 7: Synthesis of 6-(3,5-Dimethylbenzyl)-2-((pyridin-3-ylmethyl)amino)-6,7-dihydro-5H-pyrrolo[3,4-b]pyridin-5-one (Compound 6)

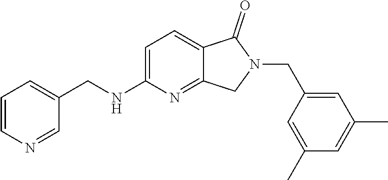 [SMILES: Cc1cc(C)cc(CN2Cc3nc(NCc4cccnc4)ccc3C2=O)c1]

Pyridin-3-ylmethanamine (264 mg, 2.44 mmol) was added to a solution of 2-chloro-6-(3,5-dimethylbenzyl)-6,7-dihydro-5H-pyrrolo[3,4-b]pyridin-5-one (70 mg, 244.11 μmol) and DIPEA (0.21 mL, 1.21 mmol) in 1-BuOH (2 mL) at room temperature. The mixture was then heated to 140° C. overnight and cooled to room temperature. The reaction mixture was partitioned between water and ethyl acetate. The layers were separated, and the aq. layer was further extracted with EA (2×25 mL). The combined organic layers were washed with brine, dried over anhydrous Na₂SO₄, filtered and concentrated. The residue was purified by Buchi Pureflash chromatography (silica, 24 g) using 0-5% 0.7N NH₃-MeOH in DCM as eluent to obtain desired product 6-(3,5-dimethylbenzyl)-2-((pyridin-3-ylmethyl)amino)-6,7-dihydro-5H-pyrrolo[3,4-b]pyridin-5-one (Compound 6) (41 mg, 47% yield) as white solid. ¹H NMR (400 MHz, DMSO) δ 8.69-8.52 (m, 1H), 8.46 (ddd, J=20.4, 4.8, 1.7 Hz, 1H), 7.90 (t, J=6.0 Hz, 1H), 7.79-7.68 (m, 1H), 7.64 (d, J=8.6 Hz, 1H), 7.36 (dddd, J=20.3, 7.8, 4.8, 1.0 Hz, 1H), 6.91-6.81 (m, 3H), 6.58 (d, J=8.6 Hz, 1H), 4.55 (d, J=4.9 Hz, 4H), 4.12 (s, 2H), 2.25-2.17 (m, 6H). MS (ESI): Calcd. For C₂₂H₂₂N₄O: 358. found 359 (M+H)⁺.

### Example 8: Synthesis of 2-Chloro-6-(2-(dimethylamino)-2-methylpropyl)-6,7-dihydro-5H-pyrrolo[3,4-b]pyridin-5-one (Compound 12A)

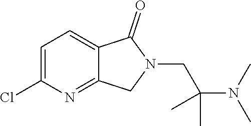 [SMILES: CN(C)C(C)(C)CN1Cc2nc(Cl)ccc2C1=O]

Methyl 6-chloro-2-(chloromethyl)nicotinate (150 mg, 681.67 μmol) and N1,N1-dimethylpropane-1,3-diamine (348 mg, 3.41 mmol) was stirred in THE (5 mL) at room temperature overnight. The reaction was concentrated, and the residue was purified by Buchi Pureflash chromatography (silica, 24 g) using 0-5% 0.7N NH₃-MeOH in DCM to obtain the desired product 2-chloro-6-(2-(dimethylamino)-2-methylpropyl)-6,7-dihydro-5H-pyrrolo[3,4-b]pyridin-5-one (Compound 12A) (123 mg, 67% yield) as white solid. ¹H NMR (400 MHz, DMSO) δ 8.13 (d, J=8.1 Hz, 1H), 7.62 (dd, J=8.1, 0.8 Hz, 1H), 4.80 (s, 2H), 3.53 (s, 2H), 2.20 (s, 6H), 0.96 (s, 6H). MS (ESI): Calcd. For C₁₃H₁₈ClN₃O: 267. found 268 (M+H)⁺.

### Example 9: Synthesis of 6-(2-(Dimethylamino)-2-methylpropyl)-2-((pyridin-3-ylmethyl)amino)-6,7-dihydro-5H-pyrrolo[3,4-b]pyridin-5-one (Compound 12)

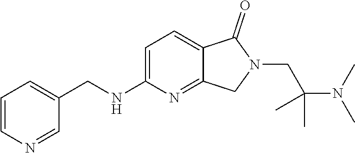 [SMILES: CN(C)C(C)(C)CN1Cc2nc(NCc3cccnc3)ccc2C1=O]

Pyridin-3-ylmethanamine (202 mg, 1.87 mmol) was added to a solution 2-chloro-6-(2-(dimethylamino)-2-methylpropyl)-6,7-dihydro-5H-pyrrolo[3,4-b]pyridin-5-one (100 mg, 373.47 μmol) and DIPEA (0.32 mL, 1.84 mmol) in 1-BuOH (4 mL) at room temperature. The mixture was then heated to 140° C. overnight and cooled to room temperature. The reaction mixture was concentrated under vacuo and the residue was purified by Buchi Pureflash chromatography (silica, 40 g) using 0-10% 0.7N NH₃-MeOH in DCM as eluent to obtain desired product 6-(2-(dimethylamino)-2-methylpropyl)-2-((pyridin-3-ylmethyl)amino)-6,7-dihydro-5H-pyrrolo[3,4-b]pyridin-5-one (Compound 12) (63 mg, 50% yield) as white solid. ¹H NMR (400 MHz, DMSO) δ 8.56 (dd, J=2.3, 0.8 Hz, 1H), 8.45 (dd, J=4.8, 1.7 Hz, 1H), 7.86 (t, J=5.9 Hz, 1H), 7.73 (dt, J=7.9, 2.0 Hz, 1H), 7.60 (d, J=8.6 Hz, 1H), 7.35 (ddd, J=7.8, 4.8, 0.9 Hz, 1H), 6.56 (d, J=8.6 Hz, 1H), 4.58 (d, J=5.9 Hz, 2H), 4.49 (s, 2H), 3.42 (s, 2H), 2.20 (s, 6H), 0.94 (s, 6H). MS (ESI): Calcd. For C₁₉H₂₅N₅O: 339. found 340 (M+H)⁺.

### Example 10: Synthesis of 2-Chloro-6-(3-(dimethylamino)propyl)-6,7-dihydro-5H-pyrrolo[3,4-b]pyridin-5-one (Compound 13A)

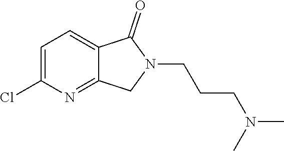 [SMILES: CN(C)CCCN1Cc2nc(Cl)ccc2C1=O]

Methyl 6-chloro-2-(chloromethyl)nicotinate (150 mg, 681.67 μmol) and N2,N2,2-trimethylpropane-1,2-diamine (348 mg, 3.41 mmol) was stirred in THE (5 mL) at room temperature overnight. The reaction was concentrated, and the residue was purified by Buchi Pureflash chromatography (silica, 24 g) using 0-5% 0.7N NH₃-MeOH in DCM to obtain the desired product 2-chloro-6-(3-(dimethylamino)propyl)-6,7-dihydro-5H-pyrrolo[3,4-b]pyridin-5-one (Compound 13A) (123 mg, 67% yield) as white solid. ¹H NMR (400 MHz, DMSO) δ 8.09 (d, J=8.0 Hz, 1H), 7.61 (d, J=8.1 Hz, 1H), 4.54 (s, 2H), 3.54 (dd, J=7.9, 6.7 Hz, 3H), 2.22 (t, J=7.1 Hz, 2H), 2.11 (s, 6H), 1.73 (p, J=7.1 Hz, 2H). MS (ESI): Calcd. For C₁₂H₁₆ClN₃O: 253. found 254 (M+H)⁺.

### Example 11: Synthesis of 6-(3-(Dimethylamino)propyl)-2-((pyridin-3-ylmethyl)amino)-6,7-dihydro-5H-pyrrolo[3,4-b]pyridin-5-one (Compound 13)

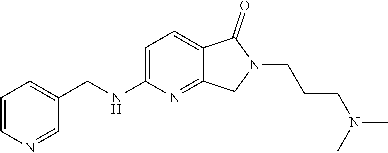 [SMILES: CN(C)CCCN1Cc2nc(NCc3cccnc3)ccc2C1=O]

Pyridin-3-ylmethanamine (213 mg, 1.97 mmol) was added to a solution 2-chloro-6-(3-(dimethylamino)propyl)-6,7-dihydro-5H-pyrrolo[3,4-b]pyridin-5-one (100 mg, 394.12 μmol) and DIPEA (0.34 mL, 1.97 mmol) in 1-BuOH (4 mL) at room temperature. The mixture was then heated to 140° C. overnight and cooled to room temperature. The reaction mixture was concentrated under vacuo and the residue was purified by Buchi Pureflash chromatography (silica, 40 g) using 0-10% 0.7N NH₃-MeOH in DCM as eluent to obtain desired product 6-(3-(dimethylamino)propyl)-2-((pyridin-3-ylmethyl)amino)-6,7-dihydro-5H-pyrrolo[3,4-b]pyridin-5-one (Compound 13) (74 mg, 58% yield) as white solid. ¹H NMR (400 MHz, DMSO) δ 8.57 (dd, J=2.3, 0.9 Hz, 1H), 8.46 (dd, J=4.8, 1.7 Hz, 1H), 8.01 (t, J=6.0 Hz, 1H), 7.80-7.71 (m, 1H), 7.60 (d, J=8.5 Hz, 1H), 7.36 (ddd, J=7.8, 4.8, 0.9 Hz, 1H), 6.60 (d, J=8.6 Hz, 1H), 4.58 (d, J=5.9 Hz, 2H), 4.29 (s, 2H), 3.51 (t, J=6.5 Hz, 2H), 2.99 (t, J=8.2 Hz, 2H), 2.70 (s, 6H), 2.05-1.89 (m, 2H). MS (ESI): Calcd. For C₁₈H₂₃N₅O: 325. found 326 (M+H)⁺.

### Example 12: Synthesis of N-(2-(2-chloro-5-oxo-5,7-dihydro-6H-pyrrolo[3,4-b]pyridin-6-yl)ethyl)acetamide (Compound 14A)

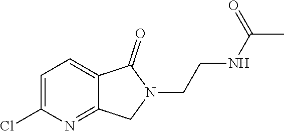 [SMILES: CC(=O)NCCN1Cc2nc(Cl)ccc2C1=O]

Methyl 6-chloro-2-(chloromethyl)nicotinate (150 mg, 681.67 μmol) and N-(2-aminoethyl)acetamide (348 mg, 3.41 mmol) was stirred in THE (5 mL) at room temperature overnight. The reaction was concentrated, and the residue was purified by Buchi Pureflash chromatography (silica, 24 g) using 0-5% 0.7N NH₃-MeOH in DCM to obtain the desired product N-(2-(2-chloro-5-oxo-5,7-dihydro-6H-pyrrolo[3,4-b]pyridin-6-yl)ethyl)acetamide (Compound 14A) (169 mg, 98% yield) as white solid. ¹H NMR (400 MHz, DMSO) δ 8.12 (d, J=8.1 Hz, 1H), 8.01-7.92 (m, 1H), 7.65-7.60 (m, 1H), 4.56 (s, 2H), 3.57 (dd, J=6.6, 5.4 Hz, 2H), 3.35-3.30 (m, 2H), 1.74 (s, 3H). MS (ESI): Calcd. For C₁₁H₁₂ClN₃O₂: 253. found 254 (M+H)⁺.

### Example 13: Synthesis of N-(2-(5-Oxo-2-((pyridin-3-ylmethyl)amino)-5,7-dihydro-6H-pyrrolo[3,4-b]pyridin-6-yl)ethyl)acetamide (Compound 14)

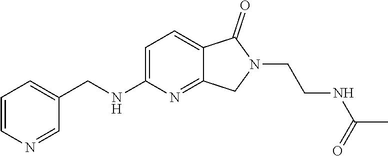 [SMILES: CC(=O)NCCN1Cc2nc(NCc3cccnc3)ccc2C1=O]

Pyridin-3-ylmethanamine (213 mg, 1.97 mmol) was added to a solution product N-(2-(2-chloro-5-oxo-5,7-dihydro-6H-pyrrolo[3,4-b]pyridin-6-yl)ethyl)acetamide (100 mg, 394.19 μmol) and DIPEA (0.34 mL, 1.97 mmol) in 1-BuOH (4 mL) at room temperature. The mixture was then heated to 140° C. overnight and cooled to room temperature. The reaction mixture was concentrated under vacuo and the residue was purified by Buchi Pureflash chromatography (silica, 40 g) using 0-10% MeOH in DCM as eluent to obtain desired product N-(2-(5-oxo-2-((pyridin-3-ylmethyl)amino)-5,7-dihydro-6H-pyrrolo[3,4-b]pyridin-6-yl)ethyl)acetamide (Compound 14) (101 mg, 79% yield) as white solid. ¹H NMR (400 MHz, DMSO) δ 8.56 (dd, J=2.3, 0.8 Hz, 1H), 8.45 (dd, J=4.8, 1.7 Hz, 1H), 7.94 (t, J=5.9 Hz, 1H), 7.87 (t, J=5.9 Hz, 1H), 7.73 (dt, J=7.8, 2.0 Hz, 1H), 7.59 (d, J=8.6 Hz, 1H), 7.34 (ddd, J=7.8, 4.8, 0.9 Hz, 1H), 6.56 (d, J=8.6 Hz, 1H), 4.57 (d, J=5.9 Hz, 2H), 4.27 (s, 2H), 3.51-3.42 (m, 2H), 3.25 (q, J=6.0 Hz, 2H), 1.74 (s, 3H). MS (ESI): Calcd. For C₁₇H₁₉N₅O₂: 325. found 326 (M+H)⁺.

### Example 14: Synthesis of N-(3-(2-chloro-5-oxo-5,7-dihydro-6H-pyrrolo[3,4-b]pyridin-6-yl)propyl)acetamide (Compound 15A)

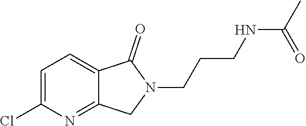 [SMILES: CC(=O)NCCCN1Cc2nc(Cl)ccc2C1=O]

Methyl 6-chloro-2-(chloromethyl)nicotinate (150 mg, 681.67 μmol) and N-(3-aminopropyl)acetamide (396 mg, 3.41 mmol) was stirred in THE (5 mL) at room temperature overnight. The reaction was concentrated, and the residue was purified by Buchi Pureflash chromatography (silica, 24 g) using 0-5% 0.7N NH₃-MeOH in DCM to obtain the desired product N-(3-(2-chloro-5-oxo-5,7-dihydro-6H-pyrrolo[3,4-b]pyridin-6-yl)propyl)acetamide (Compound 15A) (83 mg, 45% yield) as white solid. MS (ESI): Calcd. For C₁₂H₁₄ClN₃O₂: 267. found 268 (M+H)⁺.

### Example 15: Synthesis of N-(3-(5-Oxo-2-((pyridin-3-ylmethyl)amino)-5,7-dihydro-6H-pyrrolo[3,4-b]pyridin-6-yl)propyl)acetamide (Compound 15)

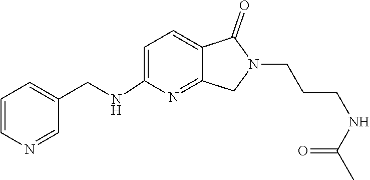 [SMILES: CC(=O)NCCCN1Cc2nc(NCc3cccnc3)ccc2C1=O]

Pyridin-3-ylmethanamine (162 mg, 1.50 mmol) was added to a solution product N-(3-(2-chloro-5-oxo-5,7-dihydro-6H-pyrrolo[3,4-b]pyridin-6-yl)propyl)acetamide (80 mg, 298.83 μmol) and DIPEA (0.26 mL, 1.49 mmol) in 1-BuOH (3 mL) at room temperature. The mixture was then heated to 140° C. overnight and cooled to room temperature. The reaction mixture was concentrated under vacuo and the residue was purified by Buchi Pureflash chromatography (silica, 40 g) using 0-10% MeOH in DCM as eluent to obtain desired product N-(3-(5-oxo-2-((pyridin-3-ylmethyl)amino)-5,7-dihydro-6H-pyrrolo[3,4-b]pyridin-6-yl)propyl)acetamide (Compound 15) (54 mg, 53% yield) as white solid. ¹H NMR (400 MHz, DMSO) δ 8.57 (dd, J=2.3, 0.9 Hz, 1H), 8.45 (dd, J=4.8, 1.7 Hz, 1H), 7.90-7.81 (m, 2H), 7.77-7.70 (m, 1H), 7.59 (d, J=8.6 Hz, 1H), 7.35 (ddd, J=7.8, 4.8, 0.9 Hz, 1H), 6.56 (d, J=8.6 Hz, 1H), 4.58 (d, J=5.9 Hz, 2H), 4.25 (s, 2H), 3.42 (t, J=7.1 Hz, 2H), 3.01 (q, J=6.7 Hz, 2H), 1.79 (s, 3H), 1.67 (p, J=7.1 Hz, 2H). MS (ESI): Calcd. For C₁₈H₂₁N₅O₂: 339. found 340 (M+H)⁺.

### Example 16: Synthesis of N-(2-(5-oxo-2-((pyridin-2-ylmethyl)amino)-5,7-dihydro-6H-pyrrolo[3,4-b]pyridin-6-yl)ethyl)acetamide (Compound 20)

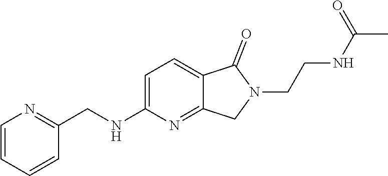 [SMILES: CC(=O)NCCN1Cc2nc(NCc3ccccn3)ccc2C1=O]

2-Pyridinemethanamine (853 mg, 7.88 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyridin-2-ylmethyl)amino)-5,7-dihydro-6H-pyrrolo[3,4-b]pyridin-6-yl)ethyl)acetamide (120 mg, 24% yield) as light yellow solid. ¹H NMR (400 MHz, CDCl₃) δ 8.56 (ddd, J=5.0, 1.8, 1.0 Hz, 1H), 7.75-7.63 (m, 2H), 7.32 (dt, J=7.8, 1.0 Hz, 1H), 7.22 (ddd, J=7.6, 4.9, 1.1 Hz, 1H), 6.57 (s, 1H), 6.49 (d, J=8.6 Hz, 1H), 6.33 (t, J=5.1 Hz, 1H), 4.72 (d, J=5.0 Hz, 2H), 4.30 (s, 2H), 3.71 (dd, J=6.8, 4.6 Hz, 2H), 3.55-3.49 (m, 2H), 1.93 (s, 3H). MS (ESI): Calcd. For C₁₇H₁₉N₅O₂: 325. found 326 (M+H)⁺.

### Example 17: Synthesis of N-(2-(5-oxo-2-((pyridin-3-ylmethyl)amino)-5,7-dihydro-6H-pyrrolo[3,4-b]pyridin-6-yl)ethyl)propionamide (Compound 21)

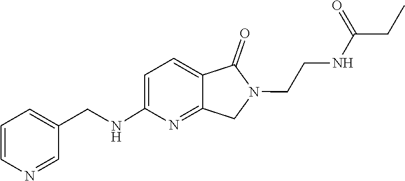 [SMILES: CCC(=O)NCCN1Cc2nc(NCc3cccnc3)ccc2C1=O]

3-Pyridinemethanamine (606 mg, 5.60 mmol) was added to a solution product N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.6 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyridin-3-ylmethyl)amino)-5,7-dihydro-6H-pyrrolo[3,4-b]pyridin-6-yl)ethyl)propionamide (90 mg, 23% yield) as light yellow solid. ¹H NMR (400 MHz, CDCl₃) δ 8.62 (d, J=2.2 Hz, 1H), 8.52 (dd, J=4.8, 1.6 Hz, 1H), 7.79-7.64 (m, 2H), 7.30-7.26 (m, 1H), 6.41 (d, J=8.6 Hz, 2H), 5.45 (t, J=5.9 Hz, 1H), 4.65 (d, J=5.9 Hz, 2H), 4.30 (s, 2H), 3.71 (dd, J=6.8, 4.6 Hz, 2H), 3.51 (q, J=5.3 Hz, 2H), 2.16 (q, J=7.6 Hz, 2H), 1.07 (t, J=7.6 Hz, 3H). MS (ESI): Calcd. For C₁₈H₂₁N₅O₂: 339. found 340 (M+H)⁺.

### Example 18: Synthesis of (R)—N-(2-(5-oxo-2-((1-(pyridin-3-yl)ethyl)amino)-5,7-dihydro-6H-pyrrolo[3,4-b]pyridin-6-yl)ethyl)acetamide (Compound 22)

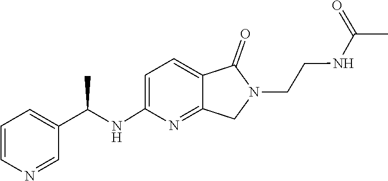 [SMILES: CC(=O)NCCN1Cc2nc(N[C@H](C)c3cccnc3)ccc2C1=O]

(R)-Methyl-3-pyridinemethanamine (578 mg, 4.73 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product (R)—N-(2-(5-oxo-2-((1-(pyridin-3-yl)ethyl)amino)-5,7-dihydro-6H-pyrrolo[3,4-b]pyridin-6-yl)ethyl)acetamide (110 mg, 21% yield) as yellow solid. ¹H NMR (400 MHz, CDCl₃) δ 8.65 (d, J=2.4 Hz, 1H), 8.51 (dd, J=4.8, 1.6 Hz, 1H), 7.68 (dd, J=8.3, 2.4 Hz, 2H), 7.31-7.26 (m, 1H), 6.42 (s, 1H), 6.29 (d, J=8.6 Hz, 1H), 5.32 (d, J=6.8 Hz, 1H), 5.06 (q, J=6.8 Hz, 1H), 4.36-4.18 (m, 2H), 3.70 (td, J=5.7, 1.9 Hz, 2H), 3.50 (p, J=5.3 Hz, 2H), 1.92 (s, 3H), 1.61 (d, J=6.8 Hz, 3H). MS (ESI): Calcd. For C₁₈H₂₁N₅O₂: 339. found 340 (M+H)⁺.

### Example 19: Synthesis of (S)—N-(2-(5-oxo-2-((1-(pyridin-3-yl)ethyl)amino)-5,7-dihydro-6H-pyrrolo[3,4-b]pyridin-6-yl)ethyl)acetamide (Compound 23)

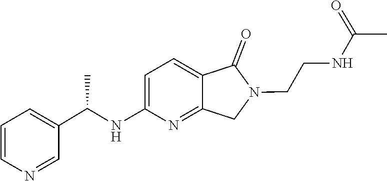 [SMILES: CC(=O)NCCN1Cc2nc(N[C@@H](C)c3cccnc3)ccc2C1=O]

(S)-Methyl-3-pyridinemethanamine (578 mg, 4.73 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product (S)—N-(2-(5-oxo-2-((1-(pyridin-3-yl)ethyl)amino)-5,7-dihydro-6H-pyrrolo[3,4-b]pyridin-6-yl)ethyl)acetamide (120 mg, 22% yield) as yellow solid. ¹H NMR (400 MHz, CDCl₃) δ 8.65 (d, J=2.3 Hz, 1H), 8.50 (dd, J=4.8, 1.6 Hz, 1H), 7.77-7.62 (m, 2H), 7.28-7.26 (m, 1H), 6.45 (s, 1H), 6.28 (d, J=8.5 Hz, 1H), 5.35 (d, J=6.7 Hz, 1H), 5.05 (p, J=6.7 Hz, 1H), 4.34-4.15 (m, 2H), 3.69 (td, J=5.7, 1.8 Hz, 2H), 3.57-3.40 (m, 2H), 1.91 (s, 3H), 1.62 (s, 3H). MS (ESI): Calcd. For C₁₈H₂₁N₅O₂: 339. found 340 (M+H)⁺.

### Example 20: Synthesis of N-(2-(2-(((6-methylpyridin-3-yl)methyl)amino)-5-oxo-5,7-dihydro-6H-pyrrolo[3,4-b]pyridin-6-yl)ethyl)acetamide (Compound 24)

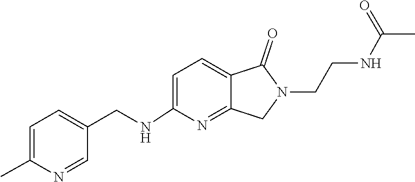 [SMILES: CC(=O)NCCN1Cc2nc(NCc3ccc(C)nc3)ccc2C1=O]

6-Methyl-3-pyridinemethanamine (771 mg, 6.31 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((6-methylpyridin-3-yl)methyl)amino)-5-oxo-5,7-dihydro-6H-pyrrolo[3,4-b]pyridin-6-yl)ethyl)acetamide (130 mg, 24% yield) as white solid. ¹H NMR (400 MHz, DMSO) δ 8.39 (s, 1H), 8.29 (d, J=4.9 Hz, 1H), 7.90 (t, J=5.9 Hz, 1H), 7.67 (t, J=5.5 Hz, 1H), 7.55 (d, J=8.6 Hz, 1H), 7.17 (d, J=4.9 Hz, 1H), 6.53 (d, J=8.6 Hz, 1H), 4.51 (d, J=5.5 Hz, 2H), 4.25 (s, 2H), 3.43 (t, J=6.0 Hz, 2H), 3.22 (q, J=6.1 Hz, 2H), 2.31 (s, 3H), 1.71 (s, 3H). MS (ESI): Calcd. For C₁₈H₂₁N₅O₂: 339. found 340 (M+H)⁺.

### Example 21: Synthesis of N-(2-(2-(((4-methylpyridin-3-yl)methyl)amino)-5-oxo-5,7-dihydro-6H-pyrrolo[3,4-b]pyridin-6-yl)ethyl)acetamide (Compound 25)

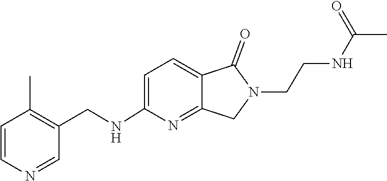 [SMILES: CC(=O)NCCN1Cc2nc(NCc3cnccc3C)ccc2C1=O]

4-Methyl-3-pyridinemethanamine (771 mg, 6.31 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((4-methylpyridin-3-yl)methyl)amino)-5-oxo-5,7-dihydro-6H-pyrrolo[3,4-b]pyridin-6-yl)ethyl)acetamide (90 mg, 17% yield) as white solid. ¹H NMR (400 MHz, DMSO) δ 8.39 (d, J=2.3 Hz, 1H), 7.89 (t, J=5.8 Hz, 1H), 7.77 (t, J=5.9 Hz, 1H), 7.64-7.50 (m, 2H), 7.16 (d, J=7.9 Hz, 1H), 6.50 (d, J=8.5 Hz, 1H), 4.48 (d, J=5.8 Hz, 2H), 4.23 (s, 2H), 3.43 (t, J=6.0 Hz, 2H), 3.22 (q, J=6.0 Hz, 2H), 2.39 (s, 3H), 1.71 (s, 3H). MS (ESI): Calcd. For C₁₈H₂₁N₅O₂: 339. found 340 (M+H)⁺.

### Example 22: Synthesis of N-(2-(2-(((5-fluoropyridin-3-yl)methyl)amino)-5-oxo-5,7-dihydro-6H-pyrrolo[3,4-b]pyridin-6-yl)ethyl)acetamide (Compound 26)

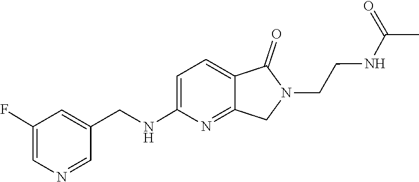 [SMILES: CC(=O)NCCN1Cc2nc(NCc3cncc(F)c3)ccc2C1=O]

5-Fluoro-3-pyridinemethanamine (597 mg, 4.73 mmol) was added to a solution product N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((5-fluoropyridin-3-yl)methyl)amino)-5-oxo-5,7-dihydro-6H-pyrrolo[3,4-b]pyridin-6-yl)ethyl)acetamide (140 mg, 34% yield) as white solid. ¹H NMR (400 MHz, DMSO) δ 8.42 (d, J=2.5 Hz, 2H), 7.87 (dt, J=16.8, 5.9 Hz, 2H), 7.69-7.53 (m, 2H), 6.54 (d, J=8.5 Hz, 1H), 4.59 (d, J=5.9 Hz, 2H), 4.23 (s, 2H), 3.43 (t, J=6.0 Hz, 2H), 3.21 (q, J=6.1 Hz, 2H), 1.71 (s, 3H). MS (ESI): Calcd. For C₁₇H₁₈FN₅O₂: 343. found 344 (M+H)⁺.

### Example 23: Synthesis of N-(2-(5-oxo-2-((pyridin-4-ylmethyl)amino)-5,7-dihydro-6H-pyrrolo[3,4-b]pyridin-6-yl)ethyl)acetamide (Compound 27)

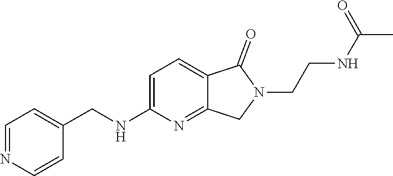 [SMILES: CC(=O)NCCN1Cc2nc(NCc3ccncc3)ccc2C1=O]

4-Pyridinemethanamine (639 mg, 5.91 mmol) was added to a solution product N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyridin-4-ylmethyl)amino)-5,7-dihydro-6H-pyrrolo[3,4-b]pyridin-6-yl)ethyl)acetamide (145 mg, 38% yield) as light yellow solid. ¹H NMR (400 MHz, CDCl₃) δ 8.64-8.48 (m, 2H), 7.75 (d, J=8.5 Hz, 1H), 7.37-7.18 (m, 2H), 6.41 (d, J=8.5 Hz, 2H), 5.46 (t, J=6.2 Hz, 1H), 4.67 (d, J=6.1 Hz, 2H), 4.29 (s, 2H), 3.71 (dd, J=6.8, 4.5 Hz, 2H), 3.51 (q, J=5.3 Hz, 2H), 1.92 (s, 3H). MS (ESI): Calcd. For C₁₇H₁₉N₅O₂: 325. found 326 (M+H)⁺.

### Example 24: Synthesis of N-(2-(2-((2-methoxybenzyl)amino)-5-oxo-5,7-dihydro-6H-pyrrolo[3,4-b]pyridin-6-yl)ethyl)acetamide (Compound 28)

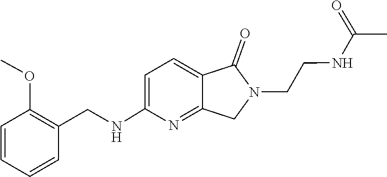 [SMILES: COc1ccccc1CNc1ccc2c(n1)CN(CCNC(C)=O)C2=O]

2-Methoxybenzylamine (1.08 g, 7.88 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2-methoxybenzyl)amino)-5-oxo-5,7-dihydro-6H-pyrrolo[3,4-b]pyridin-6-yl)ethyl)acetamide (160 mg, 29% yield) as white solid. ¹H NMR (400 MHz, DMSO) δ 7.89 (d, J=6.6 Hz, 1H), 7.65-7.48 (m, 2H), 7.19 (q, J=7.4 Hz, 2H), 6.96 (dd, J=8.2, 1.9 Hz, 1H), 6.85 (td, J=7.4, 1.9 Hz, 1H), 6.53 (d, J=8.6 Hz, 1H), 4.47 (d, J=5.2 Hz, 2H), 4.21 (d, J=2.1 Hz, 2H), 3.79 (d, J=2.1 Hz, 3H), 3.51-3.40 (m, 2H), 3.23-3.16 (m, 2H), 1.71 (d, J=2.2 Hz, 3H). MS (ESI): Calcd. For C₁₉H₂₂N₄O₃: 354. found 355 (M+H)⁺.

### Example 25: Synthesis of N-(2-(2-((5-fluoro-2-methoxybenzyl)amino)-5-oxo-5,7-dihydro-6H-pyrrolo[3,4-b]pyridin-6-yl)ethyl)acetamide (Compound 29)

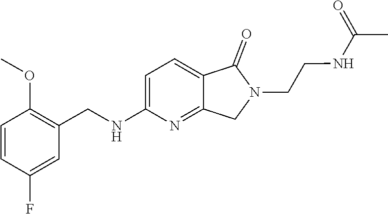 [SMILES: COc1ccc(F)cc1CNc1ccc2c(n1)CN(CCNC(C)=O)C2=O]

5-Fluoro-2-methoxybenzenemethanamine (734 mg, 4.73 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((5-fluoro-2-methoxybenzyl)amino)-5-oxo-5,7-dihydro-6H-pyrrolo[3,4-b]pyridin-6-yl)ethyl)acetamide (130 mg, 22% yield) as white solid. ¹H NMR (400 MHz, DMSO) δ 7.89 (t, J=5.9 Hz, 1H), 7.64 (t, J=6.0 Hz, 1H), 7.56 (dd, J=8.4, 1.0 Hz, 1H), 7.13-6.90 (m, 3H), 6.55 (d, J=8.6 Hz, 1H), 4.47 (d, J=5.9 Hz, 2H), 4.22 (s, 2H), 3.78 (d, J=1.1 Hz, 3H), 3.42 (t, J=6.0 Hz, 2H), 3.21 (q, J=6.0 Hz, 2H), 1.71 (d, J=1.2 Hz, 3H). MS (ESI): Calcd. For C₁₉H₂₁FN₄O₃: 372. found 373 (M+H)⁺.

### Example 26: Synthesis of N-(2-(2-((2-fluoro-6-methoxybenzyl)amino)-5-oxo-5,7-dihydro-6H-pyrrolo[3,4-b]pyridin-6-yl)ethyl)acetamide (Compound 30)

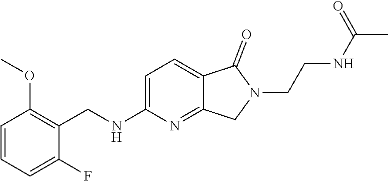 [SMILES: COc1cccc(F)c1CNc1ccc2c(n1)CN(CCNC(C)=O)C2=O]

2-Fluoro-6-methoxybenzenemethanamine (734 mg, 4.73 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2-fluoro-6-methoxybenzyl)amino)-5-oxo-5,7-dihydro-6H-pyrrolo[3,4-b]pyridin-6-yl)ethyl)acetamide (150 mg, 26% yield) as white solid. ¹H NMR (400 MHz, DMSO) δ 7.92 (d, J=6.1 Hz, 1H), 7.51 (dd, J=8.5, 2.0 Hz, 1H), 7.39-7.24 (m, 2H), 6.95-6.72 (m, 2H), 6.51 (dd, J=8.7, 2.0 Hz, 1H), 4.45 (d, J=4.6 Hz, 2H), 4.27 (d, J=2.0 Hz, 2H), 3.80 (d, J=2.0 Hz, 3H), 3.44 (t, J=6.1 Hz, 2H), 3.23 (d, J=6.8 Hz, 2H), 1.73 (d, J=2.0 Hz, 3H). MS (ESI): Calcd. For C₁₉H₂₁FN₄O₃: 372. found 373 (M+H)⁺.

### Example 27: Synthesis of N-(2-(2-((4-fluoro-2-methoxybenzyl)amino)-5-oxo-5,7-dihydro-6H-pyrrolo[3,4-b]pyridin-6-yl)ethyl)acetamide (Compound 31)

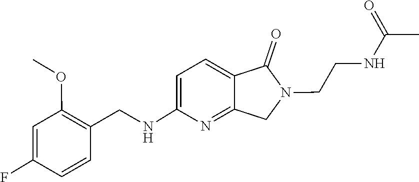 [SMILES: COc1cc(F)ccc1CNc1ccc2c(n1)CN(CCNC(C)=O)C2=O]

4-Fluoro-2-methoxybenzenemethanamine (734 mg, 4.73 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-fluoro-2-methoxybenzyl)amino)-5-oxo-5,7-dihydro-6H-pyrrolo[3,4-b]pyridin-6-yl)ethyl)acetamide (164 mg, 28% yield) as white solid. ¹H NMR (400 MHz, DMSO) δ 7.89 (t, J=5.8 Hz, 1H), 7.55 (ddd, J=10.8, 7.8, 3.6 Hz, 2H), 7.27-7.12 (m, 1H), 6.87 (dt, J=11.3, 2.6 Hz, 1H), 6.67 (tt, J=8.5, 2.6 Hz, 1H), 6.52 (d, J=8.6 Hz, 1H), 4.42 (d, J=5.7 Hz, 2H), 4.22 (d, J=2.3 Hz, 2H), 3.80 (d, J=2.3 Hz, 3H), 3.53-3.40 (m, 2H), 3.26-3.15 (m, 2H), 1.71 (d, J=2.3 Hz, 3H). MS (ESI): Calcd. For C₁₉H₂₁FN₄O₃: 372. found 373 (M+H)⁺.

### Example 28: Synthesis of N-(2-(2-(((4-methoxypyridin-3-yl)methyl)amino)-5-oxo-5,7-dihydro-6H-pyrrolo[3,4-b]pyridin-6-yl)ethyl)acetamide (Compound 32)

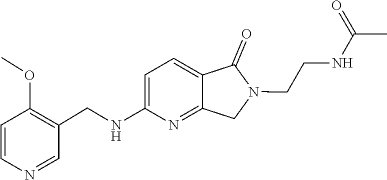 [SMILES: COc1ccncc1CNc1ccc2c(n1)CN(CCNC(C)=O)C2=O]

4-Methoxy-3-pyridinemethanamine (654 mg, 4.73 mmol) was added to a solution product N-(2-(2-chloro-5-oxo-5,7-dihydro-6H-pyrrolo[3,4-b]pyridin-6-yl)ethyl)acetamide (400 mg, 1.58 mmol) and DIPEA (1.37 mL, 7.88 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((4-methoxypyridin-3-yl)methyl)amino)-5-oxo-5,7-dihydro-6H-pyrrolo[3,4-b]pyridin-6-yl)ethyl)acetamide (98 mg, 18% yield) as white solid. ¹H NMR (400 MHz, DMSO) δ 8.33 (d, J=5.6 Hz, 1H), 8.25 (s, 1H), 7.90 (t, J=5.8 Hz, 1H), 7.61 (t, J=5.7 Hz, 1H), 7.55 (d, J=8.6 Hz, 1H), 7.01 (d, J=5.6 Hz, 1H), 6.54 (d, J=8.5 Hz, 1H), 4.46 (d, J=5.6 Hz, 2H), 4.23 (s, 2H), 3.85 (s, 3H), 3.43 (t, J=6.0 Hz, 2H), 3.20 (d, J=6.0 Hz, 2H), 1.71 (s, 3H). MS (ESI): Calcd. For C₁₈H₂₁N₅O₃: 355. found 356 (M+H)⁺.

### Example 29: Synthesis of N-(2-(2-((5-fluoro-2-methoxybenzyl)amino)-5-oxo-5,7-dihydro-6H-pyrrolo[3,4-b]pyridin-6-yl)ethyl)propionamide (Compound 33)

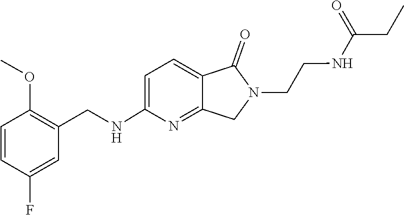 [SMILES: CCC(=O)NCCN1Cc2nc(NCc3cc(F)ccc3OC)ccc2C1=O]

5-Fluoro-2-methoxybenzenemethanamine (522 mg, 3.36 mmol) was added to a solution product N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((5-fluoro-2-methoxybenzyl)amino)-5-oxo-5,7-dihydro-6H-pyrrolo[3,4-b]pyridin-6-yl)ethyl)propionamide (165 mg, 38% yield) as white solid. ¹H NMR (400 MHz, DMSO) δ 8.33 (d, J=5.6 Hz, 1H), 8.25 (s, 1H), 7.90 (t, J=5.8 Hz, 1H), 7.61 (t, J=5.7 Hz, 1H), 7.55 (d, J=8.6 Hz, 1H), 7.01 (d, J=5.6 Hz, 1H), 6.54 (d, J=8.5 Hz, 1H), 4.46 (d, J=5.6 Hz, 2H), 4.23 (s, 2H), 3.85 (s, 3H), 3.43 (t, J=6.0 Hz, 2H), 3.20 (d, J=6.0 Hz, 2H), 1.71 (s, 3H). MS (ESI): Calcd. For C₂₀H₂₃FN₄O₃: 386. found 387 (M+H)⁺.

### Example 30: Synthesis of N-(2-(2-(((6-methylpyridin-3-yl)methyl)amino)-5-oxo-5,7-dihydro-6H-pyrrolo[3,4-b]pyridin-6-yl)ethyl)propionamide (Compound 34)

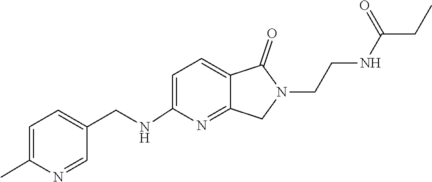 [SMILES: CCC(=O)NCCN1Cc2nc(NCc3ccc(C)nc3)ccc2C1=O]

6-Methyl-3-pyridinemethanamine (274 mg, 2.24 mmol) was added to a solution product N-(2-(2-chloro-5-oxo-5,7-dihydro-6H-pyrrolo[3,4-b]pyridin-6-yl)ethyl)propionamide (200 mg, 0.75 mmol) and DIPEA (0.65 mL, 3.74 mmol) in 1-BuOH (8 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((6-methylpyridin-3-yl)methyl)amino)-5-oxo-5,7-dihydro-6H-pyrrolo[3,4-b]pyridin-6-yl)ethyl)propionamide (85 mg, 32% yield) as white solid. ¹H NMR (400 MHz, DMSO) δ 8.38 (d, J=2.3 Hz, 1H), 7.79 (dt, J=19.8, 5.3 Hz, 2H), 7.56 (ddd, J=11.9, 8.3, 1.7 Hz, 2H), 7.16 (d, J=7.9 Hz, 1H), 6.50 (d, J=8.5 Hz, 1H), 4.48 (d, J=5.9 Hz, 2H), 4.23 (s, 2H), 3.43 (t, J=6.2 Hz, 2H), 3.22 (q, J=5.9 Hz, 2H), 2.39 (s, 3H), 1.97 (q, J=7.6 Hz, 2H), 0.89 (td, J=7.6, 1.1 Hz, 3H). MS (ESI): Calcd. For C₁₉H₂₃N₅O₂: 353. found 354 (M+H)⁺.

### Example 31: Synthesis of N-(2-(2-((4-methylbenzyl)amino)-5-oxo-5,7-dihydro-6H-pyrrolo[3,4-b]pyridin-6-yl)ethyl)acetamide (Compound 35)

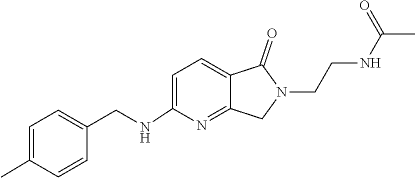 [SMILES: CC(=O)NCCN1Cc2nc(NCc3ccc(C)cc3)ccc2C1=O]

4-Methylbenzylamine (430 mg, 3.55 mmol) was added to a solution product N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-methylbenzyl)amino)-5-oxo-5,7-dihydro-6H-pyrrolo[3,4-b]pyridin-6-yl)ethyl)acetamide (110 mg, 28% yield) as white solid. ¹H NMR (400 MHz, DMSO) δ 7.89 (t, J=5.9 Hz, 1H), 7.71 (t, J=5.9 Hz, 1H), 7.53 (d, J=8.6 Hz, 1H), 7.28-7.15 (m, 2H), 7.15-6.99 (m, 2H), 6.49 (d, J=8.6 Hz, 1H), 4.46 (d, J=5.9 Hz, 2H), 4.22 (s, 2H), 3.47-3.35 (m, 2H), 3.21 (q, J=6.0 Hz, 2H), 2.23 (s, 3H), 1.71 (s, 3H). MS (ESI): Calcd. For C₁₉H₂₂N₄O₂: 338. found 339 (M+H)⁺.

### Example 32: Synthesis of N-(2-(2-((2,4-dimethylbenzyl)amino)-5-oxo-5,7-dihydro-6H-pyrrolo[3,4-b]pyridin-6-yl)ethyl)acetamide (Compound 36)

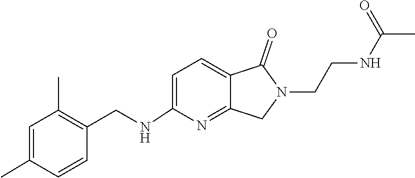 [SMILES: CC(=O)NCCN1Cc2nc(NCc3ccc(C)cc3C)ccc2C1=O]

2,4-Dimethylbenzenemethanamine (480 mg, 3.55 mmol) was added to a solution product N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2,4-dimethylbenzyl)amino)-5-oxo-5,7-dihydro-6H-pyrrolo[3,4-b]pyridin-6-yl)ethyl)acetamide (110 mg, 26% yield) as white solid. ¹H NMR (400 MHz, DMSO) δ 7.90 (t, J=6.0 Hz, 1H), 7.63-7.48 (m, 2H), 7.10 (d, J=7.7 Hz, 1H), 6.96 (d, J=1.7 Hz, 1H), 6.91 (dd, J=7.7, 1.9 Hz, 1H), 6.51 (d, J=8.6 Hz, 1H), 4.42 (d, J=5.5 Hz, 2H), 4.23 (s, 2H), 3.52-3.37 (m, 2H), 3.22 (q, J=6.0 Hz, 2H), 2.24 (s, 3H), 2.20 (s, 3H), 1.72 (s, 3H). MS (ESI): Calcd. For C₂₀H₂₄N₄O₂: 352. found 353 (M+H)⁺.

### Example 33: Synthesis of N-(2-(5-oxo-2-((2,4,6-trifluorobenzyl)amino)-5,7-dihydro-6H-pyrrolo[3,4-b]pyridin-6-yl)ethyl)acetamide (Compound 37)

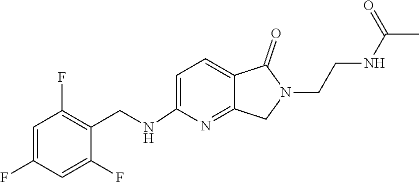 [SMILES: CC(=O)NCCN1Cc2nc(NCc3c(F)cc(F)cc3F)ccc2C1=O]

2,4,6-Trifluorobenzenemethanamine (572 mg, 3.55 mmol) was added to a solution product N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,4,6-trifluorobenzyl)amino)-5,7-dihydro-6H-pyrrolo[3,4-b]pyridin-6-yl)ethyl)acetamide (95 mg, 21% yield) as light yellow solid. ¹H NMR (400 MHz, DMSO) δ 7.90 (t, J=5.9 Hz, 1H), 7.62 (t, J=5.2 Hz, 1H), 7.54 (d, J=8.6 Hz, 1H), 7.25-7.10 (m, 2H), 6.49 (d, J=8.6 Hz, 1H), 4.49 (d, J=5.1 Hz, 2H), 4.25 (s, 2H), 3.51-3.41 (m, 2H), 3.23 (q, J=6.2 Hz, 2H), 1.72 (s, 3H). MS (ESI): Calcd. For C₁₈H₁₇F₃N₄O₂: 378. found 379 (M+H)⁺.

### Example 34: Synthesis of N-(2-methyl-1-(5-oxo-2-((pyridin-3-ylmethyl)amino)-5,7-dihydro-6H-pyrrolo[3,4-b]pyridin-6-yl)propan-2-yl)acetamide (Compound 38)

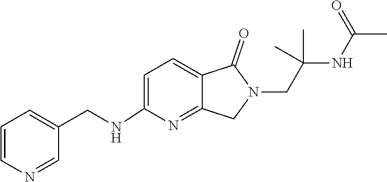 [SMILES: CC(=O)NC(C)(C)CN1Cc2nc(NCc3cccnc3)ccc2C1=O]

3-Pyridinemethanamine (576 mg, 5.32 mmol) was added to a solution product N-(1-(2-chloro-5-oxo-5,7-dihydro-6H-pyrrolo[3,4-b]pyridin-6-yl)-2-methylpropan-2-yl)acetamide (300 mg, 1.06 mmol) and DIPEA (0.93 mL, 5.32 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-methyl-1-(5-oxo-2-((pyridin-3-ylmethyl)amino)-5,7-dihydro-6H-pyrrolo[3,4-b]pyridin-6-yl)propan-2-yl)acetamide (115 mg, 31% yield) as white solid. ¹H NMR (400 MHz, DMSO) δ 8.67-8.50 (m, 1H), 8.41 (dd, J=4.8, 1.7 Hz, 1H), 7.84 (t, J=5.9 Hz, 1H), 7.80-7.66 (m, 1H), 7.58 (d, J=8.6 Hz, 1H), 7.50 (s, 1H), 7.31 (ddd, J=7.8, 4.8, 0.9 Hz, 1H), 6.54 (d, J=8.6 Hz, 1H), 4.55 (d, J=5.9 Hz, 2H), 4.25 (s, 2H), 3.63 (s, 2H), 1.77 (s, 3H), 1.17 (s, 6H). MS (ESI): Calcd. For C₁₉H₂₃N₅O₂: 353. found 354 (M+H)⁺.

### Example 35: Synthesis of N-(2-(5-oxo-2-((pyrimidin-2-ylmethyl)amino)-5,7-dihydro-6H-pyrrolo[3,4-b]pyridin-6-yl)ethyl)acetamide (Compound 39)

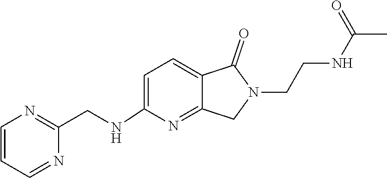 [SMILES: CC(=O)NCCN1Cc2nc(NCc3ncccn3)ccc2C1=O]

2-Pyrimidinemethanamine (258 mg, 2.37 mmol) was added to a solution product N-(2-(2-chloro-5-oxo-5,7-dihydro-6H-pyrrolo[3,4-b]pyridin-6-yl)ethyl)acetamide (300 mg, 1.18 mmol) and DIPEA (0.82 mL, 4.73 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyrimidin-2-ylmethyl)amino)-5,7-dihydro-6H-pyrrolo[3,4-b]pyridin-6-yl)ethyl)acetamide (120 mg, 31% yield) as yellow solid. ¹H NMR (400 MHz, DMSO) δ 8.73 (d, J=4.9 Hz, 2H), 7.88 (t, J=5.9 Hz, 1H), 7.80 (t, J=5.9 Hz, 1H), 7.55 (d, J=8.6 Hz, 1H), 7.35 (t, J=4.9 Hz, 1H), 6.63 (d, J=8.6 Hz, 1H), 4.73 (d, J=5.9 Hz, 2H), 4.18 (s, 2H), 3.51-3.36 (m, 2H), 3.20 (q, J=6.0 Hz, 2H), 1.70 (s, 3H). MS (ESI): Calcd. For C₁₆H₁₈N₆O₂: 326. found 327 (M+H)⁺.

### Example 36: Synthesis of N-(2-(2-(((6-cyanopyridin-3-yl)methyl)amino)-5-oxo-5,7-dihydro-6H-pyrrolo[3,4-b]pyridin-6-yl)ethyl)acetamide (Compound 40)

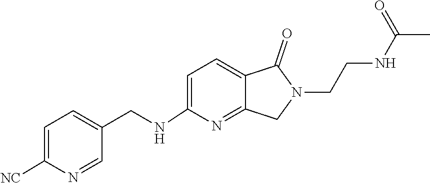 [SMILES: CC(=O)NCCN1Cc2nc(NCc3ccc(C#N)nc3)ccc2C1=O]

5-(Aminomethyl)-2-pyridinecarbonitrile (315 mg, 2.37 mmol) was added to a solution product N-(2-(2-chloro-5-oxo-5,7-dihydro-6H-pyrrolo[3,4-b]pyridin-6-yl)ethyl)acetamide (300 mg, 1.18 mmol) and DIPEA (0.82 mL, 4.73 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((6-cyanopyridin-3-yl)methyl)amino)-5-oxo-5,7-dihydro-6H-pyrrolo[3,4-b]pyridin-6-yl)ethyl)acetamide (98 mg, 24% yield) as light yellow solid. ¹H NMR (400 MHz, DMSO) δ 8.69 (dd, J=2.1, 0.9 Hz, 1H), 8.08-7.81 (m, 4H), 7.59 (d, J=8.6 Hz, 1H), 6.56 (d, J=8.6 Hz, 1H), 4.64 (d, J=6.0 Hz, 2H), 4.21 (s, 2H), 3.42 (t, J=6.1 Hz, 2H), 3.21 (p, J=6.6 Hz, 2H), 1.71 (s, 3H). MS (ESI): Calcd. For C₁₈H₁₈N₆O₂: 350. found 351 (M+H)⁺.

### Example 37: Synthesis of N-(2-(2-(((6-cyanopyridin-3-yl)methyl)amino)-5-oxo-5,7-dihydro-6H-pyrrolo[3,4-b]pyridin-6-yl)ethyl)acetamide (Compound 41)

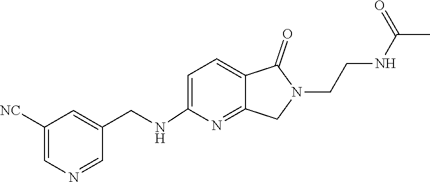 [SMILES: CC(=O)NCCN1Cc2nc(NCc3cncc(C#N)c3)ccc2C1=O]

5-(Aminomethyl)-3-pyridinecarbonitrile (315 mg, 2.37 mmol) was added to a solution product N-(2-(2-chloro-5-oxo-5,7-dihydro-6H-pyrrolo[3,4-b]pyridin-6-yl)ethyl)acetamide (300 mg, 1.18 mmol) and DIPEA (0.82 mL, 4.73 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((6-cyanopyridin-3-yl)methyl)amino)-5-oxo-5,7-dihydro-6H-pyrrolo[3,4-b]pyridin-6-yl)ethyl)acetamide (105 mg, 25% yield) as light yellow solid. ¹H NMR (400 MHz, DMSO) δ 8.87 (d, J=2.0 Hz, 1H), 8.80 (d, J=2.2 Hz, 1H), 8.20 (t, J=2.1 Hz, 1H), 7.88 (dd, J=9.7, 4.1 Hz, 2H), 7.59 (d, J=8.5 Hz, 1H), 6.55 (d, J=8.5 Hz, 1H), 4.60 (d, J=5.9 Hz, 2H), 4.23 (s, 2H), 3.51-3.39 (m, 2H), 3.21 (q, J=6.0 Hz, 2H), 1.71 (s, 3H). MS (ESI): Calcd. For C₁₈H₁₈N₆O₂: 350. found 351 (M+H)⁺.

### Example 38: Synthesis of N-(2-(5-oxo-2-((pyridin-3-ylmethyl)amino)-5,7-dihydro-6H-pyrrolo[3,4-b]pyridin-6-yl)ethyl)cyclopropanecarboxamide (Compound 42)

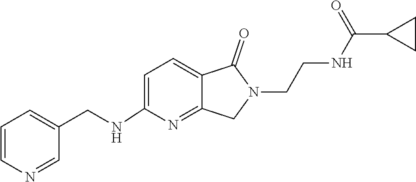 [SMILES: O=C(NCCN1Cc2nc(NCc3cccnc3)ccc2C1=O)C1CC1]

3-Pyridinemethanamine (232 mg, 2.14 mmol) was added to a solution product N-(2-(2-chloro-5-oxo-5,7-dihydro-6H-pyrrolo[3,4-b]pyridin-6-yl)ethyl)cyclopropanecarboxamide (300 mg, 1.07 mmol) and DIPEA (0.75 mL, 4.29 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyridin-3-ylmethyl)amino)-5,7-dihydro-6H-pyrrolo[3,4-b]pyridin-6-yl)ethyl)cyclopropanecarboxamide (114 mg, 30% yield) as light yellow solid. ¹H NMR (400 MHz, DMSO) δ 8.53 (dd, J=2.3, 0.9 Hz, 1H), 8.41 (dd, J=4.8, 1.7 Hz, 1H), 8.11 (t, J=5.9 Hz, 1H), 7.82 (t, J=6.0 Hz, 1H), 7.78-7.67 (m, 1H), 7.56 (d, J=8.5 Hz, 1H), 7.31 (ddd, J=7.8, 4.8, 0.9 Hz, 1H), 6.53 (d, J=8.6 Hz, 1H), 4.54 (d, J=5.9 Hz, 2H), 4.23 (s, 2H), 3.44 (t, J=6.1 Hz, 2H), 3.25 (d, J=6.0 Hz, 2H), 1.43 (tt, J=7.2, 5.6 Hz, 1H), 0.67-0.48 (m, 4H). MS (ESI): Calcd. For C₁₉H₂₁N₅O₂: 351. found 352 (M+H)⁺.

### Example 39: Synthesis of N-(2-(2-(((5-cyanopyridin-3-yl)methyl)amino)-5-oxo-5,7-dihydro-6H-pyrrolo[3,4-b]pyridin-6-yl)ethyl)cyclopropanecarboxamide (Compound 43)

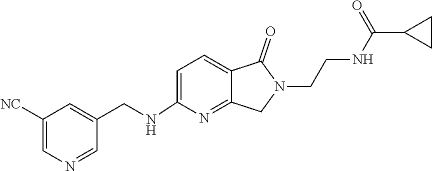 [SMILES: N#Cc1cncc(CNc2ccc3c(n2)CN(CCNC(=O)C2CC2)C3=O)c1]

5-(Aminomethyl)-3-pyridinecarbonitrile (286 mg, 2.14 mmol) was added to a solution product N-(2-(2-chloro-5-oxo-5,7-dihydro-6H-pyrrolo[3,4-b]pyridin-6-yl)ethyl)cyclopropanecarboxamide (300 mg, 1.07 mmol) and DIPEA (0.75 mL, 4.29 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((5-cyanopyridin-3-yl)methyl)amino)-5-oxo-5,7-dihydro-6H-pyrrolo[3,4-b]pyridin-6-yl)ethyl)cyclopropanecarboxamide (95 mg, 24% yield) as white solid. ¹H NMR (400 MHz, DMSO) δ 8.87 (d, J=2.0 Hz, 1H), 8.80 (d, J=2.2 Hz, 1H), 8.19 (t, J=2.1 Hz, 1H), 8.10 (t, J=5.9 Hz, 1H), 7.87 (t, J=5.9 Hz, 1H), 7.59 (d, J=8.5 Hz, 1H), 6.55 (d, J=8.5 Hz, 1H), 4.60 (d, J=5.9 Hz, 2H), 4.23 (s, 2H), 3.44 (t, J=6.1 Hz, 2H), 3.25 (q, J=5.9 Hz, 2H), 1.42 (p, J=6.3 Hz, 1H), 0.55 (d, J=5.7 Hz, 4H). MS (ESI): Calcd. For C₂₀H₂₀N₆O₂: 376. found 377 (M+H)⁺.

### Example 40: Synthesis of 2,2,2-trifluoro-N-(2-(5-oxo-2-((pyridin-3-ylmethyl)amino)-5,7-dihydro-6H-pyrrolo[3,4-b]pyridin-6-yl)ethyl)acetamide (Compound 44)

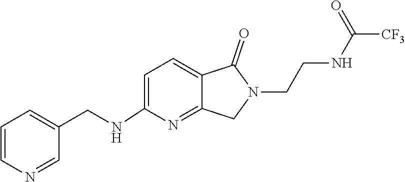 [SMILES: O=C1c2ccc(NCc3cccnc3)nc2CN1CCNC(=O)C(F)(F)F]

3-Pyridinemethanamine (316 mg, 2.93 mmol) was added to a solution product N-(2-(2-chloro-5-oxo-5,7-dihydro-6H-pyrrolo[3,4-b]pyridin-6-yl)ethyl)-2,2,2-trifluoroacetamide (300 mg, 0.98 mmol) and DIPEA (0.68 mL, 3.90 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product 2,2,2-trifluoro-N-(2-(5-oxo-2-((pyridin-3-ylmethyl)amino)-5,7-dihydro-6H-pyrrolo[3,4-b]pyridin-6-yl)ethyl)acetamide (80 mg, 22% yield) as yellow solid. ¹H NMR (400 MHz, DMSO) δ 9.46 (t, J=5.8 Hz, 1H), 8.53 (dd, J=2.4, 0.8 Hz, 1H), 8.41 (dd, J=4.8, 1.7 Hz, 1H), 7.85 (t, J=5.9 Hz, 1H), 7.79-7.66 (m, 1H), 7.57 (d, J=8.6 Hz, 1H), 7.31 (ddd, J=7.8, 4.8, 0.9 Hz, 1H), 6.53 (d, J=8.6 Hz, 1H), 4.54 (d, J=5.9 Hz, 2H), 4.23 (s, 2H), 3.55 (dd, J=6.6, 5.0 Hz, 2H), 3.38 (t, J=5.8 Hz, 2H). MS (ESI): Calcd. For C₁₇H₁₆F₃N₅O₂: 379. found 380 (M+H)⁺.

### Example 41: Synthesis of N-(2-(2-(((4-methylpyridin-3-yl)methyl)amino)-5-oxo-5,7-dihydro-6H-pyrrolo[3,4-b]pyridin-6-yl)ethyl)propionamide (Compound 45)

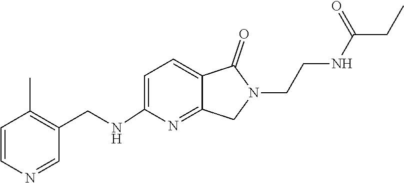 [SMILES: CCC(=O)NCCN1Cc2nc(NCc3cnccc3C)ccc2C1=O]

4-Methyl-3-pyridinemethanamine (274 mg, 2.24 mmol) was added to a solution product N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((4-methylpyridin-3-yl)methyl)amino)-5-oxo-5,7-dihydro-6H-pyrrolo[3,4-b]pyridin-6-yl)ethyl)propionamide (133 mg, 34% yield) as white solid. ¹H NMR (400 MHz, DMSO) δ 8.39 (s, 1H), 8.29 (d, J=4.9 Hz, 1H), 7.82 (t, J=5.8 Hz, 1H), 7.67 (t, J=5.5 Hz, 1H), 7.55 (d, J=8.6 Hz, 1H), 7.17 (dt, J=4.9, 0.7 Hz, 1H), 6.53 (d, J=8.6 Hz, 1H), 4.51 (d, J=5.4 Hz, 2H), 4.24 (s, 2H), 3.43 (q, J=6.4 Hz, 2H), 3.23 (q, J=6.1 Hz, 2H), 2.39-2.22 (m, 3H), 1.97 (q, J=7.6 Hz, 2H), 0.89 (t, J=7.6 Hz, 3H). MS (ESI): Calcd. For C₁₉H₂₃N₅O₂: 353. found 354 (M+H)⁺.

### Example 42: Synthesis of N-(2-(5-oxo-2-((pyrazin-2-ylmethyl)amino)-5,7-dihydro-6H-pyrrolo[3,4-b]pyridin-6-yl)ethyl)propionamide (Compound 46)

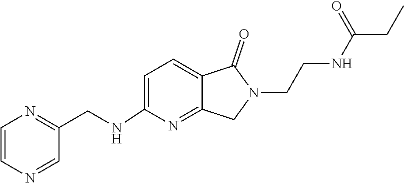 [SMILES: CCC(=O)NCCN1Cc2nc(NCc3cnccn3)ccc2C1=O]

2-Aminomethylpyrazine (245 mg, 2.24 mmol) was added to a solution product N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyrazin-2-ylmethyl)amino)-5,7-dihydro-6H-pyrrolo[3,4-b]pyridin-6-yl)ethyl)propionamide (120 mg, 31% yield) as yellow solid. ¹H NMR (400 MHz, DMSO) δ 8.65-8.54 (m, 2H), 8.49 (d, J=2.6 Hz, 1H), 7.92 (t, J=5.9 Hz, 1H), 7.80 (t, J=5.9 Hz, 1H), 7.58 (d, J=8.6 Hz, 1H), 6.59 (d, J=8.6 Hz, 1H), 4.67 (d, J=5.9 Hz, 2H), 4.20 (s, 2H), 3.42 (t, J=6.0 Hz, 2H), 3.21 (q, J=6.0 Hz, 2H), 1.96 (q, J=7.6 Hz, 2H), 0.88 (t, J=7.6 Hz, 3H). MS (ESI): Calcd. For C₁₇H₂₀N₆O₂: 340. found 341 (M+H)⁺.

### Example 43: Synthesis of N-(2-(5-oxo-2-((pyridin-3-ylmethyl)amino)-5,7-dihydro-6H-pyrrolo[3,4-b]pyridin-6-yl)ethyl)butyramide (Compound 47)

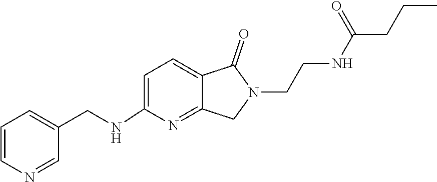 [SMILES: CCCC(=O)NCCN1Cc2nc(NCc3cccnc3)ccc2C1=O]

3-Pyridinemethanamine (230 mg, 2.13 mmol) was added to a solution product N-(2-(2-chloro-5-oxo-5,7-dihydro-6H-pyrrolo[3,4-b]pyridin-6-yl)ethyl)butyramide (300 mg, 1.06 mmol) and DIPEA (0.93 mL, 5.32 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyridin-3-ylmethyl)amino)-5,7-dihydro-6H-pyrrolo[3,4-b]pyridin-6-yl)ethyl)butyramide (125 mg, 33% yield) as white solid. ¹H NMR (400 MHz, DMSO) δ 8.53 (dd, J=2.3, 0.9 Hz, 1H), 8.41 (dd, J=4.8, 1.7 Hz, 1H), 7.83 (dt, J=9.0, 5.9 Hz, 2H), 7.69 (dt, J=7.9, 1.9 Hz, 1H), 7.56 (d, J=8.6 Hz, 1H), 7.31 (ddd, J=7.8, 4.8, 0.9 Hz, 1H), 6.52 (d, J=8.6 Hz, 1H), 4.54 (d, J=5.9 Hz, 2H), 4.23 (s, 2H), 3.43 (t, J=6.0 Hz, 2H), 3.23 (q, J=5.7 Hz, 2H), 1.93 (t, J=7.3 Hz, 2H), 1.40 (h, J=7.4 Hz, 2H), 0.73 (t, J=7.4 Hz, 3H). MS (ESI): Calcd. For C₁₉H₂₃N₅O₂: 353. found 354 (M+H)⁺.

### Example 44: Synthesis of N-(2-(5-oxo-2-((pyrazin-2-ylmethyl)amino)-5,7-dihydro-6H-pyrrolo[3,4-b]pyridin-6-yl)ethyl)butyramide (Compound 48)

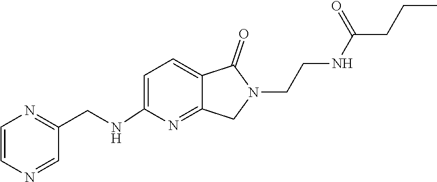 [SMILES: CCCC(=O)NCCN1Cc2nc(NCc3cnccn3)ccc2C1=O]

2-Aminomethylpyrazine (232 mg, 2.13 mmol) was added to a solution product N-(2-(2-chloro-5-oxo-5,7-dihydro-6H-pyrrolo[3,4-b]pyridin-6-yl)ethyl)butyramide (300 mg, 1.06 mmol) and DIPEA (0.93 mL, 5.32 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyrazin-2-ylmethyl)amino)-5,7-dihydro-6H-pyrrolo[3,4-b]pyridin-6-yl)ethyl)butyramide (95 mg, 25% yield) as yellow solid. ¹H NMR (400 MHz, DMSO) δ 8.65-8.45 (m, 3H), 7.92 (t, J=5.9 Hz, 1H), 7.83 (t, J=5.8 Hz, 1H), 7.57 (d, J=8.6 Hz, 1H), 6.59 (d, J=8.6 Hz, 1H), 4.67 (d, J=5.9 Hz, 2H), 4.20 (s, 2H), 3.43 (t, J=5.9 Hz, 2H), 3.24-3.14 (m, 2H), 1.93 (t, J=7.3 Hz, 2H), 1.39 (h, J=7.3 Hz, 2H), 0.72 (t, J=7.4 Hz, 3H). MS (ESI): Calcd. For C₁₈H₂₂N₆O₂: 354. found 355 (M+H)⁺.

### Example 45: Synthesis of N-(2-(5-oxo-2-((pyrazin-2-ylmethyl)amino)-5,7-dihydro-6H-pyrrolo[3,4-b]pyridin-6-yl)ethyl)cyclopropanecarboxamide (Compound 49)

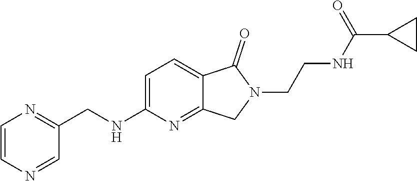 [SMILES: O=C(NCCN1Cc2nc(NCc3cnccn3)ccc2C1=O)C1CC1]

2-Aminomethylpyrazine (234 mg, 2.14 mmol) was added to a solution product N-(2-(2-chloro-5-oxo-5,7-dihydro-6H-pyrrolo[3,4-b]pyridin-6-yl)ethyl)cyclopropanecarboxamide (300 mg, 1.07 mmol) and DIPEA (0.93 mL, 5.36 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((pyrazin-2-ylmethyl)amino)-5,7-dihydro-6H-pyrrolo[3,4-b]pyridin-6-yl)ethyl)cyclopropanecarboxamide (125 mg, 33% yield) as yellow solid. ¹H NMR (400 MHz, DMSO) δ 8.67-8.54 (m, 2H), 8.49 (d, J=2.6 Hz, 1H), 8.10 (t, J=5.8 Hz, 1H), 7.93 (t, J=5.9 Hz, 1H), 7.58 (d, J=8.6 Hz, 1H), 6.60 (d, J=8.6 Hz, 1H), 4.67 (d, J=5.9 Hz, 2H), 4.20 (s, 2H), 3.47-3.38 (m, 2H), 3.26-3.15 (m, 2H), 1.42 (p, J=6.3 Hz, 1H), 0.63-0.46 (m, 4H). MS (ESI): Calcd. For C₁₈H₂₀N₆O₂: 352. found 353 (M+H)⁺.

### Example 46: N-(2-(2-((4-fluorobenzyl)amino)-5-oxo-5,7-dihydro-6H-pyrrolo[3,4-b]pyridin-6-yl)ethyl)acetamide (Compound 51)

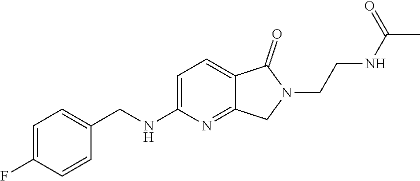 [SMILES: CC(=O)NCCN1Cc2nc(NCc3ccc(F)cc3)ccc2C1=O]

(4-Fluorophenyl)methanamine (296 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-fluorobenzyl)amino)-5-oxo-5,7-dihydro-6H-pyrrolo[3,4-b]pyridin-6-yl)ethyl)acetamide (134 mg, 33% yield) as white solid. ¹H NMR (400 MHz, DMSO) δ 7.90 (t, J=5.8 Hz, 1H), 7.77 (t, J=6.0 Hz, 1H), 7.55 (d, J=8.6 Hz, 1H), 7.33 (ddt, J=8.8, 5.4, 2.6 Hz, 2H), 7.19-7.05 (m, 2H), 6.50 (d, J=8.5 Hz, 1H), 4.50 (d, J=5.9 Hz, 2H), 4.23 (s, 2H), 3.52-3.37 (m, 2H), 3.21 (q, J=6.0 Hz, 2H), 1.71 (s, 3H). MS (ESI): Calcd. For C₁₈H₁₉FN₄O₂: 342. found 343 (M+H)⁺.

### Example 47: N-(2-(2-((2,4-difluorobenzyl)amino)-5-oxo-5,7-dihydro-6H-pyrrolo[3,4-b]pyridin-6-yl)ethyl)acetamide (Compound 52)

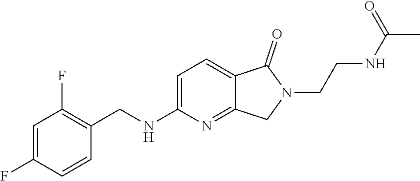 [SMILES: CC(=O)NCCN1Cc2nc(NCc3ccc(F)cc3F)ccc2C1=O]

(2,4-Difluorophenyl)methanamine (339 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2,4-difluorobenzyl)amino)-5-oxo-5,7-dihydro-6H-pyrrolo[3,4-b]pyridin-6-yl)ethyl)acetamide (140 mg, 33% yield) as white solid. ¹H NMR (400 MHz, DMSO) δ 7.90 (t, J=5.8 Hz, 1H), 7.74 (t, J=5.8 Hz, 1H), 7.56 (d, J=8.6 Hz, 1H), 7.39 (td, J=8.7, 6.7 Hz, 1H), 7.19 (ddd, J=10.6, 9.3, 2.6 Hz, 1H), 7.01 (tdd, J=8.5, 2.6, 1.0 Hz, 1H), 6.53 (d, J=8.6 Hz, 1H), 4.52 (d, J=5.7 Hz, 2H), 4.23 (s, 2H), 3.43 (t, J=6.0 Hz, 2H), 3.21 (q, J=6.0 Hz, 2H), 1.71 (s, 3H). MS (ESI): Calcd. For C₁₅H₁₈F₂N₄O₂: 360. found 361 (M+H)⁺.

### Example 48: N-(2-(5-oxo-2-((2,4,5-trifluorobenzyl)amino)-5,7-dihydro-6H-pyrrolo[3,4-b]pyridin-6-yl)ethyl)acetamide (Compound 53)

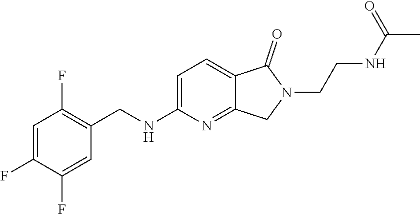 [SMILES: CC(=O)NCCN1Cc2nc(NCc3cc(F)c(F)cc3F)ccc2C1=O]

(2,4,5-Trifluorophenyl)methanamine (381 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,4,5-trifluorobenzyl)amino)-5,7-dihydro-6H-pyrrolo[3,4-b]pyridin-6-yl)ethyl)acetamide (116 mg, 26% yield) as white solid. ¹H NMR (400 MHz, DMSO) δ 7.89 (t, J=5.8 Hz, 1H), 7.77 (t, J=5.9 Hz, 1H), 7.62-7.45 (m, 2H), 7.39 (ddd, J=11.2, 9.1, 6.8 Hz, 1H), 6.54 (d, J=8.6 Hz, 1H), 4.51 (d, J=5.8 Hz, 2H), 4.23 (s, 2H), 3.43 (t, J=6.0 Hz, 2H), 3.21 (q, J=6.0 Hz, 2H), 1.71 (s, 3H). MS (ESI): Calcd. For C₁₈H₁₇F₃N₄O₂: 378. found 379 (M+H)⁺.

### Example 49: N-(2-(2-((5-chloro-2,4-difluorobenzyl)amino)-5-oxo-5,7-dihydro-6H-pyrrolo[3,4-b]pyridin-6-yl)ethyl)acetamide (Compound 54)

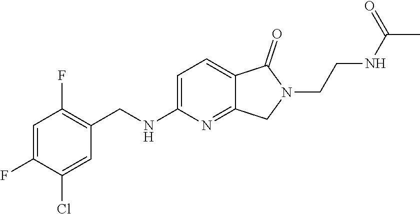 [SMILES: CC(=O)NCCN1Cc2nc(NCc3cc(Cl)c(F)cc3F)ccc2C1=O]

(5-Chloro-2,4-difluorophenyl)methanamine (420 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((5-chloro-2,4-difluorobenzyl)amino)-5-oxo-5,7-dihydro-6H-pyrrolo[3,4-b]pyridin-6-yl)ethyl)acetamide (95 mg, 20% yield) as white solid. ¹H NMR (400 MHz, DMSO) δ 7.90 (t, J=5.9 Hz, 1H), 7.77 (t, J=5.8 Hz, 1H), 7.64-7.46 (m, 3H), 6.55 (d, J=8.5 Hz, 1H), 4.52 (d, J=5.7 Hz, 2H), 4.23 (s, 2H), 3.53-3.39 (m, 2H), 3.22 (q, J=6.0 Hz, 2H), 1.71 (s, 3H). MS (ESI): Calcd. For C₁₈H₁₇F₂ClN₄O₂: 395. found 396 (M+H)⁺.

### Example 50: N-(2-(2-((2,4-difluorobenzyl)amino)-5-oxo-5,7-dihydro-6H-pyrrolo[3,4-b]pyridin-6-yl)ethyl)propionamide (Compound 55)

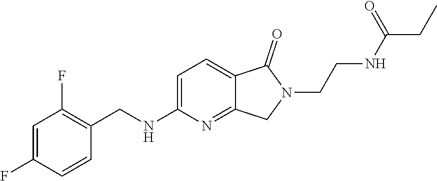 [SMILES: CCC(=O)NCCN1Cc2nc(NCc3ccc(F)cc3F)ccc2C1=O]

(2,4-Difluorophenyl)methanamine (321 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2,4-difluorobenzyl)amino)-5-oxo-5,7-dihydro-6H-pyrrolo[3,4-b]pyridin-6-yl)ethyl)propionamide (140 mg, 33% yield) as white solid. ¹H NMR (400 MHz, DMSO) δ 7.81 (t, J=5.9 Hz, 1H), 7.74 (t, J=5.8 Hz, 1H), 7.56 (d, J=8.5 Hz, 1H), 7.39 (td, J=8.7, 6.6 Hz, 1H), 7.19 (ddd, J=10.5, 9.3, 2.6 Hz, 1H), 7.01 (tdd, J=8.5, 2.6, 1.0 Hz, 1H), 6.52 (d, J=8.6 Hz, 1H), 4.52 (d, J=5.7 Hz, 2H), 4.23 (s, 2H), 3.58-3.38 (m, 2H), 3.22 (q, J=6.0 Hz, 2H), 1.97 (q, J=7.6 Hz, 2H), 0.89 (t, J=7.6 Hz, 3H). MS (ESI): Calcd. For C₁₉H₂₀F₂N₄O₂: 374. found 375 (M+H)⁺.

### Example 51: N-(2-(5-oxo-2-((2,4,5-trifluorobenzyl)amino)-5,7-dihydro-6H-pyrrolo[3,4-b]pyridin-6-yl)ethyl)propionamide (Compound 56)

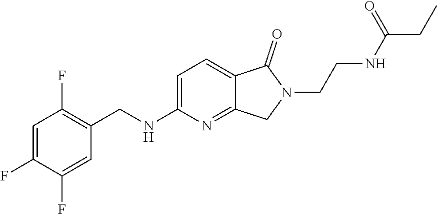 [SMILES: CCC(=O)NCCN1Cc2nc(NCc3cc(F)c(F)cc3F)ccc2C1=O]

(2,4,5-Trifluorophenyl)methanamine (361 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,4,5-trifluorobenzyl)amino)-5,7-dihydro-6H-pyrrolo[3,4-b]pyridin-6-yl)ethyl)propionamide (113 mg, 26% yield) as white solid. ¹H NMR (400 MHz, DMSO) δ 7.78 (dt, J=19.9, 5.9 Hz, 2H), 7.65-7.46 (m, 2H), 7.39 (ddd, J=11.1, 9.1, 6.8 Hz, 1H), 6.54 (d, J=8.5 Hz, 1H), 4.51 (d, J=5.8 Hz, 2H), 4.23 (s, 2H), 3.53-3.35 (m, 2H), 3.23 (dt, J=11.7, 5.1 Hz, 2H), 1.96 (q, J=7.6 Hz, 2H), 0.89 (t, J=7.6 Hz, 3H). MS (ESI): Calcd. For C₁₉H₁₉F₃N₄O₂: 392. found 393 (M+H)⁺.

### Example 52: N-(2-(2-(((5-methylpyridin-3-yl)methyl)amino)-5-oxo-5,7-dihydro-6H-pyrrolo[3,4-b]pyridin-6-yl)ethyl)propionamide (Compound 57)

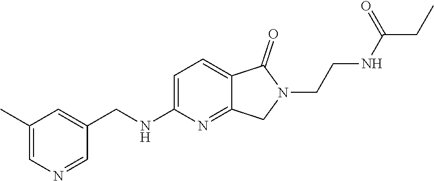 [SMILES: CCC(=O)NCCN1Cc2nc(NCc3cncc(C)c3)ccc2C1=O]

(5-Methylpyridin-3-yl)methanamine (361 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((5-methylpyridin-3-yl)methyl)amino)-5-oxo-5,7-dihydro-6H-pyrrolo[3,4-b]pyridin-6-yl)ethyl)propionamide (123 mg, 31% yield) as white solid. ¹H NMR (400 MHz, DMSO) δ 8.39-8.30 (m, 1H), 8.25 (dd, J=2.2, 0.9 Hz, 1H), 7.80 (dt, J=15.0, 5.9 Hz, 2H), 7.64-7.43 (m, 2H), 6.51 (d, J=8.6 Hz, 1H), 4.50 (d, J=5.8 Hz, 2H), 4.23 (s, 2H), 3.50-3.37 (m, 2H), 3.22 (q, J=6.0 Hz, 2H), 2.24 (t, J=0.7 Hz, 3H), 1.97 (q, J=7.6 Hz, 2H), 0.89 (t, J=7.6 Hz, 3H). MS (ESI): Calcd. For C₁₉H₂₃N₅O₂: 353. found 354 (M+H)⁺.

### Example 53: N-(2-(2-(((5-methylpyridin-3-yl)methyl)amino)-5-oxo-5,7-dihydro-6H-pyrrolo[3,4-b]pyridin-6-yl)ethyl)acetamide (Compound 58)

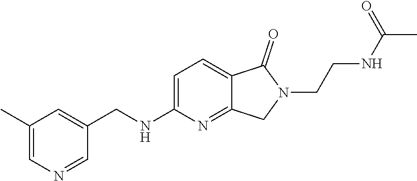 [SMILES: CC(=O)NCCN1Cc2nc(NCc3cncc(C)c3)ccc2C1=O]

(5-Methylpyridin-3-yl)methanamine (381 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((5-methylpyridin-3-yl)methyl)amino)-5-oxo-5,7-dihydro-6H-pyrrolo[3,4-b]pyridin-6-yl)ethyl)acetamide (119 mg, 30% yield) as white solid. ¹H NMR (400 MHz, DMSO) δ 8.33 (d, J=2.1 Hz, 1H), 8.26 (d, J=2.1 Hz, 1H), 7.90 (t, J=5.9 Hz, 1H), 7.78 (t, J=5.9 Hz, 1H), 7.63-7.45 (m, 2H), 6.52 (d, J=8.6 Hz, 1H), 4.51 (d, J=5.8 Hz, 2H), 4.23 (s, 2H), 3.55-3.37 (m, 2H), 3.22 (q, J=6.1 Hz, 2H), 2.24 (d, J=0.8 Hz, 3H), 1.71 (s, 3H). MS (ESI): Calcd. For C₁₈H₂₁N₅O₂: 339. found 340 (M+H)⁺.

### Example 54: N-(2-(2-(((2-methylpyridin-3-yl)methyl)amino)-5-oxo-5,7-dihydro-6H-pyrrolo[3,4-b]pyridin-6-yl)ethyl)propionamide (Compound 59)

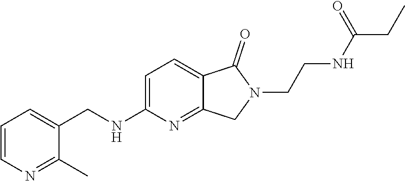 [SMILES: CCC(=O)NCCN1Cc2nc(NCc3cccnc3C)ccc2C1=O]

(2-Methylpyridin-3-yl)methanamine (361 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((2-methylpyridin-3-yl)methyl)amino)-5-oxo-5,7-dihydro-6H-pyrrolo[3,4-b]pyridin-6-yl)ethyl)propionamide (97 mg, 24% yield) as white solid. ¹H NMR (400 MHz, DMSO) δ 8.29 (dd, J=4.8, 1.7 Hz, 1H), 7.81 (t, J=5.9 Hz, 1H), 7.72 (t, J=5.7 Hz, 1H), 7.55 (dd, J=8.9, 1.7 Hz, 2H), 7.25-7.09 (m, 1H), 6.54 (d, J=8.6 Hz, 1H), 4.50 (d, J=5.6 Hz, 2H), 4.22 (s, 2H), 3.54-3.38 (m, 2H), 3.22 (q, J=6.0 Hz, 2H), 2.48 (s, 3H), 1.97 (q, J=7.6 Hz, 2H), 0.89 (t, J=7.6 Hz, 3H). MS (ESI): Calcd. For C₁₉H₂₃N₅O₂: 353. found 354 (M+H)⁺.

### Example 55: N-(2-(2-(((2-methylpyridin-3-yl)methyl)amino)-5-oxo-5,7-dihydro-6H-pyrrolo[3,4-b]pyridin-6-yl)ethyl)acetamide (Compound 60)

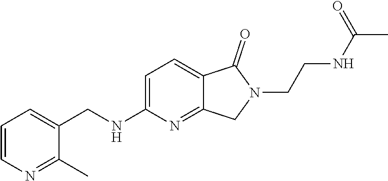 [SMILES: CC(=O)NCCN1Cc2nc(NCc3cccnc3C)ccc2C1=O]

(2-Methylpyridin-3-yl)methanamine (381 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((2-methylpyridin-3-yl)methyl)amino)-5-oxo-5,7-dihydro-6H-pyrrolo[3,4-b]pyridin-6-yl)ethyl)acetamide (103 mg, 26% yield) as white solid. ¹H NMR (400 MHz, DMSO) δ 8.29 (dd, J=4.8, 1.7 Hz, 1H), 7.89 (t, J=5.8 Hz, 1H), 7.73 (t, J=5.7 Hz, 1H), 7.62-7.50 (m, 2H), 7.14 (dd, J=7.6, 4.8 Hz, 1H), 6.54 (d, J=8.5 Hz, 1H), 4.50 (d, J=5.6 Hz, 2H), 4.23 (s, 2H), 3.43 (t, J=6.0 Hz, 2H), 3.23-3.16 (m, 2H), 2.48 (s, 3H), 1.71 (s, 3H). MS (ESI): Calcd. For C₁₈H₂₁N₅O₂: 339. found 340 (M+H)⁺.

### Example 56: N-(2-(2-(((4,6-dimethylpyridin-3-yl)methyl)amino)-5-oxo-5,7-dihydro-6H-pyrrolo[3,4-b]pyridin-6-yl)ethyl)acetamide (Compound 61)

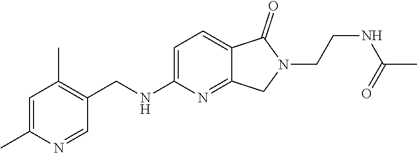 [SMILES: CC(=O)NCCN1Cc2nc(NCc3cnc(C)cc3C)ccc2C1=O]

(4,6-Dimethylpyridin-3-yl)methanamine (322 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((4,6-dimethylpyridin-3-yl)methyl)amino)-5-oxo-5,7-dihydro-6H-pyrrolo[3,4-b]pyridin-6-yl)ethyl)acetamide (145 mg, 35% yield) as white solid. ¹H NMR (400 MHz, DMSO) δ 8.25 (s, 1H), 7.90 (t, J=5.8 Hz, 1H), 7.61 (t, J=5.5 Hz, 1H), 7.54 (d, J=8.6 Hz, 1H), 7.03 (d, J=1.1 Hz, 1H), 6.51 (d, J=8.6 Hz, 1H), 4.46 (d, J=5.4 Hz, 2H), 4.24 (s, 2H), 3.51-3.38 (m, 2H), 3.22 (q, J=6.0 Hz, 2H), 2.35 (s, 3H), 2.26 (d, J=0.7 Hz, 3H), 1.72 (s, 3H). MS (ESI): Calcd. for C₁₉H₂₃N₅O₂: 353. found 354 (M+H)⁺.

### Example 57: N-(2-(5-oxo-2-((3,4,5-trifluorobenzyl)amino)-5,7-dihydro-6H-pyrrolo[3,4-b]pyridin-6-yl)ethyl)acetamide (Compound 71)

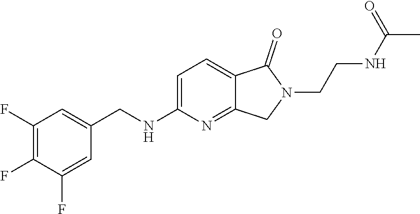 [SMILES: CC(=O)NCCN1Cc2nc(NCc3cc(F)c(F)c(F)c3)ccc2C1=O]

(3,4,5-Trifluorophenyl)methanamine (381 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((3,4,5-trifluorobenzyl)amino)-5,7-dihydro-6H-pyrrolo[3,4-b]pyridin-6-yl)ethyl)acetamide (138 mg, 31% yield) as white solid. ¹H NMR (400 MHz, DMSO) δ 7.86 (dt, J=25.8, 6.0 Hz, 2H), 7.58 (d, J=8.6 Hz, 1H), 7.31-7.17 (m, 2H), 6.54 (d, J=8.6 Hz, 1H), 4.51 (d, J=6.0 Hz, 2H), 4.22 (s, 2H), 3.55-3.37 (m, 2H), 3.21 (q, J=6.1 Hz, 2H), 1.71 (s, 3H). MS (ESI): Calcd. for C₁₈H₁₇F₃N₄O₂: 378. found 379 (M+H)⁺.

### Example 58: N-(2-(5-oxo-2-((3,4,5-trifluorobenzyl)amino)-5,7-dihydro-6H-pyrrolo[3,4-b]pyridin-6-yl)ethyl)propionamide (Compound 72)

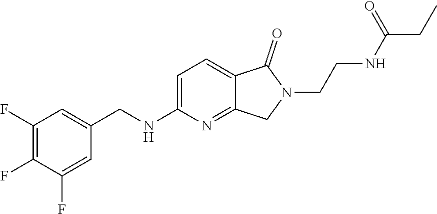 [SMILES: CCC(=O)NCCN1Cc2nc(NCc3cc(F)c(F)c(F)c3)ccc2C1=O]

(3,4,5-Trifluorophenyl)methanamine (361 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((3,4,5-trifluorobenzyl)amino)-5,7-dihydro-6H-pyrrolo[3,4-b]pyridin-6-yl)ethyl)propionamide (145 mg, 33% yield) as white solid. ¹H NMR (400 MHz, DMSO) δ 7.82 (q, J=5.8 Hz, 2H), 7.58 (d, J=8.5 Hz, 1H), 7.33-7.14 (m, 2H), 6.53 (d, J=8.5 Hz, 1H), 4.51 (d, J=6.0 Hz, 2H), 4.22 (s, 2H), 3.53-3.37 (m, 2H), 3.22 (q, J=6.0 Hz, 2H), 1.96 (q, J=7.6 Hz, 2H), 0.88 (t, J=7.6 Hz, 3H). MS (ESI): Calcd. for C₁₉H₁₉F₃N₄O₂: 392. found 393 (M+H)⁺.

### Example 59: N-(2-(5-oxo-2-((2,4,6-trifluorobenzyl)amino)-5,7-dihydro-6H-pyrrolo[3,4-b]pyridin-6-yl)ethyl)propionamide (Compound 73)

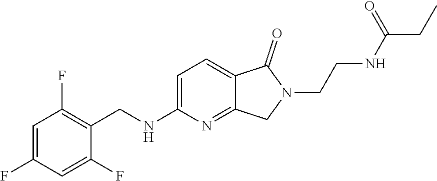 [SMILES: CCC(=O)NCCN1Cc2nc(NCc3c(F)cc(F)cc3F)ccc2C1=O]

(2,4,6-Trifluorophenyl)methanamine (361 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,4,6-trifluorobenzyl)amino)-5,7-dihydro-6H-pyrrolo[3,4-b]pyridin-6-yl)ethyl)propionamide (108 mg, 25% yield) as white solid. ¹H NMR (400 MHz, DMSO) δ 7.83 (t, J=5.9 Hz, 1H), 7.62 (t, J=5.2 Hz, 1H), 7.53 (d, J=8.6 Hz, 1H), 7.24-7.07 (m, 2H), 6.48 (d, J=8.6 Hz, 1H), 4.48 (d, J=5.1 Hz, 2H), 4.25 (s, 2H), 3.53-3.40 (m, 2H), 3.24-3.13 (m, 2H), 1.97 (q, J=7.6 Hz, 2H), 0.89 (t, J=7.6 Hz, 3H). MS (ESI): Calcd. for C₁₉H₁₉F₃N₄O₂: 392. found 393 (M+H)⁺.

### Example 60: N-(2-(5-oxo-2-(((4-(trifluoromethyl)pyridin-3-yl)methyl)amino)-5,7-dihydro-6H-pyrrolo[3,4-b]pyridin-6-yl)ethyl)acetamide (Compound 62)

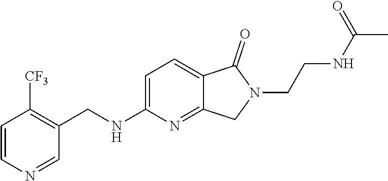 [SMILES: CC(=O)NCCN1Cc2nc(NCc3cnccc3C(F)(F)F)ccc2C1=O]

(4-(Trifluoromethyl)pyridin-3-yl)methanamine (381 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-(((4-(trifluoromethyl)pyridin-3-yl)methyl)amino)-5,7-dihydro-6H-pyrrolo[3,4-b]pyridin-6-yl)ethyl)acetamide (95 mg, 20% yield) as white solid. ¹H NMR (400 MHz, DMSO) δ 8.77 (s, 1H), 8.76-8.65 (m, 1H), 7.88 (q, J=5.5 Hz, 2H), 7.70 (d, J=5.1 Hz, 1H), 7.60 (d, J=8.6 Hz, 1H), 6.59 (d, J=8.6 Hz, 1H), 4.73 (d, J=5.6 Hz, 2H), 4.22 (s, 2H), 3.51-3.37 (m, 2H), 3.21 (q, J=6.0 Hz, 2H), 1.70 (s, 3H). MS (ESI): Calcd. for C₁₈H₁₈F₃N₅O₂: 393. found 394 (M+H)⁺.

### Example 61: N-(2-(5-oxo-2-(((4-(trifluoromethyl)pyridin-3-yl)methyl)amino)-5,7-dihydro-6H-pyrrolo[3,4-b]pyridin-6-yl)ethyl)propionamide (Compound 74)

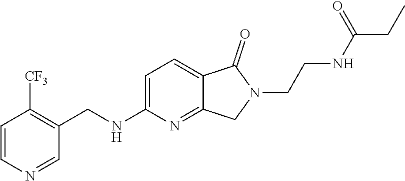 [SMILES: CCC(=O)NCCN1Cc2nc(NCc3cnccc3C(F)(F)F)ccc2C1=O]

(4-(Trifluoromethyl)pyridin-3-yl)methanamine (361 mg, 2.24 mmol) was added to a solution of N-(2-(2-chloro-5-oxo-5,7-dihydro-6H-pyrrolo[3,4-b]pyridin-6-yl)ethyl) propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-(((4-(trifluoromethyl)pyridin-3-yl)methyl)amino)-5,7-dihydro-6H-pyrrolo[3,4-b]pyridin-6-yl)ethyl)propionamide (102 mg, 22% yield) as white solid. ¹H NMR (400 MHz, DMSO) δ 8.77 (s, 1H), 8.77-8.66 (m, 1H), 7.88 (t, J=5.7 Hz, 1H), 7.81 (t, J=5.9 Hz, 1H), 7.70 (d, J=5.1 Hz, 1H), 7.60 (d, J=8.5 Hz, 1H), 6.59 (d, J=8.5 Hz, 1H), 4.73 (d, J=5.5 Hz, 2H), 4.22 (s, 2H), 3.53-3.37 (m, 2H), 3.21 (q, J=6.0 Hz, 2H), 1.96 (q, J=7.6 Hz, 2H), 0.88 (t, J=7.6 Hz, 3H). MS (ESI): Calcd. for C₁₉H₂₀F₃N₅O₂: 407. found 408 (M+H)⁺.

### Example 62: N-(2-(5-oxo-2-((2,3,4-trifluorobenzyl)amino)-5,7-dihydro-6H-pyrrolo[3,4-b]pyridin-6-yl)ethyl)acetamide (Compound 75)

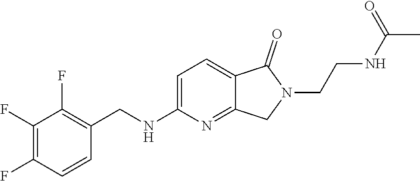 [SMILES: CC(=O)NCCN1Cc2nc(NCc3ccc(F)c(F)c3F)ccc2C1=O]

(2,3,4-Trifluorophenyl)methanamine (381 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,3,4-trifluorobenzyl)amino)-5,7-dihydro-6H-pyrrolo[3,4-b]pyridin-6-yl)ethyl)acetamide (120 mg, 27% yield) as white solid. ¹H NMR (400 MHz, DMSO) δ 7.90 (t, J=5.9 Hz, 1H), 7.81 (t, J=5.9 Hz, 1H), 7.57 (d, J=8.6 Hz, 1H), 7.32-7.12 (m, 2H), 6.54 (d, J=8.5 Hz, 1H), 4.56 (d, J=5.8 Hz, 2H), 4.23 (s, 2H), 3.50-3.39 (m, 2H), 3.21 (q, J=6.0 Hz, 2H), 1.71 (s, 3H). MS (ESI): Calcd. for C₁₈H₁₇F₃N₄O₂: 378. found 379 (M+H)⁺.

### Example 63: N-(2-(5-oxo-2-((2,3,4-trifluorobenzyl)amino)-5,7-dihydro-6H-pyrrolo[3,4-b]pyridin-6-yl)ethyl)propionamide (Compound 76)

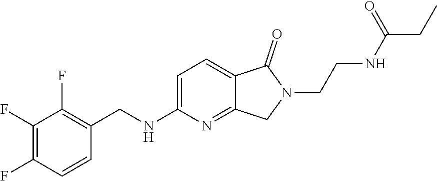 [SMILES: CCC(=O)NCCN1Cc2nc(NCc3ccc(F)c(F)c3F)ccc2C1=O]

(2,3,4-Trifluorophenyl)methanamine (361 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,3,4-trifluorobenzyl)amino)-5,7-dihydro-6H-pyrrolo[3,4-b]pyridin-6-yl)ethyl)propionamide (117 mg, 27% yield) as white solid. ¹H NMR (400 MHz, DMSO) δ 7.81 (td, J=5.7, 2.3 Hz, 2H), 7.57 (d, J=8.6 Hz, 1H), 7.35-7.12 (m, 2H), 6.53 (d, J=8.6 Hz, 1H), 4.56 (d, J=5.8 Hz, 2H), 4.22 (s, 2H), 3.54-3.37 (m, 2H), 3.22 (q, J=6.0 Hz, 2H), 1.96 (q, J=7.6 Hz, 2H), 0.89 (t, J=7.6 Hz, 3H). MS (ESI): Calcd. for C₁₉H₁₉F₃N₄O₂: 392. found 393 (M+H)⁺.

### Example 64: N-(2-(2-((4-fluoro-2-methylbenzyl)amino)-5-oxo-5,7-dihydro-6H-pyrrolo[3,4-b]pyridin-6-yl)ethyl)acetamide (Compound 77)

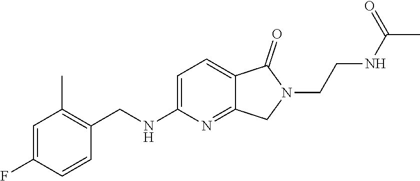 [SMILES: CC(=O)NCCN1Cc2nc(NCc3ccc(F)cc3C)ccc2C1=O]

(4-Fluoro-2-methyphenyl)methanamine (329 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-fluoro-2-methylbenzyl)amino)-5-oxo-5,7-dihydro-6H-pyrrolo[3,4-b]pyridin-6-yl)ethyl)acetamide (145 mg, 34% yield) as white solid. ¹H NMR (400 MHz, DMSO) δ 7.90 (t, J=5.8 Hz, 1H), 7.62 (t, J=5.5 Hz, 1H), 7.54 (d, J=8.6 Hz, 1H), 7.24 (dd, J=8.5, 6.1 Hz, 1H), 7.12-6.98 (m, 1H), 6.92 (tt, J=8.7, 3.1 Hz, 1H), 6.52 (d, J=8.6 Hz, 1H), 4.44 (d, J=5.5 Hz, 2H), 4.23 (s, 2H), 3.47-3.36 (m, 2H), 3.22 (q, J=6.0 Hz, 2H), 2.29 (s, 3H), 1.72 (s, 3H). MS (ESI): Calcd. for C₁₉H₂₁FN₄O₂: 356. found 357 (M+H)⁺.

### Example 66: N-(2-(2-((4-fluoro-2-methylbenzyl)amino)-5-oxo-5,7-dihydro-6H-pyrrolo[3,4-b]pyridin-6-yl)ethyl)propionamide (Compound 78)

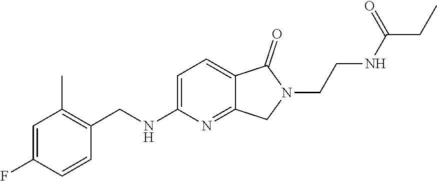 [SMILES: CCC(=O)NCCN1Cc2nc(NCc3ccc(F)cc3C)ccc2C1=O]

(4-Fluoro-2-methyphenyl)methanamine (312 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-fluoro-2-methylbenzyl)amino)-5-oxo-5,7-dihydro-6H-pyrrolo[3,4-b]pyridin-6-yl)ethyl)propionamide (113 mg, 27% yield) as white solid. ¹H NMR (400 MHz, DMSO) δ 7.82 (t, J=5.9 Hz, 1H), 7.61 (t, J=5.6 Hz, 1H), 7.54 (d, J=8.6 Hz, 1H), 7.24 (dd, J=8.5, 6.1 Hz, 1H), 7.01 (ddd, J=10.0, 2.8, 0.8 Hz, 1H), 6.92 (td, J=8.8, 2.8 Hz, 1H), 6.51 (d, J=8.6 Hz, 1H), 4.44 (d, J=5.5 Hz, 2H), 4.23 (s, 2H), 3.49-3.37 (m, 2H), 3.22 (q, J=6.0 Hz, 2H), 2.29 (s, 3H), 1.97 (q, J=7.6 Hz, 2H), 0.89 (t, J=7.6 Hz, 3H). MS (ESI): Calcd. for C₂₀H₂₃FN₄O₂: 370. found 371 (M+H)⁺.

### Example 66: N-(2-(2-((2-chloro-4-fluorobenzyl)amino)-5-oxo-5,7-dihydro-6H-pyrrolo[3,4-b]pyridin-6-yl)ethyl)acetamide (Compound 79)

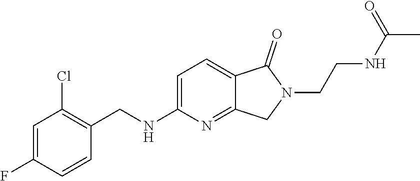 [SMILES: CC(=O)NCCN1Cc2nc(NCc3ccc(F)cc3Cl)ccc2C1=O]

(2-Chloro-4-fluorophenyl)methanamine (377 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2-chloro-4-fluorobenzyl)amino)-5-oxo-5,7-dihydro-6H-pyrrolo[3,4-b]pyridin-6-yl)ethyl)acetamide (133 mg, 30% yield) as white solid. ¹H NMR (400 MHz, DMSO) δ 7.90 (t, J=5.9 Hz, 1H), 7.78 (t, J=5.8 Hz, 1H), 7.58 (d, J=8.5 Hz, 1H), 7.47-7.30 (m, 2H), 7.16 (td, J=8.5, 2.6 Hz, 1H), 6.56 (d, J=8.5 Hz, 1H), 4.55 (d, J=5.8 Hz, 2H), 4.22 (s, 2H), 3.53-3.38 (m, 2H), 3.21 (q, J=6.0 Hz, 2H), 1.71 (s, 3H). MS (ESI): Calcd. for C₁₈H₁₈ClFN₄O₂: 377. found 378 (M+H)⁺.

### Example 67: N-(2-(2-((2-chloro-4-fluorobenzyl)amino)-5-oxo-5,7-dihydro-6H-pyrrolo[3,4-b]pyridin-6-yl)ethyl)propionamide (Compound 80)

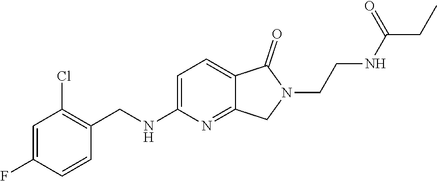 [SMILES: CCC(=O)NCCN1Cc2nc(NCc3ccc(F)cc3Cl)ccc2C1=O]

(2-Chloro-4-fluorophenyl)methanamine (358 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2-chloro-4-fluorobenzyl)amino)-5-oxo-5,7-dihydro-6H-pyrrolo[3,4-b]pyridin-6-yl)ethyl)propionamide (145 mg, 33% yield) as white solid. ¹H NMR (400 MHz, DMSO) δ 7.79 (dt, J=15.2, 5.8 Hz, 2H), 7.57 (d, J=8.6 Hz, 1H), 7.46-7.30 (m, 2H), 7.16 (td, J=8.5, 2.7 Hz, 1H), 6.56 (d, J=8.6 Hz, 1H), 4.55 (d, J=5.7 Hz, 2H), 4.22 (s, 2H), 3.43 (t, J=6.0 Hz, 2H), 3.21 (q, J=6.0 Hz, 2H), 1.96 (q, J=7.6 Hz, 2H), 0.89 (t, J=7.6 Hz, 3H). MS (ESI): Calcd. for C₁₉H₂₀ClFN₄O₂: 391. found 392 (M+H)⁺.

### Example 68: N-(2-(2-((5-fluoro-2-methylbenzyl)amino)-5-oxo-5,7-dihydro-6H-pyrrolo[3,4-b]pyridin-6-yl)ethyl)acetamide (Compound 81)

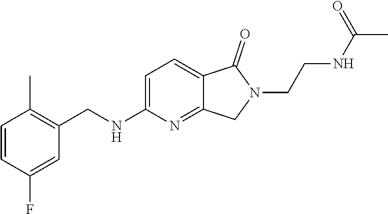 [SMILES: CC(=O)NCCN1Cc2nc(NCc3cc(F)ccc3C)ccc2C1=O]

(5-Fluoro-2-methyphenyl)methanamine (329 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((5-fluoro-2-methylbenzyl)amino)-5-oxo-5,7-dihydro-6H-pyrrolo[3,4-b]pyridin-6-yl)ethyl)acetamide (139 mg, 33% yield) as white solid. ¹H NMR (400 MHz, DMSO) δ 7.90 (t, J=5.8 Hz, 1H), 7.70 (t, J=5.8 Hz, 1H), 7.57 (d, J=8.6 Hz, 1H), 7.30-7.13 (m, 1H), 7.09-6.90 (m, 2H), 6.55 (d, J=8.5 Hz, 1H), 4.47 (d, J=5.7 Hz, 2H), 4.23 (s, 2H), 3.52-3.37 (m, 2H), 3.21 (q, J=6.0 Hz, 2H), 2.25 (s, 3H), 1.71 (s, 3H). MS (ESI): Calcd. for C₁₉H₂₁FN₄O₂: 356. found 357 (M+H)⁺.

### Example 69: N-(2-(2-((5-fluoro-2-methylbenzyl)amino)-5-oxo-5,7-dihydro-6H-pyrrolo[3,4-b]pyridin-6-yl)ethyl)propionamide (Compound 82)

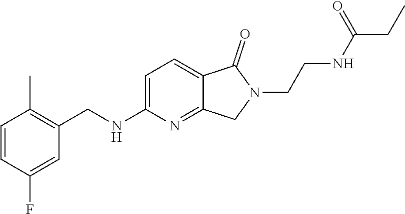 [SMILES: CCC(=O)NCCN1Cc2nc(NCc3cc(F)ccc3C)ccc2C1=O]

(5-Fluoro-2-methyphenyl)methanamine (312 mg, 2.24 mmol) was added to a solution of N-(2-(2-chloro-5-oxo-5,7-dihydro-6H-pyrrolo[3,4-b]pyridin-6-yl)ethyl)propionamide (300 mg, 1.12 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((5-fluoro-2-methylbenzyl)amino)-5-oxo-5,7-dihydro-6H-pyrrolo[3,4-b]pyridin-6-yl)ethyl)propionamide (105 mg, 25% yield) as white solid. ¹H NMR (400 MHz, DMSO) δ 7.81 (t, J=5.9 Hz, 1H), 7.69 (t, J=5.8 Hz, 1H), 7.56 (d, J=8.6 Hz, 1H), 7.17 (ddd, J=8.4, 5.9, 0.8 Hz, 1H), 7.06-6.88 (m, 2H), 6.54 (d, J=8.6 Hz, 1H), 4.47 (d, J=5.7 Hz, 2H), 4.22 (s, 2H), 3.53-3.37 (m, 2H), 3.22 (q, J=6.0 Hz, 2H), 2.25 (s, 3H), 1.97 (q, J=7.6 Hz, 2H), 0.89 (t, J=7.6 Hz, 3H). MS (ESI): Calcd. for C₂₀H₂₃FN₄O₂: 370. found 371 (M+H)⁺.

### Example 70: N-(2-(2-((2-methylbenzyl)amino)-5-oxo-5,7-dihydro-6H-pyrrolo[3,4-b]pyridin-6-yl)ethyl)acetamide (Compound 83)

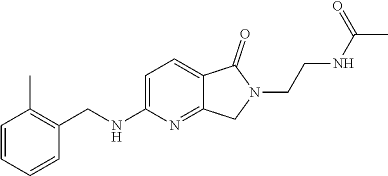 [SMILES: CC(=O)NCCN1Cc2nc(NCc3ccccc3C)ccc2C1=O]

(2-Methyphenyl)methanamine (287 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2-methylbenzyl)amino)-5-oxo-5,7-dihydro-6H-pyrrolo[3,4-b]pyridin-6-yl)ethyl)acetamide (113 mg, 28% yield) as white solid. ¹H NMR (400 MHz, DMSO) δ 7.90 (t, J=5.9 Hz, 1H), 7.62 (t, J=5.6 Hz, 1H), 7.54 (d, J=8.6 Hz, 1H), 7.22 (dd, J=6.3, 2.3 Hz, 1H), 7.17-7.06 (m, 3H), 6.53 (d, J=8.6 Hz, 1H), 4.47 (d, J=5.5 Hz, 2H), 4.23 (s, 2H), 3.50-3.38 (m, 2H), 3.22 (q, J=6.0 Hz, 2H), 2.28 (s, 3H), 1.72 (s, 3H). MS (ESI): Calcd. for C₁₉H₂₂N₄O₂: 338. found 339 (M+H)⁺.

### Example 71: N-(2-(2-((2-methylbenzyl)amino)-5-oxo-5,7-dihydro-6H-pyrrolo[3,4-b]pyridin-6-yl)ethyl)propionamide (Compound 84)

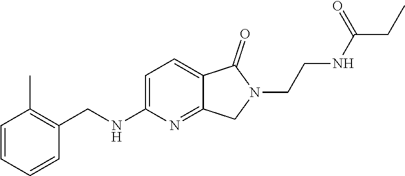 [SMILES: CCC(=O)NCCN1Cc2nc(NCc3ccccc3C)ccc2C1=O]

(2-Methyphenyl)methanamine (181 mg, 1.49 mmol) was added to a solution of N-(2-(2-chloro-5-oxo-5,7-dihydro-6H-pyrrolo[3,4-b]pyridin-6-yl)ethyl)propionamide (200 mg, 0.75 mmol) and DIPEA (0.65 mL, 3.74 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2-methylbenzyl)amino)-5-oxo-5,7-dihydro-6H-pyrrolo[3,4-b]pyridin-6-yl)ethyl)propionamide (101 mg, 38% yield) as white solid. ¹H NMR (400 MHz, DMSO) δ 7.82 (t, J=5.8 Hz, 1H), 7.61 (t, J=5.5 Hz, 1H), 7.54 (d, J=8.6 Hz, 1H), 7.31-7.18 (m, 1H), 7.16-7.02 (m, 3H), 6.52 (d, J=8.6 Hz, 1H), 4.47 (d, J=5.5 Hz, 2H), 4.23 (s, 2H), 3.52-3.37 (m, 2H), 3.22 (q, J=7.0 Hz, 2H), 2.28 (s, 3H), 1.97 (q, J=7.6 Hz, 2H), 0.89 (t, J=7.6 Hz, 3H). MS (ESI): Calcd. For C₂₀H₂₄N₄O₂: 352. found 353 (M+H)⁺.

### Example 72: N-(2-(2-((3-fluorobenzyl)amino)-5-oxo-5,7-dihydro-6H-pyrrolo[3,4-b]pyridin-6-yl)ethyl)acetamide (Compound 85)

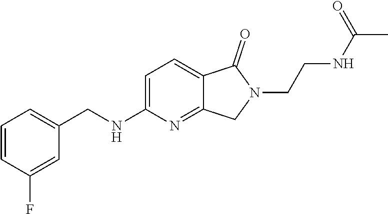 [SMILES: CC(=O)NCCN1Cc2nc(NCc3cccc(F)c3)ccc2C1=O]

(3-Fluorophenyl)methanamine (296 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((3-fluorobenzyl)amino)-5-oxo-5,7-dihydro-6H-pyrrolo[3,4-b]pyridin-6-yl)ethyl)acetamide (149 mg, 37% yield) as white solid. ¹H NMR (400 MHz, DMSO) δ 7.90 (t, J=6.0 Hz, 1H), 7.81 (t, J=6.1 Hz, 1H), 7.56 (d, J=8.5 Hz, 1H), 7.33 (td, J=7.9, 6.1 Hz, 1H), 7.23-6.95 (m, 3H), 6.52 (d, J=8.6 Hz, 1H), 4.54 (d, J=6.0 Hz, 2H), 4.22 (s, 2H), 3.53-3.36 (m, 2H), 3.21 (q, J=6.0 Hz, 2H), 1.71 (s, 3H). MS (ESI): Calcd. for C₁₈H₁₉FN₄O₂: 342. found 343 (M+H)⁺.

### Example 73: N-(2-(2-((3-fluorobenzyl)amino)-5-oxo-5,7-dihydro-6H-pyrrolo[3,4-b]pyridin-6-yl)ethyl)propionamide (Compound 86)

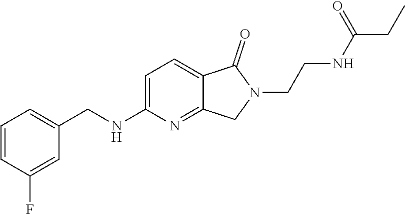 [SMILES: CCC(=O)NCCN1Cc2nc(NCc3cccc(F)c3)ccc2C1=O]

(3-Fluorophenyl)methanamine (187 mg, 1.49 mmol) was added to a solution of N-(2-(2-chloro-5-oxo-5,7-dihydro-6H-pyrrolo[3,4-b]pyridin-6-yl)ethyl)propionamide (200 mg, 0.75 mmol) and DIPEA (0.65 mL, 3.74 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((3-fluorobenzyl)amino)-5-oxo-5,7-dihydro-6H-pyrrolo[3,4-b]pyridin-6-yl)ethyl)propionamide (104 mg, 39% yield) as white solid. ¹H NMR (400 MHz, DMSO) δ 7.81 (dt, J=5.9, 3.0 Hz, 2H), 7.56 (d, J=8.6 Hz, 1H), 7.33 (td, J=8.0, 6.1 Hz, 1H), 7.21-6.97 (m, 3H), 6.52 (d, J=8.6 Hz, 1H), 4.54 (d, J=6.0 Hz, 2H), 4.22 (s, 2H), 3.54-3.38 (m, 2H), 3.22 (q, J=6.0 Hz, 2H), 1.96 (q, J=7.6 Hz, 2H), 0.89 (t, J=7.6 Hz, 3H). MS (ESI): Calcd. for C₁₉H₂₁FN₄O₂: 356. found 357 (M+H)⁺.

### Example 74: N-(2-(2-(((6-methylpyridin-3-yl)methyl)amino)-5-oxo-5,7-dihydro-6H-pyrrolo[3,4-b]pyridin-6-yl)ethyl)cyclopropanecarboxamide (Compound 87)

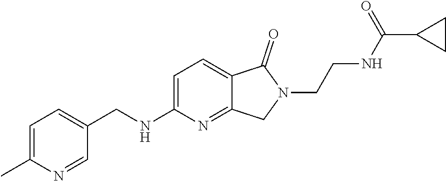 [SMILES: Cc1ccc(CNc2ccc3c(n2)CN(CCNC(=O)C2CC2)C3=O)cn1]

(6-Methylpyridin-3-yl)methanamine (262 mg, 2.14 mmol) was added to a solution of N-(2-(2-chloro-5-oxo-5,7-dihydro-6H-pyrrolo[3,4-b]pyridin-6-yl)ethyl)cyclopropanecarboxamide (300 mg, 1.07 mmol) and DIPEA (0.93 mL, 5.36 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((6-methylpyridin-3-yl)methyl)amino)-5-oxo-5,7-dihydro-6H-pyrrolo[3,4-b]pyridin-6-yl)ethyl)cyclopropanecarboxamide (109 mg, 28% yield) as white solid. ¹H NMR (400 MHz, DMSO) δ 8.39 (dd, J=2.4, 0.8 Hz, 1H), 8.11 (t, J=5.9 Hz, 1H), 7.77 (t, J=5.9 Hz, 1H), 7.66-7.47 (m, 2H), 7.23-7.08 (m, 1H), 6.50 (d, J=8.6 Hz, 1H), 4.48 (d, J=5.9 Hz, 2H), 4.23 (s, 2H), 3.44 (t, J=6.3 Hz, 2H), 3.27-3.20 (m, 2H), 2.39 (s, 3H), 1.43 (tt, J=7.2, 5.7 Hz, 1H), 0.66-0.43 (m, 4H). MS (ESI): Calcd. for C₂₀H₂₃N₅O₂: 365. found 366 (M+H)⁺.

### Example 75: N-(2-(2-(((4-methylpyridin-3-yl)methyl)amino)-5-oxo-5,7-dihydro-6H-pyrrolo[3,4-b]pyridin-6-yl)ethyl)cyclopropanecarboxamide (Compound 88)

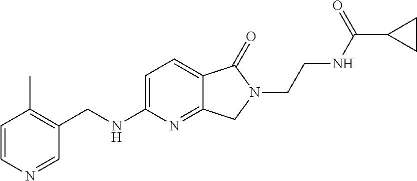 [SMILES: Cc1ccncc1CNc1ccc2c(n1)CN(CCNC(=O)C1CC1)C2=O]

(4-Methylpyridin-3-yl)methanamine (262 mg, 2.14 mmol) was added to a solution of N-(2-(2-chloro-5-oxo-5,7-dihydro-6H-pyrrolo[3,4-b]pyridin-6-yl)ethyl)cyclopropanecarboxamide (300 mg, 1.07 mmol) and DIPEA (0.93 mL, 5.36 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((4-methylpyridin-3-yl)methyl)amino)-5-oxo-5,7-dihydro-6H-pyrrolo[3,4-b]pyridin-6-yl)ethyl)cyclopropanecarboxamide (89 mg, 23% yield) as white solid. ¹H NMR (400 MHz, DMSO) δ 8.39 (s, 1H), 8.29 (d, J=4.9 Hz, 1H), 8.11 (t, J=5.8 Hz, 1H), 7.67 (t, J=5.5 Hz, 1H), 7.56 (d, J=8.6 Hz, 1H), 7.17 (dt, J=4.8, 0.8 Hz, 1H), 6.53 (d, J=8.6 Hz, 1H), 4.51 (d, J=5.5 Hz, 2H), 4.25 (s, 2H), 3.44 (t, J=6.1 Hz, 2H), 3.27-3.23 (m, 2H), 2.31 (d, J=0.6 Hz, 3H), 1.43 (tt, J=7.1, 5.7 Hz, 1H), 0.65-0.48 (m, 4H). MS (ESI): Calcd. For C₂₀H₂₃N₅O₂: 365. found 366 (M+H)⁺.

### Example 76: N-(2-(2-(((2-methylpyridin-3-yl)methyl)amino)-5-oxo-5,7-dihydro-6H-pyrrolo[3,4-b]pyridin-6-yl)ethyl)cyclopropanecarboxamide (Compound 89)

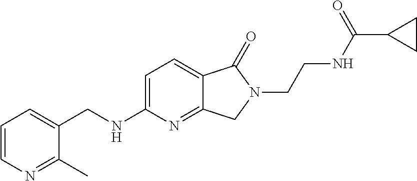 [SMILES: Cc1ncccc1CNc1ccc2c(n1)CN(CCNC(=O)C1CC1)C2=O]

(2-Methylpyridin-3-yl)methanamine (262 mg, 2.14 mmol) was added to a solution of N-(2-(2-chloro-5-oxo-5,7-dihydro-6H-pyrrolo[3,4-b]pyridin-6-yl)ethyl)cyclopropanecarboxamide (300 mg, 1.07 mmol) and DIPEA (0.93 mL, 5.36 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((2-methylpyridin-3-yl)methyl)amino)-5-oxo-5,7-dihydro-6H-pyrrolo[3,4-b]pyridin-6-yl)ethyl)cyclopropanecarboxamide (96 mg, 25% yield) as white solid. ¹H NMR (400 MHz, DMSO) δ 8.28 (dd, J=4.9, 1.8 Hz, 1H), 8.10 (t, J=5.9 Hz, 1H), 7.73 (t, J=5.7 Hz, 1H), 7.56 (dd, J=8.2, 4.2 Hz, 2H), 7.23-7.06 (m, 1H), 6.54 (d, J=8.6 Hz, 1H), 4.50 (d, J=5.6 Hz, 2H), 4.23 (s, 2H), 3.44 (t, J=6.1 Hz, 2H), 3.23 (d, J=6.2 Hz, 2H), 2.48 (s, 3H), 1.43 (tt, J=7.2, 5.7 Hz, 1H), 0.66-0.47 (m, 4H). MS (ESI): Calcd. for C₂₀H₂₃N₅O₂: 365. found 366 (M+H)⁺.

### Example 77: N-(2-(5-oxo-2-((2,4,6-trifluorobenzyl)amino)-5,7-dihydro-6H-pyrrolo[3,4-b]pyridin-6-yl)ethyl)cyclopropanecarboxamide (Compound 90)

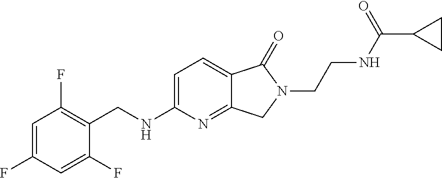 [SMILES: O=C(NCCN1Cc2nc(NCc3c(F)cc(F)cc3F)ccc2C1=O)C1CC1]

(2,4,6-Trifluorophenyl)methanamine (346 mg, 2.14 mmol) was added to a solution of N-(2-(2-chloro-5-oxo-5,7-dihydro-6H-pyrrolo[3,4-b]pyridin-6-yl)ethyl)cyclopropanecarboxamide (300 mg, 1.07 mmol) and DIPEA (0.93 mL, 5.36 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,4,6-trifluorobenzyl)amino)-5,7-dihydro-6H-pyrrolo[3,4-b]pyridin-6-yl)ethyl)cyclopropanecarboxamide (115 mg, 27% yield) as white solid. ¹H NMR (400 MHz, DMSO) δ 8.11 (t, J=5.9 Hz, 1H), 7.62 (t, J=5.2 Hz, 1H), 7.54 (d, J=8.6 Hz, 1H), 7.29-7.05 (m, 2H), 6.49 (d, J=8.6 Hz, 1H), 4.49 (d, J=5.1 Hz, 2H), 4.25 (s, 2H), 3.45 (t, J=6.0 Hz, 2H), 3.27-3.17 (m, 2H), 1.43 (tt, J=7.1, 5.7 Hz, 1H), 0.66-0.48 (m, 4H). MS (ESI): Calcd. for C₂₀H₁₉F₃N₄O₂: 404. found 405 (M+H)⁺.

### Example 78: N-(2-(2-((4-chloro-2-fluorobenzyl)amino)-5-oxo-5,7-dihydro-6H-pyrrolo[3,4-b]pyridin-6-yl)ethyl)acetamide (Compound 91)

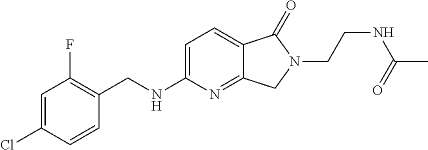 [SMILES: CC(=O)NCCN1Cc2nc(NCc3ccc(Cl)cc3F)ccc2C1=O]

(4-Chloro-2-fluorophenyl)methanamine (381 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-chloro-2-fluorobenzyl)amino)-5-oxo-5,7-dihydro-6H-pyrrolo[3,4-b]pyridin-6-yl)ethyl)acetamide (119 mg, 27% yield) as white solid. ¹H NMR (400 MHz, DMSO) δ 7.89 (t, J=5.9 Hz, 1H), 7.77 (t, J=5.9 Hz, 1H), 7.57 (dd, J=8.6, 1.4 Hz, 1H), 7.47-7.29 (m, 2H), 7.21 (ddd, J=8.2, 2.1, 0.7 Hz, 1H), 6.54 (d, J=8.5 Hz, 1H), 4.53 (d, J=5.8 Hz, 2H), 4.22 (s, 2H), 3.43 (t, J=6.1 Hz, 2H), 3.21 (q, J=5.9 Hz, 2H), 1.71 (d, J=1.5 Hz, 3H). MS (ESI): Calcd. for C₁₈H₁₈ClFN₄O₂: 376. found 377 (M+H)⁺.

### Example 79: N-(2-(2-((4-chloro-2,6-difluorobenzyl)amino)-5-oxo-5,7-dihydro-6H-pyrrolo[3,4-b]pyridin-6-yl)ethyl)acetamide (Compound 92)

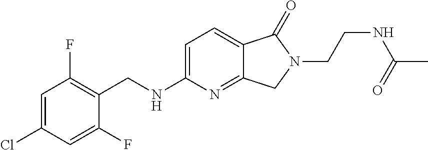 [SMILES: CC(=O)NCCN1Cc2nc(NCc3c(F)cc(Cl)cc3F)ccc2C1=O]

(4-Chloro-2,6-difluorophenyl)methanamine (364 mg, 2.05 mmol) was added to a solution of N-(2-(2-chloro-5-oxo-5,7-dihydro-6H-pyrrolo[3,4-b]pyridin-6-yl)ethyl)acetamide (260 mg, 1.02 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-chloro-2,6-difluorobenzyl)amino)-5-oxo-5,7-dihydro-6H-pyrrolo[3,4-b]pyridin-6-yl)ethyl)acetamide (119 mg, 29% yield) as white solid. ¹H NMR (400 MHz, DMSO) δ 7.89 (t, J=5.9 Hz, 1H), 7.65 (t, J=5.3 Hz, 1H), 7.54 (d, J=8.5 Hz, 1H), 7.41-7.24 (m, 2H), 6.49 (d, J=8.6 Hz, 1H), 4.50 (d, J=5.2 Hz, 2H), 4.24 (s, 2H), 3.54-3.37 (m, 2H), 3.22 (q, J=6.0 Hz, 2H), 1.72 (s, 3H). MS (ESI): Calcd. for C₁₈H₁₇ClF₂N₄O₂: 394. found 395 (M+H)⁺.

### Example 80: N-(2-(2-((2-fluoro-4-methylbenzyl)amino)-5-oxo-5,7-dihydro-6H-pyrrolo[3,4-b]pyridin-6-yl)ethyl)acetamide (Compound 93)

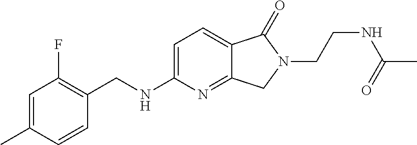 [SMILES: CC(=O)NCCN1Cc2nc(NCc3ccc(C)cc3F)ccc2C1=O]

(2-Fluoro-4-methylphenyl)methanamine (329 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2-fluoro-4-methylbenzyl)amino)-5-oxo-5,7-dihydro-6H-pyrrolo[3,4-b]pyridin-6-yl)ethyl)acetamide (120 mg, 28% yield) as white solid. ¹H NMR (400 MHz, DMSO) δ 7.89 (t, J=5.9 Hz, 1H), 7.69 (t, J=5.8 Hz, 1H), 7.55 (d, J=8.6 Hz, 1H), 7.22 (t, J=8.0 Hz, 1H), 7.04-6.83 (m, 2H), 6.52 (d, J=8.6 Hz, 1H), 4.50 (d, J=5.6 Hz, 2H), 4.23 (s, 2H), 3.43 (t, J=6.1 Hz, 2H), 3.21 (q, J=6.0 Hz, 2H), 2.25 (s, 3H), 1.71 (s, 3H). MS (ESI): Calcd. for C₁₉H₂₁FN₄O₂: 356. found 357 (M+H)⁺.

### Example 81: 3-(2-(2-((2,4-Difluorobenzyl)amino)-5-oxo-5,7-dihydro-6H-pyrrolo[3,4-b]pyridin-6-yl)ethyl)-1,1-dimethylurea (Compound 94)

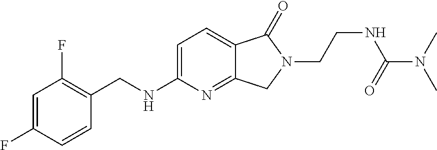 [SMILES: CN(C)C(=O)NCCN1Cc2nc(NCc3ccc(F)cc3F)ccc2C1=O]

(2,4-Diluorophenyl)methanamine (304 mg, 2.12 mmol) was added to a solution of 3-(2-(2-chloro-5-oxo-5,7-dihydro-6H-pyrrolo[3,4-b]pyridin-6-yl)ethyl)-1,1-dimethylurea (300 mg, 1.06 mmol) and DIPEA (0.98 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product 3-(2-(2-((2,4-difluorobenzyl)amino)-5-oxo-5,7-dihydro-6H-pyrrolo[3,4-b]pyridin-6-yl)ethyl)-1,1-dimethylurea (113 mg, 27% yield) as white solid. ¹H NMR (400 MHz, DMSO) δ 7.66 (d, J=8.8 Hz, 1H), 7.20 (td, J=8.7, 6.6 Hz, 1H), 7.10 (ddd, J=10.5, 9.3, 2.6 Hz, 1H), 6.88 (tdd, J=8.6, 2.6, 1.0 Hz, 1H), 6.62 (d, J=8.8 Hz, 1H), 6.36 (t, J=6.0 Hz, 1H), 6.06 (t, J=5.9 Hz, 1H), 4.26 (s, 2H), 4.12 (d, J=5.9 Hz, 2H), 3.45 (dd, J=6.6, 5.3 Hz, 2H), 3.25-3.20 (m, 2H), 3.07 (s, 6H). MS (ESI): Calcd. for C₁₉H₂₁F₂N₅O₂: 389. found 390 (M+H)⁺.

### Example 82: 1-(2-(2-((2,4-Difluorobenzyl)amino)-5-oxo-5,7-dihydro-6H-pyrrolo[3,4-b]pyridin-6-yl)ethyl)-3-methylurea (Compound 95)

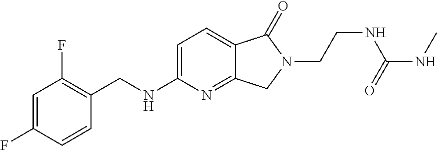 [SMILES: CNC(=O)NCCN1Cc2nc(NCc3ccc(F)cc3F)ccc2C1=O]

(2,4-Difluorophenyl)methanamine (187 mg, 1.49 mmol) was added to a solution of 1-(2-(2-chloro-5-oxo-5,7-dihydro-6H-pyrrolo[3,4-b]pyridin-6-yl)ethyl)-3-methylurea (200 mg, 0.75 mmol) and DIPEA (0.65 mL, 3.74 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product 1-(2-(2-((2,4-difluorobenzyl)amino)-5-oxo-5,7-dihydro-6H-pyrrolo[3,4-b]pyridin-6-yl)ethyl)-3-methylurea (104 mg, 29% yield) as white solid. ¹H NMR (400 MHz, DMSO) δ 7.73 (t, J=5.8 Hz, 1H), 7.55 (d, J=8.5 Hz, 1H), 7.39 (td, J=8.7, 6.6 Hz, 1H), 7.18 (ddd, J=10.5, 9.3, 2.6 Hz, 1H), 7.00 (tdd, J=8.6, 2.6, 1.0 Hz, 1H), 6.53 (d, J=8.5 Hz, 1H), 5.93 (t, J=5.9 Hz, 1H), 5.69 (q, J=4.6 Hz, 1H), 4.52 (d, J=5.8 Hz, 2H), 4.23 (s, 2H), 3.48-3.37 (m, 2H), 3.17 (q, J=6.0 Hz, 2H), 2.45 (s, 3H). MS (ESI): Calcd. for C₁₈H₁₉F₂N₅O₂: 375. found 376 (M+H)⁺.

### Example 83: 1-Methyl-3-(2-(5-oxo-2-((2,4,6-trifluorobenzyl)amino)-5,7-dihydro-6H-pyrrolo[3,4-b]pyridin-6-yl)ethyl)urea (Compound 96)

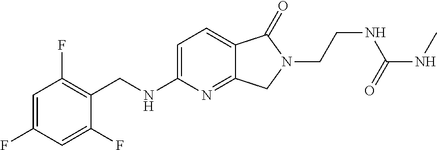 [SMILES: CNC(=O)NCCN1Cc2nc(NCc3c(F)cc(F)cc3F)ccc2C1=O]

(2,4,6-Tirfluorophenyl)methanamine (187 mg, 1.49 mmol) was added to a solution of 1-(2-(2-chloro-5-oxo-5,7-dihydro-6H-pyrrolo[3,4-b]pyridin-6-yl)ethyl)-3-methylurea (200 mg, 0.75 mmol) and DIPEA (0.65 mL, 3.74 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product 1-methyl-3-(2-(5-oxo-2-((2,4,6-trifluorobenzyl)amino)-5,7-dihydro-6H-pyrrolo[3,4-b]pyridin-6-yl)ethyl)urea (96 mg, 25% yield) as white solid. ¹H NMR (400 MHz, DMSO) δ 7.61 (t, J=5.2 Hz, 1H), 7.53 (d, J=8.6 Hz, 1H), 7.14 (ddd, J=11.7, 8.7, 2.8 Hz, 2H), 6.49 (d, J=8.6 Hz, 1H), 5.94 (t, J=5.9 Hz, 1H), 5.70 (q, J=4.6 Hz, 1H), 4.49 (d, J=5.1 Hz, 2H), 4.25 (s, 2H), 3.48-3.38 (m, 2H), 3.18 (q, J=6.0 Hz, 2H), 2.47 (d, J=4.6 Hz, 3H). MS (ESI): Calcd. for C₁₈H₁₈F₃N₅O₂: 393. found 394 (M+H)⁺.

### Example 84: 1-Methyl-3-(2-(5-oxo-2-((pyridin-3-ylmethyl)amino)-5,7-dihydro-6H-pyrrolo[3,4-b]pyridin-6-yl)ethyl)urea (Compound 97)

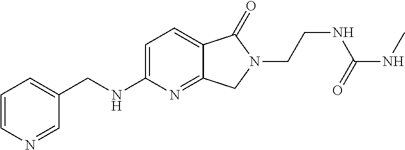 [SMILES: CNC(=O)NCCN1Cc2nc(NCc3cccnc3)ccc2C1=O]

Pyridin-3-ylmethanamine (187 mg, 1.49 mmol) was added to a solution of 1-(2-(2-chloro-5-oxo-5,7-dihydro-6H-pyrrolo[3,4-b]pyridin-6-yl)ethyl)-3-methylurea (200 mg, 0.75 mmol) and DIPEA (0.65 mL, 3.74 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product 1-methyl-3-(2-(5-oxo-2-((pyridin-3-ylmethyl)amino)-5,7-dihydro-6H-pyrrolo[3,4-b]pyridin-6-yl)ethyl)urea (104 mg, 39% yield) as white solid. ¹H NMR (400 MHz, DMSO) δ 8.53 (d, J=2.6 Hz, 1H), 8.41 (dd, J=4.9, 1.7 Hz, 1H), 7.82 (t, J=5.9 Hz, 1H), 7.70 (dt, J=7.9, 2.0 Hz, 1H), 7.56 (d, J=8.5 Hz, 1H), 7.30 (ddd, J=7.8, 4.8, 0.9 Hz, 1H), 6.53 (d, J=8.6 Hz, 1H), 5.95 (t, J=5.9 Hz, 1H), 5.71 (dd, J=5.3, 3.7 Hz, 1H), 4.54 (d, J=5.9 Hz, 2H), 4.23 (s, 2H), 3.41 (t, J=6.0 Hz, 2H), 3.18 (q, J=6.0 Hz, 2H), 2.46 (s, 3H). MS (ESI): Calcd. for C₁₇H₂₀N₆O₂: 340. found 341 (M+H)⁺.

### Example 85: N-(2-(2-((2,6-difluoro-4-methoxybenzyl)amino)-5-oxo-5,7-dihydro-6H-pyrrolo[3,4-b]pyridin-6-yl)ethyl)acetamide (Compound 98)

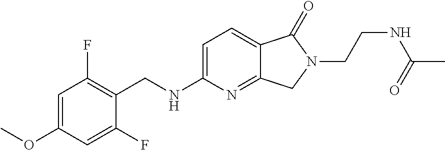 [SMILES: COc1cc(F)c(CNc2ccc3c(n2)CN(CCNC(C)=O)C3=O)c(F)c1]

(2,6-Difluoro-4-methoxyphenyl)methanamine (391 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2,6-difluoro-4-methoxybenzyl)amino)-5-oxo-5,7-dihydro-6H-pyrrolo[3,4-b]pyridin-6-yl)ethyl)acetamide (109 mg, 24% yield) as white solid. ¹H NMR (400 MHz, DMSO) δ 7.90 (t, J=5.9 Hz, 1H), 7.63-7.44 (m, 2H), 6.80-6.66 (m, 2H), 6.49 (d, J=8.6 Hz, 1H), 4.44 (d, J=5.0 Hz, 2H), 4.25 (s, 2H), 3.74 (s, 3H), 3.44 (t, J=6.0 Hz, 2H), 3.25-3.17 (m, 2H), 1.72 (s, 3H). MS (ESI): Calcd. for C₁₉H₂₀F₂N₄O₃: 390. found 391 (M+H)⁺.

### Example 86: N-(2-(2-(((4-methylthiophen-2-yl)methyl)amino)-5-oxo-5,7-dihydro-6H-pyrrolo[3,4-b]pyridin-6-yl)ethyl)acetamide (Compound 99)

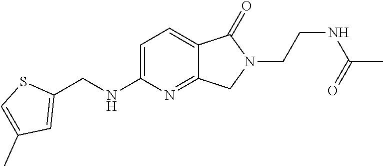 [SMILES: CC(=O)NCCN1Cc2nc(NCc3cc(C)cs3)ccc2C1=O]

(4-Methylthiophen-2-yl)methanamine (301 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-(((4-methylthiophen-2-yl)methyl)amino)-5-oxo-5,7-dihydro-6H-pyrrolo[3,4-b]pyridin-6-yl)ethyl)acetamide (115 mg, 28% yield) as white solid. ¹H NMR (400 MHz, DMSO) δ 7.90 (t, J=5.8 Hz, 1H), 7.77 (t, J=5.9 Hz, 1H), 7.55 (d, J=8.6 Hz, 1H), 6.89 (p, J=1.1 Hz, 1H), 6.81 (d, J=1.5 Hz, 1H), 6.50 (d, J=8.6 Hz, 1H), 4.64-4.54 (m, 2H), 4.26 (s, 2H), 3.56-3.37 (m, 2H), 3.23 (q, J=6.0 Hz, 2H), 2.11 (d, J=1.1 Hz, 3H), 1.72 (s, 3H). MS (ESI): Calcd. for C₁₇H₂₀N₄O₂S: 344. found 345 (M+H)⁺.

### Example 87: N-(2-(5-oxo-2-((thiophen-3-ylmethyl)amino)-5,7-dihydro-6H-pyrrolo[3,4-b]pyridin-6-yl)ethyl)acetamide (Compound 100)

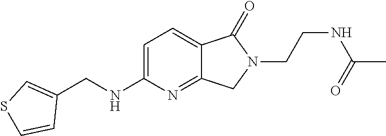 [SMILES: CC(=O)NCCN1Cc2nc(NCc3ccsc3)ccc2C1=O]

Thiophen-3-ylmethanamine (268 mg, 2.37 mmol) was added to a solution of N-(2-(2-chloro-5-oxo-5,7-dihydro-6H-pyrrolo[3,4-b]pyridin-6-yl)ethyl)acetamide (300 mg, 1.18 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((thiophen-3-ylmethyl)amino)-5,7-dihydro-6H-pyrrolo[3,4-b]pyridin-6-yl)ethyl)acetamide (135 mg, 35% yield) as white solid. ¹H NMR (400 MHz, DMSO) δ 7.89 (t, J=5.2 Hz, 1H), 7.66 (t, J=5.8 Hz, 1H), 7.61-7.50 (m, 1H), 7.45 (dd, J=4.9, 2.9 Hz, 1H), 7.38-7.25 (m, 1H), 7.06 (dd, J=4.9, 1.3 Hz, 1H), 6.60-6.44 (m, 1H), 4.50 (d, J=5.7 Hz, 2H), 4.25 (d, J=3.0 Hz, 2H), 3.44 (t, J=6.0 Hz, 2H), 3.22 (q, J=6.0 Hz, 2H), 1.72 (d, J=2.9 Hz, 3H). MS (ESI): Calcd. for C₁₆H₁₈N₄O₂S: 330. found 331 (M+H)⁺.

### Example 88: N-(2-(2-((cyclopropylmethyl)amino)-5-oxo-5,7-dihydro-6H-pyrrolo[3,4-b]pyridin-6-yl)ethyl)acetamide (Compound 101)

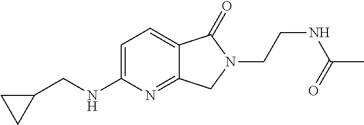 [SMILES: CC(=O)NCCN1Cc2nc(NCC3CC3)ccc2C1=O]

Cyclopropylmethanamine (140 mg, 1.98 mmol) was added to a solution of N-(2-(2-chloro-5-oxo-5,7-dihydro-6H-pyrrolo[3,4-b]pyridin-6-yl)ethyl)acetamide (250 mg, 0.99 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((cyclopropylmethyl)amino)-5-oxo-5,7-dihydro-6H-pyrrolo[3,4-b]pyridin-6-yl)ethyl)acetamide (143 mg, 50% yield) as white solid. ¹H NMR (400 MHz, DMSO) δ 7.89 (t, J=5.9 Hz, 1H), 7.50 (d, J=8.6 Hz, 1H), 7.35 (t, J=5.5 Hz, 1H), 6.47 (d, J=8.6 Hz, 1H), 4.22 (s, 2H), 3.43 (t, J=6.1 Hz, 2H), 3.22 (q, J=6.0 Hz, 2H), 3.15 (dd, J=6.8, 5.5 Hz, 2H), 1.72 (s, 3H), 1.11-0.93 (m, 1H), 0.54-0.32 (m, 2H), 0.26-0.10 (m, 2H). MS (ESI): Calcd. for C₁₅H₂₀N₄O₂: 288. found 289 (M+H)⁺.

### Example 89: N-(2-(2-((cyclobutylmethyl)amino)-5-oxo-5,7-dihydro-6H-pyrrolo[3,4-b]pyridin-6-yl)ethyl)acetamide (Compound 102)

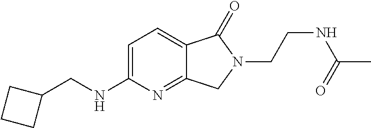 [SMILES: CC(=O)NCCN1Cc2nc(NCC3CCC3)ccc2C1=O]

Cyclobutylmethanamine (168 mg, 1.98 mmol) was added to a solution of N-(2-(2-chloro-5-oxo-5,7-dihydro-6H-pyrrolo[3,4-b]pyridin-6-yl)ethyl)acetamide (250 mg, 0.99 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((cyclobutylmethyl)amino)-5-oxo-5,7-dihydro-6H-pyrrolo[3,4-b]pyridin-6-yl)ethyl)acetamide (123 mg, 41% yield) as white solid. ¹H NMR (400 MHz, DMSO) δ 7.89 (t, J=5.8 Hz, 1H), 7.49 (d, J=8.6 Hz, 1H), 7.23 (t, J=5.5 Hz, 1H), 6.44 (d, J=8.6 Hz, 1H), 4.22 (s, 2H), 3.43 (t, J=6.1 Hz, 2H), 3.34-3.29 (m, 2H), 3.23-3.16 (m, 2H), 2.58-2.48 (m, 1H), 2.10-1.90 (m, 2H), 1.90-1.76 (m, 2H), 1.72 (s, 3H), 1.66 (ddd, J=9.7, 7.5, 2.2 Hz, 2H). MS (ESI): Calcd. for C₁₆H₂₂N₄O₂: 302. found 303 (M+H)⁺.

### Example 90: N-(2-(2-((cyclohexylmethyl)amino)-5-oxo-5,7-dihydro-6H-pyrrolo[3,4-b]pyridin-6-yl)ethyl)acetamide (Compound 103)

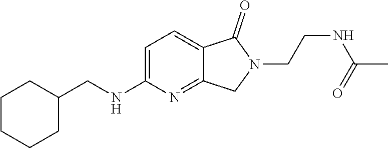 [SMILES: CC(=O)NCCN1Cc2nc(NCC3CCCCC3)ccc2C1=O]

Cyclohexylmethanamine (223 mg, 1.98 mmol) was added to a solution of N-(2-(2-chloro-5-oxo-5,7-dihydro-6H-pyrrolo[3,4-b]pyridin-6-yl)ethyl)acetamide (250 mg, 0.99 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((cyclohexylmethyl)amino)-5-oxo-5,7-dihydro-6H-pyrrolo[3,4-b]pyridin-6-yl)ethyl)acetamide (105 mg, 32% yield) as white solid. ¹H NMR (400 MHz, DMSO) δ 7.89 (t, J=5.8 Hz, 1H), 7.48 (d, J=8.6 Hz, 1H), 7.24 (t, J=5.6 Hz, 1H), 6.45 (d, J=8.6 Hz, 1H), 4.21 (s, 2H), 3.42 (t, J=6.1 Hz, 2H), 3.26-3.16 (m, 2H), 3.12 (t, J=6.3 Hz, 2H), 1.85-1.45 (m, 9H), 1.15 (h, J=11.1 Hz, 3H), 0.89 (q, J=10.5 Hz, 2H). MS (ESI): Calcd. for C₁₈H₂₆N₄O₂: 330. found 331 (M+H)⁺.

### Example 91: N-(2-(5-oxo-2-(((tetrahydro-2H-pyran-4-yl)methyl)amino)-5,7-dihydro-6H-pyrrolo[3,4-b]pyridin-6-yl)ethyl)acetamide (Compound 108)

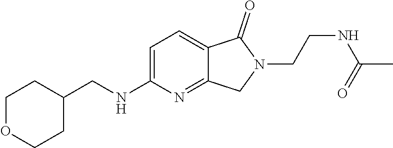 [SMILES: CC(=O)NCCN1Cc2nc(NCC3CCOCC3)ccc2C1=O]

(Tetrahydro-2H-pyran-4-yl)methanamine (227 mg, 1.98 mmol) was added to a solution of N-(2-(2-chloro-5-oxo-5,7-dihydro-6H-pyrrolo[3,4-b]pyridin-6-yl)ethyl)acetamide (250 mg, 0.99 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-(((tetrahydro-2H-pyran-4-yl)methyl)amino)-5,7-dihydro-6H-pyrrolo[3,4-b]pyridin-6-yl)ethyl)acetamide (56 mg, 17% yield) as white solid. ¹H NMR (400 MHz, DMSO) δ 7.89 (h, J=5.8 Hz, 1H), 7.49 (dd, J=12.1, 8.2 Hz, 1H), 7.30 (h, J=6.0 Hz, 1H), 6.64-6.34 (m, 1H), 4.32-4.10 (m, 2H), 3.79 (dt, J=11.6, 5.8 Hz, 2H), 3.43 (dt, J=12.8, 6.3 Hz, 2H), 3.26-3.02 (m, 6H), 1.89-1.67 (m, 4H), 1.58 (t, J=10.9 Hz, 2H), 1.17 (td, J=12.5, 7.1 Hz, 2H). MS (ESI): Calcd. for C₁₇H₂₄N₄O₃: 332. found 333 (M+H)⁺.

### Example 92: N-(2-(5-oxo-2-((thiazol-5-ylmethyl)amino)-5,7-dihydro-6H-pyrrolo[3,4-b]pyridin-6-yl)ethyl)acetamide (Compound 114)

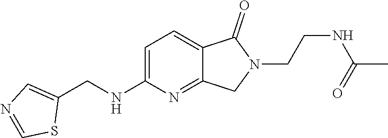 [SMILES: CC(=O)NCCN1Cc2nc(NCc3cncs3)ccc2C1=O]

Thiazol-5-ylmethanamine (225 mg, 1.98 mmol) was added to a solution of N-(2-(2-chloro-5-oxo-5,7-dihydro-6H-pyrrolo[3,4-b]pyridin-6-yl)ethyl)acetamide (250 mg, 0.99 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((thiazol-5-ylmethyl)amino)-5,7-dihydro-6H-pyrrolo[3,4-b]pyridin-6-yl)ethyl)acetamide (36 mg, 11% yield) as white solid. ¹H NMR (400 MHz, DMSO) δ 8.90 (s, 1H), 7.88 (dt, J=17.7, 5.9 Hz, 2H), 7.80 (s, 1H), 7.57 (d, J=8.5 Hz, 1H), 6.50 (d, J=8.5 Hz, 1H), 4.73 (d, J=5.9 Hz, 2H), 4.28 (s, 2H), 3.45 (t, J=6.1 Hz, 2H), 3.25-3.18 (m, 2H), 1.72 (s, 3H). MS (ESI): Calcd. for C₁₅H₁₇N₅O₂S: 331. found 332 (M+H)⁺.

### Example 93: N-(2-(2-((cyclopentylmethyl)amino)-5-oxo-5,7-dihydro-6H-pyrrolo[3,4-b]pyridin-6-yl)ethyl)acetamide (Compound 115)

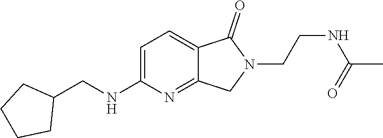 [SMILES: CC(=O)NCCN1Cc2nc(NCC3CCCC3)ccc2C1=O]

Cyclopentylmethanamine (195 mg, 1.98 mmol) was added to a solution of N-(2-(2-chloro-5-oxo-5,7-dihydro-6H-pyrrolo[3,4-b]pyridin-6-yl)ethyl)acetamide (250 mg, 0.99 mmol) and DIPEA (1.03 mL, 5.91 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((cyclopentylmethyl)amino)-5-oxo-5,7-dihydro-6H-pyrrolo[3,4-b]pyridin-6-yl)ethyl)acetamide (116 mg, 37% yield) as white solid. ¹H NMR (400 MHz, DMSO) δ 7.89 (q, J=5.8 Hz, 1H), 7.48 (dd, J=11.5, 8.4 Hz, 1H), 7.27 (q, J=5.7 Hz, 1H), 6.44 (dd, J=11.5, 8.6 Hz, 1H), 4.20 (d, J=11.3 Hz, 2H), 3.41 (dt, J=11.5, 6.1 Hz, 2H), 3.18 (tt, J=11.8, 3.6 Hz, 4H), 2.09 (dp, J=11.3, 7.4 Hz, 1H), 1.92-1.63 (m, 5H), 1.60-1.37 (m, 4H), 1.20 (dq, J=14.2, 6.9 Hz, 2H). MS (ESI): Calcd. for C₁₇H₂₄N₄O₂: 316. found 317 (M+H)⁺.

### Example 94: Synthesis of N-(2-(2-((2,4-difluorobenzyl)amino)-5-oxo-5,7-dihydro-6H-pyrrolo[3,4-b]pyridin-6-yl)ethyl)acetamide-2,2,2-d3 (Compound 201)

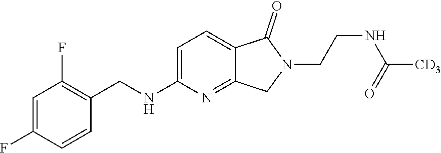 [SMILES: [2H]C([2H])([2H])C(=O)NCCN1Cc2nc(NCc3ccc(F)cc3F)ccc2C1=O]

(2,4-Difluorophenyl)methanamine (262 mg, 1.84 mmol) was added to a solution of N-(2-(2-chloro-5-oxo-5,7-dihydro-6H-pyrrolo[3,4-b]pyridin-6-yl)ethyl)acetamide-2,2,2-d3 (235 mg, 0.92 mmol) and DIPEA (1.03 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2,4-difluorobenzyl)amino)-5-oxo-5,7-dihydro-6H-pyrrolo[3,4-b]pyridin-6-yl)ethyl)acetamide-2,2,2-d3 (113 mg, 31% yield) as white solid. ¹H NMR (400 MHz, DMSO) δ 7.89 (t, J=5.8 Hz, 1H), 7.74 (t, J=5.8 Hz, 1H), 7.56 (d, J=8.6 Hz, 1H), 7.39 (td, J=8.7, 6.6 Hz, 1H), 7.19 (ddd, J=10.5, 9.3, 2.6 Hz, 1H), 7.01 (tdd, J=8.5, 2.6, 1.1 Hz, 1H), 6.53 (d, J=8.6 Hz, 1H), 4.52 (d, J=5.7 Hz, 2H), 4.23 (s, 2H), 3.42 (q, J=5.5 Hz, 2H), 3.24-3.18 (m, 2H). MS (ESI): Calcd. for C₁₈H₁₅D₃F₂N₄O₂: 363. found 364 (M+H)⁺.

### Example 95: Synthesis of N-(2-(5-oxo-2-((2,4,6-trifluorobenzyl)amino)-5,7-dihydro-6H-pyrrolo[3,4-b]pyridin-6-yl)ethyl)acetamide-2,2,2-d3 (Compound 202)

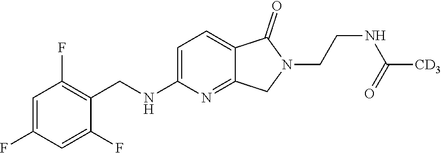 [SMILES: [2H]C([2H])([2H])C(=O)NCCN1Cc2nc(NCc3c(F)cc(F)cc3F)ccc2C1=O]

(2,4,6-Tirfluorophenyl)methanamine (295 mg, 1.83 mmol) was added to a solution of N-(2-(2-chloro-5-oxo-5,7-dihydro-6H-pyrrolo[3,4-b]pyridin-6-yl)ethyl)acetamide-2,2,2-d3 (235 mg, 0.92 mmol) and DIPEA (1.03 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,4,6-trifluorobenzyl)amino)-5,7-dihydro-6H-pyrrolo[3,4-b]pyridin-6-yl)ethyl)acetamide-2,2,2-d3 (103 mg, 30% yield) as white solid. ¹H NMR (400 MHz, DMSO) δ 7.89 (t, J=5.9 Hz, 1H), 7.61 (t, J=5.2 Hz, 1H), 7.54 (d, J=8.6 Hz, 1H), 7.24-7.07 (m, 2H), 6.49 (d, J=8.6 Hz, 1H), 4.49 (d, J=5.1 Hz, 2H), 4.25 (s, 2H), 3.44 (t, J=6.0 Hz, 2H), 3.24-3.16 (m, 2H). MS (ESI): Calcd. for C₁₈H₁₄D₃F₃N₄O₂: 381. found 382 (M+H)⁺.

### Example 96: Synthesis of N-(2-(2-((2,4-difluorobenzyl)amino)-5-oxo-5,7-dihydro-6H-pyrrolo[3,4-b]pyridin-6-yl)ethyl)propenamide-d5 (Compound 203)

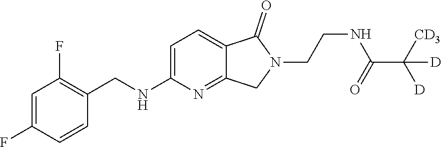 [SMILES: [2H]C([2H])([2H])C([2H])([2H])C(=O)NCCN1Cc2nc(NCc3ccc(F)cc3F)ccc2C1=O]

(2,4-Difluorophenyl)methanamine (315 mg, 2.20 mmol) was added to a solution of N-(2-(2-chloro-5-oxo-5,7-dihydro-6H-pyrrolo[3,4-b]pyridin-6-yl)ethyl)propenamide (300 mg, 1.10 mmol) and DIPEA (0.65 mL, 3.74 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((2,4-difluorobenzyl)amino)-5-oxo-5,7-dihydro-6H-pyrrolo[3,4-b]pyridin-6-yl)ethyl)propenamide-d5 (101 mg, 24% yield) as white solid. 1H NMR (400 MHz, DMSO) δ 7.80 (t, J=5.9 Hz, 1H), 7.73 (t, J=5.8 Hz, 1H), 7.56 (d, J=8.6 Hz, 1H), 7.39 (td, J=8.7, 6.7 Hz, 1H), 7.18 (ddd, J=10.5, 9.3, 2.6 Hz, 1H), 7.00 (tdd, J=8.5, 2.6, 1.0 Hz, 1H), 6.52 (d, J=8.6 Hz, 1H), 4.52 (d, J=5.7 Hz, 2H), 4.23 (s, 2H), 3.52-3.38 (m, 2H), 3.22 (q, J=6.0 Hz, 2H). MS (ESI): Calcd. for C₁₉H₁₅D₅F₂N₄O₂: 379. found 380 (M+H)⁺.

### Example 97: Synthesis of N-(2-(5-oxo-2-((2,4,6-trifluorobenzyl)amino)-5,7-dihydro-6H-pyrrolo[3,4-b]pyridin-6-yl)ethyl)propenamide-d5 (Compound 204)

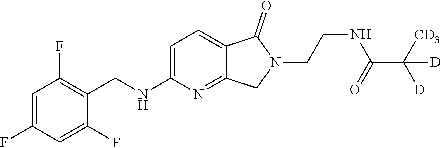 [SMILES: [2H]C([2H])([2H])C([2H])([2H])C(=O)NCCN1Cc2nc(NCc3c(F)cc(F)cc3F)ccc2C1=O]

(2,4,6-Tirfluorophenyl)methanamine (354 mg, 2.20 mmol) was added to a solution of N-(2-(2-chloro-5-oxo-5,7-dihydro-6H-pyrrolo[3,4-b]pyridin-6-yl)ethyl)propenamide (300 mg, 1.10 mmol) and DIPEA (0.65 mL, 3.74 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(5-oxo-2-((2,4,6-trifluorobenzyl)amino)-5,7-dihydro-6H-pyrrolo[3,4-b]pyridin-6-yl)ethyl)propenamide-d5 (86 mg, 20% yield) as white solid. ¹H NMR (400 MHz, DMSO) δ 7.81 (t, J=5.8 Hz, 1H), 7.61 (t, J=5.2 Hz, 1H), 7.53 (d, J=8.5 Hz, 1H), 7.15 (ddd, J=11.6, 8.7, 2.7 Hz, 2H), 6.49 (d, J=8.6 Hz, 1H), 4.49 (d, J=5.1 Hz, 2H), 4.25 (s, 2H), 3.53-3.37 (m, 2H), 3.23 (q, J=5.1 Hz, 2H). MS (ESI): Calcd. for C₁₉H₁₄D₅F₃N₄O₂: 397. found 398 (M+H)⁺.

### Example 98: Synthesis of N-(2-(2-((4-chloro-2,6-difluorobenzyl)amino)-5-oxo-5,7-dihydro-6H-pyrrolo[3,4-b]pyridin-6-yl)ethyl)propenamide-d5 (Compound 205)

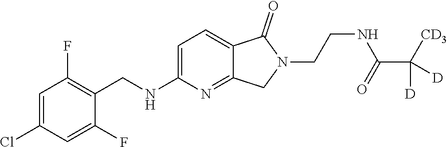 [SMILES: [2H]C([2H])([2H])C([2H])([2H])C(=O)NCCN1Cc2nc(NCc3c(F)cc(Cl)cc3F)ccc2C1=O]

(4-Chloro-2,6-difluorophenyl)methanamine (326 mg, 1.84 mmol) was added to a solution of N-(2-(2-chloro-5-oxo-5,7-dihydro-6H-pyrrolo[3,4-b]pyridin-6-yl)ethyl)propenamide (250 mg, 0.92 mmol) and DIPEA (0.65 mL, 3.74 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-chloro-2,6-difluorobenzyl)amino)-5-oxo-5,7-dihydro-6H-pyrrolo[3,4-b]pyridin-6-yl)ethyl)propenamide-d5 (101 mg, 27% yield) as white solid. ¹H NMR (400 MHz, DMSO) δ 7.81 (t, J=5.9 Hz, 1H), 7.64 (t, J=5.2 Hz, 1H), 7.54 (d, J=8.6 Hz, 1H), 7.39-7.26 (m, 2H), 6.48 (d, J=8.5 Hz, 1H), 4.50 (d, J=5.2 Hz, 2H), 4.24 (s, 2H), 3.57-3.37 (m, 2H), 3.22 (t, J=6.0 Hz, 2H). MS (ESI): Calcd. for C₁₉H₁₄D₅F₂ClN₄O₂: 413. found 414 (M+H)⁺.

### Example 99: Synthesis of N-(2-(2-((4-chloro-2,6-difluorobenzyl)amino)-5-oxo-5,7-dihydro-6H-pyrrolo[3,4-b]pyridin-6-yl)ethyl)acetamide-2,2,2-d3 (Compound 206)

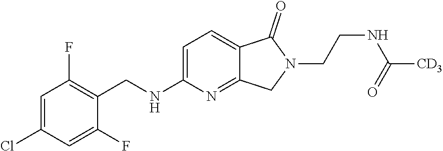 [SMILES: [2H]C([2H])([2H])C(=O)NCCN1Cc2nc(NCc3c(F)cc(Cl)cc3F)ccc2C1=O]

(4-Chloro-2,6-difluorophenyl)methanamine (277 mg, 1.56 mmol) was added to a solution of N-(2-(2-chloro-5-oxo-5,7-dihydro-6H-pyrrolo[3,4-b]pyridin-6-yl)ethyl)acetamide-2,2,2-d3 (200 mg, 0.78 mmol) and DIPEA (1.03 mL, 5.60 mmol) in 1-BuOH (10 mL) at room temperature. The mixture was then heated to 140° C. overnight and cooled to room temperature. The reaction mixture was concentrated under vacuo and the residue was purified by Buchi Pureflash chromatography (silica, 80 g) using 0-10% MeOH in DCM as eluent to obtain desired product N-(2-(2-((4-chloro-2,6-difluorobenzyl)amino)-5-oxo-5,7-dihydro-6H-pyrrolo[3,4-b]pyridin-6-yl)ethyl)acetamide-2,2,2-d3 (113 mg, 36% yield) as white solid. ¹H NMR (400 MHz, DMSO) δ 7.89 (t, J=5.9 Hz, 1H), 7.65 (t, J=5.3 Hz, 1H), 7.54 (d, J=8.5 Hz, 1H), 7.39-7.23 (m, 2H), 6.49 (d, J=8.6 Hz, 1H), 4.50 (d, J=5.2 Hz, 2H), 4.24 (s, 2H), 3.51-3.35 (m, 2H), 3.23 (q, J=6.0 Hz, 2H). MS (ESI): Calcd. for C₁₈H₁₄D₃F₂ClN₄O₂: 397. found 398 (M+H)⁺.

### Example 100: VNN1 Inhibitor Binding Assay

Vanin-1 inhibitor binding assays for selected compounds as described herein, and reference compound RR6 (also “(R)-2,4-dihydroxy-3,3-dimethyl-N-(3-oxo-4-phenylbutyl)butanamide”) were examined and the IC₅₀ values of were determined.

5 μL of 3× (45 μM) of Pantothenate-AMC to achieve final concentrations of 15 μM (0.25% DMSO final) were added in a 384 well assay plate followed by the addition of 5 μL of 3× the desired concentration of the tested compounds (Compound 1; Compound 2; and RR6) in 0.75% DMSO to designated wells (0.25% of DMSO final). The range of the compound concentrations tested was 10, 3.33, 1.11, 0.37, 0.12, 0.04, 0.01, 0.0046, 0.001, and 0.0005 μM. 5 μL of 0.75% DMSO were also added to positive and negative control wells.

5 μL of 1.4 ng/μL Vanain-1 was added to the assay plate to achieve a final amount of 7 ng/well, and covered with plate sealer. Another 5 μL of assay buffer was added to negative controls and the assay plate was then centrifuged briefly (1000 rpm for 30 s) in a plate centrifuge. The assay plate was incubated in the dark for 20 minutes at room temperature and immediately read on an Envision 2105, PerkinElmer multimode plate reader.

The fluorescence read was captured at excitation wavelength of 355 nm and emission wavelength of 460 nm. Percent inhibition of the test compounds was calculated using the following equation:

$$Percent Inhibition = 100 - [ \frac{( A - B )}{( C - B )} * 100 ]$$

In the above equation, “A” is the relative fluorescence units of the test compound (test compound+Pantothenate-AMC+Vanin1); “B” is the relative fluorescence units of the average minimum (DMSO+Pantothenate-AMC+assay buffer); and “C” is the relative fluorescence units of the average maximum (DMSO+Pantothenate-AMC+Vanin1). The Vanin-1 inhibitory IC₅₀ of the tested compounds (Compound 1, Compound 2, and reference compound RR6) is summarized in Table 6.

TABLE 6
Vanin-1 inhibitory binding assay data.

| Compound | Structure | Vanin-1 IC₅₀ (μM) |
|---|---|---|
| RR6 | | 1-10 |
| Compound 1 | 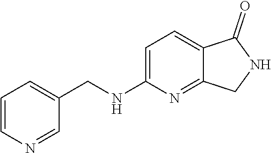 [SMILES: O=C1NCc2nc(NCc3cccnc3)ccc21] | 1-0.5 |
| Compound 2 | 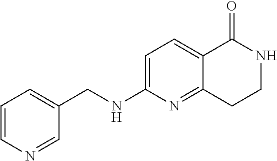 [SMILES: O=C1NCCc2nc(NCc3cccnc3)ccc21] | 1-10 |
| Compound 4 | 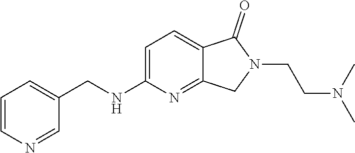 [SMILES: CN(C)CCN1Cc2nc(NCc3cccnc3)ccc2C1=O] | 1-10 |
| Compound 6 | 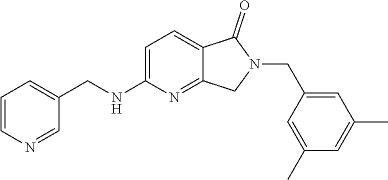 [SMILES: Cc1cc(C)cc(CN2Cc3nc(NCc4cccnc4)ccc3C2=O)c1] | 1-.05 |
| Compound 11 | 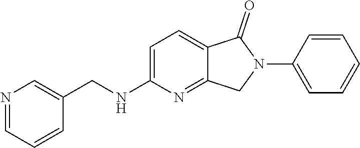 [SMILES: O=C1c2ccc(NCc3cccnc3)nc2CN1c1ccccc1] | >10 |
| Compound 12 | 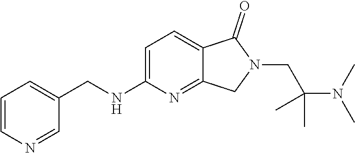 [SMILES: CN(C)C(C)(C)CN1Cc2nc(NCc3cccnc3)ccc2C1=O] | 1-10 |
| Compound 13 | 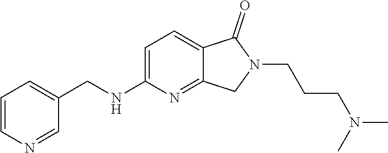 [SMILES: CN(C)CCCN1Cc2nc(NCc3cccnc3)ccc2C1=O] | 1-10 |
| Compound 14 | 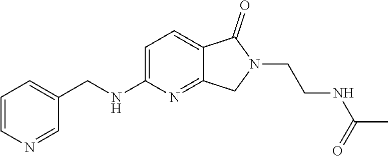 [SMILES: CC(=O)NCCN1Cc2nc(NCc3cccnc3)ccc2C1=O] | 1-0.05 |
| Compound 15 | 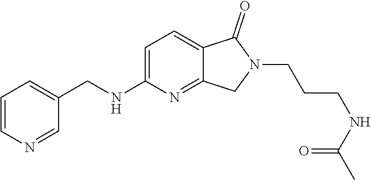 [SMILES: CC(=O)NCCCN1Cc2nc(NCc3cccnc3)ccc2C1=O] | 1-10 |
| Compound 19 | 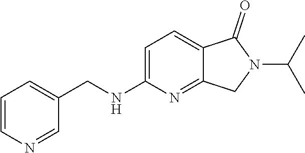 [SMILES: CC(C)N1Cc2nc(NCc3cccnc3)ccc2C1=O] | 1-10 |
| Compound 17 | 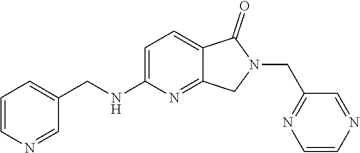 [SMILES: O=C1c2ccc(NCc3cccnc3)nc2CN1Cc1cnccn1] | 1-10 |
| Compound 18 | 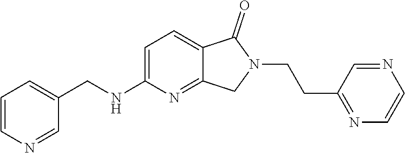 [SMILES: O=C1c2ccc(NCc3cccnc3)nc2CN1CCc1cnccn1] | 1-0.05 |
| Compound 50 | 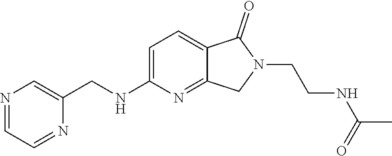 [SMILES: CC(=O)NCCN1Cc2nc(NCc3cnccn3)ccc2C1=O] | 1-10 |
| Compound 20 | 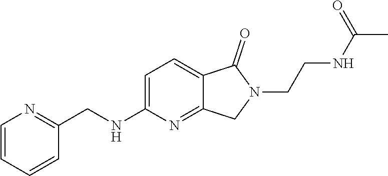 [SMILES: CC(=O)NCCN1Cc2nc(NCc3ccccn3)ccc2C1=O] | 1-0.05 |
| Compound 21 | 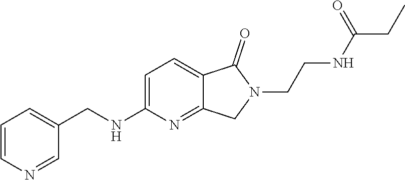 [SMILES: CCC(=O)NCCN1Cc2nc(NCc3cccnc3)ccc2C1=O] | 1-0.5 |
| Compound 22 | 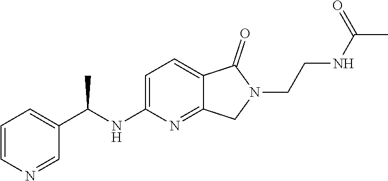 [SMILES: CC(=O)NCCN1Cc2nc(N[C@H](C)c3cccnc3)ccc2C1=O] | >10 |
| Compound 23 | 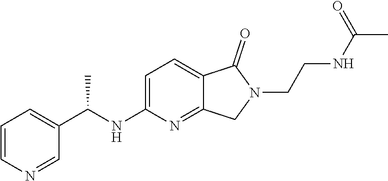 [SMILES: CC(=O)NCCN1Cc2nc(N[C@@H](C)c3cccnc3)ccc2C1=O] | 1-.05 |
| Compound 24 | 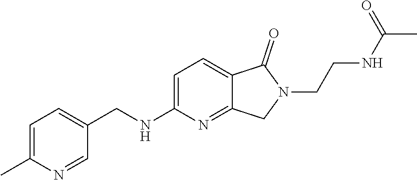 [SMILES: CC(=O)NCCN1Cc2nc(NCc3ccc(C)nc3)ccc2C1=O] | 0.05-0.001 |
| Compound 25 | 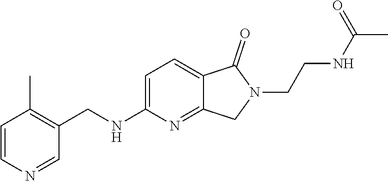 [SMILES: CC(=O)NCCN1Cc2nc(NCc3cnccc3C)ccc2C1=O] | 1-0.05 |
| Compound 26 | 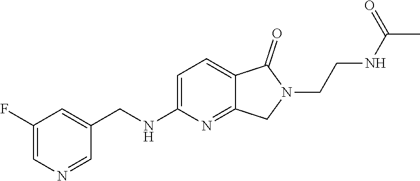 [SMILES: CC(=O)NCCN1Cc2nc(NCc3cncc(F)c3)ccc2C1=O] | 1-10 |
| Compound 27 | 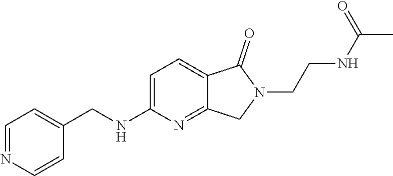 [SMILES: CC(=O)NCCN1Cc2nc(NCc3ccncc3)ccc2C1=O] | 1-10 |
| Compound 28 | 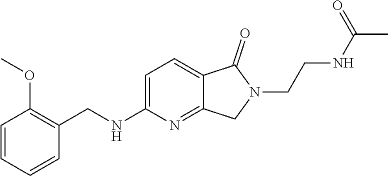 [SMILES: COc1ccccc1CNc1ccc2c(n1)CN(CCNC(C)=O)C2=O] | 1-10 |
| Compound 29 | 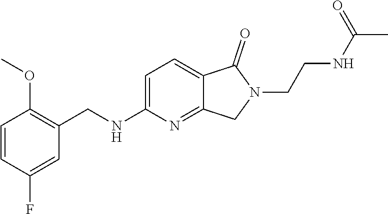 [SMILES: COc1ccc(F)cc1CNc1ccc2c(n1)CN(CCNC(C)=O)C2=O] | 1-10 |
| Compound 30 | 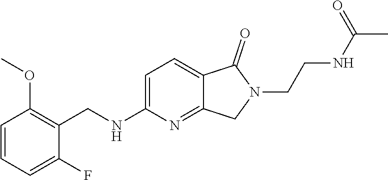 [SMILES: COc1cccc(F)c1CNc1ccc2c(n1)CN(CCNC(C)=O)C2=O] | 1-0.05 |
| Compound 31 | 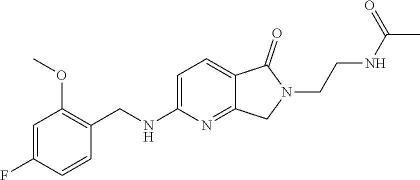 [SMILES: COc1cc(F)ccc1CNc1ccc2c(n1)CN(CCNC(C)=O)C2=O] | 1-0.05 |
| Compound 32 | 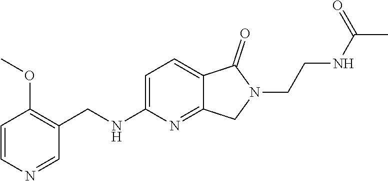 [SMILES: COc1ccncc1CNc1ccc2c(n1)CN(CCNC(C)=O)C2=O] | 1-0.05 |
| Compound 33 | 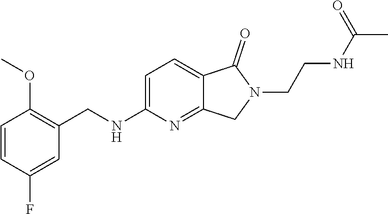 [SMILES: COc1ccc(F)cc1CNc1ccc2c(n1)CN(CCNC(C)=O)C2=O] | 1-0.05 |
| Compound 34 | 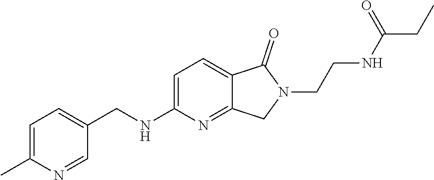 [SMILES: CCC(=O)NCCN1Cc2nc(NCc3ccc(C)nc3)ccc2C1=O] | 0.05-0.001 |
| Compound 35 | 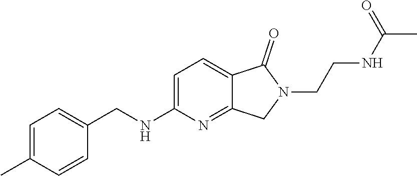 [SMILES: CC(=O)NCCN1Cc2nc(NCc3ccc(C)cc3)ccc2C1=O] | 1-0.05 |
| Compound 36 | 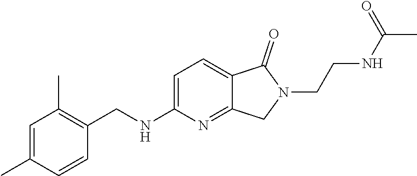 [SMILES: CC(=O)NCCN1Cc2nc(NCc3ccc(C)cc3C)ccc2C1=O] | 1-0.05 |
| Compound 37 | 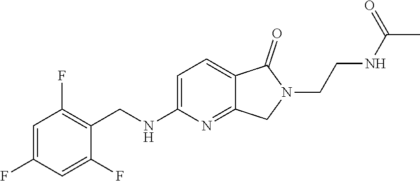 [SMILES: CC(=O)NCCN1Cc2nc(NCc3c(F)cc(F)cc3F)ccc2C1=O] | 0.05-0.001 |
| Compound 38 | 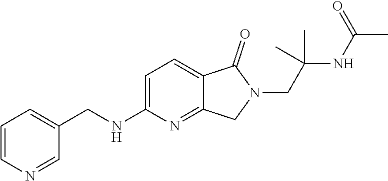 [SMILES: CC(=O)NC(C)(C)CN1Cc2nc(NCc3cccnc3)ccc2C1=O] | 1-10 |
| Compound 39 | 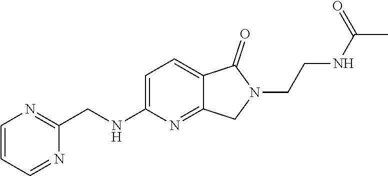 [SMILES: CC(=O)NCCN1Cc2nc(NCc3ncccn3)ccc2C1=O] | 1-0.05 |
| Compound 40 | 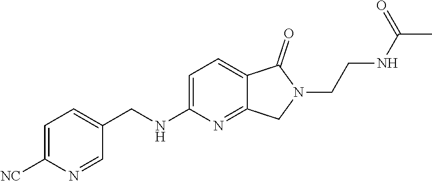 [SMILES: CC(=O)NCCN1Cc2nc(NCc3ccc(C#N)nc3)ccc2C1=O] | 1-10 |
| Compound 41 | 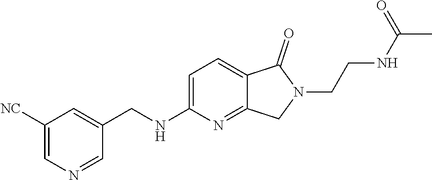 [SMILES: CC(=O)NCCN1Cc2nc(NCc3cncc(C#N)c3)ccc2C1=O] | >10 |
| Compound 42 | 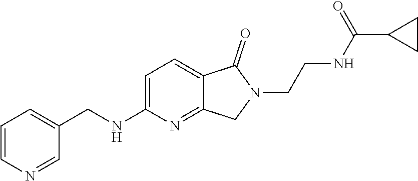 [SMILES: O=C(NCCN1Cc2nc(NCc3cccnc3)ccc2C1=O)C1CC1] | 1-0.05 |
| Compound 43 | 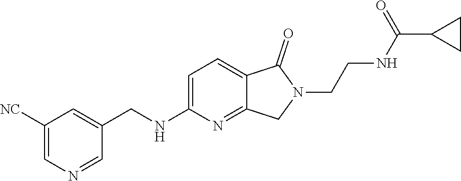 [SMILES: N#Cc1cncc(CNc2ccc3c(n2)CN(CCNC(=O)C2CC2)C3=O)c1] | >10 |
| Compound 44 | 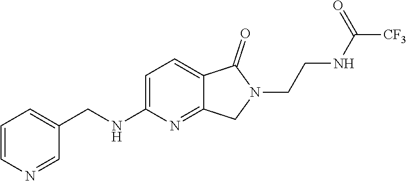 [SMILES: O=C1c2ccc(NCc3cccnc3)nc2CN1CCNC(=O)C(F)(F)F] | 1-0.05 |
| Compound 45 | 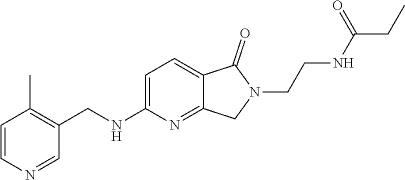 [SMILES: CCC(=O)NCCN1Cc2nc(NCc3cnccc3C)ccc2C1=O] | 0.05-0.001 |
| Compound 46 | 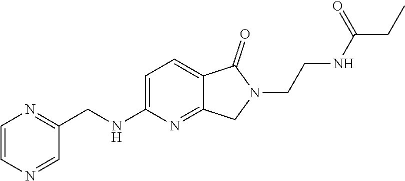 [SMILES: CCC(=O)NCCN1Cc2nc(NCc3cnccn3)ccc2C1=O] | 1-0.05 |
| Compound 47 | 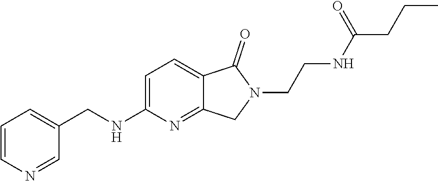 [SMILES: CCCC(=O)NCCN1Cc2nc(NCc3cccnc3)ccc2C1=O] | 0.05-0.001 |
| Compound 48 | 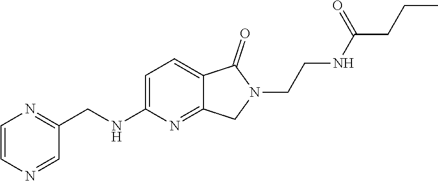 [SMILES: CCCC(=O)NCCN1Cc2nc(NCc3cnccn3)ccc2C1=O] | 1-0.05 |
| Compound 49 | 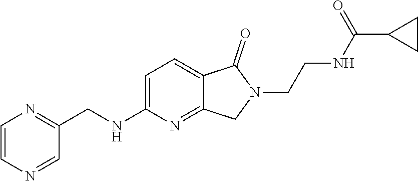 [SMILES: O=C(NCCN1Cc2nc(NCc3cnccn3)ccc2C1=O)C1CC1] | 1-0.05 |
| Compound 51 | 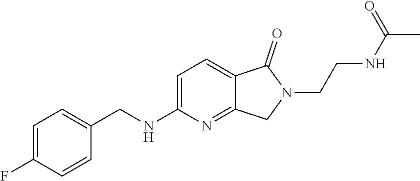 [SMILES: CC(=O)NCCN1Cc2nc(NCc3ccc(F)cc3)ccc2C1=O] | 1-0.05 |
| Compound 52 | 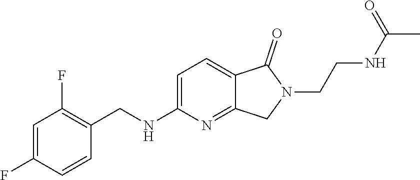 [SMILES: CC(=O)NCCN1Cc2nc(NCc3ccc(F)cc3F)ccc2C1=O] | 0.05-0.001 |
| Compound 53 | 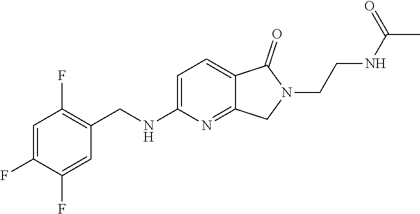 [SMILES: CC(=O)NCCN1Cc2nc(NCc3cc(F)c(F)cc3F)ccc2C1=O] | 1-0.05 |
| Compound 54 | 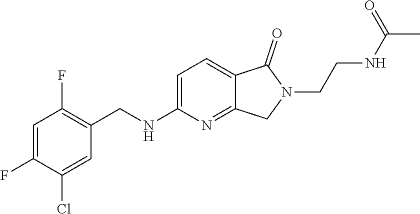 [SMILES: CC(=O)NCCN1Cc2nc(NCc3cc(Cl)c(F)cc3F)ccc2C1=O] | 1-0.05 |
| Compound 55 | 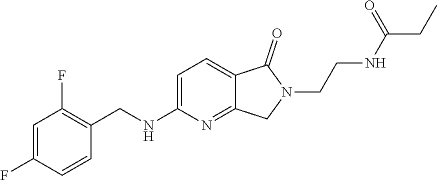 [SMILES: CCC(=O)NCCN1Cc2nc(NCc3ccc(F)cc3F)ccc2C1=O] | 0.05-0.001 |
| Compound 56 | 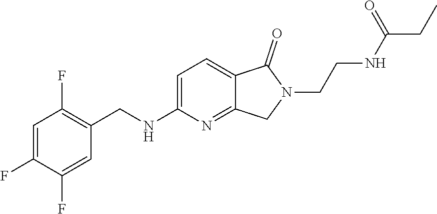 [SMILES: CCC(=O)NCCN1Cc2nc(NCc3cc(F)c(F)cc3F)ccc2C1=O] | 1-0.05 |
| Compound 57 | 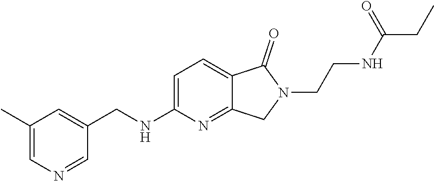 [SMILES: CCC(=O)NCCN1Cc2nc(NCc3cncc(C)c3)ccc2C1=O] | 1-0.05 |
| Compound 58 | 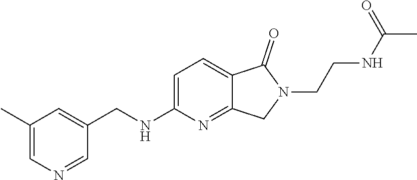 [SMILES: CC(=O)NCCN1Cc2nc(NCc3cncc(C)c3)ccc2C1=O] | 1-0.05 |
| Compound 59 | 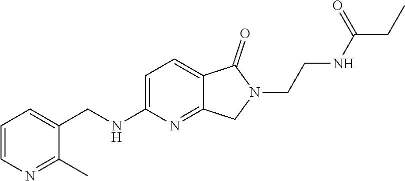 [SMILES: CCC(=O)NCCN1Cc2nc(NCc3cccnc3C)ccc2C1=O] | 1-0.05 |
| Compound 60 | 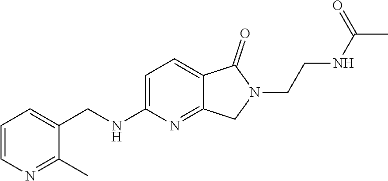 [SMILES: CC(=O)NCCN1Cc2nc(NCc3cccnc3C)ccc2C1=O] | 1-0.05 |
| Compound 61 | 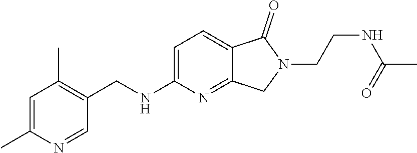 [SMILES: CC(=O)NCCN1Cc2nc(NCc3cnc(C)cc3C)ccc2C1=O] | 0.05-0.001 |
| Compound 62 | 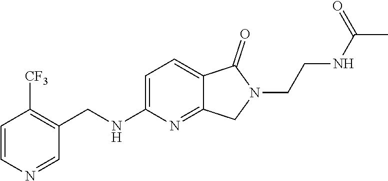 [SMILES: CC(=O)NCCN1Cc2nc(NCc3cnccc3C(F)(F)F)ccc2C1=O] | 1-0.05 |
| Compound 69 | 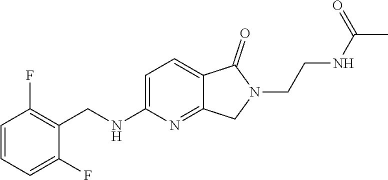 [SMILES: CC(=O)NCCN1Cc2nc(NCc3c(F)cccc3F)ccc2C1=O] | 0.05-0.001 |
| Compound 70 | 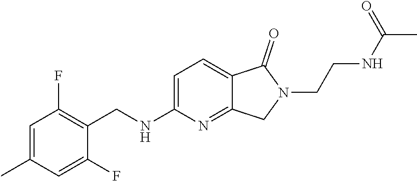 [SMILES: CC(=O)NCCN1Cc2nc(NCc3c(F)cc(C)cc3F)ccc2C1=O] | 0.05-0.001 |
| Compound 71 | 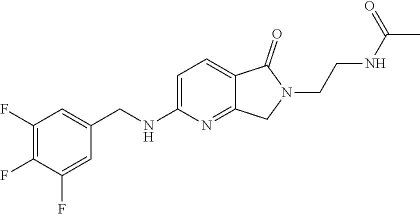 [SMILES: CC(=O)NCCN1Cc2nc(NCc3cc(F)c(F)c(F)c3)ccc2C1=O] | 1-10 |
| Compound 72 | 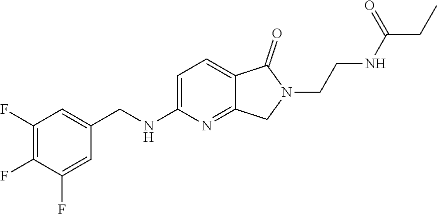 [SMILES: CCC(=O)NCCN1Cc2nc(NCc3cc(F)c(F)c(F)c3)ccc2C1=O] | 1-10 |
| Compound 73 | 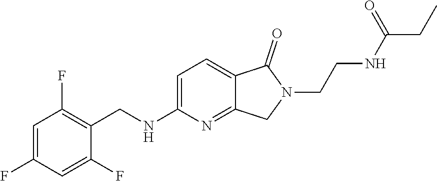 [SMILES: CCC(=O)NCCN1Cc2nc(NCc3c(F)cc(F)cc3F)ccc2C1=O] | 0.05-0.001 |
| Compound 74 | 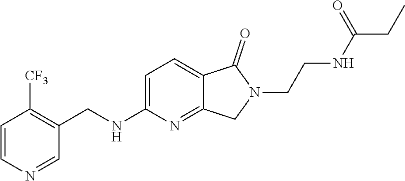 [SMILES: CCC(=O)NCCN1Cc2nc(NCc3cnccc3C(F)(F)F)ccc2C1=O] | 1-10 |
| Compound 75 | 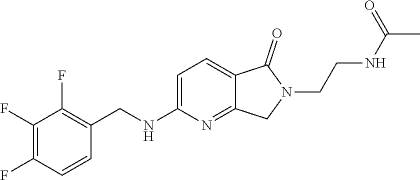 [SMILES: CC(=O)NCCN1Cc2nc(NCc3ccc(F)c(F)c3F)ccc2C1=O] | 1-0.05 |
| Compound 76 | 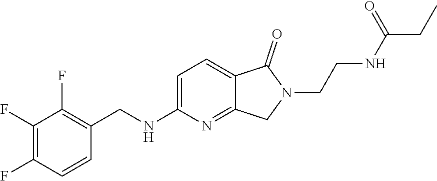 [SMILES: CCC(=O)NCCN1Cc2nc(NCc3ccc(F)c(F)c3F)ccc2C1=O] | 1-0.05 |
| Compound 77 | 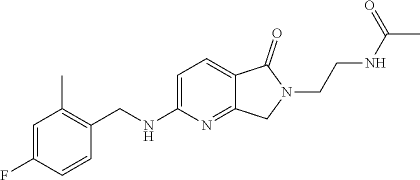 [SMILES: CC(=O)NCCN1Cc2nc(NCc3ccc(F)cc3C)ccc2C1=O] | 1-0.05 |
| Compound 78 | 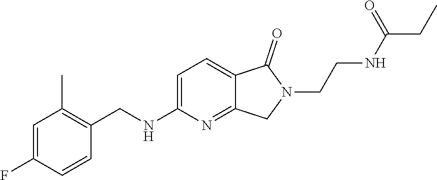 [SMILES: CCC(=O)NCCN1Cc2nc(NCc3ccc(F)cc3C)ccc2C1=O] | 1-10 |
| Compound 79 | 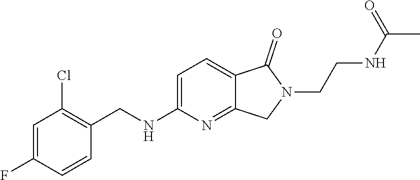 [SMILES: CC(=O)NCCN1Cc2nc(NCc3ccc(F)cc3Cl)ccc2C1=O] | 1-0.05 |
| Compound 80 | 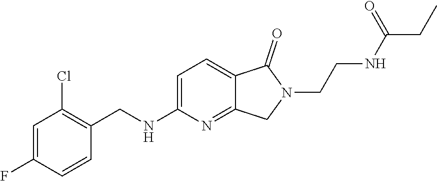 [SMILES: CCC(=O)NCCN1Cc2nc(NCc3ccc(F)cc3Cl)ccc2C1=O] | 1-0.05 |
| Compound 81 | 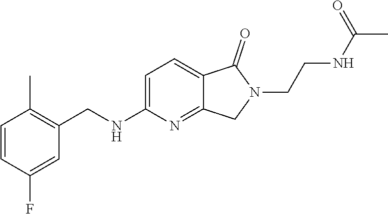 [SMILES: CC(=O)NCCN1Cc2nc(NCc3cc(F)ccc3C)ccc2C1=O] | 1-0.05 |
| Compound 82 | 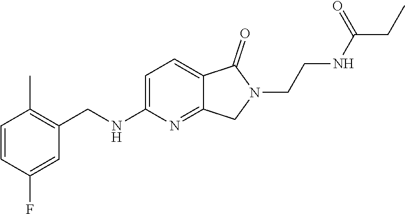 [SMILES: CCC(=O)NCCN1Cc2nc(NCc3cc(F)ccc3C)ccc2C1=O] | 1-0.05 |
| Compound 83 | 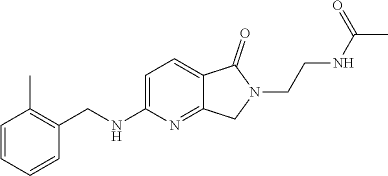 [SMILES: CC(=O)NCCN1Cc2nc(NCc3ccccc3C)ccc2C1=O] | 1-0.05 |
| Compound 84 | 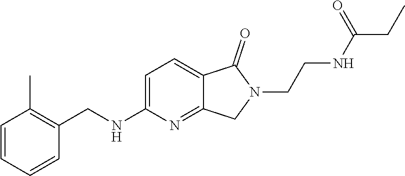 [SMILES: CCC(=O)NCCN1Cc2nc(NCc3ccccc3C)ccc2C1=O] | 1-0.05 |
| Compound 85 | 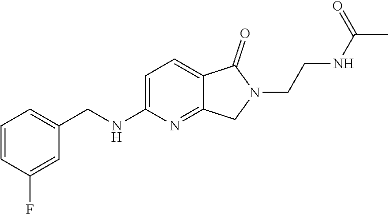 [SMILES: CC(=O)NCCN1Cc2nc(NCc3cccc(F)c3)ccc2C1=O] | 1-0.05 |
| Compound 86 | 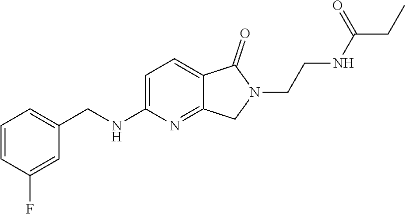 [SMILES: CCC(=O)NCCN1Cc2nc(NCc3cccc(F)c3)ccc2C1=O] | 1-0.05 |
| Compound 87 | 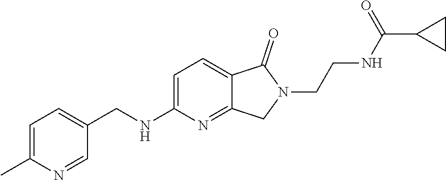 [SMILES: Cc1ccc(CNc2ccc3c(n2)CN(CCNC(=O)C2CC2)C3=O)cn1] | 1-0.05 |
| Compound 88 | 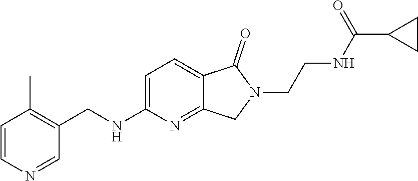 [SMILES: Cc1ccncc1CNc1ccc2c(n1)CN(CCNC(=O)C1CC1)C2=O] | 1-0.05 |
| Compound 89 | 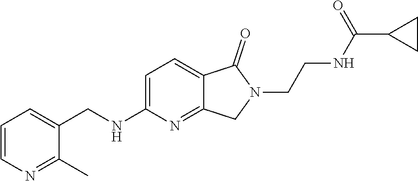 [SMILES: Cc1ncccc1CNc1ccc2c(n1)CN(CCNC(=O)C1CC1)C2=O] | 1-0.05 |
| Compound 90 | 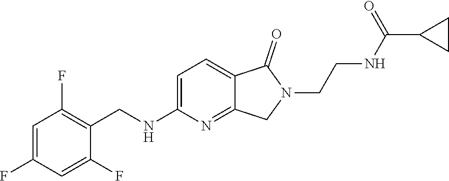 [SMILES: O=C(NCCN1Cc2nc(NCc3c(F)cc(F)cc3F)ccc2C1=O)C1CC1] | 1-0.05 |
| Compound 91 | 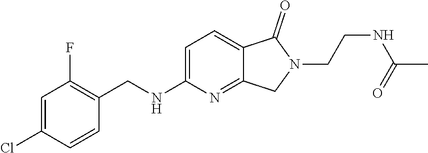 [SMILES: CC(=O)NCCN1Cc2nc(NCc3ccc(Cl)cc3F)ccc2C1=O] | 0.05-0.001 |
| Compound 92 | 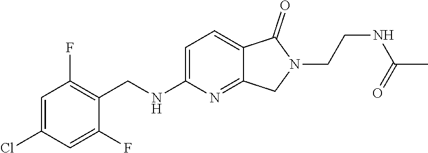 [SMILES: CC(=O)NCCN1Cc2nc(NCc3c(F)cc(Cl)cc3F)ccc2C1=O] | 0.05-0.001 |
| Compound 93 | 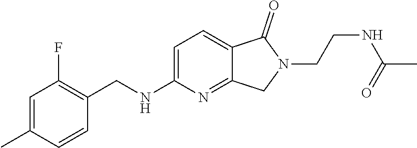 [SMILES: CC(=O)NCCN1Cc2nc(NCc3ccc(C)cc3F)ccc2C1=O] | 0.05-0.001 |
| Compound 201 | 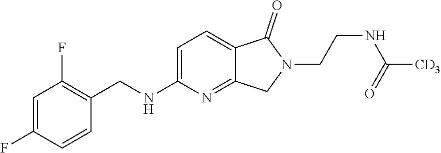 [SMILES: [2H]C([2H])([2H])C(=O)NCCN1Cc2nc(NCc3ccc(F)cc3F)ccc2C1=O] | 0.05-0.001 |
| Compound 202 | 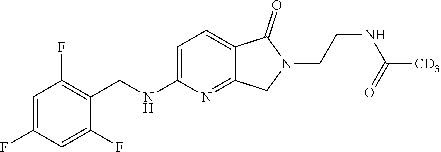 [SMILES: [2H]C([2H])([2H])C(=O)NCCN1Cc2nc(NCc3c(F)cc(F)cc3F)ccc2C1=O] | 0.05-0.001 |
| Compound 203 | 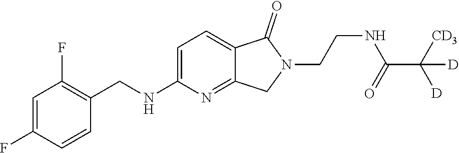 [SMILES: [2H]C([2H])([2H])C([2H])([2H])C(=O)NCCN1Cc2nc(NCc3ccc(F)cc3F)ccc2C1=O] | 0.05-0.001 |
| Compound 204 | 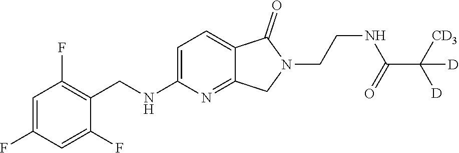 [SMILES: [2H]C([2H])([2H])C([2H])([2H])C(=O)NCCN1Cc2nc(NCc3c(F)cc(F)cc3F)ccc2C1=O] | 0.05-0.001 |
| Compound 205 | 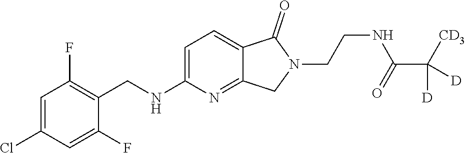 [SMILES: [2H]C([2H])([2H])C([2H])([2H])C(=O)NCCN1Cc2nc(NCc3c(F)cc(Cl)cc3F)ccc2C1=O] | 0.05-0.001 |
| Compound 206 | 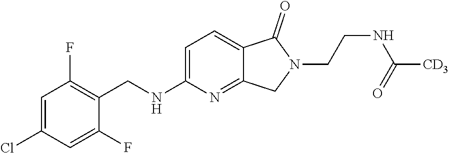 [SMILES: [2H]C([2H])([2H])C(=O)NCCN1Cc2nc(NCc3c(F)cc(Cl)cc3F)ccc2C1=O] | 0.05-0.001 |

As demonstrated in Table 6, the half-maximal inhibitory concentration of Compound 2 was comparable to RR6, which is widely known as a highly potent and selective Vanin-1 inhibitor. More so, Compound 1 provided a potency of up to two orders of magnitude greater than RR6.

### Example 101: PAMPA Assay

Parallel artificial membrane permeability (PAMPA) assay is a non-cell based assay designed to predict in vivo biological membrane permeability of drugs in early stage of drug discovery. The 96-well Corning Gentest Pre-coated PAMPA Plate System (catalog no. 353015) was used to perform the assay. The Corning Gentest Pre-coated PAMPA plate system was designed by 96-well insert system with a 0.45 m PVDF (polyvinylidene fluoride) filter plate which has been pre-coated with structured tri-layers of phospholipids (lipid/oil/lipid) and a matched receiver microplate.

Donor solutions of test compounds (300 μL, 20 μM in PBS/MeOH 90:10) were added to each well of the donor plate. 200 μL of PBS/MeOH 90:10 was added to each well of the acceptor plate. The acceptor plate was coupled with the donor plate and incubated for 5 hours at room temperature (RT) without agitation. In each plate, compounds were tested in triplicate. At the end of the incubation, drug concentration in the initial donor solution, acceptor and the donor wells were determined using LC/MS/MS. A five (5) points standard curve from 0.1 to 1000 nM for each test compound were prepared. Analyte samples were diluted to within standard curve concentration with ACN/H₂O 50:50 prior analysis.

All samples were analyzed by electrospray ionization (ESI) liquid chromtograph/mass spectrometry (LC/MS) system utilizing Sciex 5500 quadropule ion trap (Qtrap) mass spectrometry with Shidmazu Nexera X2 UHPLC. The LC-MS/MS instrument monitored each of the test compound in the study based of their respective mass-to-charge (m/z) transitions and MS parameters. Chromatographic separation was achieved on Phenomenex Luna C₁₈ column (50×2.0 mm, 3 μm particle size) by using a gradient elution: mobile phase A contains 0.1% formic acid in water and mobile phase B contains 0.1% formic acid in acetonitrile; A/B (99:1) from 0 to 1 minutes; to A/B (1:99) from 1 to 4 minutes; A/B (1:99) from 4 to 8 minutes; to A/B (99:1) from 8 to 8.20 minutes; A/B (99:1) from 8.2 to 9 minutes. The flow rate was 0.4 mL/min and the column temperature was maintained at 35° C. and autosampler temperature at 4° C. For detection, the electrospray ionization operated in the positive mode using multiple reaction monitoring (MRM).

Data acquisition, and peak integrations were obtained by Analyst® Version 1.7.1 (Sciex) operating with Windows® (Microsoft). The standard curve regressions and sample concentrations were generated and calculated by Analyst®. Concentrations in the sample solutions were determined based on the measured peak area ratios from the response of the analyte to the internal standard with reference to the standard calibration curve. All sample calculations were calculated using a linear regression with 1/x₂ weighting (where x is the concentration of given calibration standard level).

Permeability of the test compounds was calculated using the following formula:

$$Permeability ( cm / s ) : {P}_{e} = \{ - ln [ 1 - {C}_{A} ( t ) / {C}_{equilibrium} ] \} / [ A ⋆ ( 1 / {V}_{D} + 1 / {V}_{A} ) ⋆ t ]$$

Where:
A=filter area=0.3 cm₃
V_{A}=acceptor well volume (0.2 mL)
V_{D}=donor well volume (0.2 mL),
t=incubation time=5 hours=18000 seconds,
C_{A}(t)=compound concentration in acceptor well at time t (nM),
C_{D}(t)=compound concentration in donor well at time t (nM), and

$${C}_{equilibrium} = [ {C}_{D} ( t ) ⋆ {V}_{D} + {C}_{A} ( t ) ⋆ {V}_{A} ] / ( {V}_{D} + {V}_{A} )$$

TABLE 7
PAMPA permeability assay data.

| Compound | Structure | PAMPA (cm/s) |
|---|---|---|
| Compound 37 | 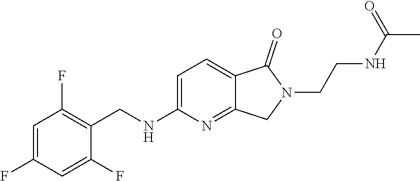 [SMILES: CC(=O)NCCN1Cc2nc(NCc3c(F)cc(F)cc3F)ccc2C1=O] | 6.80 × 10⁻⁷ |
| Compound 202 | 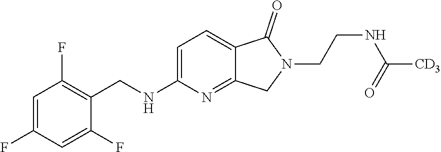 [SMILES: [2H]C([2H])([2H])C(=O)NCCN1Cc2nc(NCc3c(F)cc(F)cc3F)ccc2C1=O] | 1.83 × 10⁻⁶ |
| Compound 73 | 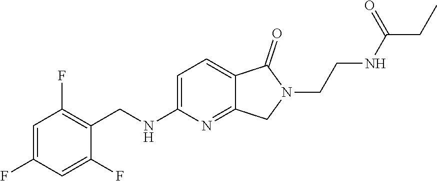 [SMILES: CCC(=O)NCCN1Cc2nc(NCc3c(F)cc(F)cc3F)ccc2C1=O] | 7.90 × 10⁻⁷ |
| Compound 204 | 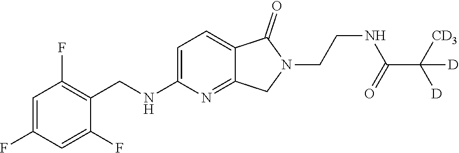 [SMILES: [2H]C([2H])([2H])C([2H])([2H])C(=O)NCCN1Cc2nc(NCc3c(F)cc(F)cc3F)ccc2C1=O] | 1.07 × 10⁻⁶ |

As shown by the data in Table 7, deuterated Compounds 202 and 204 demonstrated improved permeability when compared to their non-deuterated counterparts (Compounds 37 and 73, respectively). This indicates that Compounds 202 and 204 have a higher oral absorption potential than Compounds 37 and 73.

### Example 102: Prophetic Example of Treating IBD Patients

This is a prophetic example. A VNN1 inhibitor as disclosed herein is used to treat patients with Ulcerative Colitis and Crohn's Disease patients with active disease, who were sensitive or refractory to previous anti-TNFA treatments. The inhibitor is administered orally as a capsule at a dose of 50-200 mg daily for twelve weeks. After the completion of the treatment, the patients have both clinical and histological improvement, assessed by Mayo or CDAI clinical score, endoscopic appearance, and histological evaluation.

### Example 103: Prophetic Example of Treating Cancer Patients

This is a prophetic example. A VNN1 inhibitor as disclosed herein is used to treat patients with colon cancer, liver cancer, pancreatic cancer, gastric cancer, esophageal cancer, prostate cancer, breast cancer, cholangiocarcinomas, sarcomas, or acute myeloid leukemia. The inhibitor is administered orally as a capsule at a dose of 50-200 mg daily in patients with or without chemotherapy. After the completion of the treatment, the patients have clinical improvement, characterized by increased survival after prognosis.

What is claimed is:

1. A compound, or a pharmaceutically acceptable salt thereof, having a structure represented by Formula (I): 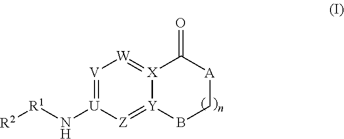
wherein:
A, B, U, V, W, X, Y, and Z are each independently selected from the group consisting of C, C(R⁵-R³), N, N(R⁵-R⁴), S, and S(R⁵-R⁴) and at least one instance of A, B, U, V, W, X, Y, and Z is N, N(R⁵-R⁴), S, or S(R⁵-R⁴) with the remaining variables being C or C(R⁵-R³);
each instance of R¹ and R² is independently selected from the group consisting of optionally substituted C₁₋₆ alkyl, optionally substituted C₁₋₂₀ alkylene, optionally substituted C₁₋₆ heteroalkyl, optionally substituted C₆₋₁₀ aryl, optionally substituted 3-10 membered arylalkyl, optionally substituted 5-10 membered heteroaryl, optionally substituted 3-10 membered heteroarylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent;
each instance of R³ and R⁴, where present, is independently selected from the group consisting of —H, optionally substituted C₁₋₆ alkyl, optionally substituted C₁₋₂₀ alkylene, optionally substituted C₁₋₆ heteroalkyl, optionally substituted C₆₋₁₀ aryl, optionally substituted 5-10 membered heteroaryl, optionally substituted 3-10 membered arylalkyl, optionally substituted 3-20 membered carbocyclyl, optionally substituted 3-20 membered (carbocyclyl)alkyl, optionally substituted 3-20 membered heterocyclyl, optionally substituted 3-20 membered (heterocyclyl)alkyl, and absent;
each instance of R⁵, where present, is independently selected from the group consisting of —(CR^{x}₂)ₘ and optionally substituted C₁₋₂₀ alkylene, or is absent;
each R^{x} is independently —H, -D, or an optionally substituted C₁₋₆ alkyl;
m is an integer in the range of 0 to 20;
n is 0, 1, or 2.

2. The compound of claim 1, wherein the compound contains one or more deuterium atom(s).

3. The compound of claim 1, wherein n is 0.

4. The compound of claim 1, wherein A is N(R⁵R⁴).

5. The compound of claim 1, wherein R⁵ is an optionally substituted C₁₋₂₀ alkylene.

6. The compound of claim 1, wherein the structure of Formula (I) is further represented by a formula selected from any one of Formulae (Ia) or (Ib):

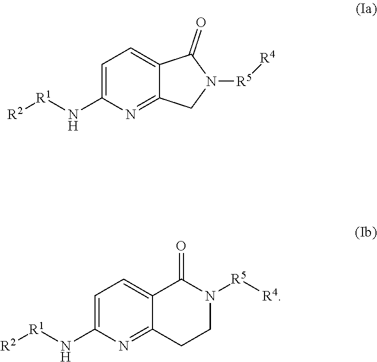

7. The compound of claim 1, wherein R⁵ is —(CH₂)ₘ—, —(CD₂)ₘ-, or —(CR^{x}₂)ₘ.

8. The compound of claim 7, wherein m is 2.

9. The compound of claim 1, wherein R⁴ is an optionally substituted C₁₋₆ alkyl or an optionally substituted C₁₋₆ heteroalkyl.

10. The compound of claim 1, wherein R⁴ is selected from the group consisting of —H,

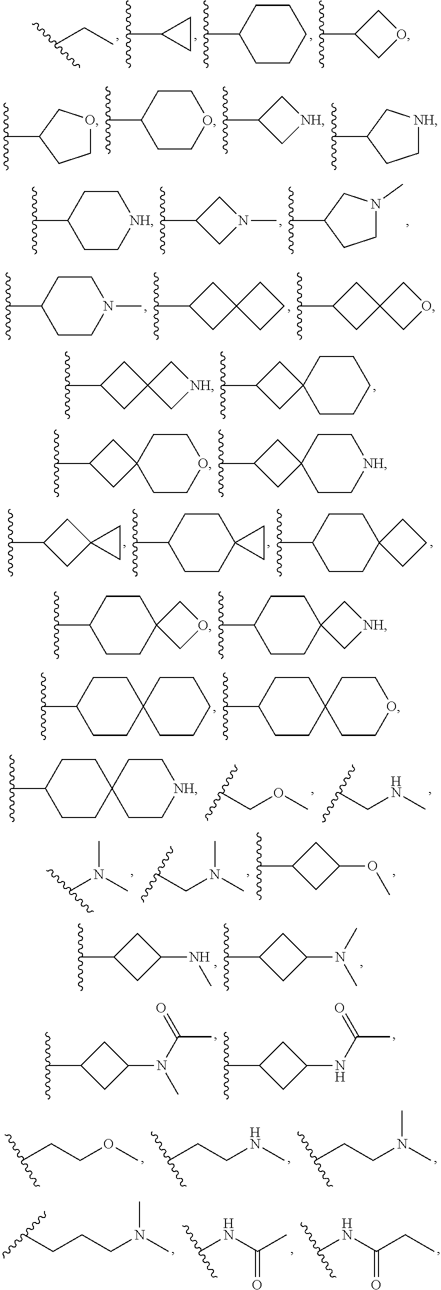

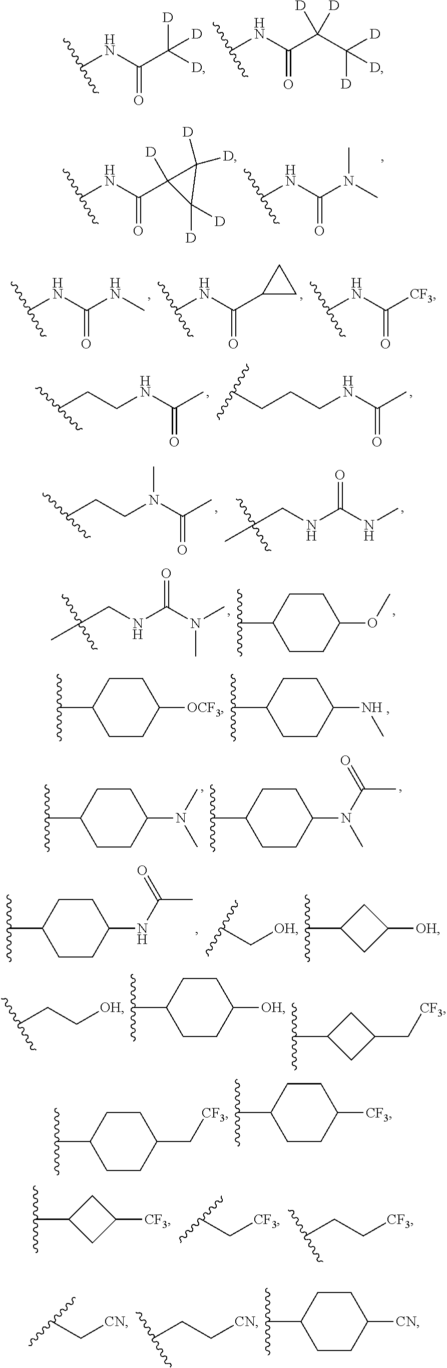

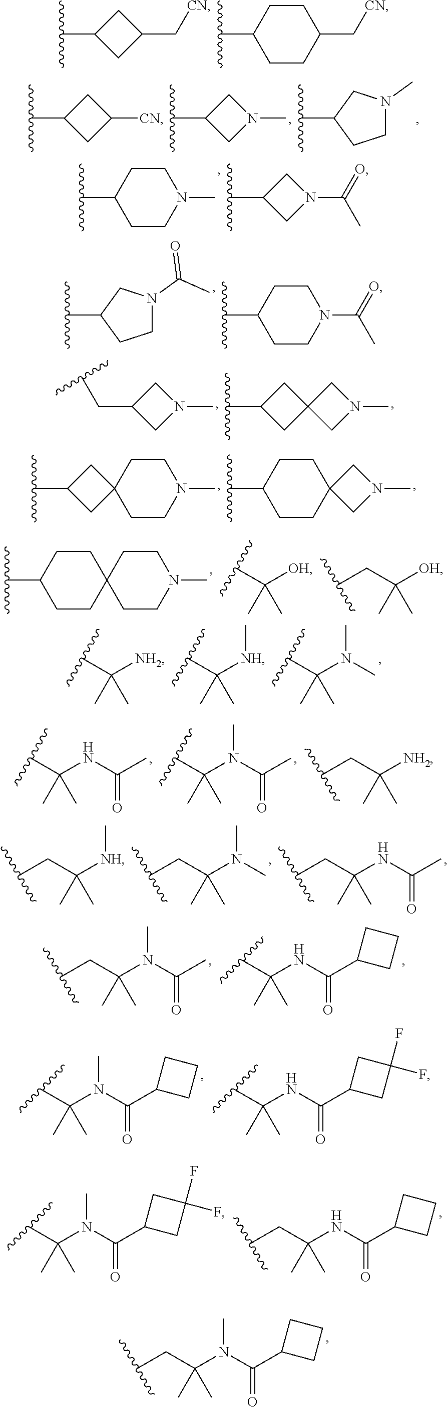

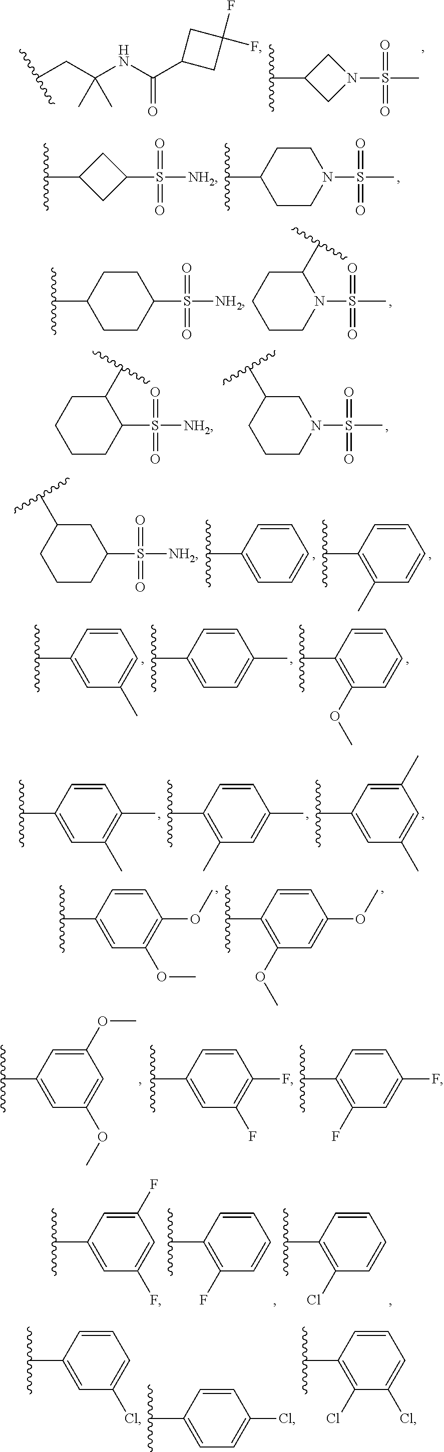

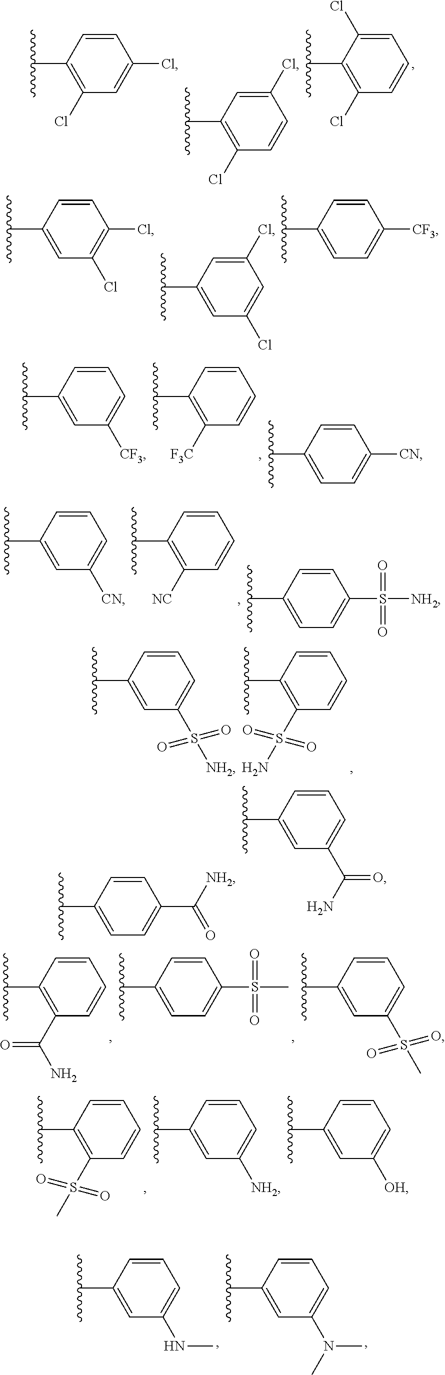

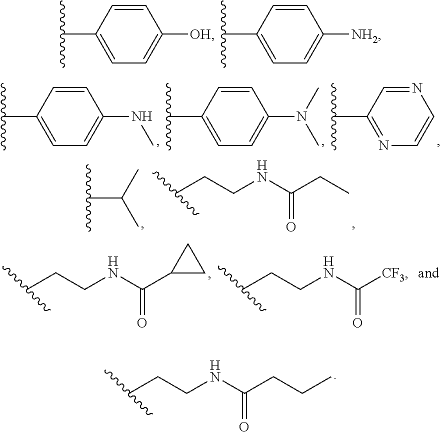

11. The compound of claim 1, wherein R⁴ is selected from the group consisting of —H,

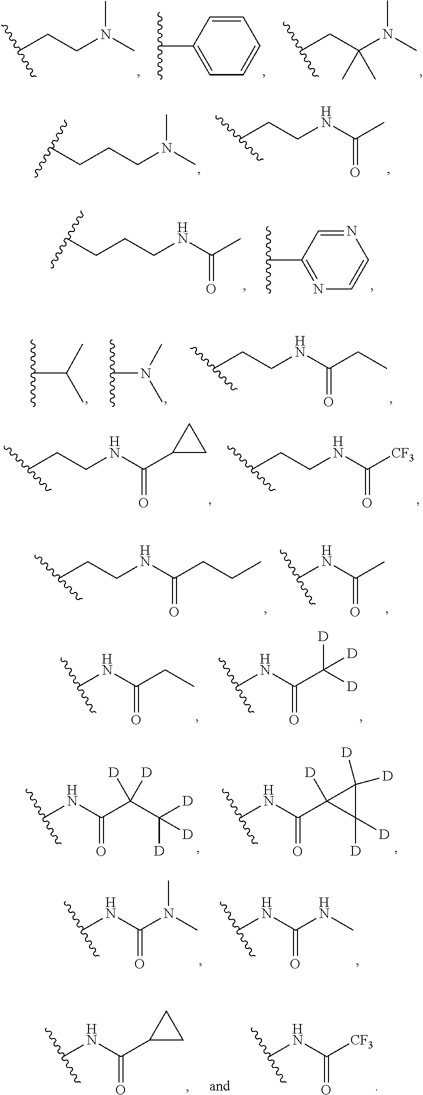

12. The compound of claim 11, wherein R⁴ is H.

13. The compound of claim 1, wherein R¹ is an optionally substituted C₁₋₂₀ alkylene.

14. The compound of claim 1, wherein R¹ is selected from the group consisting of —CHD-, CD₂-, —CH₂—, —CH₂CH₂—, —CH(CH₃)—, and —CHCH₂(OH)—.

15. The compound of claim 1, wherein R¹ is —(CR^{y}₂)ₚ—, wherein each R^{y} is independently —H or -D, and p is an integer in the range of 0 to 20.

16. The compound of claim 1, wherein the structure of Formula (I) is further represented by Formula (Ic): 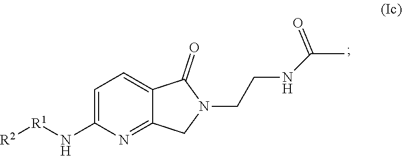
or a pharmaceutically acceptable salt thereof.

17. The compound of claim 1, wherein the structure of Formula (I) is further represented by Formula (Id): 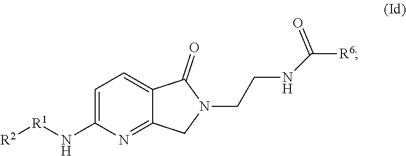
or a pharmaceutically acceptable salt thereof, wherein:
R⁶ is selected from the group consisting of —CD₃, —CD₂CD₃, 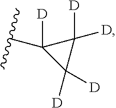
methyl, —N(CH₃)₂, —NHCH₃, and cyclopropyl.

18. The compound of claim 17, wherein R¹ is —CH₂—, —CHD-, or —CD₂-.

19. The compound of claim 17, wherein R¹ is absent.

20. The compound of claim 17, wherein R² is an optionally substituted phenyl or an optionally substituted 5- or 6-membered heterocyclyl with 1 to 3 heteroatoms selected from N, O, and S.

21. The compound of claim 17, wherein R² is pyridinyl, pyrimidinyl, or pyrazinyl.

22. The compound of claim 20, wherein the optionally substituted group is substituted with between one and five groups selected from: —F, —Cl, -D, methyl, methoxy, oxo, —CN, and —CF₃.

23. The compound of claim 1, wherein R² is selected from the group consisting of:

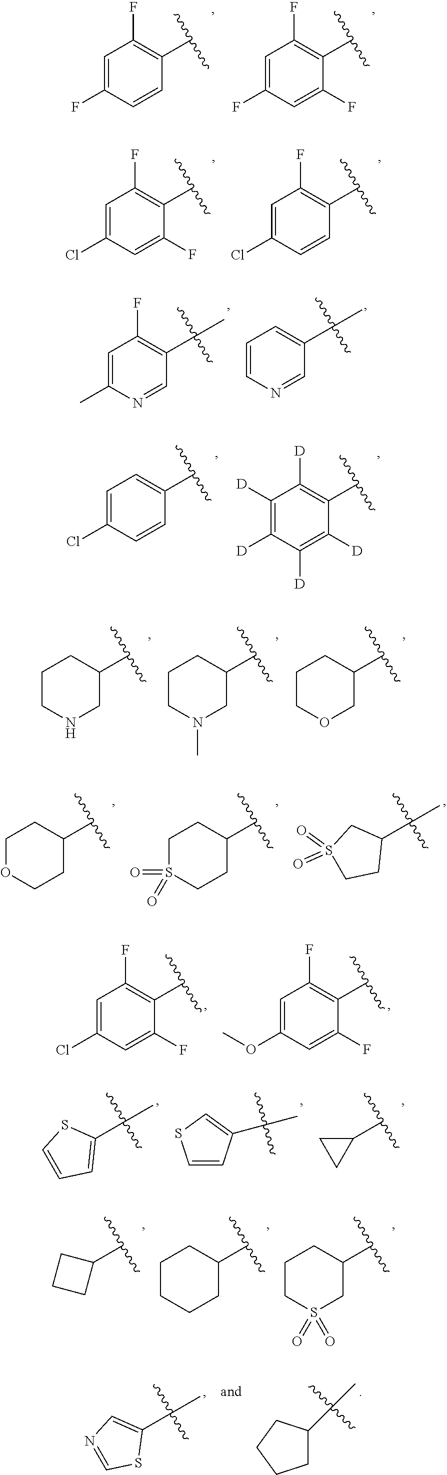

24. The compound of claim 1, wherein R is selected from the group consisting of:

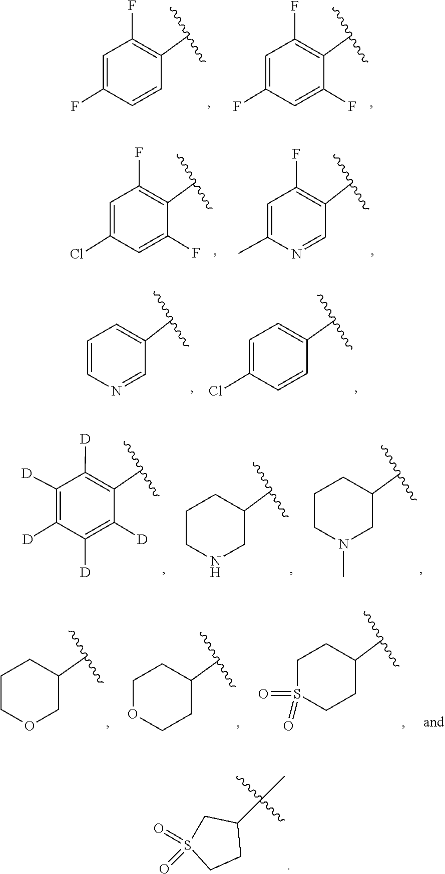

25. The compound of claim 1, or a pharmaceutically acceptable salt thereof, wherein the compound is selected from:

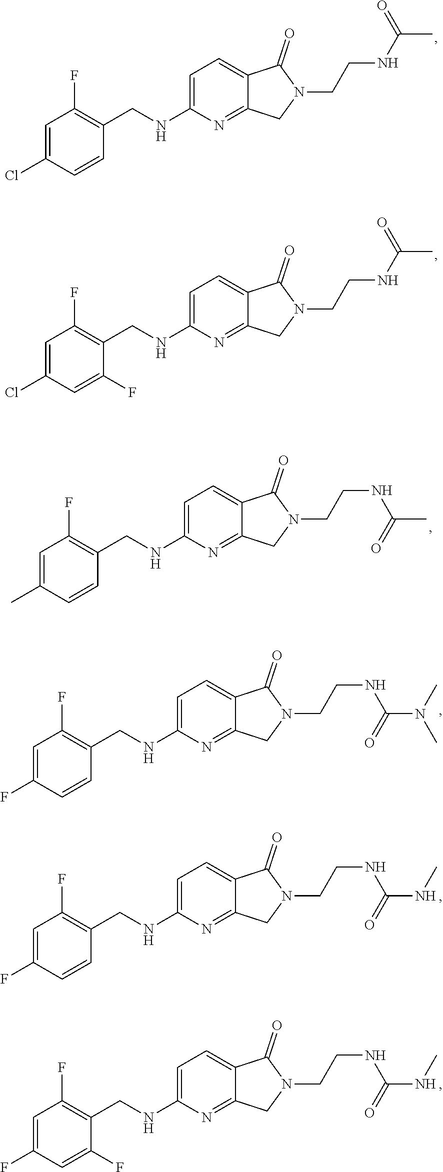

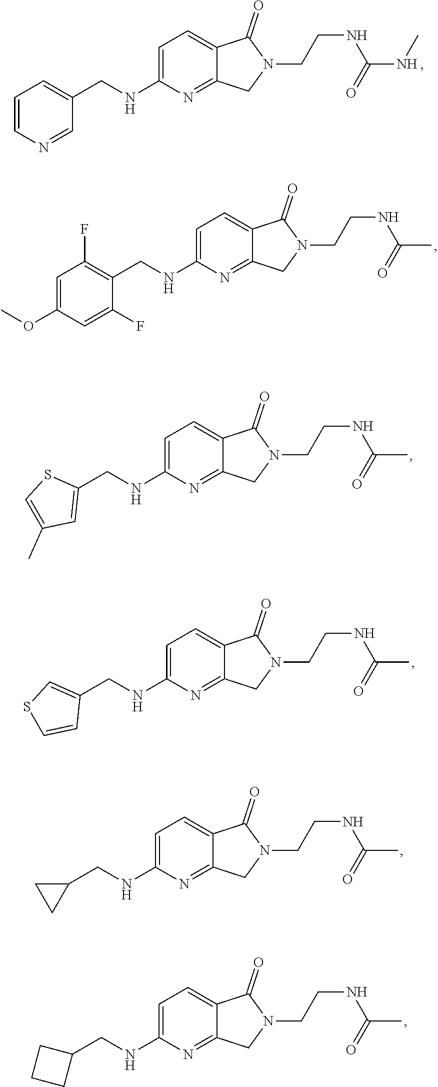

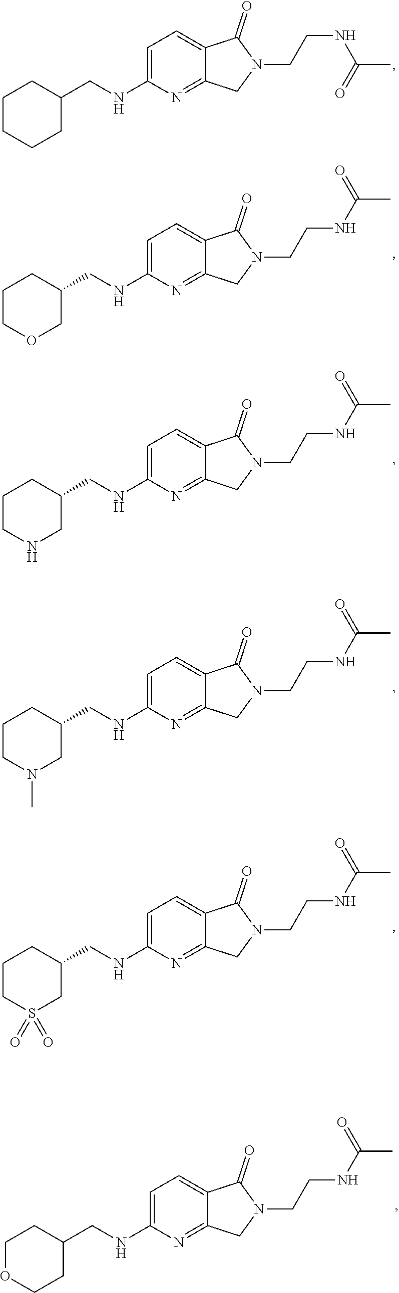 [SMILES: CC(=O)NCCN1Cc2nc(NCC3CCCCC3)ccc2C1=O ; CC(=O)NCCN1Cc2nc(NC[C@H]3CCCOC3)ccc2C1=O ; CC(=O)NCCN1Cc2nc(NC[C@H]3CCCNC3)ccc2C1=O ; CC(=O)NCCN1Cc2nc(NC[C@H]3CCCN(C)C3)ccc2C1=O ; CC(=O)NCCN1Cc2nc(NC[C@H]3CCCS(=O)(=O)C3)ccc2C1=O ; CC(=O)NCCN1Cc2nc(NCC3CCOCC3)ccc2C1=O]

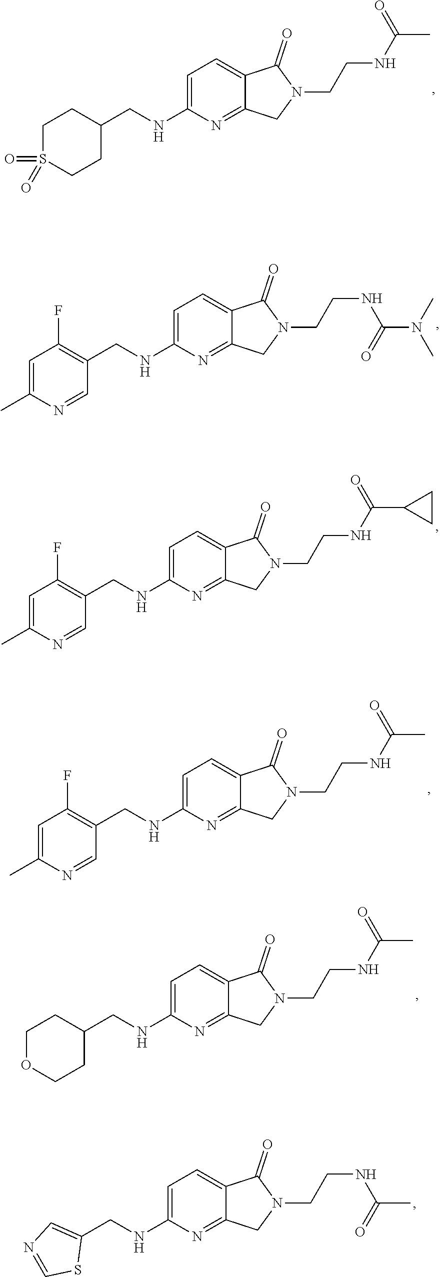

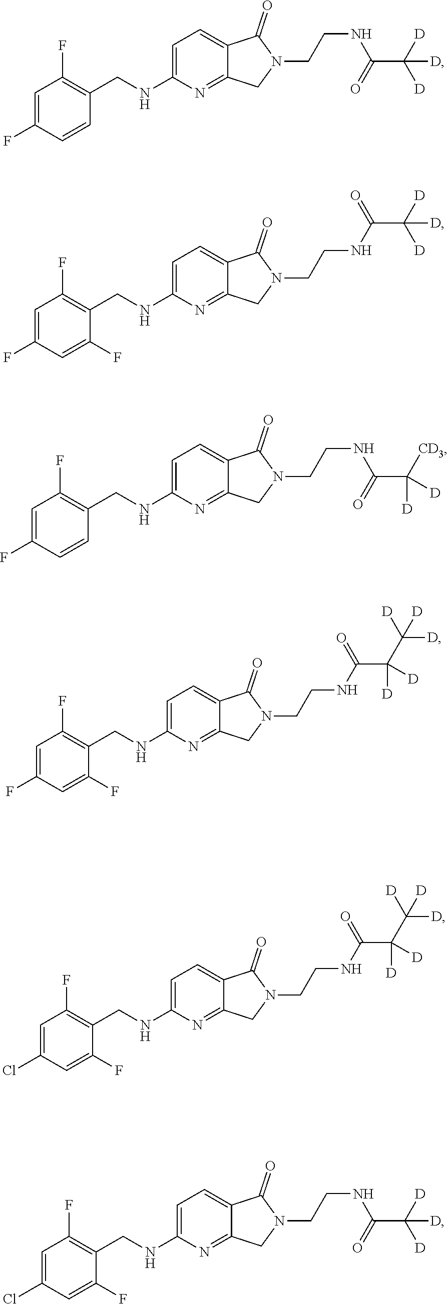

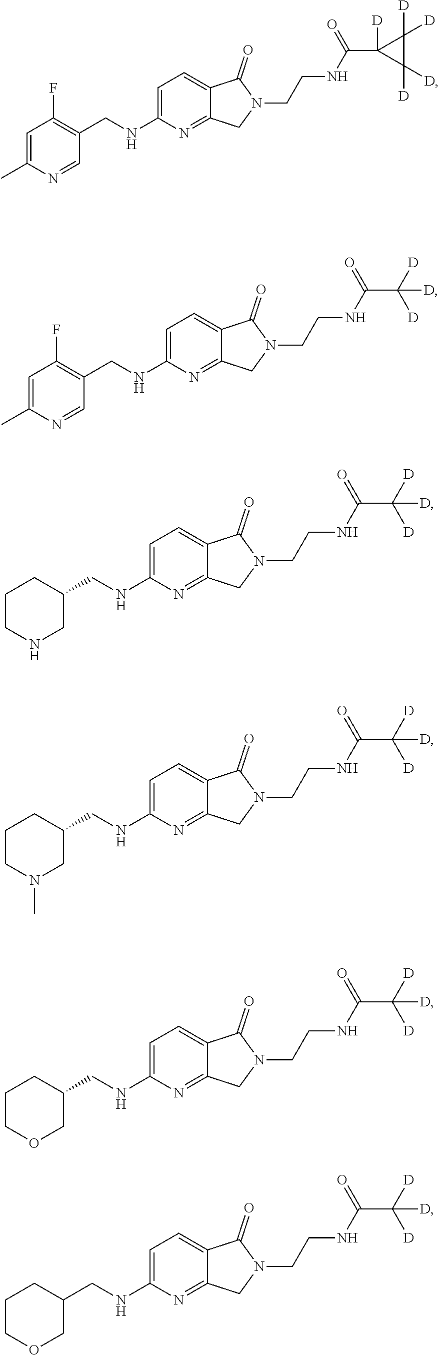

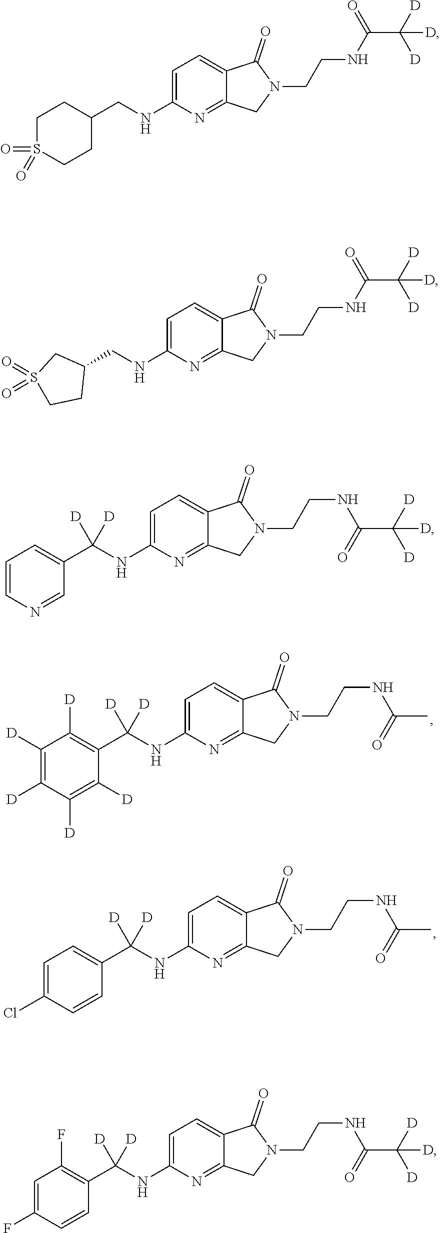

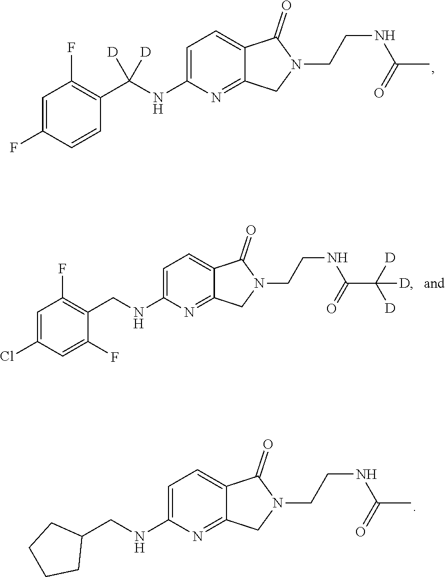

26. A method of treating a VNN1 mediated condition comprising administering to a subject requiring treatment a compound as disclosed in claim 1.

27. The method of claim 26, wherein the subject is suffering from an autoimmune disorder, inflammatory disorder, or a form of cancer.

28. A method of manufacturing a compound of claim 1, the method comprising functionalizing a bicyclic compound starting material with one or more substitutents.

29. Use of a compound according to claim 1, for the preparation of a medicament to treat an autoimmune disorder, inflammatory disorder, or a form of cancer.

30. Use of a compound according to claim 1, for the treatment of an autoimmune disorder, inflammatory disorder, or a form of cancer.
